(19) United States
(12) Patent Application Publication
(10) Pub. No.: US 2005/0233328 A1
(43) Pub. Date: Oct. 20, 2005

(54) Methods of identifying compounds that modulate protein activity

(76) Inventors: Constance Berghs, New Haven, CT (US); Karen Ellerman, Branford, CT (US); Xiaojia (Sasha) Guo, Branford, CT (US); Li Li, Branford, CT (US); Tatiana Ort, Milford, CT (US); Daniel Rieger, Branford, CT (US); Corine A.M. Vernet, Chernex (CH); Mei Zhong, Branford, CT (US)

Correspondence Address: Jenell Lawson;Intellectual Property, CuraGen Corportation, 555 Long Wharf Drive, New Haven, CT, 06551, US

(21) Appl. No.: 10/635,407

(22) Filed: Aug. 6, 2003

Related U.S. Application Data
(63) Continuation-in-part of application No. 10/307,817, filed on Dec. 2, 2002.
(60) Provisional application No. 60/336,881, filed on Dec. 3, 2001.
(60) Provisional application No. 60/336,820, filed on Dec. 5, 2001.
(60) Provisional application No. 60/361,770, filed on Mar. 5, 2002.
(60) Provisional application No. 60/364,238, filed on Mar. 13, 2002.
(60) Provisional application No. 60/338,285, filed on Dec. 7, 2001.
(60) Provisional application No. 60/383,829, filed on May 29, 2002.
(60) Provisional application No. 60/383,534, filed on May 28, 2002.
(60) Provisional application No. 60/338,318, filed on Dec. 7, 2001.
(60) Provisional application No. 60/404,676, filed on Aug. 20, 2002.
(60) Provisional application No. 60/353,288, filed on Feb. 1, 2002.
(60) Provisional application No. 60/362,230, filed on Mar. 5, 2002.
(60) Provisional application No. 60/364,181, filed on Mar. 13, 2002.
(60) Provisional application No. 60/339,022, filed on Dec. 10, 2001.
(60) Provisional application No. 60/353,286, filed on Feb. 1, 2002.
(60) Provisional application No. 60/364,978, filed on Mar. 15, 2002.
(60) Provisional application No. 60/338,989, filed on Dec. 10, 2001.
(60) Provisional application No. 60/359,956, filed on Feb. 27, 2002.
(60) Provisional application No. 60/360,964, filed on Feb. 28, 2002.
(60) Provisional application No. 60/405,698, filed on Aug. 23, 2002.
(60) Provisional application No. 60/339,314, filed on Dec. 11, 2001.
(60) Provisional application No. 60/339,517, filed on Dec. 11, 2001.
(60) Provisional application No. 60/361,256, filed on Feb. 28, 2002.
(60) Provisional application No. 60/339,611, filed on Dec. 11, 2001.
(60) Provisional application No. 60/359,914, filed on Feb. 27, 2002.
(60) Provisional application No. 60/405,400, filed on Aug. 23, 2002.
(60) Provisional application No. 60/339,516, filed on Dec. 11, 2001.
(60) Provisional application No. 60/359,626, filed on Feb. 26, 2002.
(60) Provisional application No. 60/361,264, filed on Feb. 28, 2002.
(60) Provisional application No. 60/365,025, filed on Mar. 15, 2002.
(60) Provisional application No. 60/405,684, filed on Aug. 23, 2002.
(60) Provisional application No. 60/340,981, filed on Dec. 12, 2001.
(60) Provisional application No. 60/340,565, filed on Dec. 14, 2001.
(60) Provisional application No. 60/359,671, filed on Feb. 26, 2002.
(60) Provisional application No. 60/360,924, filed on Feb. 28, 2002.
(60) Provisional application No. 60/381,004, filed on May 16, 2002.
(60) Provisional application No. 60/401,315, filed on Aug. 6, 2002.
(60) Provisional application No. 60/340,608, filed on Dec. 14, 2001.
(60) Provisional application No. 60/405,687, filed on Aug. 23, 2002.
(60) Provisional application No. 60/340,440, filed on Dec. 14, 2001.
(60) Provisional application No. 60/361,028, filed on Feb. 28, 2002.
(60) Provisional application No. 60/341,144, filed on Dec. 14, 2001.
(60) Provisional application No. 60/359,599, filed on Feb. 26, 2002.
(60) Provisional application No. 60/393,332, filed on Jul. 2, 2002.
(60) Provisional application No. 60/341,346, filed on Dec. 12, 2001.
(60) Provisional application No. 60/341,477, filed on Dec. 17, 2001.
(60) Provisional application No. 60/381,495, filed on May 17, 2002.
(60) Provisional application No. 60/401,788, filed on Aug. 7, 2002.
(60) Provisional application No. 60/341,540, filed on Dec. 17, 2001.
(60) Provisional application No. 60/383,744, filed on May 28, 2002.
(60) Provisional application No. 60/342,592, filed on Dec. 20, 2001.
(60) Provisional application No. 60/340,390, filed on Dec. 14, 2001.
(60) Provisional application No. 60/344,903, filed on Dec. 31, 2001.
(60) Provisional application No. 60/384,024, filed on May 29, 2002.
(60) Provisional application No. 60/373,288, filed on Apr. 17, 2002.
(60) Provisional application No. 60/380,981, filed on May 15, 2002.
(60) Provisional application No. 60/406,353, filed on Aug. 26, 2002.
(60) Provisional application No. 60/341,768, filed on Dec. 18, 2001.

(51) Int. Cl. C12Q 1/68; G01N 33/53; C07H 21/04; C12N 9/64; C12N 15/09; C12P 21/06

(52) U.S. Cl. 435/6; 435/7.1; 435/69.1; 435/226; 435/320.1; 435/325; 536/23.2; 530/388.26

(57) ABSTRACT

Methods of identifying compounds that modulate target polypeptide activity, where a test compound is combined with a target polypeptide and a substrate of the target polypeptide and where a determination is made as to whether the test compound modulates activity of the target polypeptide. The test compounds could be small molecule drugs used for treatment of obesity, diabetes, insulin resistance, and for enhancement of insulin secretion. Target polypeptides and their corresponding nucleic acids as well as their variants are also disclosed.

### RELATED APPLICATIONS

This application is a continuation-in-part of U.S. Ser. No. 10/307,817 filed Dec. 2, 2002, which claims the benefit of U.S. Ser. No. 60/336,881, filed Dec. 3, 2001; U.S. Ser. No. 60/336,820, filed Dec. 5, 2001; U.S. Ser. No. 60/361,770, filed Mar. 5, 2002; U.S. Ser. No. 60/364,238, filed Mar. 13, 2002; U.S. Ser. No. 60/338,285, filed Dec. 7, 2001; U.S. Ser. No. 60/383,829, filed May 29, 2002; U.S. Ser. No. 60/383,534, filed May 28, 2002; U.S. Ser. No. 60/338,318, filed Dec. 7, 2001; U.S. Ser. No. 60/404,676, filed Aug. 20, 2002; U.S. Ser. No. 60/353,288, filed Feb. 1, 2001; U.S. Ser. No. 60/362,230, filed Mar. 5, 2002; U.S. Ser. No. 60/364,181, filed Mar. 13, 2002; U.S. Ser. No. 60/339,022, filed Dec. 10, 2001; U.S. Ser. No. 60/353,286, filed Feb. 1, 2002; U.S. Ser. No. 60/364,978, filed Mar. 15, 2002; U.S. Ser. No. 60/338,989, filed Dec. 10, 2001; U.S. Ser. No. 60/359,956, filed Feb. 27, 2002; U.S. Ser. No. 60/360,964, filed Feb. 28, 2002; U.S. Ser. No. 60/405,698, filed Aug. 23, 2002; U.S. Ser. No. 60/339,314, filed Dec. 11, 2001; U.S. Ser. No. 60/339,517, filed Dec. 11, 2001; U.S. Ser. No. 60/361,256, filed Feb. 28, 2002; U.S. Ser. No. 60/339,611, filed Dec. 11, 2001; U.S. Ser. No. 60/359,914, filed Feb. 27, 2002; U.S. Ser. No. 60/405,400, filed Aug. 23, 2002; U.S. Ser. No. 60/339,516, filed Dec. 11, 2001; U.S. Ser. No. 60/359,626, filed Feb. 26, 2002; U.S. Ser. No. 60/361,264, filed Feb. 28, 2002; U.S. Ser. No. 60/365,025, filed Mar. 15, 2002; U.S. Ser. No. 60/405,684, filed Aug. 23, 2002; U.S. Ser. No. 60/340,981, filed Dec. 12, 2001; U.S. Ser. No. 60/340,565, filed Dec. 14, 2001; U.S. Ser. No. 60/359,671, filed Feb. 26, 2002; U.S. Ser. No. 60/360,924, filed Feb. 28, 2002; U.S. Ser. No. 60/381,004, filed May 16, 2002; U.S. Ser. No. 60/401,315, filed Aug. 6, 2002; U.S. Ser. No. 60/340,608, filed Dec. 14, 2001; U.S. Ser. No. 60/405,687, filed Aug. 23, 2002; U.S. Ser. No. 60/340,440, filed Dec. 14, 2001; U.S. Ser. No. 60/361,028, filed Feb. 28, 2002; U.S. Ser. No. 60/341,144, filed Dec. 14, 2001; U.S. Ser. No. 60/359,599, filed Feb. 26, 2002; U.S. Ser. No. 60/393,332, filed Jul. 2, 2002; U.S. Ser. No. 60/341,346, filed Dec. 12, 2001; U.S. Ser. No. 60/341,477, filed Dec. 17, 2001; U.S. Ser. No. 60/381,495, filed May 17, 2002; U.S. Ser. No. 60/401,788, filed Aug. 7, 2002; U.S. Ser. No. 60/341,540, filed Dec. 17, 2001; U.S. Ser. No. 60/383,744, filed May 28, 2002; U.S. Ser. No. 60/342,592, filed Dec. 20, 2001; U.S. Ser. No. 60/340,390, filed Dec. 14, 2001; U.S. Ser. No. 60/344,903, filed Dec. 31, 2001; U.S. Ser. No. 60/384,024, filed May 29, 2002; U.S. Ser. No. 60/373,288, filed Apr. 17, 2002; U.S. Ser. No. 60/380,981, filed May 15, 2002; U.S. Ser. No. 60/406,353, filed Aug. 26, 2002; U.S. Ser. No. 60/422,756, filed Oct. 31, 2002; and U.S. Ser. No. 60/341,768, filed Dec. 18, 2001; and is a continuation-in-part of 10/188,186, filed Jul. 2, 2003, which claims the benefit of 60/303,046, filed Jul. 5, 2001; 60/360,814, filed Mar. 1, 2002; 60/303,828, filed Jul. 9, 2001; 60/323,380, filed Sep. 19, 2001; 60/361,133, filed Mar. 1, 2002; 60/304,016, filed Jul. 9, 2001; 60/304,502, filed Jul. 11, 2001; 60/305,262, filed Jul. 13, 2001; 60/373,881, filed Apr. 19, 2002; 60/305,673, filed Jul. 16, 2001; 60/323,969, filed Sep. 21, 2001; 60/372,326, filed Apr. 12, 2002; 60/361,677, filed Mar. 5, 2002; 60/345,022, filed Jan. 4, 2002; 60/363,637, filed Mar. 12, 2002; 60/373,921, filed Apr. 19, 2002; 60/307,536, filed Jul. 24, 2001; 60/360,830, filed Mar. 1, 2002; 60/306,085, filed Jul. 17, 2001; 60/308,228, filed Jul. 27, 2001; 60/372,990, filed Apr. 16, 2002; 60/361,147, filed Mar. 1, 2002; 60/308,877, filed Jul. 30, 2001; 60/345,038, filed Jan. 4, 2002; 60/361,172, filed Feb. 28, 2002; 60/313,328, filed Aug. 17, 2001; 60/318,711, filed Sep. 12, 2001; and 60/309,255, filed Aug. 1, 2001; and claims priority to U.S. Ser. No. 60/403,620, filed Aug. 15, 2002; U.S. Ser. No. 60/401,316, filed Aug. 6, 2002; U.S. Ser. No. 60/405,232, filed Aug. 22, 2002; U.S. Ser. No. 60/401,627, filed Aug. 6, 2002; U.S. Ser. No. 60/405,121, filed Aug. 22, 2002; U.S. Ser. No. 60/404,649, filed Aug. 20, 2002; U.S. Ser. No. 60/404,674, filed Aug. 20, 2002; U.S. Ser. No. 60/454,479, filed Mar. 13, 2003; U.S. Ser. No. 60/406,131, filed Aug. 27, 2002; U.S. Ser. No. 60/409,366, filed Sep. 9, 2002; U.S. Ser. No. 60/406,130, filed Aug. 27, 2002; U.S. Ser. No. 60/407,919, filed Sep. 3, 2002; each of which is incorporated herein by reference in its entirety.

### TECHNICAL FIELD

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass screening, diagnostic, and prognostic assay procedures as well as methods of treating diverse pathological conditions.

### BACKGROUND

Obesity and diabetes are major public health concerns in the developed and developing world. It is estimated that over half of the adult US population is overweight.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass screening, diagnostic and prognostic assay procedures as well as methods of treating diverse pathological conditions.

### BACKGROUND

Obesity and diabetes are major public health concerns in the developed and developing world. It is estimated that over half of the adult US population is overweight.

This includes those with a body mass index (BMI) greater than the upper limit of normal (25) where the BMI is defined as the weight (Kg)/[height (m)]². A common consequence of being overweight is hyperlipidemia and the development of insulin resistance. This is followed by the development of hyperglycemia, a hallmark of Type II diabetes. Left untreated, the hyperglycemia leads to microvascular disease and end organ damage that includes retinopathy, renal disease, cardiac disease, peripheral neuropathy and peripheral vascular compromise. Currently, over 16 million adults in the US are affected by Type II diabetes and the condition has now become rampant among school-age children as a consequence of the epidemic of obesity in that age group.

Diabetes mellitus is a disorder in which blood levels of glucose (a simple sugar) are abnormally high because the body doesn't release or respond to insulin adequately. Blood sugar (glucose) levels vary throughout the day, rising after a meal and returning to normal within 2 hours. Blood sugar levels are normally between 70 and 110 milligrams per deciliter (mg/dL) of blood in the morning after an overnight fast. They are usually lower than 120 to 140 mg/dL 2 hours after eating foods or drinking liquids containing sugar or other carbohydrates.

Insulin, a hormone released from the pancreas, is the primary substance responsible for maintaining appropriate blood sugar levels. Insulin allows glucose to be transported into cells so that they can produce energy or store glucose-derived enrgy until it's needed. The rise in blood sugar levels after eating or drinking stimulates the pancreas to produce insulin, preventing a greater rise in blood sugar levels and causing them to fall gradually. Because muscles use glucose for energy, blood sugar levels can also fall during physical activity.

Diabetes results when the body doesn't produce enough insulin to maintain normal blood sugar levels or when cells don't respond appropriately to insulin. In type II diabetes mellitus, the pancreas continues to manufacture insulin, sometimes even at higher than normal levels. However, the body develops resistance to its effects, resulting in a relative insulin deficiency.

The main goal of diabetes treatment is to keep blood sugar levels within the normal range as much as possible. Completely normal levels are difficult to maintain, but the more closely they can be kept within the normal range, the less likely that temporary or long-term complications will develop.

Therefore, a therapeutic that decreases insulin resistance and/or enhances insulin secretion would be beneficial in treatment of obesity and/or diabetes. Additionally, such a therapeutic would be beneficial in treatment of insulin resistance, a condition that often leads to the development of diabetes.

In order to treat diseases, pathologies and other abnormal states or conditions in which a mammalian organism has been diagnosed as being, or as being at risk for becoming, other than in a normal state or condition, it is important to identify new therapeutic agents.

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n-1, wherein n is an integer between 1 and 85, or polypeptide sequences, which represents the group consisting of SEQ ID NO: 2n, wherein n is an integer between 1 and 85.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that modulates a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates that the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the purpose of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

It is a purpose of this invention to provide cell lines that recombinantly or endogenously express the target biopolymer or an isolated target biopolymer that is intended to serve as the macromolecular component in a screening assay for identifying candidate pharmaceutical agents.

It is another purpose of the present invention to provide screening assays that positively identify candidate pharmaceutical agents from among a combinatorial library of low molecular weight substances or compounds.

It is still a further aspect of this invention to employ the candidate pharmaceutical agents in any of a variety of in vitro, ex vivo and in vivo assays in order to identify pharmaceutical agents with advantageous therapeutic applications in the treatment of a disease, pathology, or abnormal state or condition in a mammal.

In another aspect, the present invention provides a method of identifying a test compound as a candidate therapeutic agent, for treating a disease, pathology, or an abnormal state or condition using a target polypeptide (NOVX) having a specific association with the disease. This method includes:
(a) combining a test compound with a target polypeptide and a substrate of the target polypeptide; and
(b) determining whether the test compound modulates the activity of the target polypeptide.

In one embodiment of this method, the chemical compound is a member of a combinatorial library of compounds; the combining in step (a) is conducted on one or more replicate samples of the biopolymer; and the replicate sample is contacted with at least one member of the combinatorial library. In additional embodiments of this method, the biopolymer is included within a cell and is functionally expressed therein. In still a further embodiment, the binding of the compound modulates the function of the biopolymer, and it is the modulation that provides the identification that the compound is a potential therapeutic agent. In yet further embodiments of this method, the target biopolymer is a polypeptide.

As used herein, a “substrate” includes any compound capable of binding to or interacting with a target polypeptide, including but not limited to a peptide, a polypeptide, a nucleic acid, a carbohydrate moiety, a lipid, a small molecule (e.g., cyclic AMP, ATP), an agonist, an antagonist, and an inhibitor.

In another aspect of the invention, a method for identifying a pharmaceutical agent for treating a disease, pathology, or an abnormal state or condition is provided. The method includes the steps of:
(1) identifying a candidate therapeutic agent for treating said disease, pathology, or abnormal state or condition by the method described in the preceding paragraphs;
(2) contacting a biological sample associated with the disease, pathology, or abnormal state or condition with the candidate therapeutic agent;
(3) determining whether the candidate induces an effect on the biological sample associated with a therapeutic response therein; and
(4) identifying a candidate exerting such an effect as a pharmaceutical agent.

In significant embodiments of the method, the biological sample includes a cell, a tissue or organ, or is a nonhuman mammal.

Several cellular, animal and clinical studies were performed to elucidate the genetic contribution to the etiology and pathogenesis of these conditions in a variety of physiologic, pharmacologic or native states. These studies utilized the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify various therapeutic interventions in order to prevent, treat the consequences or cure the conditions of obesity and/or diabetes.

The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by the inventors in certain cases. Additionally, the current invention embodies the use of recombinantly expressed and/or endogenously expressed protein in various screens to identify therapeutic antibodies and/or therapeutic small molecules which modulate activity of the disclosed NOVX polypeptides.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n-1, wherein n is an integer between 1 and 85, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

### BRIEF DESCRIPTION OF THE FIGURES

FIG. 1 is a schematic diagram of the non-oxidative stage of the pentose phosphate pathway indicating the function of transketolase in the pathway.

FIG. 2 is a schematic diagram illustrating the roles of SREBP-regulated genes during excess citrate production.

FIG. 3 is a schematic diagram illustrating representative pathways relevant to the etiology and pathogenesis of obesity and/or diabetes.

FIG. 4 is a schematic diagram illustrating the pyruvate sythesis pathway.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides.

TABLE 1
Sequences and Corresponding SEQ ID Numbers

| | | SEQ ID | SEQ ID | |
|---|---|---|---|---|
| | | NO | NO | |
| NOVX | Internal | (nucleic | (amino | |
| Assignment | Identification | acid) | acid) | Homology |
| NOV1a | CG101190-01 | 1 | 2 | Phosphoenolpyruvate carboxykinase, |
| | | | | cytosolic [GTP] (EC 4.1.1.32) |
| | | | | (Phosphoenolpyruvate carboxylase) |
| | | | | (PEPCK-C) - Homo sapiens |
| NOV1b | 278992806 | 3 | 4 | Phosphoenolpyruvate carboxykinase, |
| | | | | cytosolic [GTP] (EC 4.1.1.32) |
| | | | | (Phosphoenolpyruvate carboxylase) |
| | | | | (PEPCK-C) - Homo sapiens |
| NOV1c | 278992862 | 5 | 6 | Phosphoenolpyruvate carboxykinase, |
| | | | | cytosolic [GTP] (EC 4.1.1.32) |
| | | | | (Phosphoenolpyruvate carboxylase) |
| | | | | (PEPCK-C) - Homo sapiens |
| NOV2a | CG175387-01 | 7 | 8 | Transketolase (EC 2.2.1.1) (TK) - Homo sapiens |
| NOV2b | CG175387-03 | 9 | 10 | Transketolase (EC 2.2.1.1) (TK) - Homo sapiens |
| NOV2c | 267254044 | 11 | 12 | Transketolase (EC 2.2.1.1) (TK) - Homo sapiens |
| NOV2d | CG175387-02 | 13 | 14 | Transketolase (EC 2.2.1.1) (TK) - Homo sapiens |
| NOV2e | CG175387-04 | 15 | 16 | Transketolase (EC 2.2.1.1) (TK) - Homo sapiens |
| NOV3a | CG180320-01 | 17 | 18 | Hypothetical protein FLJ23378 - Homo sapiens |
| NOV3b | CG180320-02 | 19 | 20 | Hypothetical protein FLJ23378 - Homo sapiens |
| NOV3c | CG180320-03 | 21 | 22 | Hypothetical protein FLJ23378 - Homo |
| | | | | sapiens |
| NOV3d | CG180320-04 | 23 | 24 | Hypothetical protein FLJ23378 - Homo sapiens |
| NOV3e | 305263028 | 25 | 26 | Hypothetical protein FLJ23378 - Homo sapiens |
| NOV3f | CG180320-05 | 27 | 28 | Hypothetical protein FLJ23378 - Homo sapiens |
| NOV4a | CG181387-01 | 29 | 30 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV4b | 282274427 | 31 | 32 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV4c | CG181387-02 | 33 | 34 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV4d | 306268235 | 35 | 36 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV4e | CG181387-03 | 37 | 38 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV4f | CG181387-04 | 39 | 40 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV4g | CG181387-05 | 41 | 42 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV4h | CG181387-06 | 43 | 44 | 3-ketoacyl-CoA thiolase, mitochondrial |
| | | | | (EC 2.3.1.16) (Beta-ketothiolase) |
| | | | | (Acetyl-CoA acyltransferase) |
| | | | | (Mitochondrial 3-oxoacyl-CoA thiolase) |
| | | | | (T1) - Homo sapiens |
| NOV5a | CG186640-02 | 45 | 46 | phosphoglycerate mutase (EC 5.4.2.1) M |
| NOV5b | 311980359 | 47 | 48 | phosphoglycerate mutase (EC 5.4.2.1) M |
| NOV5c | CG186640-01 | 49 | 50 | phosphoglycerate mutase (EC 5.4.2.1) M |
| NOV5d | CG186640-03 | 51 | 52 | phosphoglycerate mutase (EC 5.4.2.1) M |
| NOV5e | CG186640-04 | 53 | 54 | phosphoglycerate mutase (EC 5.4.2.1) M |
| NOV6a | CG58655-01 | 55 | 56 | Adenosine A1 receptor - Homo sapiens |
| NOV6b | 268368558 | 57 | 58 | Adenosine A1 receptor - Homo sapiens |
| NOV7a | CG96859-03 | 59 | 60 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7b | 223317153 | 61 | 62 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7c | CG96859-01 | 63 | 64 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7d | CG96859-02 | 65 | 66 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7e | CG96859-04 | 67 | 68 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7f | CG96859-05 | 69 | 70 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7g | CG96859-06 | 71 | 72 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7h | CG96859-07 | 73 | 74 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7i | CG96859-08 | 75 | 76 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |
| NOV7j | CG96859-09 | 77 | 78 | Hydroxymethylglutaryl-CoA lyase, |
| | | | | mitochondrial precursor (EC 4.1.3.4) |
| | | | | (HMG-CoA lyase) (HL) (3-hydroxy-3- |
| | | | | methylglutarate-CoA lyase) - Homo sapiens |

Table 1 indicates the homology of NOVX polypeptides to known protein families. Thus, the nucleic acids and polypeptides, antibodies and related compounds according to the invention corresponding to a NOVX as identified in column 1 of Table 1 will be useful in therapeutic and diagnostic applications implicated in, for example, pathologies and disorders associated with the known protein families identified in column 5 of Table 1.

Pathologies, diseases, disorders and condition and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table 1, the NOVX polypeptides of the present invention show homology to, and contain domains that are characteristic of, other members of such protein families. Details of the sequence relatedness and domain analysis for each NOVX are presented in Examples for identification of human sequence in individual sections for each NOVX polypeptide.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table 1.

The NOVX nucleic acids and polypeptides are also useful for detecting specific cell types. Details of the expression analysis for each NOVX are Examples showing expression profiles in individual sections for each NOVX polypeptide. Accordingly, the NOVX nucleic acids, polypeptides, antibodies and related compounds according to the invention will have diagnostic and therapeutic applications in the detection of a variety of diseases with differential expression in normal vs. diseased tissues, e.g., detection of a variety of cancers. SNP analysis for each NOVX, if applicable, is presented in SNP Examples in individual sections for each NOVX polypeptide.

Additional utilities for NOVX nucleic acids and polypeptides according to the invention are disclosed herein.

NOVX clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 85; (b) a variant of a mature form of the amino acid sequence selected from the group consisting of SEQ ID NO: 2n, wherein n is an integer between 1 and 85, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 85; (d) a variant of the amino acid sequence selected from the group consisting of SEQ ID NO:2n, wherein n is an integer between 1 and 85 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 85; (b) a variant of a mature form of the amino acid sequence selected from the group consisting of SEQ ID NO: 2n, wherein n is an integer between 1 and 85 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 85; (d) a variant of the amino acid sequence selected from the group consisting of SEQ ID NO: 2n, wherein n is an integer between 1 and 85, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 85 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n-1, wherein n is an integer between 1 and 85; (b) a nucleotide sequence wherein one or more nucleotides in the nucleotide sequence selected from the group consisting of SEQ ID NO: 2n-1, wherein n is an integer between 1 and 85 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n-1, wherein n is an integer between 1 and 85; and (d) a nucleic acid fragment wherein one or more nucleotides in the nucleotide sequence selected from the group consisting of SEQ ID NO: 2n-1, wherein n is an integer between 1 and 85 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n-1, wherein n is an integer between 1 and 85, or a complement of this nucleotide sequence, can be isolated using standard molecular biology techniques and the sequence information provided herein. Using all or a portion of the nucleic acid sequence of SEQ ID NO:2n-1, wherein n is an integer between 1 and 85, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n-1, wherein n is an integer between 1 and 85, or a complement thereof. Oligonucleotides may be chemically synthesized and may also be used as probes. In another embodiment, an isolated nucleic acid molecule of the invention comprises a nucleic acid molecule that is a complement of the nucleotide sequence shown in SEQ ID NO:2n-1, wherein n is an integer between 1 and 85, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n-1, wherein n is an integer between 1 and 85, is one that is sufficiently complementary to the nucleotide sequence of SEQ ID NO:2n-1, wherein n is an integer between 1 and 85, that it can hydrogen bond with few or no mismatches to the nucleotide sequence shown in SEQ ID NO:2n-1, wherein n is an integer between 1 and 85, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice. A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g., they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species. Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g., Ausubel, et al., CURRENT PROTOCOLS IN MOLECULAR BIOLOGY, John Wiley & Sons, New York, N.Y., 1993, and below. A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n-1, wherein n is an integer between 1 and 85, as well as a polypeptide possessing NOVX biological activity. Various biological activities of the NOVX proteins are described below. A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more. The nucleotide sequences determined from the cloning of the human NOVX genes allows for the generation of probes and primers designed for use in identifying and/or cloning NOVX homologues in other cell types, e.g., from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n-1, wherein n is an integer between 1 and 85; or an anti-sense strand nucleotide sequence of SEQ ID NO:2n-1, wherein n is an integer between 1 and 85; or of a naturally occurring mutant of SEQ ID NO:2n-1, wherein n is an integer between 1 and 85.

Probes based on the human NOVX nucleotide sequences can be used to detect transcripts or genomic sequences encoding the same or homologous proteins. In various embodiments, the probe has a detectable label attached, e.g., the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n-1, wherein n is an integer between 1 and 85, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n-1, wherein n is an integer between 1 and 85, due to degeneracy of the genetic code and thus encode the same NOVX proteins as that encoded by the nucleotide sequences of SEQ ID NO:2n-1, wherein n is an integer between 1 and 85. In another embodiment, an isolated nucleic acid molecule of the invention has a nucleotide sequence encoding a protein having an amino acid sequence of SEQ ID NO:2n, wherein n is an integer between 1 and 85.

In addition to the human NOVX nucleotide sequences of SEQ ID NO:2n-1, wherein n is an integer between 1 and 85, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n-1, wherein n is an integer between 1 and 85, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions. Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n-1, wherein n is an integer between 1 and 85. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n-1, wherein n is an integer between 1 and 85, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n-1, wherein n is an integer between 1 and 85, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n-1, wherein n is an integer between 1 and 85, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n-1, wherein n is an integer between 1 and 85, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 85. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are not particularly amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n-1, wherein n is an integer between 1 and 85, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 85. Preferably, the protein encoded by the nucleic acid molecule is at least about 60% homologous to SEQ ID NO:2n, wherein n is an integer between 1 and 85; more preferably at least about 70% homologous to SEQ ID NO:2n, wherein n is an integer between 1 and 85; still more preferably at least about 80% homologous to SEQ ID NO:2n, wherein n is an integer between 1 and 85; even more preferably at least about 90% homologous to SEQ ID NO:2n, wherein n is an integer between 1 and 85; and most preferably at least about 95% homologous to SEQ ID NO:2n, wherein n is an integer between 1 and 85.

An isolated nucleic acid molecule encoding a NOVX protein homologous to the protein of SEQ ID NO:2n, wherein n is an integer between 1 and 85, can be created by introducing one or more nucleotide substitutions, additions or deletions into the nucleotide sequence of SEQ ID NO:2n-1, wherein n is an integer between 1 and 85, such that one or more amino acid substitutions, additions or deletions are introduced into the encoded protein.

Mutations can be introduced any one of SEQ ID NO:2n-1, wherein n is an integer between 1 and 85, by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n-1, wherein n is an integer between 1 and 85, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g., avidin proteins). In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20 (23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs.

Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g., inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g., commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2×-proteinase-K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)). These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in-vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n-1, wherein n is an integer between 1 and 85, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 85, or antisense nucleic acids complementary to a NOVX nucleic acid sequence of SEQ ID NO:2n-1, wherein n is an integer between 1 and 85, are additionally provided.

In one embodiment, an antisense nucleic acid molecule is antisense to a “coding region” of the coding strand of a nucleotide sequence encoding a NOYX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences that flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N-6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection). The antisense nucleic acid molecules of the invention are typically administered to a subject or generated in situ such that they hybridize with or bind to cellular mRNA and/or genomic DNA encoding a NOVX protein to thereby inhibit expression of the protein (e.g. by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred. In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n-1, wherein n is an integer between 1 and 85). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15. In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675. PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996. supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)-amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization-triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 85. The invention also includes a mutant or variant protein any of whose residues may be changed from the corresponding residues shown in any one of SEQ ID NO:2n, wherein n is an integer between 1 and 85, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation. The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 85) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 85. In other embodiments, the NOVX protein is substantially homologous to SEQ ID NO:2n, wherein n is an integer between 1 and 85, and retains the functional activity of the protein of SEQ ID NO:2n, wherein n is an integer between 1 and 85, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 85, and retains the functional activity of the NOVX proteins of SEQ ID NO:2n, wherein n is an integer between 1 and 85.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n-1, wherein n is an integer between 1 and 85.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 85, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides. In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g., promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand. A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g. a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 85, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art. A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen that is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fᵥ framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96). In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals. For example, mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fᵥ molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F(ab)₂ fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986). According to another approach described in WO 96/27011, the interface between a pair of antibody molecules can be engineered to maximize the percentage of heterodimers that are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148 (5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fᵥ (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., _J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81 (19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g. a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (ie., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible. Alternatively, the effect may be one in which the antibody elicits a physiological result by virtue of binding to an effector binding site on the target molecule. In this case the target, a receptor having an endogenous ligand that may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F(ab)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, useful expression vectors in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 1 d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989. In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1 (1) 1986. Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, ie., any one of SEQ ID NO:2n-1, wherein n is an integer between 1 and 85, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n-1, wherein n is an integer between 1 and 85), but more preferably, is a non-human homologue of a human NOVX gene. For example, a mouse homologue of human NOVX gene of SEQ ID NO:2n-1, wherein n is an integer between 1 and 85, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P 1. For a description of the cre/loxP recombinase system, see, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion. The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds that bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can be of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g., a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e., intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g. luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g., stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

In yet another aspect of the invention a method for identifying compounds that modulate target polypeptide (NOVX) activity is disclosed wherein the method comprises: (a) combining a test compound with a target polypeptide and a substrate of the target polypeptide; and (b) determining whether the test compound modulates the activity of the target polypeptide; wherein the target polypeptide comprises an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

The method further comprising a step of identifying the test compound that modulates the target polypeptide activity by modulating the target polypeptide activity as modulator of the target polypetide. Such modulator could be an inhibitor, an activator, an antagonist, or an agonist of NOVX target polypeptide.

The method also further comprising a step of identifying the test compound that modulates the target polypeptide activity as an enhancer of insulin secretion, or as a therapeutic for treatment of insulin resistance, obesity and/or diabetes.

In the above described method, the target polypeptide (NOVX) could be an isolated polypetide.

The target polypeptide could be produced by a process comprising culturing a recombinant host cell, the recombinant host cell comprising a nucleic acid encoding the target polypeptide, under conditions promoting expression of the target polypeptide. In such a method, the nucleic acid comprises a nucleotide sequence selected from the group consisting of: (a) SEQ ID NO:2n-1, wherein n is an integer between 1 and 85; (b) nucleotides encoding an amino acid sequence of the at least one domain of SEQ ID NO:2n; and (c) a nucleotide sequence encoding an amino acid sequence selected from the group consisting of SEQ ID NO:2n, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

Alternatively, the target polypeptide could be produced by expression of a recombinant vector comprising a nucleic acid, the nucleic acid encoding an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n. Here, the test compound could be combined with the target polypeptide in a mammalian cell grown in culture. Also, the test compound could be combined with the target polypeptide in vitro. In this method, the nucleic acid comprises a nucleotide sequence selected from the group consisting of: (a) SEQ ID NO:2n-1, wherein n is an integer between 1 and 85; (b) nucleotides encoding an amino acid sequence of the at least one domain of SEQ ID NO:2n; and (c) a nucleotide sequence encoding an amino acid sequence selected from the group consisting of SEQ ID NO:2n, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

In yet another embodiment, the target polypeptide is produced by expression of an endogenous nucleic acid, the endogenous nucleic acid encoding an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n. Here as well, the test compound could be combined with the target polypeptide in a mammalian cell grown in culture. Also, the test compound could be combined with the target polypeptide in vitro. In this method, the nucleic acid comprises a nucleotide sequence selected from the group consisting of: (a) SEQ ID NO:2n-1, wherein n is an integer between 1 and 85; (b) nucleotides encoding an amino acid sequence of the at least one domain of SEQ ID NO:2n; and (c) a nucleotide sequence encoding an amino acid sequence selected from the group consisting of SEQ ID NO:2n, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n-1, wherein n is an integer between 1 and 85, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n-1, wherein n is an integer between 1 and 85, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n-1, wherein n is an integer between 1 and 85, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., F_{ab} or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g. Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table 1.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table 1.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table 1.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

NOVX Polypeptides

The following sections describe in detail the NOVX polypeptides of the invention and methods of screening for modulators of NOVX polypeptides.

A. NOV1—Cytosolic Phosphoenolpyruvate Carboxykinase (PEPCK)

The cytosolic isoform of PEPCK regulates glyceroneogenesis in adipose tissue. Glyceroneogenesis is an abbreviated version of gluconeogenesis in which glycerol-3-phosphate is produced from substrates such as pyruvate, lactate and alanine. The glycerol-3-phosphate thus produced is used in triglyceride synthesis. The role of glyceroneogenesis in maintaining the deposition of triglycerides in adipose tissue has been uncovered recently. Cytosolic PEPCK is also the rate-limiting enzyme for gluconeogenesis, the pathway in which glucose is produced in the liver from pyruvate, lactate and alanine. The process of hepatic gluconeogenesis is upregulated in Type 2 diabetes and is believed to contribute to the fasting hyperglycemia characteristic of this disease. The genetic and environmental causes of the complex metabolic disturbances of Type 2 diabetes, including increased hepatic glucose production, are incompletely understood.

CuraGen's GeneCalling® studies have shown that cytosolic PEPCK is upregulated in adipose tissue of obese AKR versus normal C57B1 mice. This result suggests that upregulation of PEPCK may contribute to the obese phenotype. This hypothesis is supported by the fact that transgenic overexpression of cytosolic PEPCK in adipose is associated with increased glyceroneogenesis, increased adipocyte (fat cell) size and fat mass, and higher body weight (Franckhauser S, Munoz S, Pujol A, Casellas A, Riu E, Otaegui P, Su B, Bosch F. Increased fatty acid re-esterification by PEPCK overexpression in adipose tissue leads to obesity without insulin resistance. Diabetes. 2002 March; 51 (3):624-30. PMID: 11872659). Furthermore, a mutation in the PPARgamma-binding site in the gene for cytosolic PEPCK reduces the activity of PEPCK, with a concomitamt increase in adiposity and body weight in mice (Olswang Y, Cohen H, Papo O, Cassuto H, Croniger C M, Hakimi P, Tilghman S M, Hanson R W, Reshef L. A mutation in the peroxisome proliferator-activated receptor gamma-binding site in the gene for the cytosolic form of phosphoenolpyruvate carboxykinase reduces adipose tissue size and fat content in mice. Proc Natl Acad Sci U S A. 2002 Jan. 22; 99 (2):625-30. PMID: 11792850). Thus, a reduction in the expression of cytosolic PEPCK reduced triglyceride deposition in adipose tissue, while an increase in adipose expression of PEPCK increased fat mass in mice.

Diet-induced obesity is a popular model for the study of pathways involved in the evolution of weight gain due to increased caloric intake. Mice are fed a high-fat diet (˜30-45% of calories from fat) for 12-16 weeks and then tissues are analyzed for changes in metabolic pathways as compared to control animals fed a normal rodent diet. CuraGen's GeneCalling® studies of adipose tissue from high fat-fed versus normal chow-fed mice have given results contradictory to those discussed above. Contrary to expectations, cytosolic PEPCK is downregulated in multiple adipose depots of high fat versus normal chow-fed mice. Downregulation of adipose PEPCK was documented at multiple time points in the course of the diet-induced obesity protocol. Our interpretation of these results is that downregulation of PEPCK may be a compensatory response to limit triglyceride deposition. In support of this hypothesis, a number of additional lipogenic genes are downregulated in adipose tissue under conditions of diet-induced obesity, including diacylglycerol transferase 2, monoglyceride lipase, lipoprotein lipase and aquaporin adipose.

The discordance in PEPCK gene expression in diet-induced obesity versus a genetic model of obesity may be explained by the differences in the two models. In genetic obesity, the pathophysiology is static because it is hard-wired into the organism. In contrast, in diet-induced obesity, the increase in fat mass evolves over a period of 12-16 weeks, and is a result of the increase in high-fat calories. The response to the caloric overload is dynamic (as seen in CuraGen's GeneCalling® studies), and includes compensatory responses by the organism to maintain energy homeostasis and body weight. Such a compensatory response to caloric overload appears to include a downregulation of PEPCK in adipose tissue.

CuraGen's GeneCalling® studies have also shown an upregulation of cytosolic PEPCK in the liver of mice in the diet-induced obesity model (data included below). The gene is upregulated 1.8-fold in the transition from normoglycemia to hyperglycemia in obese mice. This data strongly supports a pathogenic role for cytosolic PEPCK in the increased hepatic glucose production and hyperglycemia of Type 2 diabetes. It is important to note that more than 90% of patients with Type 2 diabetes are obese. In the diet-induced obesity model, a proportion of the obese mice also develop hyperglycemia and other metabolic disturbances characteristic of Type 2 diabetes. Thus cytosolic PEPCK is also a therapeutic target for Type 2 diabetes.

The following summarizes the biochemistry surrounding the human cytosolic Phosphoenolpyruvate Carboxykinase (PEPCK) and potential assays that may be used to screen for antibody therapeutics or small molecule drugs to treat obesity and/or diabetes.

Cytosolic Phosphoenolpyruvate carboxykinase (PEPCK) is an important enzyme in whole-body energy homeostasis and catalyzes the following reaction:
GTP+oxaloacetate=GDP+phosphoenolpyruvate+CO₂

It is the rate-limiting enzyme in gluconeogenesis in liver and glyceroneogenesis in adipose tissue, participating in the glyceroneogenic pathway in adipocytes when glucose is limiting. Adipose tissue glyceroneogenesis produces glycerol 3-phosphate, which is the substrate for triglyceride deposition. Phosphoenolpyruvate carboxykinase (PEPCK) activity is affected by a number of hormones that regulate this metabolic process, including glucagon, insulin, and glucocorticoids.

Taken in total, the data indicates that a modulator such as an inhibitor/antagonist of the human cytosolic Phosphoenolpyruvate Carboxykinase (PEPCK) would be beneficial in the treatment of obesity and/or diabetes.

Furthermore, our results indicate that a modulator of cytosolic Phosphoenolpyruvate Carboxykinase (PEPCK) activity, such as an inhibitor, activator, antagonist, or agonist of PEPCK may be useful for treatment of such disorders as obesity, diabetes, and insulin resistance, as well as for enhancement of insulin secretion.

Discovery Process

The following sections describe the study design(s) and the techniques used to identify the Cytosolic Phosphoenolpyruvate Carboxykinase (PEPCK)—encoded protein and any variants, thereof, as being suitable as diagnostic markers, targets for an antibody therapeutic and targets for a small molecule drugs for Obesity and Diabetes.

### EXAMPLE A1

### Genetically Obese Mice vs Genetically Lean Mice Study

A protocol for Genetically Obese Mice vs Genetically Lean Mice Study is disclosed in Example Q6.

A fragment of the mouse cytosolic Phosphoenolpyruvate carboxykinase (PEPCK) 1 gene (mouse strains AKR, C57BL) was initially found to be up-regulated by 3-fold in the adipose tissue of obese AKR mice relative to lean C57L/J mice using CuraGen's GeneCalling® method of differential gene expression (described in Example Q7). A differentially expressed mouse gene fragment migrating, at approximately 254 nucleotides in length was definitively identified as a component of the mouse Cytosolic Phosphoenolpyruvate carboxykinase (PEPCK) 1 cDNA. The method of competitive PCR was used for confirmation of the gene assessment. The electropherographic peaks corresponding to the gene fragment of the mouse cytosolic Phosphoenolpyruvate carboxykinase (PEPCK) 1 were ablated when a gene-specific primer (shown in Table A1) competes with primers in the linker-adaptors during the PCR amplification. The peaks at 254 nt in length were ablated in the sample from both the obese AKR mice and the normal C57L/J mice.

TABLE A1

The direct sequence of the 254 nucleotide-long gene fragment and the
gene-specific primers used for competitive PCR are indicated on the
cDNA sequence of Cytosolic Phosphoenolpyruvate Carboxykinase (PEPCK)
1 fragment (SEQ ID NO:171) are shown in bold. The gene-specific
+UZ,1/44primers at the 5′ and 3′ ends of the fragment are underlined.
Gene Sequence (fragment from 1442 to 1695 in bold. band size: 254)

961 AAGGCAAGAA GAAATACCTG GCCGCAGCCT TCCCTAGTGC CTGTGGGAAG ACTAACTTGG
1021 CCATGATGAA CCCCAGCCTG CCCGGGTGGA AGGTCGAATG TGTGGGCGAT GACATTGCCT
1081 GGATGAAGTT TGATGCCCAA GGCAACTTAA GGGCTATCAA CCCAGAAAAC GGGTTTTTTG
1141 GAGTTGCTCC TGGCACCTCA GTGAAGACAA ATCCAAATGC CATTAAAACC ATCCAGAAAA
1201 ACACCATCTT CACCAACGTG GCCGAGACTA GCGATGGGGG TGTTTACTGG GAAGGCATCG
1261 ATGAGCCGCT GGCCCCGGGA GTCACCATCA CCTCCTGGAA GAACAAGGAG TGGAGACCGC
1321 AGGACGCGGA ACCATGTGCC CATCCCAACT CGAGATTCTG CACCCCTGCC AGCCAGTGCC
1381 CCATTATTGA CCCTGCCTGG GAATCTCCAG AAGGAGTACC CATTGAGGGT ATCATCTTTG
1441 GTGGCCGTAG ACCTGAAG GT GTCCCCCTTG TCTATGAAGC CCTCACCTGG CAGCATGGGG
1501 TGTTTGTACG AGCAGCCATG AGATCTGAGG CCACACCTCC TCCACAACAC AAGGGCAAGA
1561 TCATCATGCA CGACCCCTTT GCCATCCGAC CCTTCTTCGG CTACAACTTC GGCAAATACC
1621 TGGCCCACTG GCTGACCATC GCCCACCGCC CACCAGCCAA GTTGCCCAAG ATCTTCCATC
1681 TCAACTGCTT CCGGAAGGAC AAAGATGGCA AGTTCCTCTG GCCAGGCTTT GGCGAGAACT
1741 CCCGGGTGCT GGAGTGGATG TTCGGGCGGA TTGAAGGGGA AGACAGCGCC AAGCTCACGC
1801 CCATCGGCTA CATCCCTAAG GAAAACGCCT TGAACCTGAA AGGCCTGGGG GGCGTCAACG
1861 TGGAGGAGCT GTTTGGGATC TCTAAGGAGT TCTGGGAGAA GGAGGTGGAG GAGATCGACA
1921 GGTATCTGGA GGACCAGOTC AACACCGACC TCCCTTACGA AATTGAGAGG GAGCTCCGAG
1981 CCCTGAAACA GAGAATCAGC CAGATGTAAA TCCCAATGGG GGCGTCTCGA GAGTCACCCC
2041 TTCCCACTCA CAGCATCGCT GAGATCTAGG AGAAAGCCAG CCTGCTCCAG CTTTGAGATA
2101 GCGGCACAAT CGTGAGTAGA TCAGAAAAGC ACCTTTTAAT AGTCAGTTGA GTAGCACAGA
2161 GAACAGGCTA GGGGC

(gene length is 2617, only region from 961 to 2175 shown)

### EXAMPLE A2

### Mouse Dietary-Induced Obesity Study

A protocol for Mouse Dietary-Induced Obesity study is disclosed in Example Q1.

A large number of mouse strains have been identified that differ in body mass and composition. The AKR and NZB strains are obese, the SWR, C57BL and C57BL/6 strains are of average weight whereas the SM/J and Cast/E1 strains are lean. Understanding the gene expression differences in the major metabolic tissues from these strains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design sought to identify the factors that lead to the ability of certain individuals to resist the effects of a high fat diet and thereby prevent obesity. The sample groups for the study had body weights +1 S.D., +4 S.D. and +7 S.D. of the chow-fed controls. In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues revealed genes and pathways that can be used as therapeutic targets for obesity. Protocol for differential gene expression analysis, GeneCalling®, is disclosed in Example Q7.

Results

A gene fragment of mouse cytosolic Phosphoenolpyruvate carboxykinase (PEPCK) was found to be down-regulated by 7-fold in the epididymal fat pad (efp) of Ngsd7 (normal glycemic, obese) vs. chow-fed mice in a diet-induced obesity study using CuraGen's GeneCalling® method of differential gene expression. A differentially expressed mouse gene fragment migrating, at approximately 349 nucleotides in length was definitively identified as a component of the mouse cytosolic Phosphoenolpyruvate carboxykinase (PEPCK) cDNA. The method of competitive PCR was used for confirmation of the gene assessment. The electropherographic peaks corresponding to the gene fragment of the mouse cytosolic Phosphoenolpyruvate carboxykinase (PEPCK) were ablated when a gene-specific primer (shown in Table A2) competes with primers in the linker-adaptors during the PCR amplification. The peaks at 349 nt in length were ablated in the sample from both the Ngsd7 and chow-fed mice. In addition, the mouse cytosolic Phosphoenolpyruvate carboxykinase (PEPCK) gene is down-regulated in the retroperitoneal fat pad (rfp) of Ngsd7 (normal glycemic, obese) and Hgsd7 (hyperglycemic, obese) versus Sd1 or chow-fed mice. It should be noted that the downregulation of this gene in adipose in a dietary-induced obesity model may be a compensatory response to limit triglyceride deposition and adiposity.

TABLE A2

The direct sequence of the 349 nucleotide-long gene fragment and the
gene-specific primers used for competitive PCR are indicated on the
cDNA sequence of the cytosolic Phosphoenolpyruvate Carboxykinase
(PEPCK) fragment (SEQ ID NO:172) are shown in bold. The gene-specific
primers at the 5′ and 3′ ends of the fragment are underlined.
Gene Sequence (fragment from 2077 to 2425 in bold. band size: 349)
1596 CCATGCGACC CTTCTTCGGC TACAACTTCG GCAAATACCT GGCCCACTGG CTGAGCATGG
1656 CCCACCGCCC AGCAGCCAAG TTGCCCAAGA TCTTCCATGT CAACTGGTTC CGGAAGGACA
1716 AAGATGGCAA GTTCCTCTGG CCAGGCTTTG GCGAGAACTC CCGGGTGCTG GAGTGGATGT
3776 TCGGGCGGAT TGAAGGGGAA GACAGCGCCA AGCTCACGCC CATCGGCTAC ATCCCTAAGG
1836 AAAACGCCTT GAACCTGAAA GGCCTGGGGG GCGTCAACGT GGAGGAGCTG TTTGGGATCT
1896 CTAAGGAGTT CTGGGAGAAG GAGGTGGAGG AGATCGACAG GTATCTGGAG GACCAGGTCA
1956 ACACCGACCT CCCTTACGAA ATTGAGAGGG AGCTCCGAGC CCTGAAACAG AGAATCAGCC
2016 AGATGTAAAT CCCAATGGGG GCGTCTCGAG AGTCACCCCT TCCCACTCAC AGCATGCGCT
2076 GAGATCTACG AGAAAGCCAG CCTGCTCCAG CTTTGAGATA CCGGCACAAT GCTGAGTAGA
2136 TCAGAAAAGC ACCTTTTAAT AGTCACTTGA GTAGCACACA GAACAGGCTA GGGGCAAATA
2196 AGATTGGGAG GGGAAATCAC CGCATAGTCT CTGAAGTTTG CATTTGACAC CAATGCGGGT
2256 TTTGGTTCCA CTTCAACGTC ACTCAGGAAT CCAGTTCTTC ACGTTAGCTG TACCAGTTAG
2316 CTAAAATGCA CAGAAAACAT ACTTGAGCTG TATATATGTG TGTGAACGTG TCTCTGTGTG
2376 AGCATGTGTG TGTGTGTGTG TGTGTGTGTG TGTGTGTGTG TGTCTGTACA TGCCTGTCTG
2436 TCCCATTGTC CACAGTATAT TTAAAACCTT TGGGGAAAAA TCTTGGGCAA ATTTGTAGCT
2496 GTAACTAGAG AGTCATGTTG CTTTGTTGCT AGTATGTATG TTTAAATTAT TTTTATACAC
2556 CGCCCTTCCT TACCTTTCTT TACATAATTG AAATTGGTAT CCGGACCACT TCTTGGGAAA
2616 AAAATTACAA AATAAACTTT TATAGAAAAA GTAAAAAAAA AAAAAAAA

(gene length is 2663, only region from 1596 to 2663 shown)

Our data also showed that cytosolic PEPCK is downregulated 1.8-fold in the liver of Ngsd7 (normal glycemia and obese) versus Hgsd7 (hyperglycemic and obese) mice in the diet-induced obesity study. Identitiy of the PEPCK as the downregulated gene was then confirmed by TrapPing technology (disclosed in Example Q7). Therefore, PEPCK is upregulated in Hgsd7 liver versus Ngsd7 liver. Thus, PEPCK upregulation occurs at the transition between normal glycemia and the pathologic change to hyperglycemia. Since PEPCK is the rate-controlling step in the process of hepatic gluconeogenesis (production of glucose from non-glucose substrates), the data suggests that upregulation of PEPCK is a critical step in the evolution to hyperglycemia and Type 2 diabetes.

### EXAMPLE A3

### Identification of Human PEPCK Sequence

The sequence of Human PEPCK (Acc. No. CG101190-01) was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof. The protocol for identification of human sequence(s) is disclosed in Example Q8.

Table A3 shows an alignment (ClustalW) of the protein sequences of the human (CG101190-01), rat (PPCC_MOUSE=Q9Z2V4) and mouse (PPCC_RAT=P07379) versions of the cytosolic Phosphoenolpyruvate Carboxykinase (PEPCK). Table A4 shows sequences of rat (PPCC_MOUSE=Q9Z2V4; SEQ ID NO:174) and mouse (PPCC_RAT=P07379; SEQ ID NO:173) versions of the cytosolic Phosphoenolpyruvate Carboxykinase (PEPCK).

[image]

TABLE A5

>PPCC_MOUSE (SEQ ID NO:173)
MPPQLHNGLDFSAKVIQGSLDSLPQAVRKFVEGNAQLCQPEYIHICDGSEEEYGQLLAHMQEEGVIRKLK
KYDNCWLALTDPRDVARIESKTVIITQEQRDTVPIPKTGLSQLGRWMSEEDFEKAFNARFPGCMKGRTMY
VIPFSMGPLGSPLAKIGIELTDSPYVVASMRIMTRMGISVLEALGDGEFIKCLHSVGCPLPLKKPLVNNW
ACNPELTLIAHLPDRREIISFGSGYGGNSLLGKKCFALRIASRLAKEEGWLAEHMLILGITNPEGKKKYL
AAAFPSACGKTNLAMMNPSLPGWKVECVGDDIAWMKFDAQGNLRAINPENGFFGVAPGTSVKTNPNAIKT
IQKNTIFTNVAETSDGGVYWEGIDEPLAPGVTITSWKNKEWRPQDAEPCAHPNSRFCTPASQCPILDPAW
ESPEGVPIEGIIFGGRRPEGVPLVYEALSWQHGVFVGAAMRSEATAAAEHKGKIIMHDPFAMRPFFGYNF
GKYLAHWLSMAHRPAAKLPKIFHVNWFRKDKDGKFLWPGFGENSRVLEWMFGRIEGEDSAKLTPIGYIPK
ENALNLKGLGGVNVEELFGISKEFWEKEVEEIDRYLEDQVNTDLPYEIERELRALKQRISQM
>PPCC_RAT (SEQ IN NO:174)
MPPQLHNGLDFSAKVIQGSLDSLPQEVRKFVEGNAQLCQPEYIHICDGSEEEYGRLLAHMQEEGVIRKLK
KYDNCWLALTDPRDVARIESKTVIITQEQRDTVPIPKSGQSQLGRWMSEEDFEKAFNARFPGCMKGRTMY
VIPFSMGPLGSPLAKIGIELTDSPYVVASMRIMTRMGTSVLEALGDGEFIKCLHSVGCPLPLKKPLVNNW
ACNPELTLIAHLPDRREIISFGSGYGGNSLLGKKCFALRIASRLAKEEGWLAEHMLILGITNPEGKKKYL
AAAFPSACGKTNLAMMNPTLPGWKVECVGDDIAWMKFDAQGNLRAINPENGFFGVAPGTSVKTNPNAIKT
IQKNTIFTNVAETSDGGVYWEGLDEPLAPGVTITSWKNKEWRPQDEEPCAHPNSRFCTPASQCPIIDPAW
ESPEGVPIEGIIFGGRRPAGVPLVYEALSWQHGVFVGAAMRSEATAAAEHKGKVIMHDPFAMRPFFGYNF
GKYLAHWLSMAHRPAAKLPKIFHVNWFRKDKNGKFLWPGFGENSRVLEWMFGRLEGEDSAKLTPIGYVPK
EDALNLKGLGDVNVEELFGISKEFWEKEVEELDKYLEDQVNADLPYELERELRALKQRISQM

The laboratory cloning was performed using one or more of the methods summarized in Example Q8. The NOV1 clone was analyzed, and the nucleotide and encoded polypeptide sequences are shown in Table A6.

TABLE A6
NOV1 Sequence Analysis

| | | |
|---|---|---|
| NOV1a, CG101190-01 | SEQ ID NO:1 | 2070 bp |
| DNA Sequence | ORF Start: ATG at 119 | ORF Stop: TAA at 1985 |

GAACACAAACTTGCTGGCGGGAAGAGCCCGGAAAGAAACCTGTGGATCTCCCTTCGAGATCATCCAAA
GAGAAGAAAGGTGACCTCACATTCGTGCCCCTTAGCAGCACTCTGCAGAAATGCCTCCTCAGCTGCAA
AACGGCCTGAACCTCTCGGCCAAAGTTGTCCAGGGAAGCCTGGACAGCCTGCCCCAGGCAGTGAGGGA
GTTTCTCGAGAATAACGCTGAGCTGTGTCAGCCTGATCACATCCACATCTGTGACGGCTCTGAGGAGG
AGAATGGGCGGCTTCTGGGCCAGATGGAGGAAGAGGGCATCCTCAGGCGGCTGAAGAAGTATGACAAC
TGCTGGTTGGCTCTCACTGACCCCAGGGATGTGGCCAGGATCGAAAGCAAGACGGTTATCGTCACCCA
AGAGCAAAGAGACACAGTGCCCATCCCCAAAACAGGCCTCAGCCAGCTCGGTCGCTGGATGTCAGAGG
AGGATTTTGAGAAAGCGTTCAATGCCAGGTTCCCAGGGTGCATGAAAGGTCGCACCATGTACGTCATC
CCATTCAGCATGGGGCCGCTGGGCTCACCTCTGTCGAAGATCGGCATCGAGCTGACGGATTCGCCCTA
CGTGGTGGCCAGCATGCGGATCATGACGCGGATGGGCACGCCCGTCCTGGAAGCACTGGGCGATGGGG
AGTTTGTCAAATGCCTCCATTCTGTGGGGTGCCCTCTGCCTTTACAAAAGCCTTTGGTCAACAACTGG
CCCTGCAACCCGGAGCTGACGCTCATCGCCCACCTGCCTGACCGCAGAGAGATCATCTCCTTTGGCAG
TGGGTACGGCGGGAACTCGCTGCTCGGGAAGAAGTGCTTTGCTCTCAGGATGGCCAGCCGGCTGGCCA
AGGAGGAAGGGTGGCTGGCAGAGCACATGCTGATTCTGGGTATAACCAACCCTGAGGGTGAGAAGAAG
TACCTGGCGGCCGCATTTCCCAGCGCCTGCGGGAAGACCAACCTGGCCATGATGAACCCCAGCCTCCC
CGGGTGGAAGGTTGAGTGCGTCGGGGATGACATTGCCTGGATGAAGTTTGACGCACAAGGTCATTTAA
GGGCCATCAACCCAGAAAATGGCTTTTTCGGTGTCGCTCCTGGGACTTCAGTGAAGACCAACCCCAAT
GCCATCAAGACCATCCAGAAGAACACAATCTTTACCAATGTGGCCGAGACCAGCGACGGGGGCGTTTA
CTGGGAAGGTATTGATGAGCCGCTAGCTTCAGGCGTCACCATCACGTCCTGGAAGAATAAGGAGTGGA
GCTCAGAGGATGGGGAACCTTGTGCCCACCCCAACTCGAGGTTCTGCACCCCTGCCAGCCAGTGCCCC
ATCATTGATGCTGCCTGGGAGTCTCCGGAAGGTGTTCCCATTGAAGGCATTATCTTTGGAGGCCGTAG
ACCTGCTGGTGTCCCTCTAGTCTATGAAGCTCTCAGCTGGCAACATGGAGTCTTTGTGGGGGCGGCCA
TGAGATCAGAGGCCACAGCGGCTGCAGAACATAAAGGCAAAATCATCATGCATGACCCCTTTGCCATG
CGGCCCTTCTTTGGCTACAACTTCGGCAAATACCTGGCCCACTGGCTTAGCATGGCCCAGCACCCAGC
AGCCAAACTGCCCAAGATCTTCCATGTCAACTGGTTCCGGAAGGACAAGGAAGGCAAATTCCTCTGGC
CAGGCTTTGGAGAGAACTCCAGGGTGCTGGAGTGGATGTTCAACCGGATCGATGGAAAAGCCAGCACC
AAGCTCACGCCCATAGGCTACATCCCCAAGGAGGATGCCCTGAACCTGAAAGGCCTGGGGCACATCAA
CATGATGGAGCTTTTCAGCATCTCCAAGGAATTCTGGGAGAAGGAGGTGGAAGACATCGAGAAGTATC
TGGAGGATCAAGTCAATGCCGACCTCCCCTGTGAAATCGAGAGAGAGATCCTTGCCTTGAAGCAAAGA
ATAAGCCAGATGTAATCAGGGCCTGAGTGCTTTACCTTTAAAATCATTCCCTTTCCCATCCATAAGGT
GCAGTAGGAGCAAGAGAGGGCAAGTGTTCC

| | | | |
|---|---|---|---|
| NOV1a, CG101190-01 | | | |
| Protein Sequence | SEQ ID NO:2 | 622 aa | MW at 69207.8 kD |

MPPQLQNGLNLSAKVVQGSLDSLPQAVREFLENNAELCQPDHIHICDGSEEENGRLLGQMEEEGILRR
LKKYDNCWLALTDPRDVARIESKTVIVTQEQRDTVPIPKTGLSQLGRWMSEEDFEKAFNARFPGCMKG
RTMYVIPFSMGPLGSPLSKIGIELTDSPYVVASMRIMTRMGTPVLEALGDGEFVKCLHSVGCPLPLQK
PLVNNWPCNPELTLIAHLPDRREIISFGSGYGGNSLLGKKCFALRMASRLAKEEGWLAEHMLILGITN
PEGEKKYLAAAFPSACGKTNLAMNNPSLPGWKVECVGDDIAWMKFDAQGHLRAINPENGFFGVAPGTS
VKTNPNAIKTIQKNTIFTNVAETSDGGVYWEGIDEPLASGVTITSWKNKEWSSEDGEPCAHPNSRFCT
PASQCPIIDAAWESPEGVPIEGIIFGGRRPAGVPLVYEALSWQHGVFVGAAMRSEATAAAEHKGKIIM
HDPFAMRPFFGYNFGKYLAHWLSMAQHPAAKLPKIFHVNWFRKDKEGKFLWPGFGENSRVLEWMFNRI
DGKASTKLTPIGYIPKEDALNLKGLGHINMMELFSISKEFWEKEVEDIEKYLEDQVNADLPCEIEREI
LALKQRISQM

| | | |
|---|---|---|
| NOV1b, 278992806 | SEQ ID NO:3 | 1888 bp |
| DNA Sequence | ORF Start: at 2 | ORF Stop: end of sequence |

CACCGGATCCACCATGCCTCCTCAGCTGCAAAACGGCCTGAACCTCTCGGCCAAAGTTGTCCAGGGAA
GCCTGGACAGCCTGCCCCAGGCAGTGAGGGAGTTTCTCGAGAATAACGCTGAGCTGTGTCAGCCTGAT
CACATCCACATCTGTGACGGCTCTGAGGAGGAGAATGGGCGGCTTCTGGGCCAGATGGAGGAAGAGGG
CATCCTCAGGCGACTGAAGAAGTATGACAACTGCTGGTTGGCTCTCACTGACCCCAGGGATGTGGCCA
GGATCGAAAGCAAGACGGTTATCGTCACCCAAGAGCAAAGAGACACAGTGCCCATCCCCAAAACAGGC
CTCAGCCAGCTCGGTCGCTGGATGTCAGAGGAGGATTTTGAGAAAGCGTTCAATGCCAGGTTCCCAGG
GTGCATGAAAGGTCGCACCATGTACGTCATCCCATTCAGCATGGGGCCGCTGGGCTCACCTCTGTCGA
AGATCGGCATCGAGCTGACGGATTCGCCCTACGTGGTGGCCAGCATGCGGATCATGACGCGGATGGGC
ACGCCCGTCCTGGAAGCACTGGGCGATGGGGAGTTTGTCAAATGCCTCCATTCTGTGGGGTGCCCTCT
GCCTTTACAAAAGCCTTTGGTCAACAACTGGCCCTGCAACCCGGAGCTGACGCTCATCGCCCACCTGC
CTGACCGCAGAGAGATCATCTCCTTTGGCAGTGGGTACGGCGGGAACTCGCTGCTCGGGAAGAAGTGC
TTTGCTCTCAGGATGGCCAGCCGGCTGGCCAAGGAGGAAGGGTGGCTGGCAGAGCACATGCTGATTCT
GGGTATAACCAACCCTGAGGGTGAGAAGAAGTACCTGGCGGCCGCATTTCCCAGCGCCTGCGGGAAGA
CCAACCTGGCCATGATGAACCCCAGCCTCCCCGGGTGGAAGGTTGAGTGCGTCGGGGATGACATTGCC
TGGATGAAGTTTGACGCACAAGGTCATTTAAGGGCCATCAACCCAGAAAATGGCTTTTTCGGTGTCGC
TCCTGGGACTTCAGTGAAGACCAACCCCAATGCCATCAAGACCATCCAGAAGAACACAATCTTTACCA
ATGTGGCCGAGACCAGCGACGGGGGCGTTTACTGGGAAGGTATTGATGAGCCGCTAGCTTCAGGCGTC
ACCATCACGTCCTGGAAGAATAAGGAGTGGAGCTCAGAGGATGGGGAACCTTGTGCCCACCCCAACTC
GAGGTTCTGCACCCCTGCCAGCCAGTGCCCCATCATTGATGCTGCCTGGGAGTCTCCGGAAGGTGTTC
CCATTGAAGGCATTATCTTTGGAGGCCGTAGACCTGCTGGTGTCCCTCTAGTCTATGAAGCTCTCAGC
TGGCAACATGGAGTCTTTGTGGGGGCGGCCATGAGATCAGAGGCCACAGCGGCTGCAGAACATAAAGG
CAAAATCATCATGCATGACCCCTTTGCCATGCGGCCCTTCTTTGGCTACAACTTCGGCAAATACCTGG
CCCACTGGCTTAGCATGGCCCAGCACCCAGCAGCCAAACTGCCCAAGATCTTCCATGTCAACTGGTTC
CGGAAGGACAAGGAAGGCAAATTCCTCTGGCCAGGCTTTGGAGAGAACTCCAGGGTGCTGGAGTGGAT
GTTCAACCGGATCGATGGAAAAGCCAGCACCAAGCTCACGCCCATAGGCTACATCCCCAAGGAGGATG
CCCTGAACCTGAAAGGCCTGGGGCACATCAACATGATGGAGCTTTTCAGCATCTCCAAGGAATTCTGG
GAGAAGGAGGTGGAGACATCGAGAAGTATCTGGAGGATCAAGTCAATGCCGACCTCCCCTGTGAAAT
CGAGAGAGAGATCCTTGCCTTGAAGCAAAGAATAAGCCAGATGGTCGACGGC

| | | | |
|---|---|---|---|
| NOV1b, 278992806 | | | |
| Protein Sequence | SEQ ID NO:4 | 629 aa | MW at 69825.4 kD |

TGSTMPPQLQNGLNLSAKVVQGSLDSLPQAVREFLENNAELCQPDHIHICDGSEEENGRLLGQMEEEG
ILRRLKKYDNCWLALTDPRDVARIESKTVIVTQEQRDTVPIPKTGLSQLGRWMSEEDFEKAFNARFPG
CMKGRTMYVIPFSMGPLGSPLSKIGIELTDSPYVVASMRIMTRMGTPVLEALGDGEFVKCLHSVGCPL
PLQKPLVNNWPCNPELTLIAHLPDRREIISFGSGYGGNSLLGKKCFALRMASRLAKEEGWLAEHMLIL
GITNPEGEKKYLAAAFPSACGKThLAMMNPSLPGWKVECVGDDIAWMKFDAQGHLRAINPENGFFGVA
PGTSVKTNPNAIKTIQKNTIFTNVAETSDGGVYWEGIDEPLASGVTITSWKNKEWSSEDGEPCAHPNS
RFCTPASQCPIIDAAWESPEGVPIEGIIFGGRRPAGVPLVYEALSWQHGVFVGAANRSEATAAAEHKG
KIIMHDPFAMRPFFGYNFGKYLAHWLSMAQHPAAKLPKIFHVNWFRKDKEGKFLWPGFGENSRVLEWM
FNRIDGKASTKLTPIGYIPKEDALNLKGLGHINMMELFSISKEFWEKEVEDIEKYLEDQVNADLPCEI
EREILALKQRISQMVDG

| | | |
|---|---|---|
| NOV1c, 278992862 | SEQ ID NO:5 | 1801 bp |
| DNA Sequence | ORF Start: at 2 | ORF Stop: end of sequence |

CACCGGATCCGAGTTTCTCGAGAATAACGCTGAGCTGTGTCAGCCTGATCACATCCACATCTGTGACG
GCTCTGAGGAGGAGAATGGGCGGCTTCTGGGCCAGATGGAGGAAGAGGGCATCCTCAGGCGACTGAAG
AAGTATGACAACTGCTGGTTGGCTCTCACTGACCCCAGGGATGTGGCCAGGATCGAAAGCAAGACGGT
TATCGTCACCCAAGAGCAAAGAGACACAGTGCCCATCCCCAAAACAGGCCTCAGCCAGCTCGGTCGCT
GGATGTCAGAGGAGGATTTTGAGAAAGCGTTCAATGCCAGGTTCCCAGGGTGCATGAAAGGTCGCACC
ATGTACGTCATCCCATTCAGCATGGGGCCGCTGGGCTCACCTCTGTCGAAGATCGGCATCGAGCTGAC
GGATTCGCCCTACGTGGTGGCCAGCATGCGGATCATGACGCGGATGGGCACGCCCGTCCTGGAAGCAC
TGGGCGATGGGGAGTTTGTCAAATGCCTCCATTCTGTGGGGTGCCCTCTGCCTTTACAAAAGCCTTTG
GTCAACAACTGGCCCTGCAACCCGGAGCTGACGCTCATCGCCCACCTGCCTGACCGCAGAGAGATCAT
CTCCTTTGGCAGTGGGTACGGCGGGAACTCGCTGCTCGGGAAGAAGTGCTTTGCTCTCAGGATGGCCA
GCCGGCTGGCCAAGGAGGAAGGGTGGCTGGCAGAGCACATGCTGATTCTGGGTATAACCAACCCTGAG
GGTGAGAAGAAGTACCTGGCGGCCGCATTTCCCAGCGCCTGCGGGAAGACCAACCTGGCCATGATGAA
CCCCAGCCTCCCCGGGTGGAAGGTTGAGTGCGTCGGGGATGACATTGCCTGGATGAAGTTTGACGCAC
AAGGTCATTTAAGGGCCATCAACCCAGAAAATGGCTTTTTCGGTGTCGCTCCTGGGACTTCAGTGAAG
ACCAACCCCAATGCCATCAAGACCATCCAGAAGAACACAATCTTTACCAATGTGGCCGAGACCAGCGA
CGGGGGCGTTTACTGGGAAGGTATTGATGAGCCGCTAGCTTCAGGCGTCACCATCACGTCCTGGAAGA
ATAAGGAGTGGAGCTCAGAGGATGGGGAACCTTGTGCCCACCCCAACTCGAGGTTCTGCACCCCTGCC
AGCCAGTGCCCCATCATTGATGCTGCCTGGGAGTCTCCGGAAGGTGTTCCCATTGAAGGCATTATCTT
TGGAGGCCGTAGACCTGCTGGTGTCCCTCTAGTCTATGAAGCTCTCAGCTGGCAACATGGAGTCTTTG
TGGGGGCGGCCATGAGATCAGAGGCCACAGCGGCTGCAGAACATAAAGGCAAAATCATCATGCATGAC
CCCTTTGCCATGCGGCCCTTCTTTGGCTACAACTTCGGCAAATACCTGGCCCACTGGCTTAGCATGGC
CCAGCACCCAGCAGCCAAACTGCCCAAGATCTTCCATGTCAACTGGTTCCGGAAGGACAAGGAAGGCA
AATTCCTCTGGCCAGGCTTTGGAGAGAACTCCAGGGTGCTGGAGTGGATGTTCAACCGGATCGATGGA
AAAGCCAGCACCAAGCTCACGCCCATAGGCTACATCCCCAAGGAGGATGCCCTGAACCTGAAAGGCCT
GGGGCACATCAACATGATGGAGCTTTTCAGCATCTCCAAGGAATTCTGGGAGAAGGAGGTGGAAGACA
TCGAGAAGTATCTGGAGGATCAAGTCAATGCCGACCTCCCCTGTGAAATCGAGAGAGAGATCCTTGCC
TTGAAGCAAAGAATAAGCCAGATGGTCGACGGC

| | | | |
|---|---|---|---|
| NOV1c, 278992862 | | | |
| Protein Sequence | SEQ ID NO:6 | 600 aa | MW at 66780.9 kD |

TGSEFLENNAELCQPDHIHICDGSEEEMGRLLGQMEEEGILRRLKKYDNCWLALTDPRDVARIESKTV
IVTQEQRDTVPIPKTGLSQLGRWMSEEDFEKAFNARFPGCMKGRTMYVIPFSMGPLGSPLSKIGIELT
DSPYVVASMRIMTRMGTPVLEALGDGEFVKCLHSVGCPLPLQKPLVNNWPCNPELTLIAHLPDRREII
SFGSGYGGNSLLGKKCFALRMASRLAKEEGWLAEHMLILGITNPEGEKKYLAAAFPSACGKTNLANMN
PSLPGWKVECVGDDIAWMKFDAQGHLRAINPENGFFGVAPGTSVKTNPNAIKTIQKNTIFTNVAETSD
GGVYWEGIDEPLASGVTITSWKNKEWSSEDGEPCAHPNSRFCTPASQCPIIDAAWESPEGVPIEGIIF
GGRRPAGVPLVYEALSWQHGVFVGAAMRSEATAAAEHKGKIIMHDPFAMRPFFGYNFGKYLAHWLSMA
QHPAAKLPKIFHVNWFRKDKEGKFLWPGFGENSRVLEWMFNRIDGKASTKLTPIGYIPKEDALNLKGL
GHINMNELFSISKEFWEKEVEDIEKYLEDQVNADLPCEIEREILALKQRISQMVDG

A ClustalW comparison of the above protein sequences yields the following sequence alignment shown in Table A7.

TABLE A7
Comparison of the NOV1 protein sequences

| | |
|---|---|
| NOV1a | ————MPPQLQNGLNLSAKVVQGSLDSLPQAVREFLENNAELCQPDHIHICDGSEEENGRL |
| NOV1b | TGSTMPPQLQNGLNLSAKVVQGSLDSLPQAVREFLENNAELCQPDHIHICDGSEEENGRL |
| NOV1c | —————————————————————————————TGSEFLENNAELCQPDHIHICDGSEEENGRL |
| NOV1a | LGQMEEEGILRRLKKYDNCWLALTDPRDVARIESKTVIVTQEQRDTVPIPKTGLSQLGRW |
| NOV1b | LGQMEEEGILRRLKKYDNCWLALTDPRDVARIESKTVIVTQEQRDTVPIPKTGLSQLGRW |
| NOV1c | LGQMEEEGILRRLKKYDNCWLALTDPRDVARIESKTVIVTQEQRDTVPIPKTGLSQLGRW |
| NOV1a | MSEEDFEKAFNARFPGCMKGRTMYVIPFSMGPLGSPLSKIGIELTDSPYVVASMRIMTRM |
| NOV1b | MSEEDFEKAFNARFPGCMKGRTMYVIPFSMGPLGSPLSKIGIELTDSPYVVASMRIMTRM |
| NOV1c | MSEEDFEKAFNARFPGCMKGRTMYVIPFSMGPLGSPLSKIGIELTDSPYVVASMRIMTRM |
| NOV1a | GTPVLEALGDGEFVKCLHSVGCPLPLQKPLVNNWPCNPELTLIAHLPDRREIISFGSGYG |
| NOV1b | GTPVLEALGDGEFVKCLHSVGCPLPLQKPLVNNWPCNPELTLIAHLPDRREIISFGSGYG |
| NOV1c | GTPVLEALGDGEFVKCLHSVGCPLPLQKPLVNNWPCNPELTLIAHLPDRREIISFGSGYG |
| NOV1a | GNSLLGKKCFALRMASRLAKEEGWLAEHMLILGITNPEGEKKYLAAAFPSACGKTNLAMM |
| NOV1b | GNSLLGKKCFALRMASRLAKEEGWLAEHMLILGITNPEGEKKYLAAAFPSACGKTNLAMM |
| NOV1c | GNSLLGKKCFALRMASRLAKEEGWLAEHMLILGITNPEGEKKYLAAAFPSACGKTNLAMM |
| NOV1a | NPSLPGWKVECVGDDIAWMKFDAQGHLRAINPENGFFGVAPGTSVKTNPNAIKTIQKNTI |
| NOV1b | NPSLPGWKVECVGDDIAWMKFDAQGHLRAINPENGFFGVAPGTSVKTNPNAIKTIQKNTI |
| NOV1c | NPSLPGWKVECVGDDIAWMKFDAQGHLRAINPENGFFGVAPGTSVKTNPNAIKTIQKNTI |
| NOV1a | FTNVAETSDGGVYWEGIDEPLASGVTITSWKNKEWSSEDGEPCAHPNSRFCTPASQCPII |
| NOV1b | FTNVAETSDGGVYWEGIDEPLASGVTITSWKNKEWSSEDGEPCAHPNSRFCTPASQCPII |
| NOV1c | FTNVAETSDGGVYWEGIDEPLASGVTITSWKNKEWSSEDGEPCAHPNSRFCTPASQCPII |
| NOV1a | DAAWESPEGVPIEGIIFGGRRPAGVPLVYEALSWQHGVFVGAANRSEATAAAEHKGKIIM |
| NOV1b | DAAWESPEGVPIEGIIFGGRRPAGVPLVYEALSWQHGVFVGAAMRSEATAAAEHKGKIIM |
| NOV1c | DAAWESPEGVPIEGIIFGGRRPAGVPLVYEALSWQHGVFVGAAMRSEATAAAEHKGKIIM |
| NOV1a | HDPFAMRPFFGYNFGKYLAHWLSMAQHPAAKLPKIFHVNWFRKDKEGKFLWPGFGENSRV |
| NOV1b | HDPFAMRPFFGYNFGKYLAHWLSMAQHPAAKLPKIFHVNWFRKDKEGKFLWPGFGENSRV |
| NOV1c | HDPFAMRPFFGYNFGKYLAHWLSMAQHPAAKLPKIFHVNWFRKDKEGKFLWPGFGENSRV |
| NOV1a | LEWMFNRIDGKASTKLTPIGYIPKEDALNLKGLGHINMMELFSISKEFWEKEVEDIEKYL |
| NOV1b | LEWMFNRIDGKASTKLTPIGYIPKEDALNLKGLGHINMMELFSISKEFWEKEVEDIEKYL |
| NOV1c | LEWMFNRIDGKASTKLTPIGYIPKEDALNLKGLGHINMMELFSISKEFWEKEVEDIEKYL |
| NOV1a | EDQVNADLPCEIEREILALKQRISQM——— |
| NOV1b | EDQVNADLPCEIEREILALKQRISQMVDG |
| NOV1C | EDQVNADLPCEIEREILALKQRISQMVDG |
| NOV1a | (SEQ ID NO:2) |
| NOV1b | (SEQ ID NO:4) |
| NOV1c | (SEQ ID NO:6) |

Further analysis of the NOV1a protein yielded the following properties shown in Table A8.

TABLE A8
Protein Sequence Properties NOV1a

| SignalP | No Known Signal Sequence Predicted | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 13; peak value −1.95 | | | |
| | PSG score: −6.35 | | | |
| | GvH: von Heijne's method for signal seq. recognition | | | |
| | GvH score (threshold: −2.1): −10.13 | | | |
| | possible cleavage site: between 17 and 18 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 6.42 (at 280) | | | |
| | ALOM score: 6.42 (number of TMSs: 0) | | | |
| | MITDISC: discrimination of mitochondrial targeting seq | | | |
| | R content: | 0 | Hyd Moment(75): | 3.20 |
| | Hyd Moment(95): | 6.24 | G content: | 2 |
| | D/E content: | 1 | S/T content: | 2 |

Score: −5.73
Gavel: prediction of cleavage sites for mitochondrial preseq
cleavage site motif not found
NUCDISC: discrimination of nuclear localization signals
pat4: none
pat7: none
bipartite: none
content of basic residues: 10.6%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: found
KLPK at 507
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear
discrimination
Prediction: cytoplasmic
Reliability: 89
COIL: Lupas's algorithm to detect coiled-coil regions
total: 0 residues
Final Results (k = 9/23):
43.5%: nuclear
34.8%: cytoplasmic
17.4%: mitochondrial
4.3%: vacuolar
>> prediction for CG101190-01 is nuc (k = 23)

A search of the NOV1a protein against the Geneseq database, a proprietary database that contains sequences published in patents and patent publication, yielded several homologous proteins shown in Table A9.

TABLE A9
Geneseq Results for NOV1a

| | | NOV1a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB71880 | Human PEPCK-cytosolic protein - | 1 . . . 622 | 617/622 (99%) | 0.0 |
| | Homo sapiens, 622 aa. [US6187545- | 1 . . . 622 | 620/622 (99%) | |
| | B1, 13-FEB-2001] | | | |
| AAB71890 | Mouse PEPCK-cytosolic protein - | 1 . . . 622 | 566/622 (90%) | 0.0 |
| | Mus musculus, 622 aa. [US6187545- | 1 . . . 622 | 596/622 (94%) | |
| | B1, 13-FEB-2001] | | | |
| AAY80296 | Human mitochondrial | 14 . . . 622 | 437/610 (71%) | 0.0 |
| | phosphoenolpyruvate carboxykinase | 31 . . . 640 | 514/610 (83%) | |
| | SEQ ID NO: 1 - Homo sapiens, 640 | | | |
| | aa. [US6030837-A, 29-FEB-2000] | | | |
| ABJ37938 | NOVX protein sequence SEQ ID No | 14 . . . 622 | 411/610 (67%) | 0.0 |
| | 121 - Unidentified, 608 aa. | 31 . . . 608 | 485/610 (79%) | |
| | [WO200281517-A2, 17-OCT-2002] | | | |
| ABB65318 | Drosophila melanogaster polypeptide | 18 . . . 622 | 397/610 (65%) | 0.0 |
| | SEQ ID NO 22746 - Drosophila | 43 . . . 647 | 477/610 (78%) | |
| | melanogaster, 647 aa. | | | |
| | [WO200171042-A2, 27-SEP-2001] | | | |

In a BLAST search of public sequence databases, the NOV1a protein was found to have homology to the proteins shown in the BLASTP data in Table A10.

TABLE A10
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P35558 | Phosphoenolpyruvate carboxykinase, | 1 . . . 622 | 622/622 (100%) | 0.0 |
| | cytosolic [GTP] (EC 4.1.1.32) | 1 . . . 622 | 622/622 (100%) | |
| | (Phosphoenolpyruvate carboxylase) | | | |
| | (PEPCK-C) - Homo sapiens | | | |
| | (Human), 622 aa. | | | |
| A45746 | phosphoenolpyruvate carboxykinase | 1 . . . 622 | 620/622 (99%) | 0.0 |
| | (GTP) (EC 4.1.1.32) 1 - human, 622 aa. | 1 . . . 622 | 621/622 (99%) | |
| P07379 | Phosphoenolpyruvate carboxykinase, | 1 . . . 622 | 566/622 (90%) | 0.0 |
| | cytosolic [GTP] (EC 4.1.1.32) | 1 . . . 622 | 596/622 (94%) | |
| | (Phosphoenolpyruvate carboxylase) | | | |
| | (PEPCK-C) - Rattus norvegicus | | | |
| | (Rat), 622 aa. | | | |
| Q9Z2V4 | Phosphoenolpyruvate carboxykinase, | 1 . . . 622 | 566/622 (90%) | 0.0 |
| | cytosolic [GTP] (EC 4.1.1.32) | 1 . . . 622 | 596/622 (94%) | |
| | (Phosphoenolpyruvate carboxylase) | | | |
| | (PEPCK-C) - Mus musculus | | | |
| | (Mouse), 622 aa. | | | |
| Q8BSX3 | Phosphoenolpyruvate carboxykinase | 1 . . . 622 | 566/622 (90%) | 0.0 |
| | 1 - Mus musculus (Mouse), 622 aa. | 1 . . . 622 | 595/622 (94%) | |

PFam analysis predicts that the NOV1a protein contains the domains shown in the Table A11.

TABLE A11
Domain Analysis of NOV1a

| | | Identities/ | |
|---|---|---|---|
| Pfam | NOV1a | Similarities for | Expect |
| Domain | Match Region | the Matched Region | Value |
| PEPCK | 29 . . . 622 | 434/612 (71%) | 0 |
| | | 567/612 (93%) | |

### EXAMPLE A4

### Human Cytosolic Phosphoenolpyruvate Carboxykinase (PEPCK) Gene Variants and SNPs

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing. Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265.

In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

Results

The DNA and protein sequences for the novel single nucleotide polymorphic variants of the phosphoenolpyruvate carboxykinase-like gene of CuraGen Acc. No. CG101190-01 are reported in Table A12. Variants are reported individually but any combination of all or a select subset of variants are also included. In Table A12, the positions of the variant bases and the variant amino acid residues are underlined. In summary, there are 16 variants reported in Table A12. Variant 13374203 is a G to A SNP at 187 bp of the nucleotide sequence that results in no change in the protein sequence (silent), variant 13374204 is a G to A SNP at 322 bp of the nucleotide sequence that results in no change in the protein sequence (silent), variant 13374205 is a C to T SNP at 400 bp of the nucleotide sequence that results in no change in the protein sequence (silent), variant 13374206 is a C to G SNP at 668 bp of the nucleotide sequence that results in a Leu to Val change at amino acid 184 of protein sequence, variant 13378862 is a G to A SNP at 868 bp of the nucleotide sequence that results in a Met to Ile change at amino acid 250 of protein sequence, variant 13380271 is an A to G SNP at 917 bp of the nucleotide sequence that results in an Ile to Val change at amino acid 267 of protein sequence, variant 13378861 is a G to A SNP at 944 bp of the nucleotide sequence that results in a Glu to Lys change at amino acid 276 of protein sequence, variant 13379542 is a C to T SNP at 1258 bp of the nucleotide sequence that results in no change in the protein sequence (silent), variant 13378352 is a C to A SNP at 1339 bp of the nucleotide sequence that results in a Cys to Stop change at amino acid 407 of protein sequence, variant 13375322 is an A to G SNP at 1680 bp of the nucleotide sequence that results in a Lys to Arg change at amino acid 521 of protein sequence, variant 13375321 is an A to G SNP at 1731 bp of the nucleotide sequence that results in a Glu to Gly change at amino acid 538 of protein sequence, variant 13375320 is a T to C SNP at 1773 bp of the nucleotide sequence that results in a Leu to Pro change at amino acid 552 of protein sequence, variant 13375319 is a T to C SNP at 1848 bp of the nucleotide sequence that results in a Leu to Pro change at amino acid 577 of protein sequence, variant 13375318 is a T to C SNP at 1857 bp of the nucleotide sequence that results in an Ile to Thr change at amino acid 580 of protein sequence, variant 13377375 is an A to G SNP at 1887 bp of the nucleotide sequence that results in a Glu to Gly change at amino acid 590 of protein sequence, and variant 13377374 is a T to C SNP at 1929 bp of the nucleotide sequence that results in a Leu to Pro change at amino acid 604 of protein sequence.

TABLE A12
Variants of nucleotide sequence of
Acc. No. CG101190-01 (SEQ ID NO: 1)

| | Nucleotides | | | Amino Acids | | |
|---|---|---|---|---|---|---|
| Variant | Position | Initial | Modified | Position | Initial | Modified |
| 13374203 | 187 | G | A | 23 | Leu | Leu |
| 13374204 | 322 | G | A | 68 | Arg | Arg |
| 13374205 | 400 | C | T | 94 | Ile | Ile |
| 13374206 | 668 | C | G | 184 | Leu | Val |
| 13378862 | 868 | G | A | 250 | Met | Ile |
| 13380271 | 917 | A | G | 267 | Ile | Val |
| 13378861 | 944 | G | A | 276 | Glu | Lys |
| 13379542 | 1258 | C | T | 380 | Gly | Gly |
| 13378352 | 1339 | C | A | 407 | Cys | STOP |
| 13375322 | 1680 | A | G | 521 | Lys | Arg |
| 13375321 | 1731 | A | G | 538 | Glu | Gly |
| 13375320 | 1773 | T | C | 552 | Leu | Pro |
| 13375319 | 1848 | T | C | 577 | Leu | Pro |
| 13375318 | 1857 | T | C | 580 | Ile | Thr |
| 13377375 | 1887 | A | G | 590 | Glu | Gly |
| 13377374 | 1929 | T | C | 604 | Leu | Pro |

TABLE A13
Variant Sequences

```
Table A13A1.
Nucleotide sequence of variant 13374203 NOV1a1n (underlined). G/A (SEQ ID NO:79)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTACCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGCCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGCAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13A2. Protein sequence of variant NOV1a1p (underlined). (SEQ ID NO:80)
      1 MPPQLQNGLNLSAKVVQGSLDSLPQAVREFLENNAELCQPDHIHICDGSEEENGRLLGOMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRNGTPVLEALGDGEPVKCLHSVGCPLPLQKPLVNNWPCNPELTLIAHLPDRREIISFGSGYGGNSL
  241 LGKKCFALRMASRLAKEEGWLAEHMLILGITNPEGEKXYLAAAFPSACGKTNLANMNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
401 HPNSRFCTPASQCPIIDAAWESPEGVPIEGIIFGGRRPAGVPLVYEALSWQHGVFVGAANRSEATAAAEHKGKIIMHDPF
481 ANRPFFGYNFGKYLAHWLSMAQHPAAKLPKIFHVNWFRKDKEGKFLWPGFGENSRVLEWNFNRIDGKASTKLTPIGYIPK
561 EDALNLKGLGHINMMELFSISKEFWEKEVEDIEKYLEDQVNADLPCEIEREILALKQRISQM
Table A13A3. Alteration effect
No change.
Table A13B1.
Nucleotide sequence of variant 13374204 NOV1a2n (underlined). G/A (SEQ ID NO:81)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GACTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGCTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13B2. Protein sequence of variant NOV1a2p (underlined). (SEQ ID NO:82)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRNGTPVLEALGDGEFVKCLHSVGCPLPLQKPLVNNWPCNPELTLIAHLPDRREIISFGSGYGGNSL
  241 LGKKCFALRMASRLAKEEGWLAEHMLILGITNPEGEKKYLAAAFPSACGKTNLANMNPSLPGWKVECVGDDIAWNKFDAQ
  321 GHLRAINPENGFFGVAPGTSVKTNPHAIKTIQKNTIFTNVAETSDGGVYWEGIDEPLASGVTITSWKNKEWSSEDGEPCA
  401 HPNSRFCTPASOCPIIDAAWESPEGVPIEGIIFGGRRPAGVPLVYEALSWQHGVFVGAANRSEATAAAEHKGKIIMHDPF
  481 ANRPFFGYNFGKYLAHWLSMAQHPAAKLPKIFHVNWFRKDKEGKFLWPGFGENSRVLEWMFNRIDGKASTKLTPIGYIPK
  561 EDALNLKGLGHINMMELFSISKEFWEKEVEDIEKYLEDQVNADLPCEIEREILALKQRISQM
Table A13B3. Alteration effect
No change.
Table A13C1.
Nucleotide sequence of variant 13374205 NOV1a3n (underlined). C/T (SEQ ID NO:83)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTCGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATT
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTCGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTCAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13C2. Protein sequence of variant NOV1a3p (underlined). (SEQ ID NO:84)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRMGTPVLEALGDGEFVKCLHSVGCPLPLQKPLVWNWPCNPELTLIAHLPDRREIISFGSGYGGWSL
  241 LGKKCFALRMASRLAKEECWLAEHMLILGITNPEGEKKYLAAAFPSACGKTNLANMNPSLPGWKVECVGDDIAWMKFDAQ
  321 GHLRAINPENGFFGVAPGTSVKTNPNAIKTIOKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAAMRSEATAAAEHKGKIIMHDPF
  481 AMRPFFGYNFGKYLAHWLSMAQHPAAKLPKIFHVNWFRKDKEGKFLWPGFGENSRVLEWMFNRIDGKASTKLTPIGYIPK
  561 EDALNLKGLGHINNMELPSISKEFWEKEVEDIEKYLEDQVNADLPCEIEREILALKQRISQM
Table A13C3. Alteration effect
No change.
Table A13D1.
Nucleotide sequence of variant 13374206 NOV1a4n (underlined). G/G (SEQ ID NO:85)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCAGTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGCATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCIG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAACTCAA
1921 TGCCGACCTCCCCTGTGAAATCQAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13D2. Protein sequence of variant NOV1a4p (underlined). (SEQ ID NO:86)
      1 MPPQLQNGLNLSAKVVOGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWHSEEDFEKAFNARFPGCMKGRTMYVIPFSMGPLGSPLSKIGIEL
  161 TDSPYVVASMRIMTRNGTPVLEAVGDGEFVKCLHSVGCPLPLQKPLVNNWPCNPELTLIAHLPDRREIISFGSGYGGNSL
  241 LGKKCFALRMASRLAKEEGWLAEHMLILGITNPEGEKKYLAAAFPSACGKTNLAMMNPSLPGWKVECVGDDIAWMKFDAQ
  321 GHLRAINPENGFFGVAPGTSVKTNPNAIKTIQKNTIFINVAETSDGGVYWEGIDEPLASGVTITSWKNKEWSSEDGEPCA
  401 HPHSRFCTPASQCPIIDAAWESPEGVPIEGIIFGGRRPAGVPLVYEALSWQHGVFVGAANRSEATAAAEHKGKIIMHDPF
  481 ANRPFFGYNFGKYLAHWLSMAQHPAAKLPKIFHVNWFRKDKEGKFLWPGFGENSRVLEWMFNRIDGKASTKLTPIGYIPK
  561 EDALNLKGLGHINMMELFSISKEFWEKEVEDIEKYLEDQVNADLPCEIEREILALKQRISQM
Table A13D3. Alteration effect
Leu to Val
Table A13E1.
Nucleotide sequence of variant 13378862 NOV1a5n (underlined). G/A (SEQ ID NO:87)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATAGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13E2. Protein sequence of variant (underlined) NOV1a5p. (SEQ ID NO:88)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRNGTPVLEALGDGEFVKCLHSVGCPLPLQKPLVNNWPCNPELTLIAHLPDRREIISFGSGYGGNSL
  241 LGKKCFALRIASRLAKEEGWLAEHMLILGITNPEGEKKYLAAAFPSACGKTNLANMNPSLPGWKVECVGDDIAWNKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAAMRSEATAAAEHKGKIIMHDPF
  481 ANRPFFGYNFGKYLAHWLSMAQHPAAKLPKIFHVNWFRKDKEGKFLWPGFGENSRVLEWNFNRIDGKASTKLTPIGYIPK
  561 EDALNLKGLGHINMMELFSISKEFWEKEVEDIEKYLEDOVNADLPCEIEREILALKQRISQM
Table A13E3. Alteration effect
Met to Ile
Table A13F1.
Nucleotide sequence of variant 13380271 NOV1a6n (underlined). A/G (SEQ ID NO:89)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGCCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGGTTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAQACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACcTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13F2. Protein sequence of variant NOV1a6p (underlined). (SEQ ID NO:90)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRNGTPVLEALGDGEFVKCLHSVGCPLPLQKPLVNNWPCNPELTLIAHLPDRREIISPGSGYGGNSL
  241 LGKKCFALRMASRLAKEEGWLAEHMLVLGITNPEGEKXYLAAAFPSACGKTNLAMMNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAANRSEATAAAEHKGKIIMHDPF
  481 AMRPFFGYNFGKYLAHWLSMAQHPAAKLPKIFHVNWFRKDKEGKFLWPGFGENSRVLEWMFNRIDGKASTKLTPIGYIPK
  561 EDALNLKGLGHINNHELFSISKEFWEKEVEDIEKYLEDQVNADLPCEIEREILALKQRISQM
Table A13F3. Alteration effect
Ile to Val
Table A13G1.
Nucleotide sequence of variant 13378861 NOV1a7n (underlined). G/A (SEQ ID NO:91)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGcTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGCGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTAAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13G2. Protein sequence of variant NOV1a7p (underlined). (SEQ ID NO:92)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSOLGRWMSEEDFEKAFNARFPGCMKGRTMYVIPFSMGPLGSPLSKIGIEL
  161 TDSPYVVASMRIMTRNGTPVLEALGDGEFVKCLHSVGCPLPLQKPLVNNWPCNPELTLIAHLPDRREIISFGSGYGGNSL
  241 LGKKCFALRMASRLAKEEGWLAEHNLILGITNPEGKKKYLAAAFPSACGKTNLANMNPSLPGWKVECVGDDIAWNKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAANRSEATAAAEHKGKIIMHDPF
  481 AMRPFFGYNFGKYLAHWLSMAQHPAAKLPKIFHVNMFRKDKEGKFLWPGFGENSRVLEWMFNRIDGKASTKLTPIGYIPK
  561 EDALNLKGLGHINMMELFSISKEFWEKEVEDIEKYLEDQVNADLPCEIEREILALKQRISQM
Table A13G3. Alteration effect
Glu to Lys
Table A13H1.
Nucleotide sequence of variant 13379542 NOV1a8n (underlined). C/T (SEQ ID NO:93)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CCGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGTGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATCGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13H2. Protein sequence of variant NOV1a8p (underlined). (SEQ ID NO:94)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWHSEEDFEKAFNARFPGCHKGRTMYVIPFSMGPLGSPLSKIGIEL
  161 TDSPYVVASMRIMTRMGTPVLEALGDGEFVKCLHSVGCPLPLQKPLVNNWPCNPELTLIAHLPDRREIISFGSGYGGNSL
  241 LGKKCFALRMASRLAKEEGWLAEHHLILGITNPEGEKKYLAAAFPSACGKTNLANMNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAANRSEATAAAEHKGKIIMHDPF
  481 ANRPFFGYNFGKYLAHWLSMAQHPAAKLPKIFHVNWFRKDKEGKFLWPGFGENSRVLEWHFNRIDGKASTKLTPIGYIPK
  561 EDALHLKGLGHINMMELFSISKEFWEKEVEDIEKYLEDQVNADLPCEIEREILALKQRISQM
Table A13H3. Alteration effect
No change.
Table A1311.
Nucleotide sequence of variant 13378352 NOV1a9n (underlined). C/A (SEQ ID NO:95)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGAACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A 1312. Protein sequence of variant NOV1a9p (underlined). (SEQ ID NO:96)
      1 MPPQLQNGLNLSAKVVQGSLDSLPQAVREFLENNAELCQPDHIHICDGSEEENGRLLGOMEEEGILRRLKKYDNCWLALT
    81 DPRDVARIESKTVIVTQEQRDTVPIPKTGLSQLGRWMSEEDFEKAFNARFPGCHKGRTMYVIPFSMGPLGSPLSKIGIEL
  161 TDSPYVVASMRIMTRNGTPVLEALGDGEFVKCLHSVGCPLPLQKPLVNNNPONPELTLIAHLPDRREIISFGSGYGGNSL
  241 LGKKCFALRMASRLAKEEGWLAEHMLILGITNPEGEKKYLAAAFPSACGKTNLANMNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRF*TPASQCPIIDAAWESPEGVPIEGIIFGGRRPAGVPLVYEALSWQHGVFVGAANRSEATAAAEHKGKIIMHDPF
  481 ANRPFFGYNFGKYLAHWLSMAQHPAAKLPKIFHVNWFRKDKEGKFLWPGFGEHSRVLEWHFNRIDGKASTKLTPIGYIPK
  561 EDALNLKGLGHINMMELFSISKEFWEKEVEDIEKYLEDQVNADLPCEIEREILALKQRISQM
Table A 1313. Alteration effect
Cys to STOP
Table A13J1.
Nucleotide sequence of variant 13375322 NOV1a10n (underlined). A/G (SEQ ID NO:97)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATCCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAG
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGCCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13J2. Protein sequence of variant NOV1a10p (underlined). (SEQ ID NO:98)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTNYVIPFSMGPLGSPLSKIGIEL
  161 TDSPYVVASHRIMThNGTPVLEALGDGEFVKCLHSVGCPLPLQKPLVNNWPCNPELTLIAHLPDRREIISFGSGYGGNSL
  241 LGKKCFALRMASRLAKEEGWLAEHMLILGITNPEGEKKYLAAAFPSACGKTNLANMNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAAMRSEATAAAEHKGKIIMHDPF
  481 ANRPFFCYNFGKYLAHWLSMAQHPAAKLPKIFHVHWFRKDREGKFLWPGFGENSRVLEWMFNRIDGKASTKLTPIGYIPK
  561 EDALNLKGLGHINNMELFSISKEFWEKEVEDIEKYLEDQVNADLPCEIEREILALKORISQM
Table A13J3. Alteration effect
Lys to Arg
Table A13K1.
Nucleotide sequence of variant 13375321 NOV1a11n (underlined). A/G (SEQ ID NO:99)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGGGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13K2. Protein sequence of variant NOV1a11p (underlined). (SEQ ID NO:100)
      1 HPPQLQNGLNLSAKVVQGSLDSLPQAVREFLENNAELCQPDHIHICDGSEEEHGRLLGQMEEEGILRRLKKYDNCWLALT
    81 DPRDVARIESKTVIVTQEQRDTVPIPKTGLSQLGRWMSEEDFEKAFHARFPGCMKGRTHYVIPFSMGPLGSPLSKIGIEL
  161 TDSPYVVASMRIMTRNGTPVLEALGDGEFVKCLHSVGCPLPLQKPLVNNWPCNPELTLIAHLPDRREIISFGSGYGGNSL
  241 LGKKCFALRMASRLAKEEGWLAEHMLILGITNPEGEKKYLAAAFPSACGKTNLANMNPSLPGWKVECVGDDIAWNKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAANRSEATAAAEHKGKIIMHDPF
  481 ANRPFFGYNFGKYLAHWLSMAQHPAAKLPKIFHVNWFRKDKEGKFLWPGFGEMSRVLGWMFNRIDGKASTKLTPIGYIPK
  561 EDALNLKGLGHINMMELFSISKEFWEKEVEDIEKYLEDQVNADLPCEIEREILALKQRISQM
Table A13K3. Alteration effect
Glu to Gly
Table A13L1.
Nucleotide sequence of variant 13375320 NOV1a12n (underlined). T/C (SEQ ID NO:101)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCCTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCCCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGACCAAGAGAGGGCAAGTGTTCC
Table A13L2. Protein sequence of variant NOV1a12p (underlined). (SEQ ID NO:102)
      1 MPPQLQWGLNISAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRNGTPVLEALGDGEFVKCLHSVGCPLPLQKPLVNNWPCNPELTLIAHLPDRREIISFGSGYGGNSL
  241 LGKKCFALRMASRLAKEEGWLAEHMLILGITNPEGEKKYLAAAFPSACGKTNLANMNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAANRSEATAAAEHKGKIIHHDPF
  481 AMRPFFGYNFGKYLAHWLSMAOHPAAKLPKIFHVNWFRKDKEGKFLWPGFGENSRVLEWMFNRIDGKASTKPTPIGYIPK
  561 EDALNLKGLGHINMMELFSISKEFWEKEVEDIEKYLEDQVNADLPCEIEREILALKQRISQM
Table A13L3. Alteration effect
Leu to Pro
Table A13M1.
Nucleotide sequence of variant 13375319 NOV1a13n (underlined). T/C (SEQ ID NO:103)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACcrG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCCTTTCAGCATCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13M2. Protein sequence of variant (underlined) NOV1a13p. (SEQ ID NO:104)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRMGTPVLEALGDGEFVKCLHSVGCPLPLQKPLVNNWPCNPELTLIAHLPDRREIISFGSGYGGNSL
  241 LGKKCFALRNASRLAKEEGWLAEHMLILCITNPEGEKKYIAAAFPSACGKTNLAMHNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAANRSEATAAAEHKGKIIMHDPF
  481 ANRPFFGYNFGKYLAHWLSMAQHPAAKLPKIFHVNWFRKDKEGKFLWPGFGENSRVLEWMPNRIDGKASTKLTPIGYIPK
  561 EDALNLKGLGHINMMEPFSISKEFWEKEVEDIEKYLEDQVNADLPCEIEREILALKQRISQM
Table A13M3. Alteration effect
Leu to Pro
Table A13N1.
Nucleotide sequence of variant 13375318 NOV1a14n (underlined). T/C (SEQ ID NO:105)
      1 GAACACAAACTTGCTGGCGGGAAGAGCCCGGAAAGAAACCTGTGGATCTCCCTTCGAGATCATCCAAAGAGAAGAAAGGT
    81 GACCTGACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCACCTCCAAGGAATTCTGGGAGAAGGAGGTGGAAGACATCGAGAAGTATCTGGAGGATCAAGTCAA
1921 TGCCGACCTCCCCTGTGAAATCGAGAGAGAGATCCTTGCCTTGAAGCAAAGAATAAGCCACATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13N2. Protein sequence of variant NOV1a14p (underlined). (SEQ ID NO:106)
      1 MPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTOEQRDTVPIPKTGLSQGRWIISEEDFEKAFNARFPGCMKGRTMYVIPFSMGPLGSPLSKIGIEL
  161 TDSPYVVASMRIMTRMGTPVLEALGDGEFVKCLHSVGCPLPLQKPLVNNWPCNPELTLIAHLPDRREIISFGSGYGGNSL
  241 LGKKCFALRMASRLAKEEGWLAEHNLILGITNPEGEKKYLAAAFPSACGKTNLANMNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAAMRSEATAAAEHKGKIIMHDPF
  481 AMRPFFGYNFGKYLAHWLSMAQHPAAKLPKIFHVNWFRKDKEGKFLWPGFGENSRVLEWMFNRIDGKASTKLTPIGYIPK
  561 EDALNLKGLGHINMMELFSTSKEFWEKEVEDIEKYLEDQVNADLPCEIEREILALKQRISQM
Table A13N3. Alteration effect
Ile to Thr
Table A1301.
Nucleotide sequence of variant 13377375 NOV1a15n (underlined). A/G (SEQ ID NO:107)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGGACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCTCAGCATGGCCACCCGGCTG
  881 GCCAAGGAGGAAGGGTCGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAACGCAAAATCATCATCCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGGAGACATCGAGAAGTATCTGGAGGATCAAGTCAA
1921 TGCCGACCTCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A1302. Protein sequence of variant NOV1a15p (underlined). (SEQ ID NO:108)
      1 HPPQLQNGLNLSAKVVQGSLDSLPQAVREFLENNAELCQPDHIHICDGSEEENGRLLGQMEEEGILRRLKKYDNCWLALT
    81 DPRDVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRMGTPVLEALGDGEFVKCLHSVGCPLPLQKPLVNNWPCNPELTLIAHLPDRREIISFGSGYGGNSL
  241 LGKKCFALRMASRLAKEEGWLAEHNLILGITNPEGEKKYLAAAFPSACGKTNLANMNPSLPGWKVECVGDDIAWHKFDAQ
  321 GELRAINPENGFFGVAPGTSVKTNPNAIKTIQKNTIFTNVAETSDGGVYWEGIDEPLASGVTITSWKNKEWSSEDGEPCA
  401 HPNSRFGTPASQCPIIDAAWESPEGVPIEGIIFGGRRPAGVPLVYEALSWQHGVFVGAANRSEATAAAEHKGKIIMHDPF
  481 AMRPFFGYNFGKYLAHWLSMAQHPAAKLPKIFHVNWFRKDKEGKFLWPGFGENSRVLEWMFNRIDGKASTKLTPIGYIPK
  561 EDALNLKGLGHINNMELFSISKEFWEKEVGDIEKYLEDQVNADLPCEIEREILALKQRISQM
Table A1303. Alteration effect
Glu to Gly
Table A13P1.
Nucleotide sequence of variant 13377374 NOV1a16n (underlined). T/C (SEQ IDNO:109)
      1 GAACACAAACTTGCTGGCGGGAAGAGCCCGGAAAGAAACCTGTGGATCTCCCTTCGAGATCATCCAAAGAGAAGAAAGGT
    81 GACCTCACATTCGTGCCCCTTAGCAGCACTCTGCAGAAATGCCTCCTCAGCTGCAAAACGGCCTGAACCTCTCGGCCAAA
  161 GTTGTCCAGGGAAGCCTGGACAGCCTGCCCCAGGCAGTGAGGGAGTTTCTCGAGAATAACGCTGAGCTGTGTCAGCCTGA
  241 TCACATCCACATCTGTGACGGCTCTGAGGAGGAGAATGGGCGGCTTCTGGGCCAGATGGAGGAAGAGGGCATCCTCAGGC
  321 GGCTGAAGAAGTATGACAACTGCTGGTTGGCTCTCACTGACCCCAGGGATGTGGCCAGGATCGAAAGCAAGACGGTTATC
  401 GTCACCCAAGAGCAAAGAGACACAGTGCCCATCCCCAAAACAGGCCTCAGCCAGCTCGGTCGCTGGATGTCAGAGGAGGA
  481 TTTTGAGAAAGCGTTCAATGCCAGGTTCCCAGGGTGCATGAAAGGTCGCACCATGTACGTCATCCCATTCAGCATGGGGC
  561 CGCTGGGCTCACCTCTGTCGAAGATCGGCATCGAGCTGACGGATTCGCCCTACGTGGTGGCCAGCATGCGGATCATGACG
  641 CGGATGGGCACGCCCGTCCTGGAAGCACTGGGCGATGGGGAGTTTGTCAAATGCCTCCATTCTGTGGGGTGCCCTCTGCC
  721 TTTACAAAAGCCTTTGGTCAACAACTGGCCCTGCAACCCGGAGCTGACGCTCATCGCCCACCTGCCTGACCGCAGAGAGA
  801 TCATCTCCTTTGGCAGTGGGTACGGCGGGAACTCGCTGCTCGGGAAGAAGTGCTTTGCTCACAGGATGGCCAGCCGGCTG
  881 GCCAAGGAGGAAGGGTGGCTGGCAGAGCACATGCTGATTCTGGGTATAACCAACCCTGAGGGTGAGAAGAAGTACCTGGC
  961 GGCCGCATTTCCCAGCGCCTGCGGGAAGACCAACCTGGCCATGATGAACCCCAGCCTCCCCGGGTGGAAGGTTGAGTGCG
1041 TCGGGGATGACATTGCCTGGATGAAGTTTGACGCACAAGGTCATTTAAGGGCCATCAACCCAGAAAATGGCTTTTTCGGT
1121 GTCGCTCCTGGGACTTCAGTGAAGACCAACCCCAATGCCATCAAGACCATCCAGAAGAACACAATCTTTACCAATGTGGC
1201 CGAGACCAGCGACGGGGGCGTTTACTGGGAAGGTATTGATGAGCCGCTAGCTTCAGGCGTCACCATCACGTCCTGGAAGA
1281 ATAAGGAGTGGAGCTCAGAGGATGGGGAACCTTGTGCCCACCCCAACTCGAGGTTCTGCACCCCTGCCAGCCAGTGCCCC
1361 ATCATTGATGCTGCCTGGGAGTCTCCGGAAGGTGTTCCCATTGAAGGCATTATCTTTGGAGGCCGTAGACCTGCTGGTGT
1441 CCCTCTAGTCTATGAAGCTCTCAGCTGGCAACATGGAGTCTTTGTGGGGGCGGCCATGAGATCAGAGGCCACAGCGGCTG
1521 CAGAACATAAAGGCAAAATCATCATGCATGACCCCTTTGCCATGCGGCCCTTCTTTGGCTACAACTTCGGCAAATACCTG
1601 GCCCACTGGCTTAGCATGGCCCAGCACCCAGCAGCCAAACTGCCCAAGATCTTCCATGTCAACTGGTTCCGGAAGGACAA
1681 GGAAGGCAAATTCCTCTGGCCAGGCTTTGGAGAGAACTCCAGGGTGCTGGAGTGGATGTTCAACCGGATCGATGGAAAAG
1761 CCAGCACCAAGCTCACGCCCATAGGCTACATCCCCAAGGAGGATGCCCTGAACCTGAAAGGCCTGGGGCACATCAACATG
1841 ATGGAGCTTTTCAGCATCTCCAAGGAATTCTGGGAGAAGGAGGTGGAAGACATCGAGAAGTATCTGGAGGATCAAGTCAA
1921 TGCCGACCCCCCCTGTGAAATCGAGAGAGAGATCCTTGCCTTGAAGCAAAGAATAAGCCAGATGTAATCAGGGCCTGAGT
2001 GCTTTACCTTTAAAATCATTCCCTTTCCCATCCATAAGGTGCAGTAGGAGCAAGAGAGGGCAAGTGTTCC
Table A13P2. Protein sequence of variant NOV1a16p (underlined). (SEQ ID NO:110)
      1 MPPQLQNGLNLSAKVVQGSLDSLPQAVREFLENNAELCQPDHIHICDGSEEENGRLLGQMEEEGILRRLKKYDNCWLALT
    81 OP DVARIESKTVIVTQEQRDTVPIPKTGLSQLGRWMSEEDFEKAFNARFPGCMKGRTMYVIPFSMGPLGSPLSKIGIEL
  161 TDSPYVVASMRIMTRNGTPVLEALGDGEFVKCLHSVGCPLPLQKPLVNNWPCNPELTLIAHLPDRREIISFGSGYGGNSL
  241 LGKKCFALRNASRLAKEEGWLAEHMLILGITNPEGEKKYLAAAPPSACGKTNLAMNNPSLPGWKVECVGDDIAWMKFDAQ
  321 GHLRAINPENGFFGVAPGTSVKTNPNAIKTIQKNTIFTNVAETSDGGVYWEGIDEPLASGVTITSWKNKEWSSEDGEPCA
  401 HPNSRFCTPASQCPIIDAAWESPEGVPIEGIIFGGRRPAGVPLVYEALSWQHGVFVGAANRSEATAAAEHKGKIIMHDPF
  481 AMRPFFGYNFGKYLAHWLSMAQHPAAKLPKIFHVNWFRKDKEGKFLWPGFGENSRVLEWMFNRIDGKASTKLTPIGYIPK
  561 EDALNLKGLGHINMMELFSISKEFWEKEVEDIEKYLEDQVNADPPCEIEREILALKQRISQM
Table A13P3. Alteration effect
Leu to Pro
```

### EXAMPLE A5

### Expression Profile of the Human Cytosolic Phosphoenolpyruvate Carboxykinase (PEPCK) Gene

The protocol for quantitative expression analysis is disclosed in Example Q9.

Expression of gene CG101190-01 was assessed using the primer-probe set Ag1769, described in Table A14. Results of the RTQ-PCR runs are shown in Tables A15, A16, and A17.

TABLE A14
Probe Name Ag1769

| | | | Start | SEQ ID |
|---|---|---|---|---|
| Primers | Sequences | Length | Position | No |
| Forward | 5′-gcagaacataaaggcaaaatca-3′ | 22 | 1520 | 175 |
| Probe | TET-5′-tcatgcatgacccctttgccatg-3′- | 23 | 1542 | 176 |
| | TAMRA | | | |
| Reverse | 5′-caggtatttgccgaagttgtag-3′ | 22 | 1579 | 177 |

TABLE A15
Panel 1.3D

| | Rel. Exp. (%) |
|---|---|
| | Ag1769, Run |
| Tissue Name | 156228299 |
| Liver adenocarcinoma | 0.0 |
| Pancreas | 0.0 |
| Pancreatic ca. CAPAN 2 | 0.0 |
| Adrenal gland | 0.7 |
| Thyroid | 0.1 |
| Salivary gland | 0.0 |
| Pituitary gland | 5.0 |
| Brain (fetal) | 0.0 |
| Brain (whole) | 0.1 |
| Brain (amygdala) | 0.0 |
| Brain (cerebellum) | 0.0 |
| Brain (hippocampus) | 0.0 |
| Brain (substantia nigra) | 0.0 |
| Brain (thalamus) | 0.1 |
| Cerebral Cortex | 0.0 |
| Spinal cord | 0.0 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.0 |
| Heart (fetal) | 0.0 |
| Heart | 0.3 |
| Skeletal muscle (fetal) | 0.2 |
| Skeletal muscle | 0.0 |
| Bone marrow | 0.1 |
| Thymus | 0.2 |
| Spleen | 0.0 |
| Lymph node | 0.2 |
| Colorectal | 11.7 |
| Stomach | 0.1 |
| Small intestine | 2.0 |
| Colon ca. SW480 | 0.0 |
| Colon ca.* SW620(SW480 met) | 0.1 |
| Colon ca. HT29 | 0.0 |
| Colon ca. HCT-116 | 0.0 |
| Colon ca. CaCo-2 | 0.2 |
| Colon ca. tissue(ODO3866) | 1.0 |
| Colon ca. HCC-2998 | 0.0 |
| Gastric ca.* (liver met) NCI-N87 | 0.0 |
| Bladder | 0.0 |
| Trachea | 0.8 |
| Kidney | 29.5 |
| Kidney (fetal) | 32.3 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 100.0 |
| Liver (fetal) | 34.6 |
| Liver ca. (hepatoblast) HepG2 | 0.6 |
| Lung | 0.3 |
| Lung (fetal) | 0.1 |
| Lung ca. (small cell) LX-1 | 0.3 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.1 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 4.4 |
| Breast ca.* (pl. ef) MCF-7 | 0.1 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.3 |
| Ovarian ca. OVCAR-3 | 0.5 |
| Ovarian ca. OVCAR-4 | 0.0 |
| Ovarian ca. OVCAR-5 | 0.1 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 0.0 |
| Placenta | 0.0 |
| Prostate | 0.0 |
| Prostate ca.* (bone met)PC-3 | 0.0 |
| Testis | 0.0 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 3.8 |

TABLE A16
Panel 5 Islet

| | Rel. Exp. (%) |
|---|---|
| | Ag1769, Run |
| Tissue Name | 174269005 |
| 97457_Patient-02go_adipose | 3.1 |
| 97476_Patient-07sk_skeletal muscle | 0.1 |
| 97477_Patient-07ut_uterus | 0.0 |
| 97478_Patient-07pl_placenta | 0.0 |
| 99167_Bayer Patient 1 | 1.4 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.1 |
| 97493_Patient-10pl_placenta | 0.0 |
| 97495_Patient-11go_adipose | 3.2 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 6.4 |
| 97501_Patient-12sk_skeletal muscle | 0.2 |
| 97502_Patient-12ut_uterus | 0.0 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.9 |
| 94710_Donor 2 AM - B_adipose | 0.7 |
| 94711_Donor 2 AM - C_adipose | 0.7 |
| 94712_Donor 2 AD - A_adipose | 3.1 |
| 94713_Donor 2 AD - B_adipose | 5.8 |
| 94714_Donor 2 AD - C_adipose | 4.3 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.3 |
| 94731_Donor 3 AM - B_adipose | 0.0 |
| 94732_Donor 3 AM - C_adipose | 0.0 |
| 94733_Donor 3 AD - A_adipose | 1.4 |
| 94734_Donor 3 AD - B_adipose | 0.4 |
| 94735_Donor 3 AD - C_adipose | 0.5 |
| 77138 Liver HepG2untreated | 18.7 |
| 73556_Heart_Cardiac stromal cells (primary) | 0.0 |
| 81735_Small Intestine | 16.6 |
| 72409_Kidney_Proximal Convoluted Tubule | 0.1 |
| 82685_Small intestine_Duodenum | 100.0 |
| 90650_Adrenal_Adrenocortical adenoma | 0.0 |
| 72410_Kidney_HRCE | 0.2 |
| 72411_Kidney_HRE | 0.0 |
| 73139_Uterus_Uterine smooth muscle cells | 0.0 |

TABLE A17
General_screening_panel_v1.7

| Tissue Name | A |
|---|---|
| Adipose | 13.4 |
| HUVEC | 0.0 |
| Melanoma* Hs688(A).T | 0.0 |
| Melanoma* Hs688(B).T | 0.0 |
| Melanoma (met) SK-MEL-5 | 0.0 |
| Testis | 0.2 |
| Prostate ca. (bone met) PC-3 | 0.0 |
| Prostate ca. DU145 | 0.0 |
| Prostate pool | 0.0 |
| Uterus pool | 0.0 |
| Ovarian ca. OVCAR-3 | 0.1 |
| Ovarian ca. (ascites) SK-OV-3 | 0.0 |
| Ovarian ca. OVCAR-4 | 0.4 |
| Ovarian ca. OVCAR-5 | 0.0 |
| Ovarian ca. IGROV-1 | 0.0 |
| Ovarian ca. OVCAR-8 | 0.0 |
| Ovary | 0.1 |
| Breast ca. MCF-7 | 0.0 |
| Breast ca. MDA-MB-231 | 0.0 |
| Breast ca. BT 549 | 0.0 |
| Breast ca. T47D | 0.0 |
| 113452 mammary gland | 0.0 |
| Trachea | 2.1 |
| Lung | 0.2 |
| Fetal Lung | 1.1 |
| Lung ca. NCI-N417 | 0.0 |
| Lung ca. LX-1 | 0.1 |
| Lung ca. NCI-H146 | 0.3 |
| Lung ca. SHP-77 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Lung ca. DMS-114 | 0.0 |
| Liver | 65.1 |
| Fetal Liver | 100.0 |
| Kidney pool | 55.5 |
| Fetal Kidney | 2.7 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.3 |
| Bladder | 0.1 |
| Gastric ca. (liver met.) NCI-N87 | 0.0 |
| Stomach | 0.0 |
| Colon ca. SW-948 | 2.0 |
| Colon ca. SW480 | 0.0 |
| Colon ca. (SW480 met) SW620 | 1.3 |
| Colon ca. HT29 | 0.0 |
| Colon ca. HCT-116 | 0.0 |
| Colon cancer tissue | 0.1 |
| Colon ca. SW1116 | 0.0 |
| Colon ca. Colo-205 | 11.5 |
| Colon ca. SW-48 | 0.1 |
| Colon | 19.6 |
| Small Intestine | 0.6 |
| Fetal Heart | 0.1 |
| Heart | 0.1 |
| Lymph Node Pool | 0.0 |
| Lymph Node pool 2 | 10.2 |
| Fetal Skeletal Muscle | 0.5 |
| Skeletal Muscle pool | 0.0 |
| Skeletal Muscle | 0.1 |
| Spleen | 0.2 |
| Thymus | 0.3 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 0.0 |
| Brain (Cerebellum) | 0.2 |
| Brain (Fetal) | 0.0 |
| Brain (Hippocampus) | 0.0 |
| Cerebral Cortex pool | 0.0 |
| Brain (Substantia nigra) | 0.0 |
| Brain (Thalamus) | 0.0 |
| Brain (Whole) | 0.3 |
| Spinal Cord | 0.0 |
| Adrenal Gland | 0.8 |
| Pituitary Gland | 9.3 |
| Salivary Gland | 0.2 |
| Thyroid | 0.4 |
| Pancreatic ca. PANC-1 | 0.0 |
| Pancreas pool | 0.1 |

Column A - Rel. Exp. (%) Ag1769, Run 317617203

Panel 1.3D Summary: Expression of the human cytosolic PEPCK gene was highest in liver (CT=24.6). Cytosolic PEPCK was expressed at moderate to high levels in adipose, mammary gland, kidney, colon, small intestine, heart, and adrenal gland. This expression pattern is consistent with the GeneCalling® results and with reports from the literature.

Panel 5 Islet Summary: Among the samples on this panel, expression of the human cytosolic PEPCK gene was highest in small intestine (CT=28.3). Lower levels of expression were also detected in liver, adipose and pancreatic islets of Langerhans.

General_screening_panel_v1.7 Summary: Ag1769 Highest expression of this gene was detected in liver (CT=23.4), kidney (CT=24.6), adipose (CT=26.31), colon (CT=25.7), lymph node (CT=26.7), and pituitary gland (CT=26.83). This gene encodes phosphoenolpyruvate carboxykinase (PEPCK). The cytosolic isoform of PEPCK regulates glyceroneogenesis in adipose tissue. The glycerol-3-phosphate product of glyceroneogenesis is used in triglyceride synthesis. Cytosolic PEPCK is upregulated in adipose tissue of obese AKR versus normal C57B1 adipose and may contribute to the obese phenotype. This hypothesis is supported by the fact that transgenic overexpression of cytosolic PEPCK in adipose is associated with increased glyceroneogenesis, increased adipocyte (fat cell) size and fat mass, and higher body weight (Sun Y, Liu S, Ferguson S, Wang L, Klepcyk P, Yun J S, Friedman J E. Phosphoenolpyruvate carboxykinase over-expression selectively attenuates insulin signaling and hepatic insulin sensitivity in transgenic mice. J Biol. Chem. 2002). Therapeutic modulation of this gene, expressed protein and/or use of antibodies or small molecule drugs targeting the gene or gene product are useful in the treatment of endocrine/metabolically related diseases, such as obesity and diabetes.

### EXAMPLE A6

### Pathways Relevant to the Etiology and Pathogenesis of Obesity and/or Diabetes

PathCalling screening identified significant protein-protein interactions for cytosolic Phosphoenolpyruvate Carboxykinase (PEPCK) with stathmin-like 4 and heme-regulated initiation factor 2-alpha kinase (HR1). Protocol for PathCalling is disclosed in Example Q10.

Stathmin is a ubiquitous phosphorylated protein thought to act as an intracellular relay for diverse regulatory pathways (Sobel A. Stathmin: a relay phosphoprotein for multiple signal transduction? Trends Biochem Sci 1991 August; 16 (8):301-5. PMID: 1957351). HRI is also widely expressed with significant transcript levels in adipose, muscle and liver and is activated by oxidative stress (Hwang S Y, Kim M K, Kim J C. Cloning of hHRI, human heme-regulated eukaryotic initiation factor 2alpha kinase: down-regulated in epithelial ovarian cancers. Mol Cells 2000 Oct. 31; 10 (5):584-91 PMID: 11101152). Oxidative stress has been associated with diabetes. It is possible that under conditions of oxidative stress, HRI will phosphorylate and activate cytosolic Phosphoenolpyruvate Carboxykinase.

The outcome of inhibiting the action of the human cytosolic Phosphoenolpyruvate Carboxykinase (PEPCK) gene would be a potential reduction of glyceroneogenesis, triglyceride deposition in adipose tissue, adipocyte (fat cell) size and fat mass, and a reduction in body weight. Inhibition of cytosolic PEPCK would also reduce hepatic gluconeogenesis and ameliorate the fasting hyperglycemia of Type 2 diabetes.

### EXAMPLE A7

### Assays Screening for Modulators of Phosphoenolpyruvate carboxykinase

A non-exhaustive list of cell lines that express the Phosphoenolpyruvate carboxykinase (PEPCK) gene can be obtained from the RTQ-PCR results shown herein. These and other Phosphoenolpyruvate carboxykinase (PEPCK) expressing cell lines could be used for screening purposes.

Screening for an inhibitor/antagonist of PEPCK could be accomplished with an in vitro glucose production assay. H4IIE cells transfected with recombinant cytosolic PEPCK would be tested for glucose production as described in Materials and Methods in Wang J C, Stafford J M, Scott D K, Sutherland C, Granner D K. The molecular physiology of hepatic nuclear factor 3 in the regulation of gluconeogenesis. J Biol Chem 275:14717-21, 2000, PMID: 10799560.

Our results indicate that a modulator of cytosolic Phosphoenolpyruvate Carboxykinase (PEPCK) activity, such as an inhibitor, activator, antagonist, or agonist of PEPCK may be useful for treatment of such disorders as obesity, diabetes, and insulin resistance, as well as for enhancement of insulin secretion.

B. NOV2—Transketolase

Transketolase is a thiamine-dependent enzyme that links the pentose phosphate pathway with the glycolytic pathway. The pentose phosphate pathway, which is active in most tissues, provides sugar phosphates for intermediary biosynthesis, especially nucleotide metabolism. The pentose pathway also generates the biosynthetic reducing power for the cell in the form of NADPH. Transketolase is directly involved in the branch of the pathway that channels excess sugar phosphates to glycolysis, enabling the production of NADPH to be maintained under different metabolic conditions. The pentose phosphate pathway is especially active in sites where production of fatty acids and steroid synthesis occurs because they require the reducing power of NADPH (Lewandowski P A, Cameron-Smith D, Jackson C J, Kultys E R, Collier G R. The role of lipogenesis in the development of obesity and diabetes in Israeli sand rats (Psammomys obesus). J Nutr 1998 November; 128 (11):1984-8. PMID: 9808653; Parks E J, Krauss R M, Christiansen M P, Neese R A, Hellerstein M K. Effects of a low-fat, high-carbohydrate diet on VLDL-triglyceride assembly, production, and clearance. J Clin Invest. 1999 October; 104 (8):1087-96. PMID: 10525047; Marques-Lopes I, Ansorena D, Astiasaran I, Forga L, Martinez J A. Postprandial de novo lipogenesis and metabolic changes induced by a high-carbohydrate, low-fat meal in lean and overweight men. Am J Clin Nutr. 2001 February; 73 (2):253-61. PMID: 11157321).

We found transketolase to be down-regulated in brown adipose tissue derived from mice with various body weights ranging from obese (sd4 compared to chow-fed mice) to heavily obese (ngsd7 compared to chow-fed mice) and hyperglycemic, heavily obese mice (hgsd7+compared to chow-fed mice) on a high fat diet; transketolase remained unchanged in white adipose from the same groups of mice. This down-regulation of transketolase is in conjunction with a down-regulation of several enzymes in the fatty acid synthesis pathway and the anaplerotic pathway, including ATP citrate lyase, fatty acid elongase, and malic enzyme, as well as, SREBP. This suggests that in brown adipose, fatty acid synthesis and lipogenesis are down-regulated as a compensatory mechanism to the high fat diet. Such a compensatory mechanism is not present in white adipose (Swierczynski J, Goyke E, Wach L, Pankiewicz A, Kochan Z, Adamonis W, Sledzinski Z, Aleksandrowicz Z. Comparative study of the lipogenic potential of human and rat adipose tissue. Metabolism. 2000 May; 49 (5):594-9. PMID: 10831168; Hellerstein M K. De novo lipogenesis in humans: metabolic and regulatory aspects. Eur J Clin Nutr. 1999 April; 53 Suppl 1:S53-65. Review. PMID: 10365981)

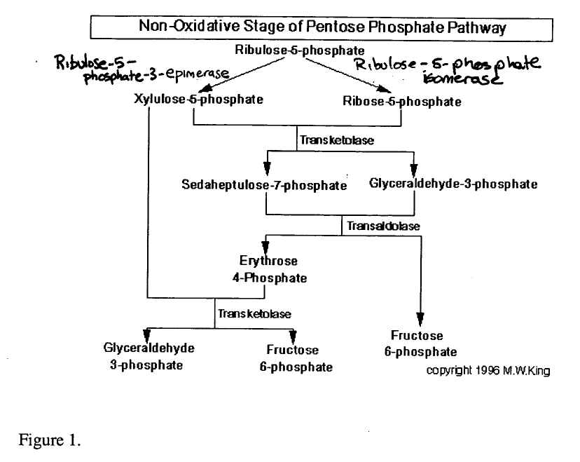

FIG. 1 summarizes the biochemistry surrounding the human Transketolase and potential assays that may be used to screen for antibody therapeutics or small molecule drugs to treat obesity and/or diabetes. FIG. 1 shows pentose phosphate pathway generating NADPH for fatty acid and steroid biosynthesis. The pathway has 1^{st} oxidative and 2^{nd} non-oxidative stage. The non-oxidative stage is link between pentose phosphate pathway and glycolysis, reactions of the pathway are cytoplasmic. Transketolase cofactors are thiamine pyrophosphate and Mg²⁺. (Frank T, Bitsch R, Maiwald J, Stein G. Alteration of thiamine pharmacokinetics by end-stage renal disease (ESRD). Int J Clin Pharmacol Ther 1999 September; 37 (9):449-55; Pietrzak I, Baczyk K. Erythrocyte transketolase activity and guanidino compounds in hemodialysis patients. Kidney Int Suppl 2001 February; 78:S97-101)

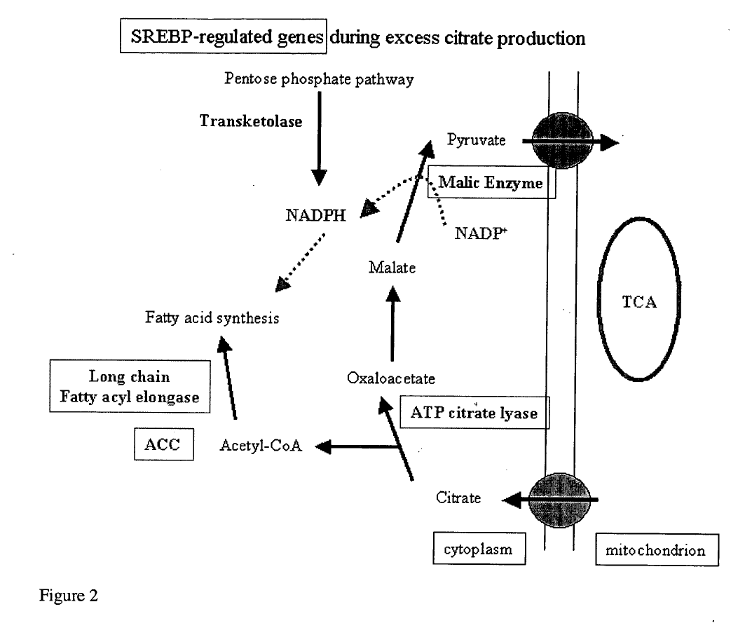

FIG. 2 suggests how alterations in expression of the human Transketolase and associated gene products function in the etiology and pathogenesis of obesity and/or diabetes. The scheme incorporates the unique findings of these discovery studies in conjunction with what has been reported in the literature. The outcome of inhibiting the action of the human Transketolase would be a reduction of Insulin Resistance, a major problem in obesity and/or diabetes.

Therefore, mimicking brown adipose in white adipose, by inhibiting transketolase, may decrease the amount of NADPH necessary for fatty acid synthesis and lipogenesis. The decrease of NADPH available for fatty acid synthesis and lipogenesis may force utilization of fat stores. Thus, a modulator of transketolase such as an antagonist or an inhibitior for transketolase may be beneficial for the treatment of obesity an/or diabetes. Furthermore, ihibition of production of fructose-6-phosphate through inhibition of transketolase may decrease the hexosamine pathway and may also have beneficial effects for insulin resistance. Cell lines expressing the Transketolase can be obtained from the RTQ-PCR results disclosed herein. These and other Transketolase expressing cell lines could be used for screening purposes.

Furthermore, our results indicate that a modulator of Transketolase activity, such as an inhibitor, activator, antagonist, or agonist of Transketolase may be useful for treatment of such disorders as obesity, diabetes, and insulin resistance, as well as for enhancement of insulin secretion.

Discovery Process

The following sections describe the study design(s) and the techniques used to identify the Transketolase encoded protein and any variants, thereof, as being suitable as diagnostic markers, targets for an antibody therapeutic and targets for a small molecule drugs for Obesity and Diabetes.

Obesity and Diabetes are major public health concerns in the developed and developing world. It is estimated that over half of the adult US population is overweight with a body mass index (BMI) greater than the upper limit of normal (25) where the BMI is defined as the weight (Kg)/[height (m)]². A common consequence of being overweight is hyperlipidemia and the development of insulin resistance. This is followed by the development of hyperglycemia, a hallmark of Type II diabetes. Left untreated, the hyperglycemia leads to microvascular disease and end organ damage that includes retinopathy, renal disease, cardiac disease, peripheral neuropathy and peripheral vascular compromise. Currently, over 16 million adults in the US are affected by Type II diabetes and the condition has now become rampant among school-age children as a consequence of the epidemic of obesity in that age group.

Diabetes mellitus is a disorder in which blood levels of glucose (a simple sugar) are abnormally high because the body doesn't release or respond to insulin adequately. Blood sugar (glucose) levels vary throughout the day, rising after a meal and returning to normal within 2 hours. Blood sugar levels are normally between 70 and 110 milligrams per deciliter (mg/dL) of blood in the morning after an overnight fast. They are usually lower than 120 to 140 mg/dL 2 hours after eating foods or drinking liquids containing sugar or other carbohydrates.

Insulin, a hormone released from the pancreas, is the primary substance responsible for maintaining appropriate blood sugar levels. Insulin allows glucose to be transported into cells so that they can produce energy or store glucose-derived enrgy until it's needed. The rise in blood sugar levels after eating or drinking stimulates the pancreas to produce insulin, preventing a greater rise in blood sugar levels and causing them to fall gradually. Because muscles use glucose for energy, blood sugar levels can also fall during physical activity.

Diabetes results when the body doesn't produce enough insulin to maintain normal blood sugar levels or when cells don't respond appropriately to insulin. In type II diabetes mellitus, the pancreas continues to manufacture insulin, sometimes even at higher than normal levels. However, the body develops resistance to its effects, resulting in a relative insulin deficiency.

The main goal of diabetes treatment is to keep blood sugar levels within the normal range as much as possible. Completely normal levels are difficult to maintain, but the more closely they can be kept within the normal range, the less likely that temporary or long-term complications will develop.

Therefore, a therapeutic that decreases insulin resistance and/or enhances insulin secretion would be beneficial in treatment of obesity and/or diabetes. Additionally, such a therapeutic would be beneficial in treatment of insulin resistance, a condition that often leads to the development of diabetes.

### EXAMPLE B1

### Mouse Dietary—Induced Obesity

A protocol for Mouse Dietary-Induced Obesity study is disclosed in Example Q1.

The predominant cause for obesity in clinical populations is excess caloric intake. This so-called diet-induced obesity (DIO) is mimicked in animal models (mouse strain C57BL/6) by feeding high fat diets of greater than 40% fat content. The DIO study was established to identify the gene expression changes contributing to the development and progression of diet-induced obesity. In addition, the study design sought to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues revealed genes and pathways that can be used as therapeutic targets for obesity and/or diabetes. Protocol for differential gene expression analysis, GeneCalling®, is disclosed in Example Q7.

Results

A fragment of the mouse (mouse strain C57BL/6) Transketolase gene was initially found to be down-regulated by 1.7 fold in the brown adipose tissue of mice fed a high fat diet who reach 4 standard deviations of body weight when compared to chow fed mice relative to brown adipose of chow fed mice using CuraGen's GeneCalling® method of differential gene expression. A differentially expressed human gene fragment migrating, at approximately 385 nucleotides in length was definitively identified as a component of the human Transketolase cDNA. The method of competitive PCR was used for confirmation of the gene assessment. The electropherographic peaks corresponding to the gene fragment of the human Transketolase were ablated when a gene-specific primer (shown in Table B 1) competes with primers in the linker-adaptors during the PCR amplification. The peaks at 385 nt in length were ablated in the sample from both the chow fed mice and the mice on a high fat diet.

TABLE B1

The direct sequence of the 385 nucleotide-long gene fragment and the
gene-specific primers used for competitive PCR are indicated on the
cDNA sequence of the Transketolase fragment (SEQ ID NO:178) are shown
in bold. The gene-specific primers at the 5′ and 3′ ends of the
fragment are underlined.
Mouse transketolase (scm gb-u05 809_4) (fragment from 1151 to 1536 in
bold. band size: 386)

```
  670 ACATCAACCG CCTGGGCCAG AGCGACCCAG CCCCGCTGCA GCACCAGGTG GACATCTACC
  730 AGAAGCGCTG TGAGGCCTTT GGCTGGCACA CCATCATCGT GGACCGACAC AGCGTGGAGG
  790 AGCTGTGCAA GGCCTTTGGT CACGCCAAGC ACCAACCAAC AGCCATCATT GCCAAGACCT
  850 TCAAGGGCCG AGGGATCACA CGGATTGAAG ACAAGGAGGC GTGGCACGGG AAGCCCCTCC
  910 CCAAAAACAT GGCCGAGCAG ATTATCCAGG AGATTTACAG CCAGGTTCAG AGCAAAAAGA
  970 AGATCCTGGC CACGCCCCCT CAGGAGGATG CCCCATCCGT GGACATTGCT AACATCCGAA
1030 TGCCTACGCC ACCCAGCTAC AAAGTGGGGG ACAAGATAGC CACCCGGAAG GCCTATGGAC
1090 TGGCCCTCGC TAAGCTGGGC CACGCCAGTG ACCGTATCAT TGCCCTGGAT GGAGACACCA
1150 AGAATTCCAC CTTCTCGGAG CTCTTCAAAA AGGAGCACCC AGACCGGTTC ATTGAGTGCT
1210 ACATTGCCGA GCAAAACATG GTGAGCATTG CCGTGGGCTG TGCCACACGT GACCGGACAG
1270 TGCCCTTCTG CAGTACTTTC GCGGCCTTCT TCACACGGGC CTTCGACCAG ATTCGCATGG
1330 CCGCCATCTC TGAGAGCAAC ATCAACCTCT GTGGCTCCCA CTGTGGTGTG TCCATTGGGG
1390 AAGACGGGCC CTCTCAGATG GCCCTCGAAG ACCTGGCCAT GTTCCGGTCA GTCCCCATGT
1450 CCACCGTCTT TTACCCAAGC GATGGAGTTG CAACAGAGAA GGCAGTGGAG TTAGCAGCCA
1510 ACACAAAGGG CATTTGCTTC ATCCGGACCA GCCGCCCAGA GAATGCCATT ATTTATAGCA
1570 ACAATGAGGA TTTCCAGGTC GGCCAAGCCA AGGTGGTCCT GAAGAGCAAG GATGACCAAG
1630 TGACAGTGAT CGGGGCTGGT GTAACTCTGC ATGAGGCCTT GGCTGCTGCA GAGAGTCTAA
1690 AGAAAGATAA GATCAGCATC CGGGTGCTGG ATCCCTTCAC TATCAAGCCC CTGGACAGGA
1750 AACTCATCCT AGACTCTGCC CGAGCAACCA AAGGCAGGAT CCTCACCGTG GAGGACCACT
1810 ACTACGAAGG TGGCATAGGA GAGGCAGTGT CTGCTGCCGT AGTGGGTGAA CCTGGAGTGA
1870 CGGTCACTCG CCTGGCTGTC AGCCAAGTAC CACGAAGTGG CAAGCCAGCT GAGCTACTGA
1930 AGATGTTCGG TATTGACAAG GACGCCATTG TGCAAGCTGT GAAAGGCCTT GTCACCAAGG
1990 GCTAGGGAGG GCATGGGATG CTGGGTG
```

(gene length is 2516, only region from 670 to 2016 shown)

### EXAMPLE B2

### Identification of Human Transketolase Sequences

The sequence of Human Transketolase (Acc. No. CG175387-01) was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof. The protocol for identification of human sequence(s) is disclosed in Example Q8.

Table B2 shows protein alignment (ClustalW) of Human Transketolase sequence (CG175387-01; SEQ ID NO:8) and Mouse Transketolase sequence (SEQ ID NO:179) Table B3 shows sequence of Mouse Transketolase (SEQ ID NO:179).

[image]

[image]

TABLE B3
Mouse Transketolase (SEQ ID NO:179).

>U05809_Mouse
MEGYHKPDQQKLQALKDTANRLRISSIQATTAAGSGHPTSCCSAAELMAVLFFHTMRYKALDPRNPHNDR
FVLSKGHAAPILYAVWAEAGFLPEAELLNLRKISSDLDGHPVPKQAFTDVATGSLGQGLGAACGMAYTGK
YFDKASYRVYCMLGDGEVSEGSVWEAMAFAGIYKLDNLVAIFDINRLGQSDPAPLQHQVDIYQKRCEAFG
WHTIIVDGHSVEELCKAFGQAKHQPTAIIAKTFKGRGITGIEDKEAWHGKPLPKNMAEQIIQEIYSQVQS
KKKILATPPQEDAPSVDIANIRMPTPPSYKVGDKIATRKAYGLALAKLGHASDRIIALDGDTKNSTFSEL
FKKEHPDRFIECYIAEQNMVSIAVGCATRDRTVPFCSTFAAFFTRAFDQJRMAAISESNLNLCGSHCGVS
IGEDGPSQMALEDLAMFRSVPMSTVFYPSDGVATEKAVELAANTKGICFLRTSRPENAIIYSNNEDFQVG
QAKVVLKSKDDQVTVIGAGVTLHEALAAAESLKKDKISIRVLDPFTIKPLDRKLLLDSARATKGRILTVE
DHYYEGGIGEAVSAAVVGEPGVTVTRLAVSQVPRSGKPAELLKMFGIDKDAIVQAVKGLVTKG

The laboratory cloning was performed using one or more of the methods summarized in Example Q8. The NOV2 clone was analyzed, and the nucleotide and encoded polypeptide sequences are shown in Table B4.

TABLE B4
NOV2 Sequence Analysis

| | | |
|---|---|---|
| NOV2a, CG175387-01 | SEQ ID NO:7 | 2078 bp |
| DNA Sequence | ORF Start: ATG at 80 | ORF Stop: TAG at 1949 |

GGCACGAGGGCCTGTCGCCGCGGGAGCAGCCGCTATCTCTGTGTGTCCGCGTGTGCGCCCGGTCCCCG
CCTGCCGCACCATGGAGAGCTACCACAAGCCTGACCAGCAGAAGCTGCAGGCCTTGAAGGACACGGCC
AACCGCCTACGTATCAGCTCCATCCAGGCCACCACTGCGGCGGGCTCTGGCCACCCCACGTCATGCTG
CAGCGCCGCAGAGATCATGGCTGTCCTCTTTTTCCACACCATGCGCTACAAGTCCCAGGACCCCCGGA
ATCCGCACAATGACCGCTTTGTGCTCTCCAAGGGCCATGCAGCTCCCATCCTCTACGCGGTCTGGGCT
GAAGCTGGTTTCCTGGCCGAGGCGGAGCTGCTGAACCTGAGGAAGATCAGCTCCGACTTGGACGGGCA
CCCGGTCCCGAAACAAGCTTTCACCGACGTGGCCACTGGCTCCCTGGGCCAGGGCCTCGGGGCCGCTT
GTGGGATGGCCTACACCGGCAAATACTTCGACAAGGCCAGCTACCGAGTCTATTGCTTGCTGGGAGAT
GGGGAGCTGTCAGAGGGCTCTGTATGGGAGGCCATGGCCTTCGCCAGCATCTATAAGCTGGACAACCT
TGTGGCCATTCTAGACATCAATCGCCTGGGCCAGAGTGACCCGGCCCCGCTGCAGCACCAGATGGACA
TCTACCAGAAGCGGTGCGAGGCCTTCGGTTGGCATGCCATCATCGTGGATGGACACAGCGTGGAGGAG
CTGTGCAAGGCCTTTGGCCAGGCCAAGCACCAGCCAACAGCCATCATTGCCAAGACCTTCAAGGGCCG
AGGGATCACGGGGGTAGAAGATAAGGAGTCTTGGCATGGGAAGCCCCTCCCCAAAAACATGGCTGAGC
AGATCATCCAGGAGATCTACAGCCAGATCCAGAGCAAAAAGAAGATCCTGGCAACCCCTCCACAGGAG
GACGCACCCTCAGTGGACATTGCCAACATCCGCATGCCCAGCCTGCCCAGCTACAAAGTTGGGGACAA
GATAGCCACCCGCAAGGCCTACGGGCAGGCACTGGCCAAGCTGGGCCATGCCAGTGACCGCATCATCG
CCCTGGATGGGGACACCAAAAATTCCACCTTCTCGGAGATCTTCAAAAAGGAGCACCCGGACCGCTTC
ATCGAGTGCTACATTGCCGAGCAGAACATGGTGAGCATCGCGGTGGGCTGTGCCACCCGCAACAGGAC
GGTGCCCTTCTGCAGCACTTTTGCAGCCTTCTTCACGCGGGCCTTTGACCAGATTCGCATGGCCGCCA
TCTCCGAGAGCAACATCAACCTCTGCGGCTCCCACTGCGGCGTTTCCATCGGGGAAGACGGGCCCTCC
CAGATGGCCCTAGAAGATCTGGCTATGTTTCGGTCAGTCCCCACATCAACTGTCTTTTACCCAAGTGA
TGGCGTTGCTACAGAGAAGGCAGTGGAACTAGCCGCCAATACAAAGGGTATCTGCTTCATCCGGACCA
GCCGCCCAGAAAATGCCATCATCTATAACAACAATGAGGACTTCCAGGTCGGACAAGCCAAGGTGGTC
CTGAAGAGCAAGGATGACCAGGTGACCGTTATCGGGGCTGGGGTGACCCTGCACGAGGCCTTGGCCGC
TGCCGAACTGCTGAAGAAAGAAAAGATCAACATCCGCGTGCTGGACCCCTTCACCATCAAGCCCCTGG
ACAGAAAACTCATTCTCGACAGCGCTCGTGCCACCAAGGGCAGGATCCTCACCGTGGAGGACCATTAT
TATGAAGGTGGCATTGGTGAGGCTGTGTCCAGTGCAGTAGTGGGCGAGCCTGGCATCACTGTCACCCA
CCTGGCAGTTAACCGGGTACCAAGAAGTGGGAAGCCAGCTGAGCTGCTGAAGATGTTTGGTATCGACA
GGGATGCCATTGCACAAGCTGTGAGGGGCCTCATCACCAAGGCCTAGGGCGGGTATGAAGTGTGGGGC
GGGGGTCTATACATTCCTGAGATTCTGGGAAAGGTGCTCAAAGATGTACTGAGAGGAGGGGTAAATAT
ATGTTTTGAGAAAAATGAAAAAAAAAAAAAAAAAAAAA

| | | | |
|---|---|---|---|
| NOV2a, CG175387-01 | | | |
| Protein Sequence | SEQ ID NO:8 | 623 aa | MW at 67876.8 kD |

MESYHKPDQQKLQALKDTANRLRISSIQATTAAGSGHPTSCCSAAEIMAVLFFHTMRYKSQDPRNPHN
DRFVLSKGHAAPILYAVWAEAGFLAEAELLNLRKISSDLDGHPVPKQAFTDVATGSLGQGLGAACGMA
YTGKYFDKASYRVYCLLGDGELSEGSVWEAMAFASIYKLDNLVAILDINRLGQSDPAPLQHQMDIYQK
RCEAFGWHAIIVDGHSVEELCKAFGQAKHQPTAIIAKTFKGRGITGVEDKESWHGKPLPKNMAEQIIQ
EIYSQIQSKKKILATPPQEDAPSVDIANIRMPSLPSYKVGDKIATRKAYGQALAKLGHASDRIIALDG
DTKNSTFSEIFKKEHPDRFIECYIAEQNNVSIAVGCATRNRTVPFCSTFAAFFTRAFDQIRMAAISES
NINLCGSHCGVSIGEDGPSQMALEDLAMFRSVPTSTVFYPSDGVATEKAVELAANTKGICFIRTSRPE
NAIIYNNNEDFQVGQAKVVLKSKDDQVTVIGAGVTLHEALAAAELLKKEKINIRVLDPFTIKPLDRKL
ILDSARATKGRILTVEDHYYEGGIGEAVSSAVVGEPGITVTHLAVNRVPRSGKPAELLKMFGIDRDAI
AQAVRGLITKA

| | | |
|---|---|---|
| NOV2b, CG175387-03 | SEQ ID NO:9 | 1927 bp |
| DNA Sequence | ORF Start: at 2 | ORF Stop: TAG at 1886 |

TCATCATCACCACCATCACGAGAGCTACCACAAGCCTGACCAGCAGAAGCTGCAGGCCTTGAAGGACA
CGGCCAACCGCCTACGTATCAGCTCCATCCAGGCCACCACTGCGGCGGGCTCTGGCCACCCCACGTCA
TGCTGCAGCGCCGCAGAGATCATGGCTGTCCTCTTTTTCCACACCATGCGCTACAAGTCCCAGGACCC
CCGGAATCCGCACAATGACCGCTTTGTGCTCTCCAAGGGCCATGCAGCTCCCATCCTCTACGCGGTCT
GGGCTGAAGCTGGTTTCCTGGCCGAGGCGGAGCTGCTGAACCTGAGGAAGATCAGCTCCGACTTGGAC
GGGCACCCGGTCCCGAAACAAGCTTTCACCGACGTGGCCACTGGCTCCCTGGGCCAGGGCCTCGGGGC
CGCTTGTGGGATGGCCTACACCGGCAAATACTTCGACAAGGCCAGCTACCGAGTCTATTGCTTGCTGG
GAGACGGGGAGCTGTCAGAGGGCTCTGTATGGGAGGCCATGGCCTTCGCCAGCATCTATAAGCTGGAC
AACCTTGTGGCCATTCTAGACATCAATCGCCTGGGCCAGAGTGACCCGGCCCCACTGCAGCACCAGAT
GGACATCTACCAGAAGCGGTGCGAGGCCTTCGGTTGGCATGCCATCATCGTGGATGGACACAGCGTGG
AGGAGCTGTGCAAGGCCTTTGGCCAGGCCAAGCACCAGCCAACAGCCATCATTGCCAAGACCTTCAAG
GGCCGAGGGATCACGGGGGTAGAAGATAAGGAGTCTTGGCATGGGAAGCCCCTCCCCAAAAACATGGC
TGAGCAGATCATCCAGGAGATCTACAGCCAGATCCAGAGCAAAAAGAAGATCCTGGCAACCCCTCCAC
AGGAGGACGCACCCTCAGTGGACATTGCCAACATCCGCATGCCCAGCCTGCCCAGCTACAAAGTTGGG
GACAAGATAGCCACCCGCAAGGCCTACGGGCAGGCACTGGCCAAGCTGGGCCATGCCAGTGACCGCAT
CATCGCCCTGGATGGGGACACCAAAAATTCCACCTTCTCGGAGATCTTCAAAAAGGAGCACCCGGACC
GCTTCATCGAGTGCTACATTGCTGAGCAGAACATGGTGAGCATCGCGGTGGGCTGTGCCACCCGCAAC
AGGACGGTGCCCTTCTGCAGCACTTTTGCAGCCTTCTTCACGCGGGCCTTTGACCAGATTCGCATGGC
CGCCATCTCCGAGAGCAACATCAACCTCTGCGGCTCCCACTGCGGCGTTTCCATCGGGGAAGACGGGC
CCTCCCAGATGGCCCTAGAAGATCTGGCTATGTTTCGGTCAGTCCCCACATCAACTGTCTTTTACCCA
AGTGATGGCGTTGCTACAGAGAAGGCAGTGGAACTAGCCGCCAATACAAAGGGTATCTGCTTCATCCG
GACCAGCCGCCCAGAAAATGCCATCATCTATAACAACAATGAGGACTTCCAGGTCGGACAAGCCAAGG
TGGTCCTGAAGAGCAAGGATGACCAGGTGACCGTTATCGGGGCTGGGGTGACCCTGCACGAGGCCTTG
GCCGCTGCCGAACTGCTGAAGAAAGAAAAGATCAACATCCGCGTGCTGGACCCCTTCACCATCAAGCC
CCTGGACAGAAAACTCATTCTCGACAGCGCTCGTGCCACCAAGGGCAGGATCCTCACCGTGGAGGACC
ATTATTATGAAGGTGGCATTGGTGAGGCTGTGTCCAGTGCAGTAGTGGGCGAGCCTGGCATCACTGTC
ACCCACCTGGCAGTTAACCGGGTACCAAGAAGTGGGAAGCCGGCTGAGCTGCTGAAGATGTTTGGTAT
CGACAGGGATGCCATTGCACAAGCTGTGAGGGGCCTCATCACCAAGGCCTAGGCAGGTGCGGCCGCAC
TCGAGCACCACCACCACCACCAC

| | | | |
|---|---|---|---|
| NOV2b, CG175387-03 | | | |
| Protein Sequence | SEQ ID NO:10 | 628 aa | MW at 68568.4 kD |

HHHHHHESYHKPDQQKLQALKDTANRLRISSIQATTAAGSGHPTSCCSAAEIMAVLFFHTMRYKSQDP
RNPHNDRFVLSKGHAAPILYAVWAEAGFLAEAELLNLRKISSDLDGHPVPKQAFTDVATGSLGQGLGA
ACGMAYTGKYFDKASYRVYCLLGDGELSEGSVWEAMAFASIYKLDNLVAILDINRLGQSDPAPLQHQM
DIYQKRCEAFGWHAIIVDGHSVEELCKAFGQAKHQPTAIIAKTFKGRGITGVEDKESWHGKPLPKNMA
EQIIQEIYSQIQSKKKILATPPQEDAPSVDIANIRNPSLPSYKVGDKIATRKAYGQALAKLGHASDRI
IALDGDTKNSTFSEIFKKEHPDRFIECYIAEQNMVSIAVGCATRNRTVPFCSTFAAFFTRAFDQIRMA
AISESNINLCGSHCGVSIGEDGPSQMALEDLANFRSVPTSTVFYPSDGVATEKAVELAANTKGICFIR
TSRPENAIIYNNNEDFQVGQAKVVLKSKDDQVTVIGAGVTLHEALAAAELLKKEKINIRVLDPFTIKP
LDRKLILDSARATKGRILTVEDHYYEGGIGEAVSSAVVGEPGITVTHLAVNRVPRSGKPAELLKMFGI
DRDAIAQAVRGLITKA

| | | |
|---|---|---|
| NOV2c, 267254044 | SEQ ID NO:11 | 1897 bp |
| DNA Sequence | ORF Start: at 3 | ORF Stop: TAG at 1884 |

CACCGAATTCCACCATGGAGAGCTACCACAAGCCTGACCAGCAGAAGCTGCAGGCCTTGAAGGACACG
GCCAACCGCCTACGTATCAGCTCCATCCAGGCCACCACTGCGGCGGGCTCTGGCCACCCCACGTCATG
CTGCAGCGCCGCAGAGATCATGGCTGTCCTCTTTTTCCACACCATGCGCTACAAGTCCCAGGACCCCC
GGAATCCGCACAATGACCGCTTTGTGCTCTCCAAGGGCCATGCAGCTCCCATCCTCTACGCGGTCTGG
GCTGAAGCTGGTTTCCTGGCCGAGGCGGAGCTGCTGAACCTGAGGAAGATCAGCTCCGACTTGGACGG
GCACCCGGTCCCGAAACAAGCTTTCACCGACGTGGCCACTGGCTCCCTGGGCCAGGGCCTCGGGGCCG
CTTGTGGGATGGCCTACACCGGCAAATACTTCGACAAGGCCAGCTACCGAGTCTATTGCTTGCTGGGA
GATGGGGAGCTGTCAGAGGGCTCTGTATGGGAGGCCATGGCCTTCGCCAGCATCTATAAGCTGGACAA
CCTTGTGGCCATTCTAGACATCAATCGCCTGGGCCAGAGTGACCCGGCCCCGCTGCAGCACCAGATGG
ACATCTACCAGAAGCGGTGCGAGGCCTTCGGTTGGCATGCCATCATCGTGGATGGACACAGCGTGGAG
GAGCTGTGCAAGGCCTTTGGCCAGGCCAAGCACCAGCCAACAGCCATCATTGCCAAGACCTTCAAGGG
CCGAGGGATCACGGGGGTAGAAGATAAGGAGTCTTGGCATGGGAAGCCCCTCCCCAAAAACATGGCTG
AGCAGATCATCCAGGAGATCTACAGCCAGATCCAGAGCAAAAAGAAGATCCTGGCAACCCCTCCACAG
GAGGACGCACCCTCAGTGGACATTGCCAACATCCGCATGCCCAGCCTGCCCAGCTACAAAGTTGGGGA
CAAGATAGCCACCCGCAAGGCCTACGGGCAGGCACTGGCCAAGCTGGGCCATGCCAGTGACCGCATCA
TCGCCCTGGATGGGGACACCAAAAATTCCACCTTCTCGGAGATCTTCAAAAAGGAGCACCCGGACCGC
TTCATCGAGTGCTACATTGCCGAGCAGAACATGGTGAGCATCGCGGTGGGCTGTGCCACCCGCAACAG
GACGGTGCCCTTCTGCAGCACTTTTGCAGCCTTCTTCACGCGGGCCTTTGACCAGATTCGCATGGCCG
CCATCTCCGAGAGCAACATCAACCTCTGCGGCTCCCACTGCGGCGTTTCCATCGGGGAAGACGGGCCC
TCCCAGATGGCCCTAGAAGATCTGGCTATGTTTCGGTCAGTCCCCACATCAACTGTCTTTTACCCAAG
TGATGGCGTTGCTACAGAGAAGGCAGTGGAACTAGCCGCCAATACAAAGGGTATCTGCTTCATCCGGA
CCAGCCGCCCAGAAAATGCCATCATCTATAACAACAATGAGGACTTCCAGGTCGGACAAGCCAAGGTG
GTCCTGAAGAGCAAGGATGACCAGGTGACCGTTATCGGGGCTGGGGTGACCCTGCACGAGGCCTTGGC
CGCTGCCGAACTGCTGAAGAAAGAAAAGATCAACATCCGCGTGCTGGACCCCTTCACCATCAAGCCCC
TGGACAGAAAACTCATTCTCGACAGCGCTCGTGCCACCAAGGGCAGGATCCTCACCGTGGAGGACCAT
TATTATGAAGGTGGCATTGGTGAGGCTGTGTCCAGTGCAGTAGTGGGCGAGCCTGGCATCACTGTCAC
CCACCTGGCAGTTAACCGGGTACCAAGAAGTGGGAAGCCAGCTGAGCTGCTGAAGATGTTTGGTATCG
ACAGGGATGCCATTGCACAAGCTGTGAGGGGCCTCATCACCAAGGCCTAGGCGGCCGCTAT

| | | | |
|---|---|---|---|
| NOV2c, 267254044 | | | |
| Protein Sequence | SEQ ID NO:12 | 627 aa | MW at 68276.2 kD |

PMSTMESYHKPDQQKLQALKDTANRLRISSIQATTAAGSGHPTSCCSAAEIMAVLFFHTMRYKSQDPR
NPHNDRFVLSKGHAAPILYAVWAEAGFLAEAELLNLRKISSDLDGHPVPKQAFTDVATGSLGQGLGAA
CGMAYTGKYFDKASYRVYCLLGDGELSEGSVWEAMAFASIYKLDNLVAILDINRLGQSDPAPLQHQMD
IYQKRCEAFGWHAIIVDGHSVEELCKAFGQAKHQPTAIIAKTFKGRGITGVEDKESWHGKPLPKNMAE
QIIQEIYSQIQSKKKILATPPQEDAPSVDIANIRMPSLPSYKVGDKIATRKAYGQALAKLGHASDRII
ALDGDTKNSTFSEIFKKEHPDRFIECYIAEQNMVSIAVGCATRNRTVPFCSTFAAFFTRAFDQIRMAA
ISESNINLCGSHCGVSIGEDGPSQMALEDLAMFRSVPTSTVFYPSDGVATEKAVELAANTKGICFIRT
SRPENAIIYNNNEDFQVGQAKVVLKSKDDQVTVIGAGVTLHEALAAAELLKKEKINIRVLDPFTIKPL
DRKLILDSARATKGRILTVEDHYYEGGIGEAVSSAVVGEPGITVTHLAVNRVPRSGKPAELLKMFGID
RDAIAQAVRGLITKA

| | | |
|---|---|---|
| NOV2d, GG175387-02 | SEQ ID NO:13 | 1897 bp |
| DNA Sequence | ORF Start: at 3 | ORF Stop: TAG at 1884 |

CACCGAATTCCACCATGGAGAGCTACCACAAGCCTGACCAGCAGAAGCTGCAGGCCTTGAAGGACACG
GCCAACCGCCTACGTATCAGCTCCATCCAGGCCACCACTGCGGCGGGCTCTGGCCACCCCACGTCATG
CTGCAGCGCCGCAGAGATCATGGCTGTCCTCTTTTTCCACACCATGCGCTACAAGTCCCAGGACCCCC
GGAATCCGCACAATGACCGCTTTGTGCTCTCCAAGGGCCATGCAGCTCCCATCCTCTACGCGGTCTGG
GCTGAAGCTGGTTTCCTGGCCGAGGCGGAGCTGCTGAACCTGAGGAAGATCAGCTCCGACTTGGACGG
GCACCCGGTCCCGAAACAAGCTTTCACCGACGTGGCCACTGGCTCCCTGGGCCAGGGCCTCGGGGCCG
CTTGTGGGATGGCCTACACCGGCAAATACTTCGACAAGGCCAGCTACCGAGTCTATTGCTTGCTGGGA
GATGGGGAGCTGTCAGAGGGCTCTGTATGGGAGGCCATGGCCTTCGCCAGCATCTATAAGCTGGACAA
CCTTGTGGCCATTCTAGACATCAATCGCCTGGGCCAGAGTGACCCGGCCCCGCTGCAGCACCAGATGG
ACATCTACCAGAAGCGGTGCGAGGCCTTCGGTTGGCATGCCATCATCGTGGATGGACACAGCGTGGAG
GAGCTGTGCAAGGCCTTTGGCCAGGCCAAGCACCAGCCAACAGCCATCATTGCCAAGACCTTCAAGGG
CCGAGGGATCACGGGGGTAGAAGATAAGGAGTCTTGGCATGGGAAGCCCCTCCCCAAAAACATGGCTG
AGCAGATCATCCAGGAGATCTACAGCCAGATCCAGAGCAAAAAGAAGATCCTGGCAACCCCTCCACAG
GAGGACGCACCCTCAGTGGACATTGCCAACATCCGCATGCCCAGCCTGCCCAGCTACAAAGTTGGGGA
CAAGATAGCCACCCGCAAGGCCTACGGGCAGGCACTGGCCAAGCTGGGCCATGCCAGTGACCGCATCA
TCGCCCTGGATGGGGACACCAAAAATTCCACCTTCTCGGAGATCTTCAAAAAGGAGCACCCGGACCGC
TTCATCGAGTGCTACATTGCCGAGCAGAACATGGTGAGCATCGCGGTGGGCTGTGCCACCCGCAACAG
GACGGTGCCCTTCTGCAGCACTTTTGCAGCCTTCTTCACGCGGGCCTTTGACCAGATTCGCATGGCCG
CCATCTCCGAGAGCAACATCAACCTCTGCGGCTCCCACTGCGGCGTTTCCATCGGGGAAGACGGGCCC
TCCCAGATGGCCCTAGAAGATCTGGCTATGTTTCGGTCAGTCCCCACATCAACTGTCTTTTACCCAAG
TGATGGCGTTGCTACAGAGAAGGCAGTGGAACTAGCCGCCAATACAAAGGGTATCTGCTTCATCCGGA
CCAGCCGCCCAGAAAATGCCATCATCTATAACAACAATGAGGACTTCCAGGTCGGACAAGCCAAGGTG
GTCCTGAAGAGCAAGGATGACCAGGTGACCGTTATCGGGGCTGGGGTGACCCTGCACGAGGCCTTGGC
CGCTGCCGAACTGCTGAAGAAAGAAAAGATCAACATCCGCGTGCTGGACCCCTTCACCATCAAGCCCC
TGGACAGAAAACTCATTCTCGACAGCGCTCGTGCCACCAAGGGCAGGATCCTCACCGTGGAGGACCAT
TATTATGAAGGTGGCATTGGTGAGGCTGTGTCCAGTGCAGTAGTGGGCGAGCCTGGCATCACTGTCAC
CCACCTGGCAGTTAACCGGGTACCAAGAAGTGGGAAGCCAGCTGAGCTGCTGAAGATGTTTGGTATCG
ACAGGGATGCCATTGCACAAGCTGTGAGGGGCCTCATCACCAAGGCCTAGGCGGCCGCTAT

| | | | |
|---|---|---|---|
| NOV2d, CG175387-02 | | | |
| Protein Sequence | SEQ ID NO:14 | 627 aa | MW at 68276.2 kD |

PNSTMESYHKPDQQKLQALKDTANRLRISSIQATTAAGSGHPTSCCSAAEIMAVLFFHTMRYKSQDPR
NPHNDRFVLSKGHAAPILYAVWAEAGFLAEAELLNLRKISSDLDGHPVPKQAFTDVATGSLGQGLGAA
CGMAYTGKYFDKASYRVYCLLGDGELSEGSVWEAMAFASIYKLDNLVAILDINRLGQSDPAPLQHQMD
IYQKRCEAFGWHAIIVDGHSVEELCKAFGQAKHQPTAIIAKTFKGRGITGVEDKESWHGKPLPKNMAE
QIIQEIYSQIQSKKKILATPPQEDAPSVDIANIRMPSLPSYKVGDKIATRKAYGQALAKLGHASDRII
ALDGDTKNSTFSEIFKKEHPDRFIECYIAEQNMVSIAVGCATRNRTVPFCSTFAAFFTRAFDQIRMAA
ISESNINLCGSHCGVSIGEDGPSQMALEDLANFRSVPTSTVFYPSDGVATEKAVELAANTKGICFIRT
SRPENAIIYNNNEDFQVGQAKVVLKSKDDQVTVIGAGVTLHEALAAAELLKKEKINIRVLDPFTIKPL
DRKLILDSARATKGRILTVEDHYYEGGIGEAVSSAVVGEPGITVTHLAVNRVPRSGKPAELLKMFGID
RDAIAQAVRGLITKA

| | | |
|---|---|---|
| NOV2e, CG175387-04 | SEQ ID NO:15 | 1942 bp |
| DNA Sequence | ORF Start: ATG at 138 | ORF Stop: TAG at 1884 |

AGAGCTACCACAAGCCTGACCAGCAGAAGCTGCAGGCCTTGAAGGACACGGCCAACCGCCTACGTATC
AGCTCCATCCAGGCCACCACTGCGGCGGGCTCTGGCCACCCCACGTCATGCTGCAGCGCCGCAGAGAT
CATGGCTGTCCTCTTTTTCCACACCATGCGCTACAAGTCCCAGGACCCCCGGAATCCGCACAATGACC
GCTTTGTGCTCTCCAAGGGCCATGCAGCTCCCATCCTCTACGCGGTCTGGGCTGAAGCTGGTTTCCTG
GCCGAGGCGGAGCTGCTGAACCTGAGGAAGATCAGCTCCGACTTGGACGGGCACCCGGTCCCGAAACA
AGCTTTCACCGACGTGGCCACTGGCTCCCTGGGCCAGGGCCTCGGGGCCGCTTGTGGGATGGCCTACA
CCGGCAAATACTTCGACAAGGCCAGCTACCGAGTCTATTGCTTGCTGGGAGATGGGGAGCTGTCAGAG
GGCTCTGTATGGGAGGCCATGGCCTTCGCCAGCATCTATAAGCTGGACAACCTTGTGGCCATTCTAGA
CATCAATCGCCTGGGCCAGAGTGACCCGGCCCCGCTGCAGCACCAGATGGACATCTACCAGAAGCGGT
GCGAGGCCTTCGGTTGGCATGCCATCATCGTGGATGGACACAGCGTGGAGGAGCTGTGCAAGGCCTTT
GGCCAGGCCAAGCACCAGCCAACAGCCATCATTGCCAAGACCTTCAAGGGCCGAGGGATCACGGGGGT
AGAAGATAAGGAGTCTTGGCATGGGAAGCCCCTCCCCAAAAACATGGCTGAGCAGATCATCCAGGAGA
TCTACAGCCAGATCCAGAGCAAAAAGAAGATCCTGGCAACCCCTCCACAGGAGGACGCACCCTCAGTG
GACATTGCCAACATCCGCATGCCCAGCCTGCCCAGCTACAAAGTTGGGGACAAGATAGCCACCCGCAA
GGCCTACGGGCAGGCACTGGCCAAGCTGGGCCATGCCAGTGACCGCATCATCGCCCTGGATGGGGACA
CCAAAAATTCCACCTTCTCGGAGATCTTCAAAAAGGAGCACCCGGACCGCTTCATCGAGTGCTACATT
GCCGAGCAGAACATGGTGAGCATCGCGGTGGGCTGTGCCACCCGCAACAGGACGGTGCCCTTCTGCAG
CACTTTTGCAGCCTTCTTCACGCGGGCCTTTGACCAGATTCGCATGGCCGCCATCTCCGAGAGCAACA
TCAACCTCTGCGGCTCCCACTGCGGCGTTTCCATCGGGGAAGACGGGCCCTCCCAGATGGCCCTAGAA
GATCTGGCTATGTTTCGGTCAGTCCCCACATCAACTGTCTTTTACCCAAGTGATGGCGTTGCTACAGA
GAAGGCAGTGGAACTAGCCGCCAATACAAAGGGTATCTGCTTCATCCGGACCAGCCGCCCAGAAAATG
CCATCATCTATAACAACAATGAGGACTTCCAGGTCGGACAAGCCAAGGTGGTCCTGAAGAGCAAAGAT
GACCAGGTGACCGTTATCGGGGCTGGGGTGACCCTGCACGAGGCCTTGGCCGCTGCCGAACTGCTGAA
GAAAGAAAAGATCAACATCCGCGTGCTGGACCCCTTCACCATCAAGCCCCTGGACAGAAAACTCATTC
TCGACAGCGCTCGTGCCACCAAGGGCAGGATCCTCACCGTGGAGGACCATTATTATGAAGGTGGCATT
GGTGAGGCTGTGTCCAGTGCAGTAGTGGGCGAGCCTGGCATCACTGTCACCCACCTGGCAGTTAACCG
GGTACCAAGAAGTGGGAAGCCAGCTGAGCTGCTGAAGATGTTTGGTATCGACAGGGATGCCATTGCAC
AAGCTGTGAGGGGCCTCATCACCAAGGCCCATCATCACCACCATCACTAGGCAGGTGCGGCCGCTCTC
GAGCACCACCACCACCACCACTGGAGATCCCGGCTGCT

| | | | |
|---|---|---|---|
| NOV2e, CG175387-04 | | | |
| Protein Sequence | SEQ ID NO:16 | 582 aa | MW at 63701.1 kD |

MAVLFFHTMRYKSQDPRNPHNDRFVLSKGHAAPILYAVWAEAGFLAEAELLNLRKISSDLDGHPVPKQ
AFTDVATGSLGQGLGAACGMAYTGKYFDKASYRVYCLLGDGELSEGSVWEAMAFASIYKLDNLVAILD
INRLGQSDPAPLQHQMDIYQKRCEAFGWHAIIVDGHSVEELCKAFGQAKHQPTAIIAKTFKGRGITGV
EDKESWHGKPLPKNMAEQIIQEIYSQIQSKKKILATPPQEDAPSVDIANIRNPSLPSYKVGDKIATRK
AYGQALAKLGHASDRIIALDGDTKNSTFSEIFKKEHPDRFIECYIAEQNMVSIAVGCATRNRTVPFCS
TFAAFFTRAFDQIRMAAISESNINLCGSHCGVSIGEDGPSQMALEDLAMFRSVPTSTVFYPSDGVATE
KAVELAANTKGICFIRTSRPENAIIYNNNEDFQVGQAKVVLKSKDDQVTVIGAGVTLHEALAAAELLK
KEKINIRVLDPFTIKPLDRKLILDSARATKGRILTVEDHYYEGGIGEAVSSAVVGEPGITVTHLAVNR
VPRSGKPAELLKMFGIDRDAIAQAVRGLITKAHHHHHH

A ClustalW comparison of the above protein sequences yields the following sequence alignment shown in Table B5.

TABLE B5
Comparison of the NOV2 protein sequences.

| | |
|---|---|
| NOV2a | —————MESYHKPDQQKLQALKDTANRLRISSIQATTAAGSGHPTSCCSAAEIMAVLFFHT |
| NOV2b | HHHHHHESYHKPDQQKLQALKDTANRLRISSIQATTAAGSGHPTSCCSAAEIMAVLFFHT |
| NOV2c | —PNSTMESYHKPDQQKLQALKDTANRLRISSIQATTAAGSGHPTSCCSAAEIMAVLFFHT |
| NOV2d | —PNSTMESYHKPDQQKLQALKDTANRLRISSIQATTAAGSGHPTSCCSAAEIMAVLFFHT |
| NOV2e | ————————————————————————————————————————————————————MAVLFFHT |
| NOV2a | MRYKSQDPRNPHNDRFVLSKGHAAPILYAVWAEAGFLAEAELLNLRKISSDLDGHPVPKQ |
| NOV2b | MRYKSQDPRNPHNDRFVLSKGHAAPILYAVWAEAGFLAEAELLNLRKISSDLDGHPVPKQ |
| NOV2c | MRYKSQDPRNPHNDRFVLSKGHAAPILYAVWAEAGFLAEAELLNLRKISSDLDGHPVPKQ |
| NOV2d | MRYKSQDPRNPHNDRFVLSKGHAAPILYAVWAEAGFLAEAELLNLRKISSDLDGHPVPKQ |
| NOV2e | MRYKSQDPRNPHNDRFVLSKGHAAPILYAVWAEAGFLAEAELLNLRKISSDLDGHPVPKQ |
| NOV2a | AFTDVATGSLGQGLGAACGMAYTGKYFDKASYRVYCLLGDGELSEGSVWEAMAFASIYKL |
| NOV2b | AFTDVATGSLGQGLGAACGMAYTGKYFDKASYRVYCLLGDGELSEGSVWEAMAFASIYKL |
| NOV2c | AFTDVATGSLGQGLGAACGMAYTGKYFDKASYRVYCLLGDGELSEGSVWEAMAFASIYKL |
| NOV2d | AFTDVATGSLGQGLGAACGMAYTGKYFDKASYRVYCLLGDGELSEGSVWEAMAFASIYKL |
| NOV2e | AFTDVATGSLGQGLGAACGMAYTGKYFDKASYRVYCLLGDGELSEGSVWEAMAFASIYKL |
| NOV2a | DNLVAILDINRLGQSDPAPLQHQMDIYQKRCEAFGWHAIIVDGHSVEELCKAFGQAKHQP |
| NOV2b | DNLVAILDINRLGQSDPAPLQHQMDIYQKRCEAFGWHAIIVDGHSVEELCKAFGQAKHQP |
| NOV2c | DNLVAILDINRLGQSDPAPLQHQMDIYQKRCEAFGWHAIIVDGHSVEELCKAFGQAKHQP |
| NOV2d | DNLVAILDINRLGQSDPAPLQHQMDIYQKRCEAFGWHAIIVDGHSVEELCKAFGQAKHQP |
| NOV2e | DNLVAILDINRLGQSDPAPLQHQMDIYQKRCEAFGWHAIIVDGHSVEELCKAFGQAKHQP |
| NOV2a | TAIIAKTFKGRGITGVEDKESWHGKPLPKNMAEQIIQEIYSQIQSKKKILATPPQEDAPS |
| NOV2b | TAIIAKTFKGRGITGVEDKESWHGKPLPKNMAEQIIQEIYSQIQSKKKILATPPQEDAPS |
| NOV2c | TAIIAKTFKGRGITGVEDKESWHGKPLPKNMAEQIIQEIYSQIQSKKKILATPPQEDAPS |
| NOV2d | TAIIAKTFKGRGITGVEDKESWHGKPLPKNMAEQIIQEIYSQIQSKKKILATPPQEDAPS |
| NOV2e | TAIIAKTFKGRGITGVEDKESWHGKPLPKNMAEQIIQEIYSQIQSKKKILATPPQEDAPS |
| NOV2a | VDIANIRMPSLPSYKVGDKIATRKAYGQALAKLGHASDRIIALDGDTKNSTFSEIFKKEH |
| NOV2b | VDIANIRMPSLPSYKVGDKIATRKAYGQALAKLGHASDRIIALDGDTKNSTFSEIFKKEH |
| NOV2c | VDIANIRMPSLPSYKVGDKIATRKAYGQALAKLGHASDRIIALDGDTKNSTFSEIFKKEH |
| NOV2d | VDIANIRMPSLPSYKVGDKIATRKAYGQALAKLGHASDRIIALDGDTKNSTFSEIFKKEH |
| NOV2e | VDIANIRMPSLPSYKVGDKIATRKAYGQALAKLGHASDRIIALDGDTKNSTFSEIFKKEH |
| NOV2a | PDRFIECYIAEQNNVSIAVGCATRNRTVPFCSTFAAFFTRAFDQIRMAAISESNINLCGS |
| NOV2b | PDRFIECYIAEQNMVSIAVGCATRNRTVPFCSTFAAFFTRAFDQIRMAAISESNINLCGS |
| NOV2c | PDRFIECYIAEQNMVSIAVGCATRNRTVPFCSTFAAFFTRAFDQIRMAAISESNINLCGS |
| NOV2d | PDRFIECYIAEQNMVSIAVGCATRNRTVPFCSTFAAFFTRAFDQIRMAAISESNINLCGS |
| NOV2e | PDRFIECYIAEQNMVSIAVGCATRNRTVPFCSTFAAFFTRAFDQIRMAAISESNINLCGS |
| NOV2a | HCGVSIGEDGPSQMALEDLAMFRSVPTSTVFYPSDGVATEKAVELAANTKGICFIRTSRP |
| NOV2b | HCGVSIGEDGPSQMALEDLAMFRSVPTSTVFYPSDGVATEKAVELAANTKGICFIRTSRP |
| NOV2c | HCGVSIGEDGPSQMALEDLANFRSVPTSTVFYPSDGVATEKAVELAANTKGICFIRTSRP |
| NOV2d | HCGVSIGEDGPSQMALEDLAMFRSVPTSTVFYPSDGVATEKAVELAANTKGICFIRTSRP |
| NOV2e | HCGVSIGEDGPSQMALEDLAMFRSVPTSTVFYPSDGVATEKAVELAANTKGICFIRTSRP |
| NOV2a | ENAIIYNNNEDFQVGQAKVVLKSKDDQVTVIGAGVTLHEALAAAELLKKEKINIRVLDPF |
| NOV2b | ENAIIYNNNEDFQVGQAKVVLKSKDDQVTVIGAGVTLHEALAAAELLKKEKINIRVLDPF |
| NOV2c | ENAIIYNNNEDFQVGQAKVVLKSKDDQVTVIGAGVTLHEALAAAELLKKEKINIRVLDPF |
| NOV2d | ENAIIYNNNEDFQVGQAKVVLKSKDDQVTVIGAGVTLHEALAAAELLKKEKINIRVLDPF |
| NOV2e | ENAIIYNNNEDFQVGQAKVVLKSKDDQVTVIGAGVTLHEALAAAELLKKEKINIRVLDPF |
| NOV2a | TIKPLDRKLILDSARATKGRILTVEDHYYEGGIGEAVSSAVVGEPGITVTHLAVNRVPRS |
| NOV2b | TIKPLDRKLILDSARATKGRILTVEDHYYEGGIGEAVSSAVVGEPGITVTHLAVNRVPRS |
| NOV2c | TIKPLDRKLILDSARATKGRILTVEDHYYEGGIGEAVSSAVVGEPGITVTHLAVNRVPRS |
| NOV2d | TIKPLDRKLILDSARATKGRILTVEDHYYEGGIGEAVSSAVVGEPGITVTHLAVNRVPRS |
| NOV2e | TIKPLDRKLILDSARATKGRILTVEDHYYEGGIGEAVSSAVVGEPGITVTHLAVNRVPRS |
| NOV2a | GKPAELLKMFGIDRDAIAQAVRGLITKA—————— |
| NOV2b | GKPAELLKMFGIDRDAIAQAVRGLITKA—————— |
| NOV2c | GKPAELLKMFGIDRDAIAQAVRGLITKA—————— |
| NOV2d | GKPAELLKMFGIDRDAIAQAVRGLITKA—————— |
| NOV2e | GKPAELLKMFGIDRDAIAQAVRGLITKAHHHHHH |
| NOV2a | (SEQ ID NO:8) |
| NOV2b | (SEQ ID NO:10) |
| NOV2c | (SEQ ID NO:12) |
| NOV2d | (SEQ ID NO:14) |
| NOV2e | (SEQ ID NO:16) |

Further analysis of the NOV2a protein yielded the following properties shown in Table B6.

TABLE B6
Protein Sequence Properties NOV2a

| SignalP | No Known Signal Sequence Predicted | | | |
|---|---|---|---|---|
| analysis: | | | | |
| PSORT II | PSG: a new signal peptide prediction method | | | |
| analysis: | N-region: length 11; pos. chg 2; neg. chg 2 | | | |
| | H-region: length 4; peak value −6.34 | | | |
| | PSG score: −10.74 | | | |
| | GvH: von Heijne's method for signal seq. recognition | | | |
| | GvH score (threshold: −2.1): −6.01 | | | |
| | possible cleavage site: between 41 and 42 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 1.11 (at 503) | | | |
| | ALOM score: 1.11 (number of TMSs: 0) | | | |
| | MITDISC: discrimination of mitochondrial targeting seq | | | |
| | R content: | 0 | Hyd Moment(75): | 4.90 |
| | Hyd Moment(95): | 4.71 | G content: | 0 |
| | D/E content: | 2 | S/T content: | 1 |

```
Score: −7.08
Gavel: prediction of cleavage sites for mitochondrial preseq
cleavage site motif not found
NUCDISC: discrimination of nuclear localization signals
pat4: none
pat7: none
bipartite: none
content of basic residues: 11.4%
NLS Score: −0.47
KDEL: ER retention motif in the C-terminus: none
ER Membrane Retention Signals:
KKXX-like motif in the C-terminus: LITK
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear
discrimination
Prediction: cytoplasmic
Reliability: 76.7
COIL: Lupas's algorithm to detect coiled-coil regions
total: 0 residues
Final Results (k = 9/23):
60.9%: cytoplasmic
26.1%: nuclear
  4.3%: mitochondrial
  4.3%: vacuolar
  4.3%: peroxisomal
>> prediction for CG175387-01 is cyt (k = 23)
```

A search of the NOV2a protein against the Geneseq database, a proprietary database that contains sequences published in patents and patent publication, yielded several homologous proteins shown in Table B7.

TABLE B7
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB08374 | Mouse transkelotase-like enzyme | 1 . . . 622 | 590/622 (94%) | 0.0 |
| | amino acid sequence - Mus sp, 623 aa. | 1 . . . 622 | 610/622 (97%) | |
| | [WO200192310-A2, 06-DEC-2001] | | | |
| ABU53000 | Human metabolism-associated | 6 . . . 616 | 582/611 (95%) | 0.0 |
| | DKFZphtes3_17117 homologue #1 - | 1 . . . 611 | 600/611 (97%) | |
| | Homo sapiens, 611 aa. | | | |
| | [WO200112659-A2, 22-FEB-2001] | | | |
| AAE33377 | Human DME-3 protein - Homo | 6 . . . 616 | 416/614 (67%) | 0.0 |
| | sapiens, 738 aa. [WO200290521-A2, | 119 . . . 732 | 499/614 (80%) | |
| | 14-NOV-2002] | | | |
| ABU52999 | Human metabolism-associated | 6 . . . 616 | 415/614 (67%) | 0.0 |
| | protein from DKFZphtes3_17117 - | 7 . . . 620 | 498/614 (80%) | |
| | Homo sapiens, 626 aa. | | | |
| | [WO200112659-A2, 22-FEB-2001] | | | |
| ABB08373 | Human transkelotase-like enzyme | 6 . . . 616 | 415/614 (67%) | 0.0 |
| | amino acid sequence - Homo sapiens, | 6 . . . 619 | 498/614 (80%) | |
| | 625 aa. [WO200192310-A2, 06-DEC-2001] | | | |

In a BLAST search of public sequence databases, the NOV2a protein was found to have homology to the proteins shown in the BLASTP data in Table B8.

TABLE B8
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P29401 | Transketolase (EC 2.2.1.1) (TK) - | 1 . . . 623 | 623/623 (100%) | 0.0 |
| | Homo sapiens (Human), 623 aa. | 1 . . . 623 | 623/623 (100%) | |
| A45050 | transketolase (EC 2.2.1.1) - | 1 . . . 623 | 619/623 (99%) | 0.0 |
| | human, 623 aa. | 1 . . . 623 | 621/623 (99%) | |
| P40142 | Transketolase (EC 2.2.1.1) (TK) | 1 . . . 622 | 590/622 (94%) | 0.0 |
| | (P68) - Mus musculus (Mouse), | 1 . . . 622 | 610/622 (97%) | |
| | 623 aa. | | | |
| P50137 | Transketolase (EC 2.2.1.1) (TK) - | 1 . . . 622 | 586/622 (94%) | 0.0 |
| | Rattus norvegicus (Rat), 623 aa. | 1 . . . 622 | 609/622 (97%) | |
| Q9ESA0 | Transketolase - Mus musculus | 65 . . . 622 | 529/558 (94%) | 0.0 |
| | (Mouse), 559 aa (fragment). | 1 . . . 558 | 548/558 (97%) | |

PFam analysis predicts that the NOV2a protein contains the domains shown in the Table B9.

TABLE B9
Domain Analysis of NOV2a

| | | Identities/ | |
|---|---|---|---|
| | | Similarities for | |
| | NOV2a | the Matched | Expect |
| Pfam Domain | Match Region | Region | Value |
| E1_dehydrog | 58 . . . 302 | 59/329 (18%) | 0.011 |
| | | 150/329 (46%) | |
| transketolase | 14 . . . 304 | 108/339 (32%) | 3.9e−159 |
| | | 290/339 (86%) | |
| transket_pyr | 314 . . . 479 | 53/188 (28%) | 1.3e−56 |
| | | 143/188 (76%) | |
| transketolase_C | 490 . . . 612 | 41/136 (30%) | 4.9e−34 |
| | | 105/136 (77%) | |

### EXAMPLE B3

### Transketolase Gene Variants and SNPs

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification:

SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing. Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265.

In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

Results

The DNA and protein sequences for the novel single nucleotide polymorphic variants of the transketolase-like gene of CuraGen Acc. No. CG175387-01 are reported in Table B10. Variants are reported individually but any combination of all or a select subset of variants are also included. In Table B10, the positions of the variant bases and the variant amino acid residues are underlined. In summary, there are 22 variants reported in Table B 10. Variant 13377687 is a G to A SNP at 134 bp of the nucleotide sequence that results in an Ala to Thr change at amino acid 19 of protein sequence, variant 13377688 is a C to T SNP at 287 bp of the nucleotide sequence that results in an Arg to Cys change at amino acid 70 of protein sequence, variant 13377684 is a G to T SNP at 566 bp of the nucleotide sequence that results in a Val to Leu change at amino acid 163 of protein sequence, variant 13377689 is an A to G SNP at 651 bp of the nucleotide sequence that results in an Asp to Gly change at amino acid 191 of protein sequence, variant 13380050 is an A to T SNP at 699 bp of the nucleotide sequence that results in a Glu to Val change at amino acid 207 of protein sequence, variant 13380051 is a T to C SNP at 741 bp of the nucleotide sequence that results in a Val to Ala change at amino acid 221 of protein sequence, variant 13377683 is an A to G SNP at 747 bp of the nucleotide sequence that results in a Glu to Gly change at amino acid 223 of protein sequence, variant 13377690 is an A to G SNP at 894 bp of the nucleotide sequence that results in a Gln to Arg change at amino acid 272 of protein sequence, variant 13377682 is a T to C SNP at 1253 bp of the nucleotide sequence that results in a Phe to Leu change at amino acid 392 of protein sequence, variant 13380092 is an A to G SNP at 1307 bp of the nucleotide sequence that results in an Ile to Val change at amino acid 410 of protein sequence, variant 13380073 is an A to G SNP at 1441 bp of the nucleotide sequence that results in no change in the protein sequence (silent), variant 13380072 is a C to T SNP at 1519 bp of the nucleotide sequence that results in no change in the protein sequence (silent), variant 13380093 is an A to G SNP at 1529 bp of the nucleotide sequence that results in an Asn to Asp change at amino acid 484 of protein sequence, variant 13380094 is a C to T SNP at 1550 bp of the nucleotide sequence that results in a Gln to Stop change at amino acid 491 of protein sequence, variant 13380095 is a T to C SNP at 1603 bp of the nucleotide sequence that results in no change in the protein sequence (silent), variant 13380052 is a G to T SNP at 1645 bp of the nucleotide sequence that results in no change in the protein sequence (silent), variant 13377691 is a T to C SNP at 1683 bp of the nucleotide sequence that results in a Phe to Ser change at amino acid 535 of protein sequence, variant 13380053 is a T to C SNP at 1783 bp of the nucleotide sequence that results in no change in the protein sequence (silent), variant 13377686 is a G to A SNP at 1802 bp of the nucleotide sequence that results in an Ala to Thr change at amino acid 575 of protein sequence, variant 13380055 is a C to T SNP at 1813 bp of the nucleotide sequence that results in no change in the protein sequence (silent), variant 13377685 is an A to G SNP at 1832 bp of the nucleotide sequence that results in a Thr to Ala change at amino acid 585 of protein sequence, and variant 13380056 is an A to G SNP at 1862 bp of the nucleotide sequence that results in a Ser to Gly change at amino acid 595 of protein sequence.

TABLE B10
Variants of nucleotide sequence of
Acc. No. CG175387-01 (SEQ ID NO: 7)

| | Nucleotides | | | Amino Acids | | |
|---|---|---|---|---|---|---|
| Variant | Position | Initial | Modified | Position | Initial | Modified |
| 13377687 | 134 | G | A | 19 | Ala | Thr |
| 13377688 | 287 | C | T | 70 | Arg | Cys |
| 13377684 | 566 | G | T | 163 | Val | Leu |
| 13377689 | 651 | A | G | 191 | Asp | Gly |
| 13380050 | 699 | A | T | 207 | Glu | Val |
| 13380051 | 741 | T | C | 221 | Val | Ala |
| 13377683 | 747 | A | G | 223 | Glu | Gly |
| 13377690 | 894 | A | G | 272 | Gln | Arg |
| 13377682 | 1253 | T | C | 392 | Phe | Leu |
| 13380092 | 1307 | A | G | 410 | Ile | Val |
| 13380073 | 1441 | A | G | 454 | Thr | Thr |
| 13380072 | 1519 | C | T | 480 | Ile | Ile |
| 13380093 | 1529 | A | G | 484 | Asn | Asp |
| 13380094 | 1550 | C | T | 491 | Gln | STOP |
| 13380095 | 1603 | T | C | 508 | Ala | Ala |
| 13380052 | 1645 | G | T | 522 | Leu | Leu |
| 13377691 | 1683 | T | C | 535 | Phe | Ser |
| 13380053 | 1783 | T | C | 568 | Ile | Ile |
| 13377686 | 1802 | G | A | 575 | Ala | Thr |
| 13380055 | 1813 | C | T | 578 | Gly | Gly |
| 13377685 | 1832 | A | G | 585 | Thr | Ala |
| 13380056 | 1862 | A | G | 595 | Ser | Gly |

TABLE B11
Variant Sequences

```
Table B11A1.
Nucleotide sequence of variant 13377687 NOV2a1n (underlined). G/A (SEQ ID NO:111)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGACCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGCTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAAGGTGCCAAAGATGTACTGAGAGGAGGGGTAAATATATGTTTTGAGAAAAATGAAAAAAAAAAAAAAAAAAAAA
Table B11A2.
Protein sequence of variant NOV2a1p (underlined). (SEQ ID NO:112)
      1 MESYHKPDQQKLQALKDTTNRLRISSIQATTAAGSGHPTSCCSAAEIMAVLFFHTMRYKSQDPRNPHNORFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSOIQSKKKILATPPQEDAPSVDIANIRNPSLPSYKVGDKIATRKA
  321 YGQALAKLGHASDRIIALDGDTKNSTFSEIFKKEHPDRFIECYIAEQNMVSIAVGCATRNRTVPFCSTEAAFFTRAFDQI
  401 RMAAISESNINLCGSHCGVSIGEDGPSQMALEDLANFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11A3. Alteration effect
Ala to Thr
Table B11B1.
Nucleotide sequence of variant 13377688 NOV2a2n (underlined). C/T (SEQ ID NO:113)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACTGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTCGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATCGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTCGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATAAAAAAAAAAAAAAAAAAAAAA
Table B11B2. Protein sequence of variant NOV2a2p (underlined). (SEQ ID NO:114)
      1 MESYHKPDQQKLQALKDTANRLRISSIQATTAAGSGHPTSCCSAAEIMAVLFFHTMRYKSQDPRNPHNDCFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQNNVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11B3. Alteration effect
Arg to Cys
Table B11C1.
Nucleotide sequence of variant 13377684 NOV2a3n (underlined). G/T (SEQ ID NO:115)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGCGAGCTGTCAGAGG
  561 GCTCTTTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11C2. Protein sequence of variant NOV2a3p (underlined). (SEQ ID NO:116)
      1 MESYHKPDQQKLQALKDTANRLRISSIQATTAAGSGHPTSCCSAAEIMAVLFFHTMRYKSQDPRNPRNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSLWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQNNVSIAVGCATRNRTVPFCSTFAAFFTRAFDQI
  401 RMAAISESNINLCGSHCGVSIGEDGPSQMALEDLANPRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11C3. Alteration effect
Val to Leu
Table B11D1.
Nucleotide sequence of variant 13377689 NOV2a4n (underlined). A/G (SEQ ID NO:117)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGGCCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAACCCCCTGGACAGAAAACTCATTCTCGACAGCGCTCGTGCCACCAAGGGCAGGATCCTCACCGTGGAGG
1761 ACCATTATTATCAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11D2. Protein sequence of variant NOV2a4p (underlined). (SEQ ID NO:118)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYPDKASYRVYCLLGDGELSE
  161 GSVWEAMAFASIYKLDNLVAILDINRLGQSGPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFIIKPLDRKLILDSARATKGRILTVE
  561 DHYYEGGIGEAVSSAVVGEPGITVTHLAVNRVPRSGKPAELLKMFGIDRDAIAQAVRGLITKA
Table B11D3. Alteration effect
Asp to Gly
Table B11E1.
Nucleotide sequence of variant 13380050 NOV2a5n (underlined). A/T (SEQ ID NO:119)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCCCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTCAACCTGAGGAAGATCAGCTCCGACTTG
  401 GACGGGCACCCGGTCCCGAAACAAGCTTTCACCGACGTGGCCACTGCCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGTGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 CAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGACGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11E2. Protein sequence of variant NOV2a5p (underlined). (SEQ ID NO:120)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCVAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11E3. Alteration effect
Glu to Val
Table B11F1.
Nucleotide sequence of variant 13380051 NOV2a6n (underlined). T/C (SEQ ID NO:121)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGCTCTGGGCTGAAGCTGGTTTCCTGGCCGAGGCGGAGCTGCTGAACGTGAGGAAGATCAGCTCCGACTTG
  401 GACGGGCACCCGGTCCCGAAACAAGCTTTCACCGACGTGGCCACTGGCTCCCTCGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGCGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAACATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTCTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11F2. Protein sequence of variant NOV2a6p (underlined). (SEQ ID NO:122)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSAEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKCRILTVE
  561 DHYYEGCIGEAVSSAVVGEPGITVTHLAVNRVPRSGKPAELLKMFGIDRDAIAQAVRGLITKA
Table B11F3. Alteration effect
Val to Ala
Table B11G1.
Nucleotide sequence of variant 13377683 NOV2a7n (underlined). A/G (SEQ ID NO:123)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGCCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGGGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11G2. Protein sequence of variant NOV2a7p (underlined). (SEQ ID NO:124)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEGLCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQNNVSIAVGCATRNRTVPFCSTFAAFFTRAFDQI
  401 RMAAISESNINLCGSHCGVSIGEDGPSQMALEDLANFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11G3. Alteration effect
Glu to Gly
Table B11H1.
Nucleotide sequence of variant 13377690 NOV2a8n (underlined). A/G (SEQ ID NO:125)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCcTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCGGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGThATATATGTTTTGAGAAAAATGAAAAAAAAAAAAAAAAAAAAAA
Table B11H2. Protein sequence of variant NOV2a8p (underlined). (SEQ ID NO:126)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIREIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLANFRSVPTSTVFYPSDGVATEKAVELAANTKGICFIRTSRPENAII
  481 YNNNEOFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVMRVPRSGKPAELLKMFGIDRDAIAQAVRGLITKA
Table B11H3. Alteration effect
Gln to Arg
Table B11I1.
Nucleotide sequence of variant 13377682 NOV2a9n (underlined). T/C (SEQ ID NO:127)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCCTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGCGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11I2. Protein sequence of variant NOV2a9p (underlined). (SEQ ID NO:128)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPOEDAPSVDIANIRMPSLPSYKVGDKIATRKA
  321 YGQALAKLGHASDRIIALDGDTKNSTFSEIFKKEHPDRFIECYIAEQNMVSIAVGCATRNRTVPPCSTFAALFTRAFDQI
  401 RMAAISESNINLCGSHCGVSIGEDGPSQMALEDLANFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11I3. Alteration effect
Phe to Leu
Table B11J1.
Nucleotide sequence of variant 13380092 NOV2a10n (underlined). A/G (SEQ ID NO:129)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGCTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACGTCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTCCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTCAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11J2. Protein sequence of variant NOV2a10p (underlined). (SEQ ID NO:130)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGOGLGAACGMAYTGKYFDKASYRVYCLLGDGELSE
  161 GSVWEANAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSBIFKKEHPDRFIECYIAEQNMVSIAVGCATRNRTVPFCSTFAAFFTRAFDQI
  401 RMAAISESNVNLCGSHCGVSIGEDGPSQMALEDLANFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11J3. Alteration effect
Ile to Val
Table B11K1.
Nucleotide sequence of variant 13380073 NOV2a11n (underlined). A/G (SEQ ID NO:131)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCcA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTCCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 GGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGCACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11K2. Protein sequence of variant NOV2a11p (underlined). (SEQ ID NO:132)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11K3. Alteration effect
No change.
Table B11L1.
Nucleotide sequence of variant 13380072 NOV2a12n (underlined). C/T (SEQ ID NO:133)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATCGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATTT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGACCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAA
Table B11L2. Protein sequence of variant NOV2a12p (underlined). (SEQ ID NO:134)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGOGLGAACGMAYTGKYFDKASYRVYCLLGDGELSE
  161 GSVWEAMAFASIYKLDNLVAILDINRLGOSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLANFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11L3. Alteration effect
No change.
Table B11M1.
Nucleotide sequence of variant 13380093 NOV2a13n (underlined). A/G (SEQ ID NO:135)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACACCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAACGGTATCTGCTTCATCCGGACCAGCCGCCCAGAAAATGCCATCATCT
1521 ATAACAACGATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11M2. Protein sequence of variant NOV2a13p (underlined). (SEQ ID NO:136)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDThNSTFSEIFKKEHPDRFIECYIAEQNMVSIAVGCATRNRTVPFCSTFAAFPTRAFDQI
  401 RMAAISESNINLCGSHCGVSIGEDGPSQMALEDLAMFRSVPTSTVFYPSDGVATEKAVELAANTKGICFIRTSRPENAII
  481 YNNDEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11M3. Alteration effect
Asn to Asp
Table B11N1.
Nucleotide sequence of variant 13380094 NOV2a14n (underlined). C/T (SEQ ID NO:137)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGATAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAACAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11N2. Protein sequence of variant NOV2a14p (underlined). (SEQ ID NO:138)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTCKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVG*AKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11N3. Alteration effect
Gln to STOP
Table B11O1.
Nucleotide sequence of variant 13380095 NOV2a15n (underlined). T/C (SEQ ID NO:139)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAACATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCCGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11O2. Protein sequence of variant NOV2a15p (underlined). (SEQ ID NO:140)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYPDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIAThKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLANFRSVPTSTVFYPSDGVATEKAVELAANTKGICFIRTSRPENAII
  481 YNNNEDFQVGQAKVVLKSKDDQWEVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKNFGIDRDAIAQAVRGLITKA
Table B11O3. Alteration effect
No change.
Table B11P1.
Nucleotide sequence of variant 13380052 NOV2a16n (underlined). G/T (SEQ ID NO:141)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAOAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTTAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAATATATGTTTTGAGAAAAATGAAAAAAAAAAAAAAAAAAAAAA
Table B11P2. Protein sequence of variant NOV2a16p (underlined). (SEQ ID NO:142)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWEAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQNMVSIAVGCATRNRTVPFCSTFAAFFTRAFDQI
  401 RMAAISESNINLCGSHCGVSIGEDGPSQMALEDLAMFRSVPTSTVPYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11P3. Alteration effect
No change.
Table B11Q1.
Nucleotide sequence of variant 13377691 NOV2a17n (underlined). T/C (SEQ ID NO:143)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGAACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTCCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11Q2. Protein sequence of variant NOV2a17p (underlined). (SEQ ID NO:144)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQNNVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPSTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11Q3. Alteration effect
Phe to Ser
Table B11R1.
Nucleotide sequence of variant 13380053 NOV2a18n (underlined). T/C (SEQ ID NO:145)
      1 GGCACGAGGGCCTGTCGCCGCGGGAGCAGCCGCTATCTCTGTGTGTCCGCGTGTGCGCCCGGTCCCCGCCTGCCGCACCA
    81 TGGACAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTCCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATCGGTGAGGCTGTGTCCAGTGCAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11R2. Protein sequence of variant NOV2a18p (underlined). (SEQ ID NO:146)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGOGLGAACGMAYTGKYFDKASYRVYCLLGDGELSE
  161 GSVWEAMAFASIYKLDNLVAILDINRLGOSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQWMVSIAVGCATRNRTVPFCSTFAAFFTRAFDQI
  401 RMAAISESNINLCGSHCGVSIGEDGPSQMALEDLAMFRSVPTSTVPYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11R3. Alteration effect
No change.
3S1.
Nucleotide sequence of variant 13377686 NOV2a19n (underlined). G/A (SEQ ID NO:147)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGAGTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCTGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTACAGTAGTGGGCGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAGGTGCTCAAAGATGTACTGAGAGGAGGGGTAAATATATGTTTTGAGAAAAATGAAAAAAAAAAAAAAAAAAAAAA
Table B11S2. Protein sequence of variant NOV2a19p (underlined). (SEQ ID NO:148)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQWMVSIAVGCATRNRTVPFCSTFAAFFTRAFDQI
  401 RMAAISESNINLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSTVVGEPGITVTHLAVNRVPRSGKPAELLKMFGIDRDAIAQAVRGLITKA
Table B11S3. Alteration effect
Ala to Thr
Table B11T1.
Nucleotide sequence of variant 13380055 NOV2a20n (underlined). C/T (SEQ ID NO:149)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CACGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGCCTGAAGCTGGTTTCCTCGCCGAGGCGGAGCTGCTGAACCTGAGGAAGATCAGCTCCGACTTG
  401 GACGGGCACCCGGTCCCGAAACAAGCTTTCACCGACGTGGCCACTGGOTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGCGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACCGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AOAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCACGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGTGAGCCTGGCATCACTGTCACCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGCGTAAATATATGTTTTGAGAAAAATAAAAAAAAAAAAAAAAAAAAAA
Table B11T2. Protein sequence of variant NOV2a20p (underlined). (SEQ ID NO:150)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGOSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLPSYKVGDKIATRKA
  321 YGQALAKLGHASDRIIALDGDTKNSTFSEIFKKEHPDRFIECYIAEQNMVSIAVGCATRNRTVPFCSTEAAFFTRAFDQI
  401 RMAAISESNINLCGSHCGVSIGEDGPSQMALEDLAMFRSVPTSTVFYPSDGVATEKAVELAANTKGICPIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRSGKPAELLKMFGIDRDAIAQAVRGLITKA
Table B11T3. Alteration effect
No change.
Table B11U1.
Nucleotide sequence of variant 13377685 NOV2a21n (underlined). A/G (SEQ ID NO:151)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTG
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATCT
1521 ATAACAACAATGAGGACTTCCAGGTCGGACAAGCCAAGGTGGTCCTGAAGAGCAAGGATGACCAGGTGACCGTTATCGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCGCCCACCTG
1841 GCAGTTAACCGGGTACCAAGAAGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11U2. Protein sequence of variant NOV2a21p (underlined). (SEQ ID NO:152)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGSLSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRMPSLPSYKVGDKIATRKA
  321 YGQALAKLGHASDRIIALDGDTKNSTFSEIFKKEHPDRFIECYIAEQNMVSIAVGCATRNRTVPPCSTFAAFFTRAFDQI
  401 RMAAISESNINLCGSHCGVSIGEDGPSQMALEDLANFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVAHLAVNRVPRSGKPAELLKMFGIDRDAIAQAVRGLITKA
Table B11U3. Alteration effect
Thr to Ala
Table B11V1.
Nucleotide sequence of variant 13380056 NOV2a22n (underlined). A/G (SEQ ID NO:153)
      1 GGCACGAGGGCCTGTCGCCGCGGGAGCAGCCGCTATCTCTGTGTGTCCGCGTGTGCGCCCGGTCCCCGCCTGCCGCACCA
    81 TGGAGAGCTACCACAAGCCTGACCAGCAGAAGCTGCAGGCCTTGAAGGACACGGCCAACCGCCTACGTATCAGCTCCATC
  161 CAGGCCACCACTGCGGCGGGCTCTGGCCACCCCACGTCATGCTGCAGCGCCGCAGAGATCATGGCTGTCCTCTTTTTCCA
  241 CACCATGCGCTACAAGTCCCAGGACCCCCGGAATCCGCACAATGACCGCTTTGTGCTCTCCAAGGGCCATGCAGCTCCCA
  321 TCCTCTACGCGGTCTGGGCTGAAGCTGGTTTCCTGGCCGAGGCGGAGCTGCTGAACCTGAGGAAGATCAGCTCCGACTTC
  401 GACGGGCACCCGGTCCCGAAACAAGCTTTCACCGACGTGGCCACTGGCTCCCTGGGCCAGGGCCTCGGGGCCGCTTGTGG
  481 GATGGCCTACACCGGCAAATACTTCGACAAGGCCAGCTACCGAGTCTATTGCTTGCTGGGAGATGGGGAGCTGTCAGAGG
  561 GCTCTGTATGGGAGGCCATGGCCTTCGCCAGCATCTATAAGCTGGACAACCTTGTGGCCATTCTAGACATCAATCGCCTG
  641 GGCCAGAGTGACCCGGCCCCGCTGCAGCACCAGATGGACATCTACCAGAAGCGGTGCGAGGCCTTCGGTTGGCATGCCAT
  721 CATCGTGGATGGACACAGCGTGGAGGAGCTGTGCAAGGCCTTTGGCCAGGCCAAGCACCAGCCAACAGCCATCATTGCCA
  801 AGACCTTCAAGGGCCGAGGGATCACGGGGGTAGAAGATAAGGAGTCTTGGCATGGGAAGCCCCTCCCCAAAAACATGGCT
  881 GAGCAGATCATCCAGGAGATCTACAGCCAGATCCAGAGCAAAAAGAAGATCCTGGCAACCCCTCCACAGGAGGACGCACC
  961 CTCAGTGGACATTGCCAACATCCGCATGCCCAGCCTGCCCAGCTACAAAGTTGGGGACAAGATAGCCACCCGCAAGGCCT
1041 ACGGGCAGGCACTGGCCAAGCTGGGCCATGCCAGTGACCGCATCATCGCCCTGGATGGGGACACCAAAAATTCCACCTTC
1121 TCGGAGATCTTCAAAAAGGAGCACCCGGACCGCTTCATCGAGTGCTACATTGCCGAGCAGAACATGGTGAGCATCGCGGT
1201 GGGCTGTGCCACCCGCAACAGGACGGTGCCCTTCTGCAGCACTTTTGCAGCCTTCTTCACGCGGGCCTTTGACCAGATTC
1281 GCATGGCCGCCATCTCCGAGAGCAACATCAACCTCTGCGGCTCCCACTGCGGCGTTTCCATCGGGGAAGACGGGCCCTCC
1361 CAGATGGCCCTAGAAGATCTGGCTATGTTTCGGTCAGTCCCCACATCAACTGTCTTTTACCCAAGTGATGGCGTTGCTAC
1441 AGAGAAGGCAGTGGAACTAGCCGCCAATACAAAGGGTATCTGCTTCATCCGGACCAGCCGCCCAGAAAATGCCATCATcT
1521 ATAACAACAATGAGGACTTCCAGGTCGGACAAGCCAAGGTGGTCCTGAAGAGCAAGGATGACCAGGTGACCGTTATcGGG
1601 GCTGGGGTGACCCTGCACGAGGCCTTGGCCGCTGCCGAACTGCTGAAGAAAGAAAAGATCAACATCCGCGTGCTGGACCC
1681 CTTCACCATCAAGCCCCTGGACAGAAAACTCATTCTCGACAGCGCTCGTGCCACCAAGGGCAGGATCCTCACCGTGGAGG
1761 ACCATTATTATGAAGGTGGCATTGGTGAGGCTGTGTCCAGTGCAGTAGTGGGCGAGCCTGGCATCACTGTCACCCACCTG
1841 GCAGTTAACCGGGTACCAAGAGGTGGGAAGCCAGCTGAGCTGCTGAAGATGTTTGGTATCGACAGGGATGCCATTGCACA
1921 AGCTGTGAGGGGCCTCATCACCAAGGCCTAGGGCGGGTATGAAGTGTGGGGCGGGGGTCTATACATTCCTGAGATTCTGG
2001 GAAAGGTGCTCAAAGATGTACTGAGAGGAGGGGTAAATATATGTTTTGAGAAAAATGAAAAAAAAAAAAAAAAAAAAA
Table B11V2. Protein sequence of variant NOV2a22p (underlined). (SEQ ID NO:154)
      1 MESYHKPDQQKLQALKDTANRLRISSIQATTAAGSGHPTSCCSAAEIMAVLFFHTMRYKSQDPRNPHNDRFVLSKGHAAP
    81 ILYAVWAEAGFLAEAELLNLRKISSDLDGHPVPKQAFTDVATGSLGQGLGAACGMAYTGKYFDKASYRVYCLLGDGELSE
  161 GSVWEAMAFASIYKLDNLVAILDINRLGQSDPAPLQHQMDIYQKRCEAFGWHAIIVDGHSVEELCKAFGQAKHQPTAIIA
  241 KTFKGRGITGVEDKESWHGKPLPKNMAEQIIQEIYSQIQSKKKILATPPQEDAPSVDIANIRNPSLpSyKvGDKIATRKA
  321 YGQALAKLGHASDRIIALDGDTKNSTFSEIFKKEHPDRFIECYIAEQNMVSIAVGCATHNRTVPFCSTFAAFFTRAPDQI
  401 RMAAISESNIHLCGSHCGVSIGEDGPSQMALEDLAMFRSVPTSTVFYPSDGVATEKAVELAANTKGICFIRTSRPENAII
  481 YNNNEDFQVGQAKVVLKSKDDQVTVIGAGVTLHEALAAAELLKKEKINIRVLDPFTIKPLDRKLILDSARATKGRILTVE
  561 DHYYEGGIGEAVSSAVVGEPGITVTHLAVNRVPRGGKPAELLKMFGIDRDAIAQAVRGLITKA
Table B11V3. Alteration effect
Ser to Gly
```

### EXAMPLE B4

### Expression Profiles of Transketolase

The protocol for quantitative expression analysis is disclosed in Example Q9.

Expression of gene CG175387-01 and G175387-03 was assessed using the primer-probe set Ag6328, described in Table B 12. Results of the RTQ-PCR runs are shown in Tables B13, B14 and B15.

TABLE B12
Probe Name Ag6328

| | | | Start | SEQ ID |
|---|---|---|---|---|
| Primers | Sequences | Length | Position | No |
| Forward | 5′-cctagggcgggtatgaagt-3′ | 19 | 1947 | 180 |
| Probe | TET-5′-ccagaatctcaggaatgtatagacccc-3′- | 27 | 1974 | 181 |
| | TAMRA | | | |
| Reverse | 5′-tcagtacatctttgagcacctttc-3′ | 24 | 2001 | 182 |

TABLE B13
General screening panel v1.5

| | Rel. Exp. (%) |
|---|---|
| | Ag6328, Run |
| Tissue Name | 259211050 |
| Adipose | 4.1 |
| Melanoma* Hs688(A).T | 7.6 |
| Melanoma* Hs688(B).T | 8.2 |
| Melanoma* M14 | 17.9 |
| Melanoma* LOXIMVI | 13.1 |
| Melanoma* SK-MEL-5 | 10.5 |
| Squamous cell carcinoma SCC-4 | 8.0 |
| Testis Pool | 2.0 |
| Prostate ca.* (bone met) PC-3 | 2.6 |
| Prostate Pool | 1.6 |
| Placenta | 3.0 |
| Uterus Pool | 0.6 |
| Ovarian ca. OVCAR-3 | 5.4 |
| Ovarian ca. SK-OV-3 | 13.6 |
| Ovarian ca. OVCAR-4 | 5.1 |
| Ovarian ca. OVCAR-5 | 11.7 |
| Ovarian ca. IGROV-1 | 29.1 |
| Ovarian ca. OVCAR-8 | 10.6 |
| Ovary | 1.8 |
| Breast ca. MCF-7 | 4.5 |
| Breast ca. MDA-MB-231 | 19.2 |
| Breast ca. BT 549 | 55.1 |
| Breast ca. T47D | 7.9 |
| Breast ca. MDA-N | 85.9 |
| Breast Pool | 1.1 |
| Trachea | 1.2 |
| Lung | 2.1 |
| Fetal Lung | 5.3 |
| Lung ca. NCI-N417 | 6.3 |
| Lung ca. LX-1 | 9.5 |
| Lung ca. NCI-H146 | 2.7 |
| Lung ca. SHP-77 | 12.4 |
| Lung ca. A549 | 92.0 |
| Lung ca. NCI-H526 | 1.9 |
| Lung ca. NCI-H23 | 2.6 |
| Lung ca. NCI-H460 | 6.7 |
| Lung ca. HOP-62 | 9.5 |
| Lung ca. NCI-H522 | 2.0 |
| Liver | 0.3 |
| Fetal Liver | 2.4 |
| Liver ca. HepG2 | 7.6 |
| Kidney Pool | 1.4 |
| Fetal Kidney | 1.4 |
| Renal ca. 786-0 | 10.8 |
| Renal ca. A498 | 27.4 |
| Renal ca. ACHN | 18.8 |
| Renal ca. UO-31 | 10.4 |
| Renal ca. TK-10 | 6.9 |
| Bladder | 2.5 |
| Gastric ca. (liver met.) NCI-N87 | 3.7 |
| Gastric ca. KATO III | 29.7 |
| Colon ca. SW-948 | 2.6 |
| Colon ca. SW480 | 20.6 |
| Colon ca.* (SW480 met) SW620 | 7.6 |
| Colon ca. HT29 | 8.8 |
| Colon ca. HCT-116 | 30.1 |
| Colon ca. CaCo-2 | 12.3 |
| Colon cancer tissue | 7.9 |
| Colon ca. SW1116 | 5.2 |
| Colon ca. Colo-205 | 5.0 |
| Colon ca. SW-48 | 3.7 |
| Colon Pool | 0.8 |
| Small Intestine Pool | 1.3 |
| Stomach Pool | 1.1 |
| Bone Marrow Pool | 1.3 |
| Fetal Heart | 0.4 |
| Heart Pool | 0.6 |
| Lymph Node Pool | 2.6 |
| Fetal Skeletal Muscle | 1.0 |
| Skeletal Muscle Pool | 0.6 |
| Spleen Pool | 1.7 |
| Thymus Pool | 2.6 |
| CNS cancer (glio/astro) U87-MG | 16.6 |
| CNS cancer (glio/astro) U-118-MG | 10.9 |
| CNS cancer (neuro; met) SK-N-AS | 6.0 |
| CNS cancer (astro) SF-539 | 3.9 |
| CNS cancer (astro) SNB-75 | 100.0 |
| CNS cancer (glio) SNB-19 | 36.1 |
| CNS cancer (glio) SF-295 | 16.3 |
| Brain (Amygdala) Pool | 3.4 |
| Brain (cerebellum) | 8.0 |
| Brain (fetal) | 3.6 |
| Brain (Hippocampus) Pool | 1.4 |
| Cerebral Cortex Pool | 3.9 |
| Brain (Substantia nigra) Pool | 2.4 |
| Brain (Thalamus) Pool | 5.1 |
| Brain (whole) | 3.0 |
| Spinal Cord Pool | 3.6 |
| Adrenal Gland | 4.5 |
| Pituitary gland Pool | 1.0 |
| Salivary Gland | 0.9 |
| Thyroid (female) | 0.5 |
| Pancreatic ca. CAPAN2 | 27.9 |
| Pancreas Pool | 3.6 |

TABLE B14
Panel 5 Islet

| | Rel. Exp. (%) |
|---|---|
| | Ag6328, Run |
| Tissue Name | 259232256 |
| 97457_Patient-02go_adipose | 19.6 |
| 97476_Patient-07sk_skeletal muscle | 25.0 |
| 97477_Patient-07ut_uterus | 8.4 |
| 97478_Patient-07pl_placenta | 26.8 |
| 99167_Bayer Patient 1 | 91.4 |
| 97482_Patient-08ut_uterus | 6.7 |
| 97483_Patient-08pl_placenta | 4.3 |
| 97486_Patient-09sk_skeletal muscle | 1.7 |
| 97487_Patient-09ut_uterus | 6.9 |
| 97488_Patient-09pl_placenta | 5.0 |
| 97492_Patient-10ut_uterus | 19.6 |
| 97493_Patient-10pl_placenta | 26.8 |
| 97495_Patient-11go_adipose | 10.7 |
| 97496_Patient-11sk_skeletal muscle | 4.2 |
| 97497_Patient-11ut_uterus | 14.8 |
| 97498_Patient-11pl_placenta | 10.2 |
| 97500_Patient-12go_adipose | 29.1 |
| 97501_Patient-12sk_skeletal muscle | 10.0 |
| 97502_Patient-12ut_uterus | 21.2 |
| 97503_Patient-12pl_placenta | 12.3 |
| 94721_Donor 2 U - A_Mesenchymal Stem Cells | 38.4 |
| 94722_Donor 2 U - B_Mesenchymal Stem Cells | 32.3 |
| 94723_Donor 2 U - C_Mesenchymal Stem Cells | 27.4 |
| 94709_Donor 2 AM - A_adipose | 15.3 |
| 94710_Donor 2 AM - B_adipose | 18.3 |
| 94711_Donor 2 AM - C_adipose | 15.3 |
| 94712_Donor 2 AD - A_adipose | 12.9 |
| 94713_Donor 2 AD - B_adipose | 19.2 |
| 94714_Donor 2 AD - C_adipose | 23.8 |
| 94742_Donor 3 U - A_Mesenchymal Stem Cells | 17.6 |
| 94743_Donor 3 U - B_Mesenchymal Stem Cells | 28.1 |
| 94730_Donor 3 AM - A_adipose | 25.3 |
| 94731_Donor 3 AM - B_adipose | 22.4 |
| 94732_Donor 3 AM - C_adipose | 24.0 |
| 94733_Donor 3 AD - A_adipose | 26.4 |
| 94734_Donor 3 AD - B_adipose | 13.7 |
| 94735_Donor 3 AD - C_adipose | 23.5 |
| 77138_Liver_HepG2untreated | 43.5 |
| 73556_Heart_Cardiac stromal cells (primary) | 77.4 |
| 81735_Small Intestine | 21.8 |
| 72409_Kidney_Proximal Convoluted Tubule | 29.5 |
| 82685_Small intestine_Duodenum | 5.1 |
| 90650_Adrenal_Adrenocortical adenoma | 13.3 |
| 72410_Kidney_HRCE | 100.0 |
| 72411_Kidney_HRE | 39.5 |
| 73139_Uterus_Uterine smooth muscle cells | 12.9 |

TABLE B15
General_screening_panel_v1.6

| Tissue Name | A |
|---|---|
| Adipose | 3.7 |
| Melanoma* Hs688(A).T | 6.9 |
| Melanoma* Hs688(B).T | 7.0 |
| Melanoma* M14 | 21.5 |
| Melanoma* LOXIMVI | 9.6 |
| Melanoma* SK-MEL-5 | 10.6 |
| Squamous cell carcinoma SCC-4 | 13.8 |
| Testis Pool | 1.2 |
| Prostate ca.* (bone met) PC-3 | 3.7 |
| Prostate Pool | 1.7 |
| Placenta | 3.1 |
| Uterus Pool | 0.4 |
| Ovarian ca. OVCAR-3 | 6.3 |
| Ovarian ca. SK-OV-3 | 29.3 |
| Ovarian ca. OVCAR-4 | 9.6 |
| Ovarian ca. OVCAR-5 | 23.0 |
| Ovarian ca. IGROV-1 | 25.7 |
| Ovarian ca. OVCAR-8 | 13.4 |
| Ovary | 1.4 |
| Breast ca. MCF-7 | 7.4 |
| Breast ca. MDA-MB-231 | 21.6 |
| Breast ca. BT 549 | 100.0 |
| Breast ca. T47D | 11.7 |
| Breast ca. MDA-N | 11.7 |
| Breast Pool | 1.9 |
| Trachea | 1.7 |
| Lung | 1.0 |
| Fetal Lung | 5.4 |
| Lung ca. NCI-N417 | 5.7 |
| Lung ca. LX-1 | 8.2 |
| Lung ca. NCI-H146 | 1.7 |
| Lung ca. SHP-77 | 17.2 |
| Lung ca. A549 | 81.2 |
| Lung ca. NCI-H526 | 2.0 |
| Lung ca. NCI-H23 | 4.3 |
| Lung ca. NCI-H460 | 10.2 |
| Lung ca. HOP-62 | 6.8 |
| Lung ca. NCI-H522 | 2.8 |
| Liver | 0.2 |
| Fetal Liver | 3.1 |
| Liver ca. HepG2 | 4.6 |
| Kidney Pool | 3.2 |
| Fetal Kidney | 1.3 |
| Renal ca. 786-0 | 11.0 |
| Renal ca. A498 | 28.5 |
| Renal ca. ACHN | 15.9 |
| Renal ca. UO-31 | 16.0 |
| Renal ca. TK- 10 | 7.4 |
| Bladder | 2.6 |
| Gastric ca. (liver met.) NCI-N87 | 3.1 |
| Gastric ca. KATO III | 25.3 |
| Colon ca. SW-948 | 6.2 |
| Colon ca. SW480 | 16.2 |
| Colon ca.* (SW480 met) SW620 | 5.3 |
| Colon ca. HT29 | 10.7 |
| Colon ca. HCT-116 | 24.5 |
| Colon ca. CaCo-2 | 12.9 |
| Colon cancer tissue | 7.3 |
| Colon ca. SW1116 | 5.4 |
| Colon ca. Colo-205 | 6.4 |
| Colon ca. SW-48 | 3.6 |
| Colon Pool | 1.6 |
| Small Intestine Pool | 1.3 |
| Stomach Pool | 1.4 |
| Bone Marrow Pool | 1.1 |
| Fetal Heart | 0.4 |
| Heart Pool | 0.7 |
| Lymph Node Pool | 1.8 |
| Fetal Skeletal Muscle | 0.9 |
| Skeletal Muscle Pool | 0.2 |
| Spleen Pool | 1.8 |
| Thymus Pool | 2.3 |
| CNS cancer (glio/astro) U87-MG | 14.0 |
| CNS cancer (glio/astro) U-118-MG | 9.5 |
| CNS cancer (neuro; met) SK-N-AS | 5.4 |
| CNS cancer (astro) SF-539 | 8.2 |
| CNS cancer (astro) SNB-75 | 92.0 |
| CNS cancer (glio)SNB- 19 | 27.2 |
| CNS cancer (glio) SF-295 | 13.3 |
| Brain (Amygdala) Pool | 2.2 |
| Brain (cerebellum) | 6.3 |
| Brain (fetal) | 3.3 |
| Brain (Hippocampus) Pool | 3.0 |
| Cerebral Cortex Pool | 3.0 |
| Brain (Substantia nigra) Pool | 2.2 |
| Brain (Thalamus) Pool | 4.5 |
| Brain (whole) | 2.9 |
| Spinal Cord Pool | 3.5 |
| Adrenal Gland | 3.3 |
| Pituitary gland Pool | 1.1 |
| Salivary Gland | 0.9 |
| Thyroid (female) | 1.9 |
| Pancreatic ca. CAPAN2 | 15.9 |
| Pancreas Pool | 2.7 |

Column A - Rel. Exp. (%) Ag6328, Run 277234226

General screening panel v1.5 Summary: Transketolase is abundantly expressed in all tissues. Transketolase is highly expressed in adipose (CT=27.42), which is the target tissue.

Panel 5 Islet Summary: Panel 5I shows the highest expression in placenta with a good expression in adipose tissue and supports the data of panel 1.5.

General screening panel v1.6 Summary: (Ag6328) High expression of this gene was detected ubiquitously in all tissues (CT=26-30). This ubiquitous pattern of expression indicates that this gene product is involved in homeostatic processes for these and other cell types and tissues.

Its expression was elevated in all the cancer cell lines (melanoma, ovarian cancer, breast cancer, lung cancer, renal cancer, pancreatic cancer, and CNS cancer) with CT=22-26. Therapeutic modulation of this gene, expressed protein and/or use of antibodies or small molecule drugs targeting the gene or gene product are useful in the treatment of melanoma, ovarian cancer, breast cancer, lung cancer, renal cancer, pancreatic cancer, and CNS cancer. In addition, the expression of this gene can be used as a detection marker for those cancers. This gene was highly expressed in adipose tissue (CT27.42). Therapeutic modulation of this gene, expressed protein and/or use of antibodies or small molecule drugs targeting the gene or gene product are useful in the treatment of obesity and diabetes.

### EXAMPLE B5

### PathCalling®

PathCalling® screening showed that Transketolase (TKT) interacts with transcriptional factor (TIF1) and the extracellular domain of semaforin (CG51896-04, See Table EB5). Both interactions have been detected several times in the screening process with different libraries. Protocol for PathCalling® technoogy is disclosed in Example Q10.

TABLE EB5

(SEQ ID NO:221)

CG51896-04-prot 1047 aa
MRSEALLLYFTLLHFAGAGFPEDSEPISISHCNYTKQYPVFVGHKPGRNTTQRHRLDIQM
IMIMNGTLYIAARDHIYTVDIDTSHTEEIYCSKKLTWKSRQADVDTCRMKGKHKDECHNF
IKVLLKKNDDALFVCGTNAFNPSCRNYKMDTLEPFGDEFSGMARCPYDAKHANVALFADG
KLYSATVTDFLAIDAVIYRSLGESPTLRTVKHDSKWLKEPYFVQAVDYGDYIYFFFREIA
VEYNTMGKVVFPRVAQVCKNDMGGSQRVLEKQWTSFLKARLNCSVPGDSHFYFNILQAVT
DVIRINGRDVVLATFSTPYNSIPGSAVCAYDMLDIASVFTGRFKEQKSPDSTWTPVPDER
VPKPRPGCCAGSSSLERYATSNEFPDDTLNFIKTHPLMDEAVPSIFNRPWFLRTMVRYRL
TKIAVDTAAGPYQNHTVVFLGSEKGIILKFLARIGNSGFLNDSLFLEEMSVYNSEKCSYD
GVEDKRIMGMQLDRASSSLYVAFSTCVIKVPLGRCERHGKGKKTGIASRDPYCGWIKEGG
ACSHLSPNSRLTFEQDIERGNTDGLGDCHNSFVALNDISTPLPDNEMSYNTVYGHSSSLL
PSTTTSDSTAQEGYESRGGMLDWKHLLDSPDSTDPLGAVSSHNHQDKKGVIRESYLKGHD
QLVPVTLLAIAVILAFVMGAVFSGITVYCVCDHRRKDVAVVQRKEKELTHSRRGSMSSVT
KLSGLFGDTQSKDPKPEAILTPLMHNGKLATPGNTAKMLIKADQHHLDLTALPTPESTPT
LQQKRKPSRGSREWERNQNLINACTKDMPPMGSPVIPTDLPLRASPSHIPSVVVLPITQQ
GYQHEYVDQPKMSEVAQMALEDQAATLEYKTIKEHLSSKSPNHGVNLVENLDSLPPKVPQ
REASLGPPGASLSQTGLSKRLEMHHSSSYGVDYKRSYPTNSLTRSHQATTLKRNNTNSSN
SSHLSRNQSFGRGDNPPPAPQRVDSIQVHSSQPSGQAVTVSRQPSLNAYNSLTRSGLKRT
PSLKPDVPPKPSFAPLSTSMKPNDACT

### EXAMPLE B6

### Assays Screening for Modulators of Transketolase

A non-exhaustive list of cell lines that express the Transketolase gene can be obtained from the RTQ-PCR results shown herein. These and other Transketolase expressing cell lines could be used for screening purposes.

Screening for a modulator of Transketolase may be accomplished by measurement of generated NADPH from the reaction in which Transketolase participates as outlined above. Additionally, Transketolase activity could be measured by a method outlined in Frank et al, High thiamine diphosphate concentrations in erythrocytes can be achieved in dialysis patients by oral administration of benfontiamine, Eur J Clin Pharmacol. 2000 June; 56 (3):251-7).

Functional/mechanistic assay for the effectiveness of the modulator of Transketolase can be performed by measurement of effect of transketolase 1 inhibitors on TG synthesis/accumulation in 3T3-L1 mouse adipocytes, human adipocytes or hepatocytes by Oil Red 0 staining. Alternatively measurement could be made of post treatment TG accumulation in cells transfected with Transketolase 1.

Our results indicate that a modulator of Transketolase activity, such as an inhibitor, activator, antagonist, or agonist of Transketolase may be useful for treatment of such disorders as obesity, diabetes, and insulin resistance, as well as for enhancement of insulin secretion.

C. NOV3—Long Chain Fatty Acyl Elongase

Long chain fatty acyl elongase (LCE) is an enzyme that uses malonyl-CoA as a 2-carbon donor for elongation of the 16-carbon fatty acid palmitic acid to the 18-carbon fatty acid stearic acid. The elongation of fatty acids occurs in the endoplasmic reticulum, where each 2-carbon addition requires 4 sequential reactions: (1) condensation between a fatty acyl-CoA and malonyl-CoA to form a 3-ketoacyl-CoA, (2) reduction of the 3-ketoacyl-CoA using NADPH to form a 3-hydroxyacyl-CoA, (3) dehydration of the 3-hydroxyacyl-CoA to trans-2-enoyl-CoA, and (4) reduction of the trans-2-enoyl-CoA to the saturated acyl-CoA. It has been shown that LCE is essential for the condensation step of fatty acid elongation, which is the rate-limiting step. LCE is regulated by SREBP, as are many enzymes in fatty acid synthesis and lipogenesis (Moon Y A, Shah N A, Mohapatra S, Warrington J A, Horton J D. Identification of a mammalian long chain fatty acyl elongase regulated by sterol regulatory element-binding proteins J Biol Chem 2001 Nov. 30; 276 (48):45358-66. PMID: 11567032).

We found LCE to be down-regulated in brown adipose tissue from mice on a high fat diet with various body weights, ranging from obese (sd4 compared to chow-fed mice), heavily obese (sd7 compared to chow-fed mice), and hyperglycemic, heavily obese mice (sd7+compared to chow-fed mice). However, LCE remained unchanged in white adipose from the same groups of mice. This down-regulation of LCE is in conjunction with a down-regulation of several enzymes in the fatty acid synthesis pathway and the anaplerotic pathway, including ATP citrate lyase, transketolase, malic enzyme, and SREBP. This suggests that in brown adipose, fatty acid synthesis and lipogenesis are down-regulated in compensation to the high fat diet. Such a compensatory mechanism is not present in white adipose. It has been clearly shown in the literature that long chain fatty acids are oxidized less than short chain fatty acids (DeLany J P, Windhauser M M, Champagne C M, Bray G A. Differential oxidation of individual dietary fatty acids in humans. Am J Clin Nutr. 2000 October; 72 (4):905-11. PMID: 11010930) and that they are more prone to be retained in muscle and liver, which may contribute to insulin resistance (Bessesen D H, Vensor S H, Jackman M R. Trafficking of dietary oleic, linolenic, and stearic acids in fasted or fed lean rats. Am J Physiol Endocrinol Metab. 2000 June; 278 (6):E1124-32. PMID: 10827016). Therefore, mimicking brown adipose in white adipose, by inhibiting LCE, will decrease the amount of long chain fatty acids and therefore, potentially promote more fatty acid oxidation of short chain fatty acids.

Thus, a modulator of LCE such as an antagonist or inhibitor for LCE may be beneficial for the treatment of obesity and/or diabetes. Potential assays are used to screen for antibody therapeutics or small molecule drugs associated with the human Long Chain Fatty Acyl Elongase that are useful to treat obesity and/or diabetes. In a screening assay for inhibitors of LCE, radiolabelled malonyl-CoA plus palmitate will yield radiolabelled stearate that can be detected by partition assay. A non-exhaustive list of cell lines that express the long chain fatty acyl elongase can be obtained from the differential gene expression (RTQ-PCR) results presented herein. These and other Long Chain Fatty Acyl Elongase expressing cell lines could be used for screening purposes.

Furthermore, our results indicate that a modulator of Long Chain Fatty Acyl Elongase activity, such as an inhibitor, activator, antagonist, or agonist of Long Chain Fatty Acyl Elongase may be useful for treatment of such disorders as obesity, diabetes, and insulin resistance, as well as for enhancement of insulin secretion.

Discovery Process

The following sections describe the study designs and the techniques used to identify the Long Chain Fatty Acyl Elongase-encoded protein and any variants, thereof, as being suitable as diagnostic markers, targets for an antibody therapeutic and targets for a small molecule drugs for Obesity and/or Diabetes.

### EXAMPLE C1

### Mouse Dietary-Induced Obesity

A protocol for Mouse Dietary-Induced Obesity study is disclosed in Example Q1.

The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design sought to identify the factors that lead to the ability of certain individuals to resist the effects of a high fat diet and thereby prevent obesity. The sample groups for the study had body weights +1 S.D., +4 S.D. and +7 S.D. of the chow-fed controls. In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues revealed genes and pathways that can be used as therapeutic targets for obesity. Protocol for differential gene expression analysis, GeneCalling®, is disclosed in Example Q7.

Results

A fragment of the mouse (mouse strain C57BL/6J) Long Chain Fatty Acyl Elongase (LCE) gene was initially found to be down-regulated by 2-fold in the brown adipose tissue of obese mice on a high fat diet (sd4) relative to normal weight mice (chow-fed) using CuraGen's GeneCalling® method of differential gene expression. A differentially expressed mouse gene fragment migrating, at approximately 285 nucleotides in length was definitively identified as a component of the mouse Long Chain Fatty Acyl Elongase cDNA. The method of competitive PCR was used for confirmation of the gene assessment. The electropherographic peaks corresponding to the gene fragment of the mouse Long Chain Fatty Acyl Elongase were ablated when a gene-specific primer (shown in Table C1) competes with primers in the linker-adaptors during the PCR amplification. The peaks at 285 nt in length were ablated in the sample from both the brown adipose tissue of obese mice on a high fat diet (sd4) and the normal weight mice (chow-fed). In addition, LCE was found to be down-regulated in brown adipose tissue of hyperinsulinemic (ngsd7) and hyperglycemic obese mice on a high fat diet compared to normal weight mice (chow-fed).

TABLE C1

The direct sequence of the 285 nucleotide-long gene fragment and the
gene-specific primers used for competitive PCR are indicated on the
cDNA sequence of the Long Chain Fatty Acyl Elongase fragment (SEQ ID
NO:183) are shown in bold. The gene-specific primers at the 5′ and 3′
ends of the fragment are underlined.
Gene Sequence (fragment from 5292 to 5576 in bold. band size: 285)

4811 CACACAGCTC CCATTTCCTG GTGCCTGAGA TCCCAGCCAT CAGAAAGTGA TTTGGGTGAG
4871 AATTCACAAC ATATATGTCA CCTCTGCATA TTGAAGTGAC ATCTAATAAA ACAAGGACGT
4931 CCTATTTTGT CTGAACCCGC TGAATGAAGC TCTGTTATCC TAGTTAGTCA TTGGGCCGCC
4991 ATCCTCTGTA CCCGATAGTG ACACAAAACA GATGTCGGTG CCTGTACAAG AATTCTCAGT
5051 GCCTGTTGTG ACAGACTGTG CTTAGAAGAA ACATTCGTGA GCCATAAAGC AGGAACCACA
5111 GATGAAAGGG CCAGTTAAAA GTCCACCTGC TCCAAGTATC ATAGAAAACC CAAAAGCCTG
5171 TTGTATAATC TGGTATTGTC CCCATCCCCA GATGCTTTGA AAACTAGGAT TCTCAGAGCA
5231 TGGATACCCA CGCTTCCATC TTCCCACAAA CATTTCCTAG AGTTGTACTG GTGGGTGCAG
5291 CCCTAGGTGG TTGGTTGGGG GAAGTCTTGG AAGCTGTACT TTGATTGCAG GTCAAGCAAA
5351 GCCAAATCCA GATATTTCTG TGTCACTCAC CAGTTGTCCA TGTCCACCCA CAAAACAATT
5411 GTATTATAGT CAAGTTGTCC TAGCTGATTG GTCCTCAAAT AAGGATGCAA CTATGTTTGC
5471 AACCCAGTTA GGACACATTT GAAAGAACCT GACTCACTAG CATCTAAACA ATATCATTTC
5531 CCCAATGCTT GGTGGCACTT CAGACTTTTG TTCTCCTGGT TGATCAAGGT GTTGCCTGGT
5591 GGTGCCGCCT CCTAGTGTGA ATATTTCAGT TAAGTGTGGG TCTGAGCATG ACCGGGCTGG
5651 GCTTAGCTCA CTGCTACTTG GAAAATGACT GGCATTCTGC TTCCTAGGCC CTAAACCCAT
5711 ATTCAGAGGG AAAATTCACT ATCAAGCCTC ACAGCGAAAT CACAGCAGTG TTGGAATTCT
5771 TATTTTCAAG TGCTTATCTC ACAACATTGA AAAATATTTT TGGTGTATTA AGATTTAAAA
5831 TAAAGTCATC ATAAACTTTT GAATTTAAAA AAAAAAAAAA AAAAAAAAAA AAAAAAAAAA
5891 AA

(gene length is 5892, only region from 4811 to 5892 shown)

### EXAMPLE C2

### Identification of Human Long Chain Fatty Acyl Elongase Sequences

The sequence of Human Long Chain Fatty Acyl Elongase (Acc. No. CG180320-01) was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof. The protocol for identification of human sequence(s) is disclosed in Example Q8.

Table C2 shows protein alignment (ClustalW) of Human Long Chain Fatty Acyl Elongase Protein Sequence (CG 180320-01; SEQ ID NO:18) against Mouse Long Chain Fatty Acyl Elongase (Q920L5; SEQ ID NO:184) and Rat Long Chain Fatty Acyl Elongase (Q920L6; SEQ ID NO:185). Table C3 shows sequences of Mouse Long Chain Fatty Acyl Elongase (Q920L5; SEQ ID NO:184) and Rat Long Chain Fatty Acyl Elongase (Q920L6; SEQ ID NO:185).

[image]

TABLE C3

Sequences of Mouse Long Chain Fatty Acyl Elongase (Q920L5; SEQ ID
NO: 184) and Rat Long Chain Fatty Acyl Elongase (Q920L6; SEQ ID NO:
185).

Mouse Long Chain Fatty Acyl Elongase (Q920L5; SEQ ID NO:184)
MNMSVLTLQEYEFEKQFNENEAIQWMQENWKKSFLFSALYAAFLFGGRHLMNKRAKFELRKPLVLWSLTL
AVFSLFGALRTGAYMLYILMTKGLKQSVCDQSFYNGPVSKFWAYAFVLSKAPELGDTLFILLRKQKLIFL
HWYHHITVLLYSWYSYKDMVAGGGWFMTMNYGVHAVMYSYYALRAAGFRVSRKFAMFITLSQITQMLMGC
VINYLVFNWMQHDNDQCYSHFQNIFWSSLMYLSYLVLFCHFFFEAYIGKVKKATKAE
Rat Long Chain Fatty Acyl Elongase (Q920L6; SEQ ID NO:185)
MNMSVLTLQEYEFEKQFNENEAIQWMQENWKKSFLFSALYAAFIFGGRHLMNKRAKFELRKPLVLWSLTL
AVFSLFGALRTGAYMLYILMTKGLKQSVCDQSFYNGPVSKFWAYAFVLSKAPELGDTIFILLRKQKLIFL
HWYHHITVLLYSWYSYKDMVAGGGWFMTMNYGVHAVMYSYYALRAAGFRVSRKFAMFITLSQITQMLMGC
VINYLVFNWMQHDNDQCYSHFQNIFWSSLMYLSYLLLFCHFFFEAYIGKVKKATKAE

The laboratory cloning was performed using one or more of the methods summarized in Example Q8. The NOV3 clone was analyzed, and the nucleotide and encoded polypeptide sequences are shown in Table C4.

TABLE C4
NOV3 Sequence Analysis

| | | |
|---|---|---|
| NOV3a, CG180320-01 | SEQ ID NO:17 | 3047 bp |
| DNA Sequence | ORF Start: ATG at 281 | ORF Stop: TAG at 1076 |

ACTAAGACCGCAAGGCATTCATTTCCTCCTACGGTGGATGCGGACGCCGGGAGGAGGAGAGCCCCAGA
GAGAGGAGCTGGGAGCGGAGGCGCAGGCAATGCTCAGCCCTGGATGTAGCTGAGAGGCTGGGAGAAGA
GACGACCGCTGGAGACCGAGCGGCGTGGGGAAGACCTAGGGGGGTGGGTGGGGGAAGCAGACAGGAGA
ACACTCGAAATCAAGCGCTTTACAGATTATTTTATTTTGTATAGAGAACACGTAGCGACTCCGAAGAT
CAGCCCCAATGAACATGTCAGTGTTGACTTTACAAGAATATGAATTCGAAAAGCAGTTCAACGAGAAT
GAAGCCATCCAATGGATGCAGGAAAACTGGAAGAAATCTTTCCTGTTTTCTGCTCTGTATGCTGCCTT
TATATTCGGTGGTCGGCACCTAATGAATAAACGAGCAAAGTTTGAACTGAGGAAGCCATTAGTGCTCT
GGTCTCTGACCCTTGCAGTCTTCAGTATATTCGGTGCTCTTCGAACTGGTGCTTATATGGTGTACATT
TTGATGACCAAAGGCCTGAAGCAGTCAGTTTGTGACCAGGGTTTTTACAATGGACCTGTCAGCAAATT
CTGGGCTTATGCATTTGTGCTAAGCAAAGCACCCGAACTAGGAGATACAATATTCATTATTCTGAGGA
AGCAGAAGCTGATCTTCCTGCACTGGTATCACCACATCACTGTGCTCCTGTACTCTTGGTACTCCTAC
AAAGACATGGTTGCCGGGGGAGGTTGGTTCATGACTATGAACTATGGCGTGCACGCCGTGATGTACTC
TTACTATGCCTTGCGGGCGGCAGGTTTCCGAGTCTCCCGGAAGTTTGCCATGTTCATCACCTTGTCCC
AGATCACTCAGATGCTGATGGGCTGTGTGGTTAACTACCTGGTCTTCTGCTGGATGCAGCATGACCAG
TGTCACTCTCACTTTCAGAACATCTTCTGGTCCTCACTCATGTACCTCAGCTACCTTGTGCTCTTCTG
CCATTTCTTCTTTGAGGCCTACATCGGCAAAATGAGGAAAACAACGAAAGCTGAATAGTGTTGGAACT
GAGGAGGAAGCCATAGCTCAGGGTCATCAAGAAAAATAATAGACAAAAGAAAATGGCACAAGGAATCA
CACGTGGTGCAGCTAAAACAAAACAAAACATGAGCAAACACAAAACCCAAGGCAGCTTAGGGATAATT
AGGTTGATTTAACCCAGTAAGTTTATGATCCTTTTAGGGTGAGGACTCACTGAGTGCACCTCCATCTC
CAAGCACTGCTGCTGGAAGACCCCATTCCCTCTTTATCTATCAACTCTAGGACAAGGGAGAACAAAAG
CAAGCCAGAAGCAGAGGAGACTAATCAAAGGCAAACAAAGGCTATTAACACATAGGAAAATATGTATT
TACTAAGTGTCACATTTCTCTAAGATGAAAGATTTTTACTCTAGAAACTGTGCGAGCACAACACACAC
AATCCTTTCTAACTTTATGGACACTAAACTGGAGCCAATAGAAAAGACAAAAATGAAAGAGACACAGG
GTGTATATCTAGAACGATAATGCTTTTGCAGAAACTAAAGCCTTTTTAAGAAATGCCAGCTGCTGTAG
ACCCCATGAGAAAAGATGTCTTAATCATCCTTATGAAAACAGATGTAAACAACTATATTTCAACTAAC
TTCATCTTCACTGCATAGCCTCAGGCTAGTGAGTTTGCCAAAACCAAAGGGGGTGAATACTTCCCCAA
GATTCTTCCTGGGAGGATGGAAACAGTGCAGCCCAGGTCCCATGGGGGCAGCTCCATCCCAGAGCATT
TCTGATAGTTGAACTGTAATTTCTACTCTTAAGTGAGATATGAAGTATTATCCTTTTGTTCAGTTGCC
CCGGGCTTTTGAACAGAAGAGTAAATACAGAATTGAAAAAGATAAACACTCAACCAAACAATGTGAAA
ACGGGTTCTGTAGTATTTGTAAAAAGGCCCGGCCCAGGACCACTGTGAGCTGGAAAAGGGAGAAAGGC
AGTGGGAAAAGAGGTGAGCCGAAGATCAATTCGACAGACAGACGGTGTGTATGCCCCTCCCTGTTTGA
CTTCACACACACTCATAACTTTCCAAATGAAACCCCACAGTATAGCGCATATTTTCGATATTTTTGTG
AATTCCAAAAGGAAATCACAGGGCTGTTCGAAATATTGGGGGAACACTGTGTTTCTGCATCATCTGCA
TTTGCTCCCCAAGCAATGTAGAGGTGTTTAAAGGGCCCTCTGCTGGCTGAGTGGCAATACTACAACAA
ACTTCAAGGCAAGTTTGGCTGAAAACAGTTGACAACAAAGGGCCCCCATACACTTATCCCTCAAATTT
TAAGTGATATGAAATACTTGTCATGTCTTTGGCCAAATCAGAAGATATTCATCCTGCTTCAAGTCAGC
TTCAGAAATGTTTTAAAAGGGACTTTAGCTCTGGAACTCAAAATCAATTTATTAAGAGCCATATTCTT
TAAAAAAAAAAGCTGGATAATATTATCTGTAATATTTCAGTCCTTTACAAGCCAAATACATGTGTCAA
TGTTTCTAGTATTTCAAAGAAGCAATTATGTAAAGTTGTTCAATGTGACATAATAGTATTATAATTGG
TTAAGTAGCTTAATGATTAGGCAAACTAGATGAAAAGATTAGGGGCTTCCACACTGCATAGATCACAC
GCACATAGCCACGCATACACACACAGACACACAGATGTGGGGTACACTGAATTTCAAAGCCCAAATGA
ATAGAAACACATTTTCTGGCTAGCAGAAAAAAACAAAACAAAACTGTTGTTTCTCTTTCTTGCTTTGA
GAGTGTACAGTAAAAGGGATTTTTTCGAATTATTTTTATATTATTTTAGCTTTAATTGTGCTGTCGTT
CATGAAACAGAGCTGCTCTGCTTTTCTGTCAGAGATGGCAAGGGCTTTTTCAGCATCTCGTTTATGTG
TGGAATTTAAAAAGAATAAAGTTTTATTCCATTCTGAAAAAAAAAAAAAAAAAGC

| | | | |
|---|---|---|---|
| NOV3a, GG180320-01 | | | |
| Protein Sequence | SEQ ID NO:18 | 265 aa | MW at 31375.7 kD |

MNMSVLTLQEYEFEKQFNENEAIQWMQENWKKSFLFSALYAAFIFGGRHLMNKRAKFELRKPLVLWSL
TLAVFSIFGALRTGAYMVYILMTKGLKQSVCDQGFYNGPVSKFWAYAFVLSKAPELGDTIFIILRKQK
LIFLHWYHHITVLLYSWYSYKDMVAGGGWFMTMNYGVHAVMYSYYALRAAGFRVSRKFAMFITLSQIT
QMLMGCVVNYLVFCWMQHDOCHSHFQNIFWSSLMYLSYLVLFCHFFFEAYIGKMRKTTKAE

| | | |
|---|---|---|
| NOV3b, GG180320-02 | SEQ ID NO:19 | 822 bp |
| DNA Sequence | ORF Start: at 2 | ORF Stop: TAG at 809 |

CACCGGATCCACCATGAACATGTCAGTGTTGACTTTACAAGAATATGAATTCGAAAAGCAGTTCAACG
AGAATGAAGCCATCCAATGGATGCAGGAAAACTGGAAGAAATCTTTCCTGTTTTCTGCTCTGTATGCT
GCCTTTATATTCGGTGGTCGGCACCTAATGAATAAACGAGCAAAGTTTGAACTGAGGAAGCCATTAGT
GCTCTGGTCTCTGACCCTTGCAGTCTTCAGTATATTCGGTGCTCTTCGAACTGGTGCTTATATGGTGT
ACATTTTGATGACCAAAGGCCTGAAGCAGTCAGTTTGTGACCAGGGTTTTTACAATGGACCTGTCAGC
AAATTCTGGGCTTATGCATTTGTGCTAAGCAAAGCACCCGAACTAGGAGATACAATATTCATTATTCT
GAGGAAGCAGAAGCTGATCTTCCTGCACTGGTATCACCACATCACTGTGCTCCTGTACTCTTGGTACT
CCTACAAAGACATGGTTGCCGGGGGAGGTTGGTTCATGACTATGAACTATGGCGTGCACGCCGTGATG
TACTCTTACTATGCCTTGCGGGCGGCAGGTTTCCGAGTCTCCCGGAAGTTTGCCATGTTCATCACCTT
GTCCCAGATCACTCAGATGCTGATGGGCTGTGTGGTTAACTACCTGGTCTTCTGCTGGATGCAGCATG
ACCAGTGTCACTCTCACTTTCAGAACATCTTCTGGTCCTCACTCATGTACCTCAGCTACCTTGTGCTC
TTCTGCCATTTCTTCTTTGAGGCCTACATCGGCAAAATGAGGAAAACAACGAAAGCTGAATAGGCGGC
CGCTAT

| | | | |
|---|---|---|---|
| NOV3b, CG180320-02 | | | |
| Protein Sequence | SEQ ID NO:20 | 269 aa | MW at 31722.1 kD |

TGSTMNMSVLTLQEYEFEKQFNENEAIQWMQENWKKSFLFSALYAAFIFGGRHLMNKRAKFELRKPLV
LWSLTLAVFSIFGALRTGAYMVYILMTKGLKQSVCDQGFYNGPVSKFWAYAFVLSKAPELGDTIFIIL
RKQKLIFLHWYHHITVLLYSWYSYKDMVAGGGWFMTMNYGVHAVMYSYYALRAAGFRVSRKFAMFITL
SQITQMLMGCVVNYLVFCWNQHDQCHSHFQNIFWSSLMYLSYLVLFCHFFFEAYIGKMRKTTKAE

| | | |
|---|---|---|
| NOV3c, CG180320-03 | SEQ ID NO:21 | 834 bp |
| DNA Sequence | ORF Start: at 1 | ORF Stop: TAG at 793 |

AACATGTCAGTGTTGACTTTACAAGAATATGAATTCGAAAAGCAGTTCAACGAGAATGAAGCCATCCA
ATGGATGCAGGAAAACTGGAAGAAATCTTTCCTGTTTTCTGCTCTGTATGCTGCCTTTATATTCGGTG
GTCGGCACCTAATGAATAAACGAGCAAAGTTTGAACTGAGGAAGCCATTAGTGCTCTGGTCTCTGACC
CTTGCAGTCTTCAGTATATTCGGTGCTCTTCGAACTGGTGCTTATATGGTGTACATTTTGATGACCAA
AGGCCTGAAGCAGTCAGTTTGTGACCAGGGTTTTTACAATGGACCTGTCAGCAAATTCTGGGCTTATG
CATTTGTGCTAAGCAAAGCACCCGAACTAGGAGATACAATATTCATTATTCTGAGGAAGCAGAAGCTG
ATCTTCCTGCACTGGTATCACCACATCACTGTGCTCCTGTACTCTTGGTACTCCTACAAAGACATGGT
TGCCGGGGGAGGTTGGTTCATGACTATGAACTATGGCGTGCACGCCGTGATGTACTCTTACTATGCCT
TGCGGGCGGCAGGTTTCCGAGTCTCCCGGAAGTTTGCCATGTTCATCACCTTGTCCCAGATCACTCAG
ATGCTGATGGGCTGTGTGGTTAACTACCTGGTCTTCTGCTGGATGCAGCATGACCAGTGTCACTCTCA
CTTTCAGAACATCTTCTGGTCCTCACTCATGTACCTCAGCTACCTTGTGCTCTTCTGCCATTTCTTCT
TTGAGGCCTACATCGGCAAAATGAGGAAAACAACGAAAGCTGAATAGGCAGGTGCGGCCGCACTCGAG
CACCACCACCACCACCAC

| | | | |
|---|---|---|---|
| NOV3c, CG 180320-03 | | | |
| Protein Sequence | SEQ ID NO:22 | 264 aa | MW at 31244.5 kD |

NMSVLTLQEYEFEKQFNENEAIQWMQENWKKSFLFSALYAAFIFGGRHLMNKRAKFELRKPLVLWSLT
LAVFSIFGALRTGAYMVYILMTKGLKQSVCDQGFYNGPVSKFWAYAFVLSKAPELGDTIFIILRKQKL
IFLHWYHHITVLLYSWYSYKDMVAGGGWFMTMNYGVHAVMYSYYALRAAGFRVSRKFAMFITLSQITQ
MLMGCVVNYLVFCWMQHDQCHSHFQNIFWSSLMYLSYLVLFCHFFFEAYIGKMRKTTKAE

| | | |
|---|---|---|
| NOV3d, CG180320-04 | SEQ ID NO:23 | 801 bp |
| DNA Sequence | ORF Start: at 1 | ORF Stop: TAG at 799 |

ACCATGAACATGTCAGTGTTGACTTTACAAGAATATGAATTCGAAAAGCAGTTCAACGAGAATGAAGC
CATCCAATGGATGCAGGAAAACTGGAAGAAATCTTTCCTGTTTTCTGCTCTGTATGCTGCCTTTATAT
TCGGTGGTCGGCACCTAATGAATAAACGAGCAAAGTTTGAACTGAGGAAGCCATTAGTGCTCTGGTCT
CTGACCCTTGCAGTCTTCAGTATATTCGGTGCTCTTCGAACTGGTGCTTATATGGTGTACATTTTGAT
GACCAAAGGCCTGAAGCAGTCAGTTTGTGACCAGGGTTTTTACAATGGACCTGTCAGCAAATTCTGGG
CTTATGCATTTGTGCTAAGCAAAGCACCCGAACTAGGAGATACAATATTCATTATTCTGAGGAAGCAG
AAGCTGATCTTCCTGCACTGGTATCACCACATCACTGTGCTCCTGTACTCTTGGTACTCCTACAAAGA
CATGGTTGCCGGGGGAGGTTGGTTCATGACTATGAACTATGGCGTGCACGCCGTGATGTACTCTTACT
ATGCCTTGCGGGCGGCAGGTTTCCGAGTCTCCCGGAAGTTTGCCATGTTCATCACCTTGTCCCAGATC
ACTCAGATGCTGATGGGCTGTGTGGTTAACTACCTGGTCTTCTGCTGGATGCAGCATGACCAGTGTCA
CTCTCACTTTCAGAACATCTTCTGGTCCTCACTCATGTACCTCAGCTACCTTGTGCTCTTCTGCCATT
TCTTCTTTGAGGCCTACATCGGCAAAATGAGGAAAACAACGAAAGCTGAATAG

| | | | |
|---|---|---|---|
| NOV3d, CG180320-04 | | | |
| Protein Sequence | SEQ ID NO:24 | 266 aa | MW at 31476.8 kD |

TMNMSVLTLQEYEFEKQFNENEAIQWMQENWKKSFLFSALYAAFIFGGRHLMNKRAKFELRKPLVLWS
LTLAVFSIFGALRTGAYMVYILMTKGLKQSVCDQGFYNGPVSKFWAYAFVLSKAPELGDTIFIILRKQ
KLIFLHWYHHITVLLYSWYSYKDMVAGGGWFMTMNYGVHAVMYSYYALRAAGFRVSRKFAMFITLSQI
TQMLMGCVVNYLVFCWMQHDQCHSHFQNIFWSSLMYLSYLVLFCHFFFEAYIGKMRKTTKAE

| | | |
|---|---|---|
| NOV3e, 305263028 | SEQ ID NO:25 | 834 bp |
| DNA Sequence | ORF Start: at 1 | ORF Stop: TAG at 793 |

AACATGTCAGTGTTGACTTTACAAGAATATGAATTCGAAAAGCAGTTCAACGAGAATGAAGCCATCCA
ATGGATGCAGGAAAACTGGAAGAAATCTTTCCTGTTTTCTGCTCTGTATGCTGCCTTTATATTCGGTG
GTCGGCACCTAATGAATAAACGAGCAAAGTTTGAACTGAGGAAGCCATTAGTGCTCTGGTCTCTGACC
CTTGCAGTCTTCAGTATATTCGGTGCTCTTCGAACTGGTGCTTATATGGTGTACATTTTGATGACCAA
AGGCCTGAAGCAGTCAGTTTGTGACCAGGGTTTTTACAATGGACCTGTCAGCAAATTCTGGGCTTATG
CATTTGTGCTAAGCAAAGCACCCGAACTAGGAGATACAATATTCATTATTCTGAGGAAGCAGAAGCTG
ATCTTCCTGCACTGGTATCACCACATCACTGTGCTCCTGTACTCTTGGTACTCCTACAAAGACATGGT
TGCCGGGGGAGGTTGGTTCATGACTATGAACTATGGCGTGCACGCCGTGATGTACTCTTACTATGCCT
TGCGGGCGGCAGGTTTCCGAGTCTCCCGGAAGTTTGCCATGTTCATCACCTTGTCCCAGATCACTCAG
ATGCTGATGGGCTGTGTGGTTAACTACCTGGTCTTCTGCTGGATGCAGCATGACCAGTGTCACTCTCA
CTTTCAGAACATCTTCTGGTCCTCACTCATGTACCTCAGCTACCTTGTGCTCTTCTGCCATTTCTTCT
TTGAGGCCTACATCGGCAAAATGAGGAAAACAACGAAAGCTGAATAGGCAGGTGCGGCCGCACTCGAG
CACCACCACCACCACCAC

| | | | |
|---|---|---|---|
| NOV3e, 305263028 | | | |
| Protein Sequence | SEQ ID NO:26 | 264 aa | MW at 31244.5 kD |

NMSVLTLQEYEFEKQFNENEAIQWMQENWKKSFLFSALYAAFIFGGRHLMNKRAKFELRKPLVLWSLT
LAVFSIFGALRTGAYMVYILMTKGLKQSVCDQGFYNGPVSKFWAYAFVLSKAPELGDTIFIILRKQKL
IFLHWYHHITVLLYSWYSYKDMVAGGGWFMTMNYGVHAVMYSYYALRAAGFRVSRKFAMFITLSQITQ
MLMGCVVNYLVFCWMQHDQCHSHFQNIFWSSLMYLSYLVLFCHFFFEAYIGKMRKTTKAE

| | | |
|---|---|---|
| NOV3f, CG180320-05 | SEQ ID NO:27 | 834bp |
| DNA Sequence | ORF Start: at 1 | ORF Stop: TAG at 832 |

TCCACCATGGGTTACCCATATGACGTTCCAGACTACGCAAACATGTCAGTGTTGACTTTACAAGAATA
TGAATTCGAAAAGCAGTTCAACGAGAATGAAGCCATCCAATGGATGCAGGAAAACTGGAAGAAATCTT
TCCTGTTTTCTGCTCTGTATGCTGCCTTTATATTCGGTGGTCGGCACCTAATGAATAAACGAGCAAAG
TTTGAACTGAGGAAGCCATTAGTGCTCTGGTCTCTGACCCTTGCAGTCTTCAGTATATTCGGTGCTCT
TCGAACTGGTGCTTATATGGTGTACATTTTGATGACCAAAGGCCTGAAGCAGTCAGTTTGTGACCAGG
GTTTTTACAATGGACCTGTCAGCAAATTCTGGGCTTATGCATTTGTGCTAAGCAAAGCACCCGAACTA
GGAGATACAATATTCATTATTCTGAGGAAGCAGAAGCTGATCTTCCTGCACTGGTATCACCACATCAC
TGTGCTCCTGTACTCTTGGTACTCCTACAAAGACATGGTTGCCGGGGGAGGTTGGTTCATGACTATGA
ACTATGGCGTGCACGCCGTGATGTACTCTTACTATGCCTTGCGGGCGGCAGGTTTCCGAGTCTCCCGG
AAGTTTGCCATGTTCATCACCTTGTCCCAGATCACTCAGATGCTGATGGGCTGTGTGGTTAACTACCT
GGTCTTCTGCTGGATGCAGCATGACCAGTGTCACTCTCACTTTCAGAACATCTTCTGGTCCTCACTCA
TGTACCTCAGCTACCTTGTGCTCTTCTGCCATTTCTTCTTTGAGGCCTACATCGGCAAAATGAGGAAA
ACAACGAAAGCTGAATAG

| | | | |
|---|---|---|---|
| NOV3f, GG180320-05 | | | |
| Protein Sequence | SEQ ID NO:28 | 277 aa | MW at 32705.1 kD |

STMGYPYDVPDYANMSVLTLQEYEFEKQFNENEAIQWMQENWKKSFLFSALYAAFIFGGRHLMNKRAK
FELRKPLVLWSLThAVFSIFGALRTGAYMVYILMTKGLKQSVCDQGFYNGPVSKFWAYAFVLSKAPEL
GDTIFIILRKQKLIFLHWYHHITVLLYSWYSYKDMVAGGGWFMTMNYGVHAVMYSYYALRAAGFRVSR
KFAMFITLSQITQMLMGCVVNYLVFCWMQHDQCHSHFQNIFWSSLMYLSYLVLFCHFFFEAYIGKMRK
TTKAE

A ClustalW comparison of the above protein sequences yields the following sequence alignment shown in Table C5.

TABLE C5
Comparison of the NOV3 protein sequences.

| | |
|---|---|
| NOV3a | ————————————MNMSVLTLQEYEFEKQFNENEAIQWMQENWKKSFLFSALYAAFIFGGR |
| NOV3b | ————————TGSTMNMSVLTLQEYEFEKQFNENEAIQWMQENWKKSFLFSAIYAAFIFGGR |
| NOV3c | —————————————NMSVLTLQEYEFEKQFNENEAIQWMQENWKKSFLFSALYAAFIFGGR |
| NOV3d | ———————————TMNMSVLTLOEYEFEKQFNEMEAIQWMQENWKKSFLFSALYAAFIFGGR |
| NOV3e | —————————————NMSVLTLQEYEFEKQFNENEAIQWMQEMWKKSFLFSALYAAFIFGGR |
| NOV3f | STMGYPYDVPDYANMSVLTLQEYEFEKQFNENEAIQWMQEMWKKSFLFSALYAAFIFGGR |
| NOV3a | HLMNKRAKFELRKPLVLWSLTLAVFSIFGALRTGAYMVYILMTKGLKQSVCDQGFYNGPV |
| NOV3b | HLMNKRAKFELRKPLVLWSLTLAVFSIFGALRTGAYMVYILMTKGLKQSVCDQGFYNGPV |
| NOV3c | HLMNKRAKFELRKPLVLWSLTLAVFSIFGALRTGAYMVYILMTKGLKQSVCDQGFYNGPV |
| NOV3d | HLMNKRAKFELRKPLVLWSLTLAVFSIFGALRTGAYMVYILMTKGLKQSVCDQGFYNGPV |
| NOV3e | HLMNKRAKFELRKPLVLWSLTLAVFSIFGALRTGAYMVYILMTKGLKQSVCDQGFYNGPV |
| NOV3f | HLMNKRAKFELRKPLVLWSLTLAVFSIFGALRTGAYMVYILMTKGLKQSVCDQGFYNGPV |
| NOV3a | SKFWAYAFVLSKAPELGDTIFIILRKQKLIFLHWYHHITVLLYSWYSYKDMVAGGGWFMT |
| NOV3b | SKFWAYAFVLSKAPELGDTIFIILRKQKLIFLHWYHHITVLLYSWYSYKDMVAGGGWFMT |
| NOV3c | SKFWAYAFVLSKAPELGDTIFIILRKQKLIFLHWYHHITVLLYSWYSYKDMVAGGGWFMT |
| NOV3d | SKFWAYAFVLSKAPELGDTIFIILRKQKLIFLHWYHHITVLLYSWYSYKDMVAGGGWFMT |
| NOV3e | SKFWAYAFVLSKAPELGDTIFIILRKQKLIFLHWYHHITVLLYSWYSYKDMVAGGGWFMT |
| NOV3f | SKFWAYAFVLSKAPELGDTIFIILRKQKLIFLHWYHHITVLLYSWYSYKDMVAGGGWFMT |
| NOV3a | MNYGVHAVMYSYYALRAAGFRVSRKFAMFITLSQITQMLMGCVVNYLVFCWMQHDQCHSH |
| NOV3b | MNYGVHAVMYSYYALRAAGFRVSRKFANFITLSQITQMLMGCVVNYLVFCWMQHDQCHSH |
| NOV3c | MNYGVHAVMYSYYALRAAGFRVSRKFAMFITLSQITQMLMGCVVNYLVFCWMQHDQCHSH |
| NOV3d | MNYGVHAVMYSYYALRAAGFRVSRKFANFITLSQITQMLMGCVVNYLVFCWMQHDQCHSH |
| NOV3e | MNYGVHAVMYSYYALRAAGFRVSRKFANFITLSQITQMLMGCVVNYLVFCWMQHDQCHSH |
| NOV3f | MNYGVHAVMYSYYALRAAGFRVSRKFAMFITLSQITQMLMGCVVNYLVFCWMQHDQCHSH |
| NOV3a | FQNIFWSSLMYLSYLVLFCHFFFEAYIGKMRKTTKAE |
| NOV3b | FQNIFWSSLMYLSYLVLFCHFFFEAYIGKMRKTTKAE |
| NOV3c | FQNIFWSSLMYLSYLVLFCHFFFEAYIGKMRKTTKAE |
| NOV3d | FQNIFWSSLMYLSYLVLFCHFFFEAYIGKMRKTTKAE |
| NOV3e | FQNIFWSSLMYLSYLVLFCHFFFEAYIGKMRKTTKAE |
| NOV3f | FQNIFWSSLMYLSYLVLFCHFFFEAYIGKMRKTTKAE |
| NOV3a | (SEQ ID NO:18) |
| NOV3b | (SEQ ID NO:20) |
| NOV3c | (SEQ ID NO:22) |
| NOV3d | (SEQ ID NO:24) |
| NOV3e | (SEQ ID NO:26) |
| NOV3f | (SEQ ID NO:28) |

Further analysis of the NOV3a protein yielded the following properties shown in Table C6.

TABLE C6
Protein Sequence Properties NOV3a

| SignalP | Cleavage site between residues 47 and 48 | | | |
|---|---|---|---|---|
| analysis: | | | | |
| PSORT II | PSG: a new signal peptide prediction method | | | |
| analysis: | N-region: length 10; pos. chg 0; neg. chg 1 | | | |
| | H-region: length 1; peak value 0.00 | | | |
| | PSG score: −4.40 | | | |
| | GvH: von Heijne's method for signal seq. recognition | | | |
| | GvH score (threshold: −2.1): −1.00 | | | |
| | possible cleavage site: between 46 and 47 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 3 | | | |
| | Number of TMS(s) for threshold 0.5: 1 | | | |
| | INTEGRAL | Likelihood = | −5.63 | Transmembrane 63-79 |
| | PERIPHERAL | Likelihood = | 2.54 (at 116) | |
| | ALOM score: −5.63 (number of TMSs: 1) | | | |
| | MTOP: Prediction of membrane topology (Hartmann et al.) | | | |
| | Center position for calculation: 70 | | | |
| | Charge difference: −3.5 C(2.0)-N(5.5) | | | |
| | N >= C: N-terminal side will be inside | | | |
| | >>> membrane topology: type 2 (cytoplasmic tail 1 to 63) | | | |
| | MITDISC: discrimination of mitochondrial targeting seq | | | |
| | R content: | 0 | Hyd Moment(75): | 1.56 |
| | Hyd Moment(95): | 3.11 | G content: | 0 |
| | D/E content: | 2 | S/T content: | 2 |

```
Score: −7.42
Gavel: prediction of cleavage sites for mitochondrial preseq
cleavage site motif not found
NUCDISC: discrimination of nuclear localization signals
pat4: none
pat7: none
bipartite: none
content of basic residues: 9.8%
NLS Score: −0.47
KDEL: ER retention motif in the C-terminus: none
ER Membrane Retention Signals:
KKXX-like motif in the C-terminus: TTKA
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
30.4%: cytoplasmic
30.4%: mitochondrial
13.0%: Golgi
  8.7%: endoplasmic reticulum
  4.3%: extracellular, including cell wall
  4.3%: vacuolar
  4.3%: nuclear
  4.3%: vesicles of secretory system
>> prediction for CG180320-01 is cyt (k = 23)
```

A search of the NOV3a protein against the Geneseq database, a proprietary database that contains sequences published in patents and patent publication, yielded several homologous proteins shown in Table C7.

TABLE C7
Geneseq Results for NOV3a

| | | NOV3a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB82962 | Human LCE related protein | 1 . . . 265 | 265/265 (100%) | e−159 |
| | (GenBanK Identifier No. | 1 . . . 265 | 265/265 (100%) | |
| | GI#10440045) - Homo sapiens, 265 | | | |
| | aa. [WO200299068-A2, 12-DEC-2002] | | | |
| ABB82961 | Human LCE related protein | 1 . . . 265 | 265/265 (100%) | e−159 |
| | (GenBanK Identifier No. | 1 . . . 265 | 265/265 (100%) | |
| | aa. [WO200299068-A2, 12-DEC-2002] | | | |
| AAG79838 | ADSL related polypeptide #2 - | 1 . . . 265 | 265/265 (100%) | e−159 |
| | Homo sapiens, 265 aa. | 1 . . . 265 | 265/265 (100%) | |
| | [WO200299038-A2, 12-DEC-2002] | | | |
| AAU87832 | Human elongase HS3 - Homo | 1 . . . 265 | 265/265 (100%) | e−159 |
| | sapiens, 265 aa. [WO200208401-A2, | 1 . . . 265 | 265/265 (100%) | |
| | 31-JAN-2002] | | | |
| AAU00476 | Human INTERCEPT 400 protein - | 1 . . . 265 | 265/265 (100%) | e−159 |
| | Homo sapiens, 265 aa. | 1 . . . 265 | 265/265 (100%) | |
| | [WO200118016-A1, 15-MAR-2001] | | | |

In a BLAST search of public sequence databases, the NOV3a protein was found to have homology to the proteins shown in the BLASTP data in Table C8.

TABLE C8
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H5J4 | Hypothetical protein FLJ23378 - | 1 . . . 265 | 265/265 (100%) | e−158 |
| | Homo sapiens (Human), 265 aa. | 1 . . . 265 | 265/265 (100%) | |
| Q920L5 | Fatty acyl elongase (Long-chain | 1 . . . 265 | 257/267 (96%) | e−152 |
| | fatty-acyl elongase) (Myelination | 1 . . . 267 | 262/267 (97%) | |
| | associated SUR4-like protein) - Mus | | | |
| | musculus (Mouse), 267 aa. | | | |
| Q920L6 | Fatty acid elongase 2 - Rattus | 1 . . . 265 | 256/267 (95%) | e−152 |
| | norvegicus (Rat), 267 aa. | 1 . . . 267 | 262/267 (97%) | |
| Q8CE45 | Long chain fatty acyl elongase - Mus | 1 . . . 265 | 256/267 (95%) | e−151 |
| | musculus (Mouse), 267 aa. | 1 . . . 267 | 261/267 (96%) | |
| Q8NCD1 | Hypothetical protein FLJ90332 - | 26 . . . 265 | 240/240 (100%) | e−143 |
| | Homo sapiens (Human), 240 aa. | 1 . . . 240 | 240/240 (100%) | |

PFam analysis predicts that the NOV3a protein contains the domains shown in the Table C9.

TABLE C9
Domain Analysis of NOV3a

| | | Identities/ | |
|---|---|---|---|
| Pfam | NOV3a | Similarities for | |
| Domain | Match Region | the Matched Region | Expect Value |
| ELO | 10 . . . 265 | 85/327 (26%) | 5.2e−46 |
| | | 168/327 (51%) | |

### EXAMPLE C3

### Expression Profile of the Human Long Chain Fatty Acyl Elongase Gene

The protocol for quantitative expression analysis is disclosed in Example Q9.

Expression of genes CG180320-01, CG180320-O₂, CG180320-03, and CG180320-04 was assessed using the primer-probe set Ag6596, described in Table C10. Results of the RTQ-PCR runs are shown in Tables C11 and C12.

TABLE C10
Probe Name Ag6596

| | | | Start | SEQ ID |
|---|---|---|---|---|
| Primers | Sequences | Length | Position | No |
| Forward | 5′-caatggatgcaggaaaactg-3′ | 20 | 350 | 186 |
| Probe | TET-5′-tttcctgttttctgctctgtatgctg-3′- | 26 | 379 | 187 |
| | TAMRA | | | |
| Reverse | 5′-ccgaccaccgaatataaagg-3′ | 20 | 405 | 188 |

TABLE C11
General screening panel v1.6

| | Rel. Exp. (%) |
|---|---|
| | Ag6596, Run |
| Tissue Name | 277256790 |
| Adipose | 2.2 |
| Melanoma* Hs688(A).T | 6.0 |
| Melanoma* Hs688(B).T | 11.9 |
| Melanoma* M14 | 11.0 |
| Melanoma* LOXIMVI | 26.1 |
| Melanoma* SK-MEL-5 | 29.1 |
| Squamous cell carcinoma SCC-4 | 24.1 |
| Testis Pool | 0.9 |
| Prostate ca.* (bone met) PC-3 | 2.0 |
| Prostate Pool | 1.9 |
| Placenta | 0.5 |
| Uterus Pool | 0.7 |
| Ovarian ca. OVCAR-3 | 25.0 |
| Ovarian ca. SK-OV-3 | 4.5 |
| Ovarian ca. OVCAR-4 | 3.6 |
| Ovarian ca. OVCAR-5 | 50.0 |
| Ovarian ca. IGROV-1 | 12.0 |
| Ovarian ca. OVCAR-8 | 14.4 |
| Ovary | 2.4 |
| Breast ca. MCF-7 | 20.7 |
| Breast ca. MDA-MB-231 | 100.0 |
| Breast ca. BT 549 | 47.3 |
| Breast ca. T47D | 4.8 |
| Breast ca. MDA-N | 10.1 |
| Breast Pool | 1.7 |
| Trachea | 1.9 |
| Lung | 0.4 |
| Fetal Lung | 5.6 |
| Lung ca. NCI-N417 | 8.2 |
| Lung ca. LX-1 | 15.7 |
| Lung ca. NCI-H146 | 4.0 |
| Lung ca. SHP-77 | 18.8 |
| Lung ca. A549 | 20.3 |
| Lung ca. NCI-H526 | 6.4 |
| Lung ca. NCI-H23 | 8.6 |
| Lung ca. NCI-H460 | 12.1 |
| Lung ca. HOP-62 | 13.7 |
| Lung ca. NCI-H522 | 2.8 |
| Liver | 3.1 |
| Fetal Liver | 38.2 |
| Liver ca. HepG2 | 10.9 |
| Kidney Pool | 2.7 |
| Fetal Kidney | 17.9 |
| Renal ca. 786-0 | 21.5 |
| Renal ca. A498 | 17.1 |
| Renal ca. ACHN | 7.7 |
| Renal ca. UO-31 | 11.3 |
| Renal ca. TK-10 | 14.4 |
| Bladder | 5.1 |
| Gastric ca. (liver met.) NCI-N87 | 28.1 |
| Gastric ca. KATO III | 55.5 |
| Colon ca. SW-948 | 14.2 |
| Colon ca. SW480 | 10.4 |
| Colon ca.* (SW480 met) SW620 | 8.6 |
| Colon ca. HT29 | 15.3 |
| Colon ca. HCT-116 | 45.4 |
| Colon ca. CaCo-2 | 22.2 |
| Colon cancer tissue | 6.9 |
| Colon ca. SW1116 | 5.9 |
| Colon ca. Colo-205 | 15.0 |
| Colon ca. SW-48 | 10.4 |
| Colon Pool | 3.5 |
| Small Intestine Pool | 1.9 |
| Stomach Pool | 2.1 |
| Bone Marrow Pool | 1.5 |
| Fetal Heart | 2.9 |
| Heart Pool | 1.0 |
| Lymph Node Pool | 4.1 |
| Fetal Skeletal Muscle | 4.1 |
| Skeletal Muscle Pool | 0.2 |
| Spleen Pool | 1.4 |
| Thymus Pool | 3.5 |
| CNS cancer (glio/astro) U87-MG | 31.2 |
| CNS cancer (glio/astro) U-118-MG | 45.1 |
| CNS cancer (neuro; met) SK-N-AS | 47.3 |
| CNS cancer (astro) SF-539 | 8.3 |
| CNS cancer (astro) SNB-75 | 10.3 |
| CNS cancer (glio) SNB-19 | 20.9 |
| CNS cancer (glio) SF-295 | 28.7 |
| Brain (Amygdala) Pool | 7.8 |
| Brain (cerebellum) | 13.6 |
| Brain (fetal) | 16.0 |
| Brain (Hippocampus) Pool | 5.6 |
| Cerebral Cortex Pool | 9.8 |
| Brain (Substantia nigra) Pool | 6.4 |
| Brain (Thalamus) Pool | 12.5 |
| Brain (whole) | 6.1 |
| Spinal Cord Pool | 12.3 |
| Adrenal Gland | 9.7 |
| Pituitary gland Pool | 1.9 |
| Salivary Gland | 0.3 |
| Thyroid (female) | 0.4 |
| Pancreatic ca. CAPAN2 | 32.5 |
| Pancreas Pool | 1.5 |

TABLE C12
Panel 5 Islet

| | Rel. Exp. (%) |
|---|---|
| | Ag6596, Run |
| Tissue Name | 279518227 |
| 97457_Patient-02go_adipose | 1.5 |
| 97476_Patient-07sk_skeletal muscle | 0.0 |
| 97477_Patient-07ut_uterus | 1.6 |
| 97478_Patient-07pl_placenta | 0.5 |
| 99167_Bayer Patient 1 | 1.3 |
| 97482_Patient-08ut_uterus | 2.0 |
| 97483_Patient-08pl_placenta | 0.9 |
| 97486_Patient-09sk_skeletal muscle | 0.6 |
| 97487_Patient-09ut_uterus | 1.4 |
| 97488_Patient-09pl_placenta | 0.5 |
| 97492_Patient-10ut_uterus | 2.1 |
| 97493_Patient-10pl_placenta | 1.4 |
| 97495_Patient-11go_adipose | 1.1 |
| 97496_Patient-11sk_skeletal muscle | 0.1 |
| 97497_Patient-11ut_uterus | 3.9 |
| 97498_Patient-11pl_placenta | 0.3 |
| 97500_Patient-12go_adipose | 1.2 |
| 97501_Patient-12sk_skeletal muscle | 1.4 |
| 97502_Patient-12ut_uterus | 1.5 |
| 97503_Patient-12pl_placenta | 0.8 |
| 94721_Donor 2 U - A_Mesenchymal Stem Cells | 8.8 |
| 94722_Donor 2 U - B_Mesenchymal Stem Cells | 8.5 |
| 94723_Donor 2 U - C_Mesenchymal Stem Cells | 8.9 |
| 94709_Donor 2 AM - A_adipose | 9.6 |
| 94710_Donor 2 AM - B_adipose | 6.5 |
| 94711_Donor 2 AM - C_adipose | 4.9 |
| 94712_Donor 2 AD - A_adipose | 24.7 |
| 94713_Donor 2 AD - B_adipose | 29.3 |
| 94714_Donor 2 AD - C_adipose | 22.2 |
| 94742_Donor 3 U - A_Mesenchymal Stem Cells | 4.8 |
| 94743_Donor 3 U - B_Mesenchymal Stem Cells | 5.6 |
| 94730_Donor 3 AM - A_adipose | 17.2 |
| 94731_Donor 3 AM - B_adipose | 27.5 |
| 94732_Donor 3 AM - C_adipose | 17.2 |
| 94733_Donor 3 AD - A_adipose | 80.1 |
| 94734_Donor 3 AD - B_adipose | 74.2 |
| 94735_Donor 3 AD - C_adipose | 24.8 |
| 77138_Liver_HepG2untreated | 100.0 |
| 73556_Heart_Cardiac stromal cells (primary) | 1.5 |
| 81735 Small Intestine | 4.0 |
| 72409_Kidney_Proximal Convoluted Tubule | 33.2 |
| 82685_Small intestine_Duodenum | 6.0 |
| 90650_Adrenal_Adrenocortical adenoma | 2.2 |
| 72410_Kidney_HRCE | 19.1 |
| 72411_Kidney_HRE | 20.0 |
| 73139_Uterus_Uterine smooth muscle cells | 16.6 |

General screening panel v1.6 Summary: (Ag6596) Moderate expression of this gene was detected ubiquitously in all tissues (CT=28-32). This ubiquitous pattern of expression indicates that this gene product is involved in homeostatic processes for these and other cell types and tissues.

Its expression was elevated in all the cancer cell lines (melanoma, ovarian cancer, breast cancer, lung cancer, renal cancer, colon cancer, pancreatic cancer, and CNS cancer) with CT=25.5-30. Therapeutic modulation of this gene, expressed protein and/or use of antibodies or small molecule drugs targeting the gene or gene product may be useful in the treatment of melanoma, ovarian cancer, breast cancer, lung cancer, renal cancer, pancreatic cancer, and CNS cancer. In addition, the expression of this gene can be used as a detection marker for those cancers.

This gene was expressed in adipose tissue (CT=30.98). Therapeutic modulation of this gene, expressed protein and/or use of antibodies or small molecule drugs targeting the gene or gene product may be useful in the treatment of obesity and diabetes.

Panel 5 Islet Summary: (Ag6596) Expression of this gene was induced in adipose differentiated from Mesenchymal Stem cells (compare AD samples with U samples from Donor 2 (CT=30.46-30.86 vs. CT=32.18-32.24) and Donor 3 (CT=29.01-30.7 vs. CT=32.84-33.07). Thus therapeutic modulation of this gene, expressed protein and/or use of antibodies or small molecule drugs targeting the gene or gene product can be useful in the treatment of obesity and diabetes.

### EXAMPLE C4

### Assays Screening for Modulators of Long Chain Fatty Acyl Elongase

A non-exhaustive list of cell lines that express the long chain fatty acyl elongase can be obtained from the differential gene expression (RTQ-PCR) results presented herein.

Potential methods for Measurement of Fatty Acyl elongation reaction in microsomes are described by Nagi et al., Evidence for two separate beta-ketoacyl CoA reductase components of the hepatic microsomal fatty acid chain elongation system in the rat, Biochem Biophys Res Commun. 1989 Dec. 29; 165 (3):1428-34, and by Moon et al., Identification of a mammalian long chain fatty acyl elongase regulated by sterol regulatory element-binding proteins, J Biol. Chem. 2001 Nov. 30; 276 (48):45358-66. Epub 2001 Sep. 20, followed by HPLC separation of radio-labeled fatty acids by HPLC.

Functional/mechanistic assay for the effectiveness of an administered modulator of Fatty Acyl Elongase could be performed by obesrving TG synthesis/accumulation in 3T3-L1 mouse adipocytes, human adipocytes or hepatocytes by Oil Red O staining. Alternatively measurement could be made of post treatment TG accumulation in cells transfected with Fatty Acyl Elongase.

Our results indicate that a modulator of Long Chain Fatty Acyl Elongase activity, such as an inhibitor, activator, antagonist, or agonist of Long Chain Fatty Acyl Elongase may be useful for treatment of such disorders as obesity, diabetes, and insulin resistance, as well as for enhancement of insulin secretion.

D. NOV4—Acetyl-Coenzyme A acyltransferase 2

Acetyl-Coenzyme A acyltransferase 2 (ACAA2) is the last enzyme in mitochondrial fatty acid beta-oxidation. It is thought that fatty acid oxidation may interfere with glucose utilization in skeletal muscle and liver, thus causing insulin resistance and hyperglycemia. (Randle P J. Regulatory interactions between lipids and carbohydrates: the glucose fatty acid cycle after 35 years. Diabetes Metab. Rev. 1998 14 (4), 263-83 PMID: 10095997; Deems R O, Anderson R C, Foley J E. Hypoglycemic effects of a novel fatty acid oxidation inhibitor in rats and monkeys. Am. J. Physiol. 1998 274 (2 Pt 2):R524-8; PMID: 9486313). GeneCalling® studies showed significant up-regulation of ACAA2 in diabetic muscle. ACAA2 was also down-regulated in fast twitch versus slow twitch diabetic skeletal muscle in response to vanadate, metformin and AICAR, compounds causing improvement of hypoglycemia and diabetes. GeneCalling® data indicate that ACAA2 may make a good target for promoting skeletal muscle glucose utilization and improving diabetes.

Shown below is the reaction catalyzed by human Acetyl-Coenzyme A acyltransferase 2.
3-oxoacyl CoA+CoA⇄Acyl CoA+Acetyl CoA
Acetyl CoA→TCA cycle

Potential assay that may be used to screen for antibody therapeutics or small molecule drugs is the measurement of NAD+/NADH production in the outlined reaction coupled with the conversion reaction of 3-hydroxyacyl CoA to 3-oxoacyl CoA by the next mitochondrial enzyme. The general inhibitor for mitochondrial thiolases (4-bromocrotonic acid) is described in literature (Schulz et al., Life Sci. (1987) 40, 1443). Cell lines expressing the Acetyl-Coenzyme A acyltransferase 2 can be obtained from the RTQ-PCR results shown herein. These and other Acetyl-Coenzyme A acyltransferase 2 expressing cell lines could be used for screening purposes.

ACAA2 catalyses the last step in acetyl-CoA production. Concerning accumulation of ACAA2 substrate, 3-oxoacyl-CoA has no reported toxicity and causes inhibition of two other enzymes in fatty acid oxidation cycle. The outcome of inhibiting the action of the human Acetyl-Coenzyme A acyltransferase 2 would be inhibition of fatty acid oxidation in skeletal muscle and liver, and promotion of glucose utilization.

Acetyl-Coenzyme A acyltransferase 2 (ACAA2) (3-oxoacyl-CoA thiolase) is up-regulated in skeletal muscle in obese/diabetic skeletal muscle. The expression level of the enzyme is down-regulated in gastrocnemius versus soleus muscle in response to vanadate, AICAR and Metformin treatments (all are known to improve glucose utilization in muscle). ACAA2 is the last enzyme in fatty acid beta-oxidation. Inhibition of ACAA2 would inhibit fatty acid oxidation in skeletal muscle and liver, promoting glucose utilization. Thus, an inhibitor/antagonist of ACAA2 would be beneficial as an antihyperglycemic agent for the treatment of obesity and/or diabetes.

Furthermore, we discovered that Acetyl-Coenzyme A acyltransferase 2 (ACAA2) is down-regulated in diabetic skeletal muscle of animals that respond favorably to rosiglitazone treatment with improved glycemic control. However, the expression of Acetyl-Coenzyme A acyltransferase 2 was found to be not down-regulated in response to rosiglitazone treatment in diabetic animals that had no detectable improvement of hyperglycemia. Because acetyl-Coenzyme A acyltransferase 2 expression is positively correlated with hyperglycemia, its down-regulation may account for the antidiabetic effect of rosiglitazone. Therefore, an attenuation of Acetyl-Coenzyme A acyltransferase 2 total enzymatic activity might account for, and be sufficient to yield, favorable clinical effects comparable to that of rosiglitazone on hyperglycemia and skeletal muscle insulin sensitivity. Our data support the development of an antagonist of Acetyl-Coenzyme A acyltransferase 2 as a therapeutic agent to treat insulin resistance and diabetes.

Furthermore, our results indicate that a modulator of ACAA2 activity, such as an inhibitor, activator, antagonist, or agonist of ACAA2 may be useful for treatment of such disorders as obesity, diabetes, and insulin resistance, as well as for enhancement of insulin secretion.

Discovery Process

The following sections describe the study design(s) and the techniques used to identify the Acetyl-Coenzyme A acyltransferase 2—encoded protein and any variants, thereof, as being suitable as diagnostic markers, targets for an antibody therapeutic and targets for a small molecule drugs for Obesity and Diabetes.

### EXAMPLE D1

### Mouse Dietary-Induced Obesity

A protocol for Mouse Dietary-Induced Obesity study is disclosed in Example Q1.

The predominant cause for obesity in clinical populations is excess caloric intake. This so-called diet-induced obesity (DIO) is mimicked in animal models (mouse strain C57BL) by feeding high fat diets of greater than 40% fat content. The DIO study was established to identify the gene expression changes contributing to the development and progression of diet-induced obesity. In addition, the study design sought to identify the factors that lead to the ability of certain individuals to resist the effects of a high fat diet and thereby prevent obesity. The sample groups for the study had body weights +1 S.D., +4 S.D. and +7 S.D. of the chow-fed controls. In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues revealed genes and pathways that can be used as therapeutic targets for obesity and/or diabetes. Protocol for differential gene expression analysis, GeneCalling®, is disclosed in Example Q7.

Results

A fragment of the mouse Acetyl-Coenzyme A acyltransferase 2 gene was initially found to be up-regulated by 1.6 fold in the muscle of the obese, diabetic mice (hgsd7) on a high fat diet as compared to mice on normal diet (chow) using CuraGen's GeneCalling® method of differential gene expression. A differentially expressed mouse gene fragment migrating, at approximately 380.4 nucleotides in length was definitively identified as a component of the mouse Acetyl-Coenzyme A acyltransferase 2 cDNA. The method of competitive PCR was used for confirmation of the gene assessment. The electropherographic peaks corresponding to the gene fragment of the mouse Acetyl-Coenzyme A acyltransferase 2 were ablated when a gene-specific primer (shown in Table D1) competes with primers in the linker-adaptors during the PCR amplification. The peaks at 380.4 nt in length were ablated in the sample from both the obese/diabetic and normal chow mouse.

TABLE D1

The direct sequence of the 380.4 nucleotide-long gene fragment and
the gene-specific primers used for competitive PCR are indicated on
the cDNA sequence of the Acetyl-Coenzyme A acyltransferase 2 fragment
(SEQ ID NO:189) and are shown in bold. The gene-specific primers at
the 5′ and 3′ ends of the fragment are underlined.
Competitive PCR Primer for the mouse Acetyl-Coenzyme A
acyltransferase 2: Gene Sequence (fragment from 507 to 886 in bold.
band size: 380)

| | |
|---|---|
| 1 | AGAGCCCCGC GGAATAGCTG AGCTTCGCCA TGGCCCTGCT ACGAGGTGTG TTCATCGTCG |
| 61 | CTGCGAAGAG ACACCCTTTG GAGCTTACGG GGGCCTTCTC AAGGACTTCT CTGCCACCGA |
| 121 | TTTAACTGAA TTTGCTGCCA GGGCTGCTCT GTCUGCTGGC AAAGTTCCAC CTGAAACCAT |
| 181 | CGATAGTGTC ATCGTGGGCA ATGTCATGCA GAGCTCTTCA GATGCGGCAT ACCTGGCGAG |
| 241 | GCATGTGGGT TTGCGAGTGG GAGTCCCAAC AGAGACTGGG GCCCTTACCC TCAACAGGCT |
| 301 | CTGTGGCTCT GGTTTCCAGT CCATCGTGAG CGGATGTCAG GAAATCTGTT CTAAAGATGC |
| 361 | TGAGGTCGTC TTGTGTGGAG GAACAGAGAG CATGAGCCAG TCCCCCTACT GTGTCAGAAA |
| 421 | TGTGCGCTTC GGAACCAAAT TTGGATTAGA TCTCAAGCTG GAAGATACTT TGTGGGCAGG |
| 481 | ATTAACGGAT CAACATGTTA AGCTGCCCAT GGGAATGACT GCAGAGAACC TTGCTGCAAA |
| 541 | ATACAACATA AGCAGAGAAG ACTGTGACAG ATACGCCTTG CAGTCTCAGC AGAGGTGGAA |
| 601 | AGCTGCTAAC GAGGCTGGCT ACTTCAATGA GGAGATGGCA CCCATTGAGG TGAAGACGAA |
| 661 | GAAAGGCAAA CAGACCATGC AAGTGGACGA GCACGCTCGA CCCCAAACCA CCCTGGAGCA |
| 721 | ACTGCAGAAG CTCCCGTCCG TGTTCAAGAA AGACGGGACA GTCACAGCAG GGAACGCCTC |
| 781 | GGGGGTGTCT GACGGTGCTG GGGCCGTCAT CATAGCCAGC GAAGATGCTG TCAAAAAACA |
| 841 | TAACTTCACG CCCCTGGCCA GAGTCGTGGG CTACTTCGTG TCCGGATGCG ATCCTACTAT |
| 901 | CATGGGTATT GGTCCAGTCC CTGCTATCAA TGGAGCATTG AAGAAAGCTG GGCTGAGTCT |
| 961 | TAAGGACATG GATTTGATAG ACGTGAACGA AGCTTTTGCC CCTCAGTTCT TGTCTGTTCA |
| 1021 | GAAGGCCCTG GATCTTGACC CCAGCAAAAC CAATGTGAGT GGAGGCGCCA TTGCCCTGGG |
| 1081 | TCACCCGCTG GGAGGATCTG GCTCCAGAAT CACCGCACAC CTGGTTCATG AGTTAAGGCG |
| 1141 | TCGAGGTGGA AAGTACGCAG TGGGATCAGC TTGCATTGGA GGTGGCCAAG GCATCGCCTT |
| 1201 | GATCATCCAG AACACAGCCT GAAGGCATCA CAAGCACACT GCCCACACTT ACTGGGCCAG |
| 1261 | GCCACGGAAC ACAGGAGACC TTCGAGTCAG CCCTGCTGAG ACAGTGATTG TATGTGACCA |
| 1321 | AGCCTTGATG AGGCAAGATG CATTGGGTTC TGTCTACTTC ATACCTGTCT GACGTGTTAG |
| 1381 | AATAAAAACA CCAACCATCG GAGGCCTTAA GAGAAATGGT ATCTGTCAGT AGTCACCACT |
| 1441 | GTATGCCTTC CATGGAGTAA TACAAACTGA ATAAATGTTG CCTTAACTCC AGCT |

### EXAMPLE D2

### Insulin Sensitivity In Rat Skeletal Muscle

A protocol for Rat Insulin Sensitivity study is disclosed in Example Q3.

ZDF rats were treated with a variety of agents that are known to alter insulin sensitivity. Metformin, vanadate, and AICAR enhance tissue response to insulin, while the free fatty acids generated by Liposyn (intravenous lipid infusion) ZDF rats or their lean littennates were treated with a variety of agents that are known to alter insulin sensitivity. Metformin, vanadate, and AICAR enhance tissue response to insulin, while the free fatty acids generated by Liposyn (intravenous lipid infusion) treatment reduces the response. A variety of tissues were harvested, including gastrocnemius and soleus muscles, liver, retroperitoneal and epididymal WAT, and IBAT. Protocol for differential gene expression analysis, GeneCalling®, is disclosed in Example Q7.

Results

A gene fragment of the rat Acetyl-Coenzyme A acyltransferase 2 was found to be down-regulated by 2.5 fold in gastrocnemius relative to soleus muscle in ZDF rats treated with vanadate, AICAR and Metformin using CuraGen's GeneCalling® method of differential gene expression. A differentially expressed rat gene fragment migrating, at approximately 353 nucleotides in length was definitively identified as a component of the rat Acetyl-Coenzyme A acyltransferase 2 cDNA. The method of competitive PCR was used for confirmation of the gene assessment. The electropherographic peaks corresponding to the gene fragment of the rat Acetyl-Coenzyme A acyltransferase 2 were ablated when a gene-specific primer (shown in Table D2) competes with primers in the linker-adaptors during the PCR amplification. The peaks at about 353 nt in length were ablated in the sample from both gastrocnemius and soleus muscle.

TABLE D2

The direct sequence of the 353 nucleotide-long gene fragment and
specific primers used for competitive PCR are indicated on the cDNA
sequence of the Acetyl-Coenzyme A acyltransferase 2 fragment (SEQ ID
NO:190) and are shown in bold. The gene-specific primers at the 5′
and 3′ ends of the fragment are underlined.
Competitive PCR Primer for the rat Acetyl-Goenzyme A
acyltransferase 2: Gene Sequence (fragment from 643 to 996 in bold.
band size: 354)

| | |
|---|---|
| 162 | GTCATGGCGC TGCTACGAGG TGTGTTTATC GTTGCTGCGA AGCGAACACC CTTTGGAGCT |
| 222 | TATGGGGGTC TTCTCAAGGA CTTCACTGCC ACTGACTTAA CTGAATTTGC TGCCAGGGCT |
| 282 | GCCCTGTCTG CTGGCAAAGT TCCACCGGAA ACCATCGATA GTGTCATCGT GGGCAATGTC |
| 342 | ATGCAGAGCT CTTCAGATGC GGCGTACCTG GCAAGGCATG TGGGTTTACG TGTGGGAGTC |
| 402 | CCGACGGAGA CTGGGGCCCT CACCCTCAAC AGACTCTGTG GCUCTGGTTT CCAGTCCATC |
| 462 | GTGAGCGGAT GTCAGGAAAT CTGCTCGAAA GACGCTGAGG TCGTCTTATG TGGAGGAACC |
| 522 | GAGAGCATGA GCCAGTCCCC CTACTCTGTC AGAAATGTGC GCTTCGGAAC CAAATTTGGG |
| 582 | TTAGATCTCA AGCTGGAAGA TACTTTGTGG GCAGGATTAA CGGATCAACA CGTGAAGCTC |
| 642 | CCCATGGGGA TGACTGCAGA GAACCTGGCT GCAAAATACA ACATAAGCAG AGAAGACTGC |
| 702 | GACAGATACG CCCTGCAGTC CCAGCAGAGG TGGAAAGCCG CTAACGAGGC TGGCTACTTT |
| 762 | AATGAGGAGA TGGCCCCCAT TGAGGTGAAG ACCAAGAAGG GCAAACAGAC CATGCAAGTG |
| 822 | GATGAGCACG CCCGGCCCCA AACGACCCTG GAGCAGCTGC AGAACCTCCC GCCAGTGTTC |
| 882 | AAGAAAGAGG GGACGGTCAC AGCAGGGAAC GCCTCGGGCA TGTCTGACGG TGCTGGGGTC |
| 942 | GTCATCATAG CCAGCGAAGA TGCTGTCAAA AAACATAACT TCACACCACT GGCCAGAGTC |
| 1002 | GTGGGCTACT TTGTGTCTGG ATGTGACCCT GCTATCATGG GGATCGGTCC AGTCCCTGCC |
| 1062 | ATCACTGGAG CATTGAAGAA AGCTGGGCTG AGCCTTAAGG ACATGGATTT GATAGACGTG |
| 1122 | AATGAAGCAT TTGCTCCTCA GTTCTTGGCT GTTCAGAAGA GCTTGGATCT CGACCCCAGT |
| 1182 | AAAACCAACG TGAGTGGAGG TGCCATAGCC CTGGGTCACC CGCTGGGAGG ATCTGGATCC |
| 1242 | AGAATCACCG CACACCTGGT TCACGAGTTA AGGCGTCGAG GTGGAAAATA CGCAGTGGGA |
| 1302 | TCAGCTTGCA TTGGAGGTGG CCAAGGCATC TCCCTGATCA TCCAGAACAC AGCCTGAAGG |
| 1362 | GATTGCAAGC ATCCTACCCA CCCTCACTTG GCCAGGCTAC GGAACACAGG CGACCTTTGA |
| 1422 | GTCAGCCCTG CTGTGACAGT AAATGCATTT GACCAAGCCT TGATGGGTTC TGTCT |

### EXAMPLE D3

### Mouse TZD Response Study

A protocol for Mouse TZD Response study is disclosed in Example Q4.

The peroxisome proliferator-activated receptor gamma (PPARg) is the member of the nuclear hormone receptor subfamily of transcription factors that plays a major role in regulation of metabolism (Lee C H, Olson P, Evans R M. Minireview: lipid metabolism, metabolic diseases, and peroxisome proliferator-activated receptors. Endocrinology. 2003 June; 144 (6):2201-7. PMID: 12746275). The thiazolidinedione (TZD) drugs, including rosiglitazone, are synthetic agonists of PPARg receptors that can normalize elevated plasma glucose levels in obese, diabetic rodents and are often quite efficacious therapeutic agents for the treatment of noninsulin-dependent diabetes mellitus in humans (Doggrell S. Do peroxisome proliferation receptor-gamma antagonists have clinical potential as combined antiobesity and antidiabetic drugs? Expert Opin Investig Drugs. 2003 April; 12 (4):713-6., PMID: 12665425; Gurnell M, Savage D B, Chatterjee V K, O'Rahilly S. The metabolic syndrome: peroxisome proliferator-activated receptor gamma and its therapeutic modulation. J Clin Endocrinol Metab. 2003 June; 88 (6):2412-21 PMID: 12788836). Diabetic animals demonstrate differential responses to TZD treatment. To understand the basis for this differential response we compared changes in gene expression between diabetic animals that responded favorably and that did not respond to TZD treatment. Female db/db mice were treated daily with 10 mg per kilogram body weight rosiglitazone for 7 days. On day 8, the mice were bled for blood glucose. Treated mice were grouped into either a ‘responder group’ that demonstrated a significant decrease of their hyperglycemia and a ‘non-responder group’ that demonstrated no change in their blood glucose level. Gene expression in skeletal muscle and adipose tissues was compared between untreated diabetic mice and the two sub-groups of TZD treated mice. Protocol for differential gene expression analysis, GeneCalling®, is disclosed in Example Q7.

Results

Using CuraGen's GeneCalling® method of differential gene expression, a fragment of the mouse Acetyl-Coenzyme A acyltransferase 2 gene was initially found to be down-regulated by 1.7 fold in the skeletal muscle of diabetic mice treated with rosiglitazone that demonstrated improvement of hyperglycemia relative to mice treated with rosiglitazone, but failed to decrease blood glucose level. The same fragment has been found to be down-regulated 1.9 fold in skeletal muscle of TZD treated mice with improved hyperglycemia relative to diabetic controls. A differentially expressed mouse gene fragment migrating at approximately 252 nucleotides in length on capillary gel electrophoresis was definitively identified as a component of the mouse Acetyl-Coenzyme A acyltransferase 2 cDNA. The method of competitive PCR was used for confirmation of the gene assessment. The electropherographic peak corresponding to the gene fragment of mouse Acetyl-Coenzyme A acyltransferase 2 was ablated when a gene-specific primer (shown in Table D3) competes with primers in the linker-adaptors during the PCR amplification. These data are suggestive of Acetyl-Coenzyme A acyltransferase 2 being involved in skeletal muscle insulin resistance and the progression of diabetes.

TABLE D3

The sequence of the 252 nucleotide-long gene fragment and the gene-
specific primers used for competitive PCR are indicated in bold on
the cDNA sequence of the mouse ACCA2 gene fragment (SEQ ID NO:191).
The gene-specific primers at the 5′ and 3′ ends of the fragment are
underlined.
Gene Sequence (fragment from 370 to 620 in bold. band size: 251)

| | |
|---|---|
| 1 | TTTTTTTTTT GGAGTTAAGG CAACATTTAT TCAGTTTGTA TTACTCCATG GAAGGCATAC |
| 61 | AGTGGTGACT ACTGACAGAT ACCATTTCTC TTAAGGCCTC CGATGGTTGG TGTTTTTATT |
| 121 | CTAACACGTC AGACAGGTAT GAAGTAGACA GAACCCAATG CATCTTGCCT CATCAAGGCT |
| 181 | TGGTCACATA CAATCACTGT CTCAGCAGGG CTGACTCGAA GGTCTCCTGT GTTCCGTGGC |
| 241 | CTGGCCCAGT AAGTGTGGGC AGTGTGCTTG TGATGCCTTC AGGCTGTGTT CTGGATGATC |
| 301 | AAGGCGATGC CTTGGCCACC TTCCATGCAA GCTGATTCCA CTGGGTACTT TCCACCTTGA |
| 361 | CGCCTTAACT CATCAACCAG GTGTGCGGTG ATTCTGGACC CAGATCCTCC CAGCGGGTGA |
| 421 | CCCAGGCCAA TGCCGCCTCC ACTCACATTG GTTTTCCTGG GGTCAAGATC CACGGCCTTC |
| 481 | TCAACAGACA AGAACTGAGG GCCAAAAGCT TCGTTCACGT CTATCAAATC CATGTCCTTA |
| 541 | AGACTCACCC CACCTTTCTT CAATGCTCCA TTGATACCAA GCACCTCACC AATACCCATG |
| 601 | ATAGTAGGAT CGCATCCGGA CACGAAGTAG CCCACGACTC TGGCCAAGGG CCGTGACGTT |
| 661 | TATGTTTTGA CAGCATCCTT GCTGGGCTTG ATGAGGGCCC CAGCACGTCA GACACCCCCG |
| 721 | AGGCGTATCC TTGTTTGACT GTCCCGTTTT TTTTAAACAC TGAGGGGAGC TCTTGCAGTA |
| 781 | GCTCCAGGGT GGATTTGGA |

(gene length is 799, only region from 1 to 799 shown)

### EXAMPLE D4

### Identification of Human Acetyl-Coenzyme A acyltransferase 2 Gene Sequences

The sequence of Human Acetyl-Coenzyme A acyltransferase 2 Gene (Acc. No. CG181387-01) was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof. The protocol for identification of human sequence(s) is disclosed in Example Q8.

Table D4 shows an alignment (ClustalW) of the protein sequences of the human (CG181387-01; SEQ ID NO:30), rat (P13437; SEQ ID NO:192) and mouse (BC028901; SEQ ID NO:193) versions of the Acetyl-Coenzyme A acyltransferase 2. Table D5 shows protein sequences of rat (P13437; SEQ ID NO:192) and mouse (BC028901; SEQ ID NO:193) versions of the Acetyl-Coenzyme A acyltransferase 2.

[image]

TABLE D5

protein sequences of rat (P13437; SEQ ID NO:192) and mouse (BC028901; SEQ
ID NO:193) versions of the Acetyl-Goenzyme A acyltransferase 2.

Rat Acetyl-Coenzyme A acyltransferase 2(P13437; SEQ ID NO:192)
MALLRGVFIVAAKRTPFGAYGGLLKDFTATDLTEFAARAALSAGKVPPETIDSVIVGNVMQSSSDAAYLA
RHVGLRVGVPTETGALTLNRLCGSGFQSIVSGCQEICSKDAEVVLCGGTESMSQSPYSVRNVRFGTKFGL
DLKLEDTLWAGLTDQHVKLPMGMTAENLAAKYNISREDCDRYALQSQQRWKAANEAGYFNEEMAPLEVKT
KKGKQTMQVDEHARPQTTLEQLQNLPPVFKKEGTVTAGNASGMSDGAGVVIIASEDAVKKHNFTPLARVV
GYFVSGCDPAIMGIGPVPAITGALKKAGLSLKDMDLIDVNEAFAPQFLAVQKSLDLDPSKTNVSGGAIAL
GHPLGGSGSRITAHLVHELRRRGGKYAVGSACIGGGQGISLIIQNTA
Mouse Acetyl-Coenzyme A acyltransferase 2 (AAH28901; BC028901; SEQ ID NO:193)
MALLRGVFIVAAKRTPFGAYGGLLKDFSATDLTEFAARAALSAGKVPPETIDSVIVGNVMQSSSDAAYLA
RHVGLRVGVPTETGALTLNRLCGSGFQSIVSGCQEICSKDAEVVLCGGTESMSQSPYCVRNVRFGTKFGL
DLKLEDTLWAGLTDQHVKLPMGMTAENLAAKYNISREDCDRYALQSQQRWKAANEAGYFNEEMAPIEVKT
KKGKQTMQVDEHARPQTTLEQLQKLPSVFKKDGTVTAGNASGVSDGAGAVIIASEDAVKKHNFTPLARVV
GYFVSGCDPTIMGIGPVPAINGALKKAGLSLKDMDLIDVNEAFAPQFLSVQKALDLDPSKTNVSGGAIAL
GHPLGGSGSRITAHLVHELRRRGGKYAVGSACIGGGQGIALIIQNTV

The laboratory cloning was performed using one or more of the methods summarized in Example Q8. The NOV4 clone was analyzed, and the nucleotide and encoded polypeptide sequences are shown in Table D6.

TABLE D6
NOV4 Sequence Analysis

| | | |
|---|---|---|
| NOV4a, CG181387-01 | SEQ ID NO:29 | 1584 bp |
| DNA Sequence | ORF Start: ATG at 49 | ORF Stop: TGA at 1240 |

GCGTCCCCCACACCACAGACCCGCGCCGCCGACGACCCAGCAGCCGCCATGCGTCTGCTCCGAGGTGT
GTTTGTAGTTGCTGCTAAGCGAACGCCCTTTGGAGCTTACGGAGGCCTTCTGAAAGACTTCACTGCTA
CTGACTTGTCTGAATTTGCTGCCAAGGCTGCCTTGTCTGCTGGCAAAGTCTCACCTGAAACAGTTGAC
AGTGTGATTATGGGCAATGTCCTGCAGAGTTCTTCAGATGCTATATATTTGGCAAGGCATGTTGGTTT
GCGTGTGGGAATCCCAAAGGAGACCCCAGCTCTCACGATTAATAGGCTCTGTGGTTCTGGTTTTCAGT
CCATTGTGAATGGATGTCAGGAAATTTGTGTTAAAGAAGCTGAAGTTGTTTTATGTGGAGGAACCGAA
AGCATGAGCCAAGCTCCCTACTGTGTCAGAAATGTGCGTTTTGGAACCAAGCTTGGATCAGATATCAA
GCTGGAAGATTCTTTATGGGTATCATTAACAGATCAGCATGTCCAGCTCCCCATGGCAATGACTGCAG
AGAATCTTACTGTAAAACACAAAATAAGCAGAGAAGAATGTGACAAATATGCCCTGCAGTCACAGCAG
AGATGGAAAGCTGCTAATGATGCTGGCTACTTTAATGATGAAATGGCACCAATTGAAGTGAAGACAAA
GAAAGGAAAACAGACAATGCAGGTAGACGAGCATGCTCGGCCCCAAACCACCCTGGAACAGTTACAGA
AACTTCCTCCAGTATTCAAGAAAGATGGAACTGTTACTGCAGGGAATGCATCGGGTGTAGCTGATGGT
GCTGGAGCTGTTATCATAGCTAGTGAAGATGCTGTTAAGAAACATAACTTCACACCACTGGCAAGAAT
TGTGGGCTACTTTGTATCTGGATGTGATCCCTCTATCATGGGTATTGGTCCTGTCCCTGCTATCAGTG
GGGCACTGAAGAAAGCAGGACTGAGTCTTAAGGACATGGATTTGGTAGAGGTGAATGAAGCTTTTGCT
CCCCAGTACTTGGCTGTTGAGAGGAGTTTGGATCTTGACATAAGTAAAACCAATGTGAATGGAGGAGC
CATTGCTTTGGGTCACCCACTGGGAGGATCTGGATCAAGAATTACTGCACACCTGGTTCACGAATTAA
GGCGTCGAGGTGGAAAATATGCCGTTGGATCAGCTTGCATTGGAGGTGGCCAAGGTATTGCTGTCATC
ATTCAGAGCACAGCCTGAAGAGACCAGTGAGCTCACTGTGACCCATCCTTACTCTACTTGGCCAGGCC
ACAGTAAAACAAGTGACCTTCAGAGCAGCTGCCACAACTGGCCATGCCCTGCCATTGAAACAGTGATT
AAGTTTGATCAAGCCATGGTGACACAAAAATGCATTGATCATGAATAGGAGCCCATGCTAGAAGTACA
TTCTCTCAGATTTGAACCAGTGAAATATGATGTATTTCTGAGCTAAAACTCAACTATAGAAGACATTA
AAAGAAATCGTATTCTTGCCAAGTAACCACCACTTCTGCCTTAGATAATATGATTATAAGGAAATCAA
ATAAATGTTGCCTTAACTTC

| | | | |
|---|---|---|---|
| NOV4a, CG181387-01 | | | |
| Protein Sequence | SEQ ID NO:30 | 397 aa | MW at 42038.9 kD |

MRLLRGVFVVAAKRTPFGAYGGLLKDFTATDLSEFAAKAALSAGKVSPETVDSVIMGNVLQSSSDAIY
LARHVGLRVGIPKETPALTINRLCGSGFQSIVNGCQEICVKEAEVVLCGGTESMSQAPYCVRNVRFGT
KLGSDIKLEDSLWVSLTDQHVQLPMAMTAENLTVKHKISREECDKYALQSQQRWKAANDAGYFNDEMA
PIEVKTKKGKQTMQVDEHARPQTTLEQLQKLPPVFKKDGTVTAGNASGVADGAGAVIIASEDAVKKHN
FTPLARIVGYFVSGCDPSIMGIGPVPAISGALKKAGLSLKDMDLVEVNEAFAPQYLAVERSLDLDISK
TNVNGGAIALGHPLGGSGSRITAHLVHELRRRGGKYAVGSACIGGGQGIAVIIQSTA

| | | |
|---|---|---|
| NOV4b, 282274427 | SEQ ID NO:31 | 1218 bp |
| DNA Sequence | ORF Start: at 2 | ORF Stop: TGA at 1205 |

CACCGGATCCACCATGCGTCTGCTCCGAGGTGTGTTTGTAGTTGCTGCTAAGCGAACGCCCTTTGGAG
CTTACGGAGGCCTTCTGAAAGACTTCACTGCTACTGACTTGTCTGAATTTGCTGCCAAGGCTGCCTTG
TCTGCTGGCAAAGTCTCACCTGAAACAGTTGACAGTGTGATTATGGGCAATGTCCTGCAGAGTTCTTC
AGATGCTATATATTTGGCAAGGCATGTTGGTTTGCGTGTGGGAATCCCAAAGGAGACCCCAGCTCTCA
CGATTAATAGGCTCTGTGGTTCTGGTTTTCAGTCCATTGTGAATGGATGTCAGGAAATTTGTGTTAAA
GAAGCTGAAGTTGTTTTATGTGGAGGAACCGAAAGCATGAGCCAAGCTCCCTACTGTGTCAGAAATGT
GCGTTTTGGAACCAAGCTTGGATCAGATATCAAGCTGGAAGATTCTTTATGGGTATCATTAACAGATC
AGCATGTCCAGCTCCCCATGGCAATGACTGCAGAGAATCTTGCTGTAAAACACAAAATAAGCAGAGAA
GAATGTGACAAATATGCCCTGCAGTCACAGCAGAGATGGAAAGCTGCTAATGATGCTGGCTACTTTAA
TGATGAAATGGCACCAATTGAAGTGAAGACAAAGAAAGGAAAACAGACAATGCAGGTAGACGAGCATG
CTCGGCCCCAAACCACCCTGGAACAGTTACAGAAACTTCCTCCAGTATTCAAGAAAGATGGAACTGTT
ACTGCAGGGAATGCATCGGGTGTAGCTGATGGTGCTGGAGCTGTTATCATAGCTAGTGAAGATGCTGT
TAAGAAACATAACTTCACACCACTGGCAAGAATTGTGGGCTACTTTGTATCTGGATGTGATCCCTCTA
TCATGGGTATTGGTCCTGTCCCTGCTATCAGTGGGGCACTGAAGAAAGCAGGACTGAGTCTTAAGGAC
ATGGATTTGGTAGAGGTGAATGAAGCTTTTGCTCCCCAGTACTTGGCTGTTGAGAGGAGTTTGGATCT
TGACATAAGTAAAACCAATGTGAATGGAGGAGCCATTGCTTTGGGTCACCCACTGGGAGGATCTGGAT
CAAGAATTACTGCACACCTGGTTCACGAATTAAGGCGTCGAGGTGGAAAATATGCCGTTGGATCAGCT
TGCATTGGAGGTGGCCAAGGTATTGCTGTCATCATTCAGAGCACAGCCTGAGCGGCCGCTAT

| | | | |
|---|---|---|---|
| NOV4b, 282274427 | | | |
| Protein Sequence | SEQ ID NO:32 | 401 aa | MW at 42355.2 kD |

TGSTMRLLRGVFVVAAKRTPFGAYGGLLKDFTATDLSEFAAKAALSAGKVSPETVDSVIMGNVLQSSS
DAIYLARHVGLRVGIPKETPALTINRLCGSGFQSIVNGCQEICVKEAEVVLCGGTESMSQAPYCVRNV
RFGTKLGSDIKLEDSLWVSLTDQHVQLPMAMTAENLAVKHKISREECDKYALQSQQRWKAANDAGYFN
DEMAPIEVKTKKGKQTMQVDEHARPQTTLEQLQKLPPVFKKDGTVTAGNASGVADGAGAVIIASEDAV
KKHNFTPLARIVGYFVSGCDPSIMGIGPVPAISGALKKAGLSLKDMDLVEVNEAFAPQYLAVERSLDL
DISKTNVNGGAIALGHPLGGSGSRITAHLVHELRRRGGKYAVGSACIGGGQGIAVIIQSTA

| | | |
|---|---|---|
| NOV4c, CG181387-02 | SEQ ID NO:33 | 1218 bp |
| DNA Sequence | ORF Start: at 2 | ORF Stop: TGA at 1205 |

CACCGGATCCACCATGCGTCTGCTCCGAGGTGTGTTTGTAGTTGCTGCTAAGCGAACGCCCTTTGGAG
CTTACGGAGGCCTTCTGAAAGACTTCACTGCTACTGACTTGTCTGAATTTGCTGCCAAGGCTGCCTTG
TCTGCTGGCAAAGTCTCACCTGAAACAGTTGACAGTGTGATTATGGGCAATGTCCTGCAGAGTTCTTC
AGATGCTATATATTTGGCAAGGCATGTTGGTTTGCGTGTGGGAATCCCAAAGGAGACCCCAGCTCTCA
CGATTAATAGGCTCTGTGGTTCTGGTTTTCAGTCCATTGTGAATGGATGTCAGGAAATTTGTGTTAAA
GAAGCTGAAGTTGTTTTATGTGGAGGAACCGAAAGCATGAGCCAAGCTCCCTACTGTGTCAGAAATGT
GCGTTTTGGAACCAAGCTTGGATCAGATATCAAGCTGGAAGATTCTTTATGGGTATCATTAACAGATC
AGCATGTCCAGCTCCCCATGGCAATGACTGCAGAGAATCTTGCTGTAAAACACAAAATAAGCAGAGAA
GAATGTGACAAATATGCCCTGCAGTCACAGCAGAGATGGAAAGCTGCTAATGATGCTGGCTACTTTAA
TGATGAAATGGCACCAATTGAAGTGAAGACAAAGAAAGGAAAACAGACAATGCAGGTAGACGAGCATG
CTCGGCCCCAAACCACCCTGGAACAGTTACAGAAACTTCCTCCAGTATTCAAGAAAGATGGAACTGTT
ACTGCAGGGAATGCATCGGGTGTAGCTGATGGTGCTGGAGCTGTTATCATAGCTAGTGAAGATGCTGT
TAAGAAACATAACTTCACACCACTGGCAAGAATTGTGGGCTACTTTGTATCTGGATGTGATCCCTCTA
TCATGGGTATTGGTCCTGTCCCTGCTATCAGTGGGGCACTGAAGAAAGCAGGACTGAGTCTTAAGGAC
ATGGATTTGGTAGAGGTGAATGAAGCTTTTGCTCCCCAGTACTTGGCTGTTGAGAGGAGTTTGGATCT
TGACATAAGTAAAACCAATGTGAATGGAGGAGCCATTGCTTTGGGTCACCCACTGGGAGGATCTGGAT
CAAGAATTACTGCACACCTGGTTCACGAATTAAGGCGTCGAGGTGGAAAATATGCCGTTGGATCAGCT
TGCATTGGAGGTGGCCAAGGTATTGCTGTCATCATTCAGAGCACAGCCTGAGCGGCCGCTAT

| | | | |
|---|---|---|---|
| NOV4c, CG181387-02 | | | |
| Protein Sequence | SEQ ID NO:34 | 401 aa | Mw at 42355.2 kD |

TGSTMRLLRGVFVVAAKRTPFGAYGGLLKDFTATDLSEFAAKAALSAGKVSPETVDSVIMGNVLQSSS
DAIYLARHVGLRVGIPKETPALTINRLCGSGFQSIVNGCQEICVKEAEVVLCGGTESMSQAPYCVRNV
RFGTKLGSDIKLEDSLWVSLTDQHVQLPMAMTAENLAVKHKISREECDKYALQSQQRWKAANDAGYFN
DEMAPIEVKTKKGKQTMQVDEHARPOTTLEQLQKLPPVFKKDGTVTAGNASGVADGAGAVIIASEDAV
KKNNFTPLARIVGYFVSGCDPSIMGIGPVPAISGALKKAGLSLKDMDLVEVNEAFAPQYLAVERSLDL
DISKTNVNGGAIALGHPLGGSGSRITAHLVHELRRRGGKYAVGSACIGGGQGIAVIIQSTA

| | | |
|---|---|---|
| NOV4d, 306268235 | SEQ ID NO:35 | 1248 bp |
| DNA Sequence | ORF Start: at 3 | ORF Stop: TGA at 1224 |

ACGCGTCTCCCATGGGACATCATCACCACCATCACCGTCTGCTCCGAGGTGTGTTTGTAGTTGCTGCT
AAGCGAACGCCCTTTGGAGCTTACGGAGGCCTTCTGAAAGACTTCACTGCTACTGACTTGTCTGAATT
TGCTGCCAAGGCTGCCTTGTCTGCTGGCAAAGTCTCACCTGAAACAGTTGACAGTGTGATTATGGGCA
ATGTCCTGCAGAGTTCTTCAGATGCTATATATTTGGCAAGGCATGTTGGTTTGCGTGTGGGAATCCCA
AAGGAGACCCCAGCTCTCACGATTAATAGGCTCTGTGGTTCTGGTTTTCAGTCCATTGTGAATGGATG
TCAGGAAATTTGTGTTAAAGAAGCTGAAGTTGTTTTATGTGGAGGAACCGAAAGCATGAGCCAAGCTC
CCTACTGTGTCAGAAATGTGCGTTTTGGAACCAAGCTTGGATCAGATATCAAGCTGGAAGATTCTTTA
TGGGTATCATTAACAGATCAGCATGTCCAGCTCCCCATGGCAATGACTGCAGAGAATCTTGCTGTAAA
ACACAAAATAAGCAGAGAAGAATGTGACAAATATGCCCTGCAGTCACAGCAGAGATGGAAAGCTGCTA
ATGATGCTGGCTACTTTAATGATGAAATGGCACCAATTGAAGTGAAGACAAAGAAAGGAAAACAGACA
ATGCAGGTAGACGAGCATGCTCGGCCCCAAACCACCCTGGAACAGTTACAGAAACTTCCTCCAGTATT
CAAGAAAGATGGAACTGTTACTGCAGGGAATGCATCGGGTGTAGCTGATGGTGCTGGAGCTGTTATCA
TAGCTAGTGAAGATGCTGTTAAGAAACATAACTTCACACCACTGGCAAGAATTGTGGGCTACTTTGTA
TCTGGATGTGATCCCTCTATCATGGGTATTGGTCCTGTCCCTGCTATCAGTGGGGCACTGAAGAAAGC
AGGACTGAGTCTTAAGGACATGGATTTGGTAGAGGTGAATGAAGCTTTTGCTCCCCAGTACTTGGCTG
TTGAGAGGAGTTTGGATCTTGACATAAGTAAAACCAATGTGAATGGAGGAGCCATTGCTTTGGGTCAC
CCACTGGGAGGATCTGGATCAAGAATTACTGCACACCTGGTTCACGAATTAAGGCGTCGAGGTGGAAA
ATATGCCGTTGGATCAGCTTGCATTGGAGGTGGCCAAGGTATTGCTGTCATCATTCAGAGCACAGCCT
GAGCAGGTGCGGCCGGAGACGAAG

| | | | |
|---|---|---|---|
| NOV4d, 306268235 | | | |
| Protein Sequence | SEQ ID NO:36 | 407 aa | MW at 43144.0 kD |

ASPMGHHHHHHRLLRGVFVVAAKRTPFGAYGGLLKDFTATDLSEFAAKAALSAGKVSPETVDSVIMGN
VLQSSSDAIYLARHVGLRVGIPKETPALTINRLCGSGFQSIVNGCQEICVKEAEVVLCGGTESMSQAP
YCVRNVRFGTKLGSDIKLEDSLWVSLTDQHVQLPMAMTAENLAVKHKISREECDKYALQSQQRWKAAN
DAGYFNDEMAPIEVKTKKGKQTMQVDEHARPQTTLEQLQKLPPVFKKDGTVTAGNASGVADGAGAVII
ASEDAVKKHNFTPLARIVGYFVSGCDPSIMGIGPVPAISGALKKAGLSLKDMDLVEVNEAFAPQYLAV
ERSLDLDISKTNVNGGAIALGHPLGGSGSRITAHLVHELRRRGGKYAVGSACIGGGQGIAVIIQSTA

| | | |
|---|---|---|
| NOV4e, CG181387-03 | SEQ ID NO:37 | 1249 bp |
| DNA Sequence | ORF Start: at 2 | ORF Stop: TGA at 1208 |

ACATCATCACCACCATCACCGTCTGCTCCGAGGTGTGTTTGTAGTTGCTGCTAAGCGAACGCCCTTTG
GAGCTTACGGAGGCCTTCTGAAAGACTTCACTGCTACTGACTTGTCTGAATTTGCTGCCAAGGCTGCC
TTGTCTGCTGGCAAAGTCTCACCTGAAACAGTTGACAGTGTGATTATGGGCAATGTCCTGCAGAGTTC
TTCAGATGCTATATATTTGGCAAGGCATGTTGGTTTGCGTGTGGGAATCCCAAAGGAGACCCCAGCTC
TCACGATTAATAGGCTCTGTGGTTCTGGTTTTCAGTCCATTGTGAATGGATGTCAGGAAATTTGTGTT
AAAGAAGCTGAAGTTGTTTTATGTGGAGGAACCGAAAGCATGAGCCAAGCTCCCTACTGTGTCAGAAA
TGTGCGTTTTGGAACCAAGCTTGGATCAGATATCAAGCTGGAAGATTCTTTATGGGTATCATTAACAG
ATCAGCATGTCCAGCTCCCCATGGCAATGACTGCAGAGAATCTTGCTGTAAAACACAAAATAAGCAGA
GAAGAATGTGACAAATATGCCCTGCAGTCACAGCAGAGATGGAAAGCTGCTAATGATGCTGGCTACTT
TAATGATGAAATGGCACCAATTGAAGTGAAGACAAAGAAAGGAAAACAGACAATGCAGGTAGACGAGC
ATGCTCGGCCCCAAACCACCCTGGAACAGTTACAGAAACTTCCTCCAGTATTCAAGAAAGATGGAACT
GTTACTGCAGGGAATGCATCGGGTGTAGCTGATGGTGCTGGAGCTGTTATCATAGCTAGTGAAGATGC
TGTTAAGAAACATAACTTCACACCACTGGCAAGAATTGTGGGCTACTTTGTATCTGGATGTGATCCCT
CTATCATGGGTATTGGTCCTGTCCCTGCTATCAGTGGGGCACTGAAGAAAGCAGGACTGAGTCTTAAG
GACATGGATTTGGTAGAGGTGAATGAAGCTTTTGCTCCCCAGTACTTGGCTGTTGAGAGGAGTTTGGA
TCTTGACATAAGTAAAACCAATGTGAATGGAGGAGCCATTGCTTTGGGTCACCCACTGGGAGGATCTG
GATCAAGAATTACTGCACACCTGGTTCACGAATTAAGGCGTCGAGGTGGAAAATATGCCGTTGGATCA
GCTTGCATTGGAGGTGGCCAAGGTATTGCTGTCATCATTCAGAGCACAGCCTGAGCAGGTGCGGCCGC
ACTCGAGCACCACCACCACCACCAC

| | | | |
|---|---|---|---|
| NOV4e, CG181387-03 | | | |
| Protein Sequence | SEQ ID NO:38 | 402 aa | MW at 42700.5 kD |

HHHHHHRLLRGVFVVAAKRTPFGAYGGLLKDFTATDLSEFAAKAALSAGKVSPETVDSVIMGNVLQSS
SDAIYLARHVGLRVGIPKETPALTINRLCGSGFQSIVNGCQEICVKEAEVVLCGGTESMSQAPYCVRN
VRFGTKLGSDIKLEDSLWVSLTDQHVQLPMAMTAENLAVKHKISREECDKYALQSQQRWKAANDAGYF
NDEMAPIEVKTKKGKQTMQVDEHARPQTTLEQLQKLPPVFKKDGTVTAGNASGVADGAGAVIIASEDA
VKKHNFTPLARIVGYFVSGCDPSIMGIGPVPAISGALKKAGLSLKDMDLVEVNEAFAPQYLAVERSLD
LDISKTNVNGGAIALGHPLGGSGSRITAHLVHELRRRGGKYAVGSACIGGGQGIAVIIQSTA

| | | |
|---|---|---|
| NOV4f, CG181387-04 | SEQ ID NO:39 | 1248 bp |
| DNA Sequence | ORF Start: at 1 | ORF Stop: TGA at 1207 |

CGTCTGCTCCGAGGTGTGTTTGTAGTTGCTGCTAAGCGAACGCCCTTTGGAGCTTACGGAGGCCTTCT
GAAAGACTTCACTGCTACTGACTTGTCTGAATTTGCTGCCAAGGCTGCCTTGTCTGCTGGCAAAGTCT
CACCTGAAACAGTTGACAGTGTGATTATGGGCAATGTCCTGCAGAGTTCTTCAGATGCTATATATTTG
GCAAGGCATGTTGGTTTGCGTGTGGGAATCCCAAAGGAGACCCCAGCTCTCACGATTAATAGGCTCTG
TGGTTCTGGTTTTCAGTCCATTGTGAATGGATGTCAGGAAATTTGTGTTAAAGAAGCTGAAGTTGTTT
TATGTGGAGGAACCGAAAGCATGAGCCAAGCTCCCTACTGTGTCAGAAATGTGCGTTTTGGAACCAAG
CTTGGATCAGATATCAAGCTGGAAGATTCTTTATGGGTATCATTAACAGATCAGCATGTCCAGCTCCC
CATGGCAATGACTGCAGAGAATCTTGCTGTAAAACACAAAATAAGCAGAGAAGAATGTGACAAATATG
CCCTGCAGTCACAGCAGAGATGGAAAGCTGCTAATGATGCTGGCTACTTTAATGATGAAATGGCACCA
ATTGAAGTGAAGACAAAGAAAGGAAAACAGACAATGCAGGTAGACGAGCATGCTCGGCCCCAAACCAC
CCTGGAACAGTTACAGAAACTTCCTCCAGTATTCAAGAAAGATGGAACTGTTACTGCAGGGAATGCAT
CGGGTGTAGCTGATGGTGCTGGAGCTGTTATCATAGCTAGTGAAGATGCTGTTAAGAAACATAACTTC
ACACCACTGGCAAGAATTGTGGGCTACTTTGTATCTGGATGTGATCCCTCTATCATGGGTATTGGTCC
TGTCCCTGCTATCAGTGGGGCACTGAAGAAAGCAGGACTGAGTCTTAAGGACATGGATTTGGTAGAGG
TGAATGAAGCTTTTGCTCCCCAGTACTTGGCTGTTGAGAGGAGTTTGGATCTTGACATAAGTAAAACC
AATGTGAATGGAGGAGCCATTGCTTTGGGTCACCCACTGGGAGGATCTGGATCAAGAATTACTGCACA
CCTGGTTCACGAATTAAGGCGTCGAGGTGGAAAATATGCCGTTGGATCAGCTTGCATTGGAGGTGGCC
AAGGTATTGCTGTCATCATTCAGAGCACAGCCCATCATCACCACCATCACTGAGCAGGTGCGGCCGCA
CTCGAGCACCACCACCACCACCAC

| | | | |
|---|---|---|---|
| NOV4f, CG181387-04 | | | |
| Protein Sequence | SEQ ID NO:40 | 402 aa | MW at 42700.5 kD |

RLLRGVFVVAAKRTPFGAYGGLLKDFTATDLSEFAAKAALSAGKVSPETVDSVIMGNVLQSSSDAIYL
ARHVGLRVGIPKETPALTINRLCGSGFQSIVNGCQEICVKEAEVVLCGGTESMSQAPYCVRNVRFGTK
LGSDIKLEDSLWVSLTDQHVQLPMAMTAENLAVKHKISREECDKYALQSQQRWKAANDAGYFNDEMAP
IEVKTKKGKQTMQVDEHARPQTTLEQLQKLPPVFKKDGTVTAGNASGVADGAGAVIIASEDAVKKHNF
TPLARIVGYFVSGCDPSIMGIGPVPAISGALKKAGLSLKDMDLVEVNEAFAPQYLAVERSLDLDISKT
NVNGGAIALGHPLGGSGSRITAHLVHELRRRGGKYAVGSACIGGGQGIAVIIQSTAHHHHHH

| | | |
|---|---|---|
| NOV4g, CG181387-05 | SEQ ID NO:41 | 1230 bp |
| DNA Sequence | ORF Start: at 1 | ORF Stop: TGA at 1189 |

CGTCTGCTCCGAGGTGTGTTTGTAGTTGCTGCTAAGCGAACGCCCTTTGGAGCTTACGGAGGCCTTCT
GAAAGACTTCACTGCTACTGACTTGTCTGAATTTGCTGCCAAGGCTGCCTTGTCTGCTGGCAAAGTCT
CACCTGAAACAGTTGACAGTGTGATTATGGGCAATGTCCTGCAGAGTTCTTCAGATGCTATATATTTG
GCAAGGCATGTTGGTTTGCGTGTGGGAATCCCAAAGGAGACCCCAGCTCTCACGATTAATAGGCTCTG
TGGTTCTGGTTTTCAGTCCATTGTGAATGGATGTCAGGAAATTTGTGTTAAAGAAGCTGAAGTTGTTT
TATGTGGAGGAACCGAAAGCATGAGCCAAGCTCCCTACTGTGTCAGAAATGTGCGTTTTGGAACCAAG
CTTGGATCAGATATCAAGCTGGAAGATTCTTTATGGGTATCATTAACAGATCAGCATGTCCAGCTCCC
CATGGCAATGACTGCAGAGAATCTTGCTGTAAAACACAAAATAAGCAGAGAAGAATGTGACAAATATG
CCCTGCAGTCACAGCAGAGATGGAAAGCTGCTAATGATGCTGGCTACTTTAATGATGAAATGGCACCA
ATTGAAGTGAAGACAAAGAAAGGAAAACAGACAATGCAGGTAGACGAGCATGCTCGGCCCCAAACCAC
CCTGGAACAGTTACAGAAACTTCCTCCAGTATTCAAGAAAGATGGAACTGTTACTGCAGGGAATGCAT
CGGGTGTAGCTGATGGTGCTGGAGCTGTTATCATAGCTAGTGAAGATGCTGTTAAGAAACATAACTTC
ACACCACTGGCAAGAATTGTGGGCTACTTTGTATCTGGATGTGATCCCTCTATCATGGGTATTGGTCC
TGTCCCTGCTATCAGTGGGGCACTGAAGAAAGCAGGACTGAGTCTTAAGGACATGGATTTGGTAGAGG
TGAATGAAGCTTTTGCTCCCCAGTACTTGGCTGTTGAGAGGAGTTTGGATCTTGACATAAGTAAAACC
AATGTGAATGGAGGAGCCATTGCTTTGGGTCACCCACTGGGAGGATCTGGATCAAGAATTACTGCACA
CCTGGTTCACGAATTAAGGCGTCGAGGTGGAAAATATGCCGTTGGATCAGCTTGCATTGGAGGTGGCC
AAGGTATTGCTGTCATCATTCAGAGCACAGCCTGAGCAGGTGCGGCCGCACTCGAGCACCACCACCAC
CACCAC

| | | | |
|---|---|---|---|
| NOV4g, CG181387-05 | | | |
| Protein Sequence | SEQ ID NO:42 | 396 aa | MW at 41877.6 kD |

RLLRGVFVVAAKRTPFGAYGGLLKDFTATDLSEFAAKAALSAGKVSPETVDSVIMGNVLQSSSDAIYL
ARHVGLRVGIPKETPALTINRLCGSGFQSIVNGCQEICVKEAEVVLCGGTESMSQAPYCVRNVRFGTK
LGSDIKLEDSLWVSLTDQHVQLPMAMTAENLAVKHKISREECDKYALQSQQRWKAANDAGYFNDEMAP
IEVKTKKGKQTMQVDEHARPQTTLEQLQKLPPVFKKDGTVTAGNASGVADGAGAVIIASEDAVKKHNF
TPLARIVGYFVSGCDPSIMGIGPVPAISGALKKAGLSLKDMDLVEVNEAFAPQYLAVERSLDLDISKT
NVNGGAIALGHPLGGSGSRITAHLVHELRRRGGKYAVGSACIGGGQGIAVIIQSTA

| | | |
|---|---|---|
| NOV4h, GG181387-06 | SEQ ID NO:43 | 1221 bp |
| DNA Sequence | ORF Start: ATG at 4 | ORF Stop: at 1195 |

ACCATGCGTCTGCTCCGAGGTGTGTTTGTAGTTGCTGCTAAGCGAACGCCCTTTGGAGCTTACGGAGG
CCTTCTGAAAGACTTCACTGCTACTGACTTGTCTGAATTTGCTGCCAAGGCTGCCTTGTCTGCTGGCA
AAGTCTCACCTGAAACAGTTGACAGTGTGATTATGGGCAATGTCCTGCAGAGTTCTTCAGATGCTATA
TATTTGGCAAGGCATGTTGGTTTGCGTGTGGGAATCCCAAAGGAGACCCCAGCTCTCACGATTAATAG
GCTCTGTGGTTCTGGTTTTCAGTCCATTGTGAATGGATGTCAGGAAATTTGTGTTAAAGAAGCTGAAG
TTGTTTTATGTGGAGGAACCGAAAGCATGAGCCAAGCTCCCTACTGTGTCAGAAATGTGCGTTTTGGA
ACCAAGCTTGGATCAGATATCAAGCTGGAAGATTCTTTATGGGTATCATTAACAGATCAGCATGTCCA
GCTCCCCATGGCAATGACTGCAGAGAATCTTGCTGTAAAACACAAAATAAGCAGAGAAGAATGTGACA
AATATGCCCTGCAGTCACAGCAGAGATGGAAAGCTGCTAATGATGCTGGCTACTTTAATGATGAAATG
GCACCAATTGAAGTGAAGACAAAGAAAGGAAAACAGACAATGCAGGTAGACGAGCATGCTCGGCCCCA
AACCACCCTGGAACAGTTACAGAAACTTCCTCCAGTATTCAAGAAAGATGGAACTGTTACTGCAGGGA
ATGCATCGGGTGTAGCTGATGGTGCTGGAGCTGTTATCATAGCTAGTGAAGATGCTGTTAAGAAACAT
AACTTCACACCACTGGCAAGAATTGTGGGCTACTTTGTATCTGGATGTGATCCCTCTATCATGGGTAT
TGGTCCTGTCCCTGCTATCAGTGGGGCACTGAAGAAAGCAGGACTGAGTCTTAAGGACATGGATTTGG
TAGAGGTGAATGAAGCTTTTGCTCCCCAGTACTTGGCTGTTGAGAGGAGTTTGGATCTTGACATAAGT
AAAACCAATGTGAATGGAGGAGCCATTGCTTTGGGTCACCCACTGGGAGGATCTGGATCAAGAATTAC
TGCACACCTGGTTCACGAATTAAGGCGTCGAGGTGGAAAATATGCCGTTGGATCAGCTTGCATTGGAG
GTGGCCAAGGTATTGCTGTCATCATTCAGAGCACAGCCCATCATCACCACCATCACTGAGCAGGT

| | | | |
|---|---|---|---|
| NOV4h, CG181387-06 | | | |
| Protein Sequence | SEQ ID NO:44 | 397 aa | MW at42008.8 kD |

MRLLRGVFVVAAKRTPFGAYGGLLKDFTATDLSEFAAKAALSAGKVSPETVDSVIMGNVLQSSSDAIY
LARHVGLRVGIPKETPALTINRLCGSGFQSIVNGCQEICVKEAEVVLCGGTESMSQAPYCVRNVRFGT
KLGSDIKLEDSLWVSLTDQHVQLPMAMTAEMLAVKHKISREECDKYALQSQQRWKAANDAGYFNDEMA
PIEVKTKKGKQTMQVDEHARPQTTLEQLQKLPPVFKKDGTVTAGNASGVADGAGAVIIASEDAVKKHN
FTPLARIVGYFVSGCDPSIMGIGPVPAISGALKKAGLSLKDMDLVEVNEAFAPQYLAVERSLDLDISK
TNVNGGAIALGHPLGGSGSRITAHLVHELRRRGGKYAVGSACIGGGQGIAVIIQSTA

A ClustalW comparison of the above protein sequences yields the following sequence alignment shown in Table D7.

TABLE D7
Comparison of the NOV4 protein sequences.

| | |
|---|---|
| NOV4a | ——————————MRLLRGVFVVAAKRTPFGAYGGLLKDFTATDLSEFAAKAALSAGKVSPET |
| NOV4b | ——————TGSTMRLLRGVFVVAAKRTPFGAYGGLLKDFTATDLSEFAAKAALSAGKVSPET |
| NOV4c | ——————TGSTMRLLRGVFVVAAKRTPFGAYGGLLKDFTATDLSEFAAKAALSAGKVSPET |
| NOV4d | ASPMGHHHHHHRLLRGVFVVAAKRTPFGAYGGLLKDFTATDLSEFAAKAALSAGKVSPET |
| NOV4e | —————HHHHHHRLLRGVFVVAAKRTPFGAYGGLLKDFTATDLSEFAAKAALSAGKVSPET |
| NOV4f | ———————————RLLRGVFVVAAKRTPFGAYGGLLKDFTATDLSEFAAKAALSAGKVSPET |
| NOV4g | ———————————RLLRGVFVVAAKRTPFGAYGGLLKDFTATDLSEFAAKAALSAGKVSPET |
| NOV4h | ——————————MRLLRGVFVVAAKRTPFGAYGGLLKDFTATDLSEFAAKAALSAGKVSPET |
| NOV4a | VDSVIMGNVLQSSSDAIYLARHVGLRVGIPKETPALTINRLCGSGFQSIVNGCQEICVKE |
| NOV4b | VDSVIMGNVLQSSSDAIYLARHVGLRVGIPKETPALTINRLCGSGFQSIVNGCQEICVKE |
| NOV4c | VDSVIMGNVLQSSSDAIYLARHVGLRVGIPKETPALTINRLCGSGFQSIVNGCQEICVKE |
| NOV4d | VDSVIMGNVLQSSSDAIYLARHVGLRVGIPKETPALTINRLCGSGFQSIVNGCQEICVKE |
| NOV4e | VDSVIMGNVLQSSSDAIYLARHVGLRVGIPKETPALTINRLCGSGFQSIVNGCQEICVKE |
| NOV4f | VDSVIMGNVLQSSSDAIYLARHVGLRVGIPKETPALTINRLCGSGFQSIVNGCQEICVKE |
| NOV4g | VDSVIMGNVLQSSSDAIYLARHVGLRVGIPKETPALTINRLCGSGFQSIVNGCQEICVKE |
| NOV4h | VDSVIMGNVLQSSSDAIYLARHVGLRVGIPKETPALTINRLCGSGFQSIVNGCQEICVKE |
| NOV4a | AEVVLCGGTESMSQAPYCVRNVRFGTKLGSDIKLEDSLWVSLTDQHVQLPMAMTAENLTV |
| NOV4b | AEVVLCGGTESMSQAPYCVRNVRFGTKLGSDIKLEDSLWVSLTDQHVQLPMAMTAENLAV |
| NOV4c | AEVVLCGGTESMSQAPYCVRNVRFGTKLGSDIKLEDSLWVSLTDQHVQLPMAMTAENLAV |
| NOV4d | AEVVLCGGTESMSQAPYCVRNVRFGTKLGSDIKLEDSLWVSLTDQHVQLPMAMTAENLAV |
| NOV4e | AEVVLCGGTESMSQAPYCVRNVRFGTKLGSDIKLEDSLWVSLTDQHVQLPMAMTAENLAV |
| NOV4f | AEVVLCGGTESMSQAPYCVRNVRFGTKLGSDIKLEDSLWVSLTDQHVQLPMAMTAENLAV |
| NOV4g | AEVVLCGGTESMSQAPYCVRNVRFGTKLGSDIKLEDSLWVSLTDQHVQLPMAMTAENLAV |
| NOV4h | AEVVLCGGTESMSQAPYCVRNVRFGTKLGSDIKLEDSLWVSLTDQHVQLPMAMTAENLAV |
| NOV4a | KHKISREECDKYALQSQQRWKAANDAGYFNDEMAPIEVKTKKGKQTMQVDEHARPQTTLE |
| NOV4b | KHKISREECDKYALQSQQRWKAANDAGYFNDEMAPIEVKTKKGKQTMQVDEHARPQTTLE |
| NOV4c | KHKISREECDKYALQSQQRWKAANDAGYFNDEMAPIEVKTKKGKQTMQVDEHARPQTTLE |
| NOV4d | KHKISREECDKYALQSQQRWKAANDAGYFNDEMAPIEVKTKKGKQTMQVDEHARPQTTLE |
| NOV4e | KHKISREECDKYALQSQQRWKAANDAGYFNDEMAPIEVKTKKGKQTMQVDEHARPQTTLE |
| NOV4f | KHKISREECDKYALQSQQRWKAANDAGYFNDEMAPIEVKTKKGKQTMQVDEHARPQTTLE |
| NOV4g | KHKISREECDKYALQSQQRWKAANDAGYFNDEMAPIEVKTKKGKQTMQVDEHARPQTTLE |
| NOV4h | KHKISREECDKYALQSQQRWKAANDAGYFNDEMAPIEVKTKKGKQTMQVDEHARPQTTLE |
| NOV4a | QLQKLPPVFKKDGTVTAGNASGVADGAGAVIIASEDAVKKHNFTPLARIVGYFVSGCDPS |
| NOV4b | QLQKLPPVFKKDGTVTAGNASGVADGAGAVIIASEDAVKKHNFTPLARIVGYFVSGCDPS |
| NOV4c | QLQKLPPVFKKDGTVTAGNASGVADGAGAVIIASEDAVKKHNFTPLARIVGYFVSGCDPS |
| NOV4d | QLQKLPPVFKKDGTVTAGNASGVADGAGAVIIASEDAVKKHNFTPLARIVGYFVSGCDPS |
| NOV4e | QLQKLPPVFKKDGTVTAGNASGVADGAGAVIIASEDAVKKHNFTPLARIVGYFVSGCDPS |
| NOV4f | QLQKLPPVFKKDGTVTAGNASGVADGAGAVIIASEDAVKKHNFTPLARIVGYFVSGCDPS |
| NOV4g | QLQKLPPVFKKDGTVTAGNASGVADGAGAVIIASEDAVKKHNFTPLARIVGYFVSGCDPS |
| NOV4h | QLQKLPPVFKKDGTVTAGNASGVADGAGAVIIASEDAVKKHNFTPLARIVGYFVSGCDPS |
| NOV4a | IMGIGPVPAISGALKKAGLSLKDMDLVEVNEAFAPQYLAVERSLDLDISKTNVNGGAIAL |
| NOV4b | IMGIGPVPAISGALKKAGLSLKDMDLVEVNEAFAPQYLAVERSLDLDISKTNVNGGAIAL |
| NOV4c | IMGIGPVPAISGALKKAGLSLKDMDLVEVNEAFAPQYLAVERSLDLDISKTNVNGGAIAL |
| NOV4d | IMGIGPVPAISGALKKAGLSLKDMDLVEVNEAFAPQYLAVERSLDLDISKTNVNGGAIAL |
| NOV4e | IMGIGPVPAISGALKKAGLSLKDMDLVEVNEAFAPQYLAVERSLDLDISKTNVNGGAIAL |
| NOV4f | IMGIGPVPAISGALKKAGLSLKDMDLVEVNEAFAPQYLAVERSLDLDISKTNVNGGAIAL |
| NOV4g | IMGIGPVPAISGALKKAGLSLKDMDLVEVNEAFAPQYLAVERSLDLDISKTNVNGGAIAL |
| NOV4h | IMGIGPVPAISGALKKAGLSLKDMDLVEVNEAFAPQYLAVERSLDLDISKTNVNGGAIAL |
| NOV4a | GHPLGGSGSRITAHLVHELRRRGGKYAVGSACIGGGQGIAVIIQSTA—————— |
| NOV4b | GHPLGGSGSRITAHLVHELRRRGGKYAVGSACIGGGQGIAVIIQSTA—————— |
| NOV4c | GHPLGGSGSRITAHLVHELRRRGGKYAVGSACIGGGQGIAVIIQSTA—————— |
| NOV4d | GHPLGGSGSRITAHLVHELRRRGGKYAVGSACIGGGQGIAVIIQSTA—————— |
| NOV4e | GHPLGGSGSRITAHLVHELRRRGGKYAVGSACIGGGQGIAVIIQSTA—————— |
| NOV4f | GHPLGGSGSRITAHLVHELRRRGGKYAVGSACIGGGQGIAVIIQSTAHHHHHH |
| NOV4g | GHPLGGSGSRITAHLVHELRRRGGKYAVGSACIGGGQGIAVIIQSTA—————— |
| NOV4h | GHPLGGSGSRITAHLVHELRRRGGKYAVGSACIGGGQGIAVIIQSTA—————— |
| NOV4a | (SEQ ID NO:30) |
| NOV4b | (SEQ ID NO:32) |
| NOV4c | (SEQ ID NO:34) |
| NOV4d | (SEQ ID NO:36) |
| NOV4e | (SEQ ID NO:38) |
| NOV4f | (SEQ ID NO:40) |
| NOV4g | (SEQ ID NO:42) |
| NOV4h | (SEQ ID NO:44) |

Further analysis of the NOV4a protein yielded the following properties shown in Table D8.

TABLE D8
Protein Sequence Properties NOV4a

| SignalP | Cleavage site between residues 19 and 20 | | | |
|---|---|---|---|---|
| analysis: | | | | |
| PSORT II | PSG: a new signal peptide prediction method | | | |
| analysis: | N-region: length 5; pos. chg 2; neg. chg 0 | | | |
| | H-region: length 7; peak value 3.62 | | | |
| | PSG score: −0.78 | | | |
| | GvH: von Heijne's method for signal seq. recognition | | | |
| | GvH score (threshold: −2.1): −7.58 | | | |
| | possible cleavage site: between 48 and 49 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 3.92 (at 279) | | | |
| | ALOM score: −0.59 (number of TMSs: 0) | | | |
| | MITDISC: discrimination of mitochondrial targeting seq | | | |
| | R content: | 3 | Hyd Moment(75): | 6.86 |
| | Hyd Moment(95): | 13.09 | G content: | 4 |
| | D/E content: | 1 | S/T content: | 1 |

```
Score: −2.94
Gavel: prediction of cleavage sites for mitochondrial preseq
R-2 motif at 24 KRT|PF
NUCDISC: discrimination of nuclear localization signals
pat4: none
pat7: none
bipartite: none
content of basic residues: 10.8%
NLS Score: −0.47
KDEL: ER retention motif in the C-terminus: none
ER Membrane Retention Signals:
XXRR-like motif in the N-terminus: RLLR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
Final Results (k = 9/23):
47.8%: mitochondrial
34.8%: cytoplasmic
  8.7%: nuclear
  4.3%: vacuolar
  4.3%: peroxisomal
>> prediction for CG181387-01 is mit (k = 23)
```

A search of the NOV4a protein against the Geneseq database, a proprietary database that contains sequences published in patents and patent publication, yielded several homologous proteins shown in Table D9.

TABLE D9
Geneseq Results for NOV4a

| | | NOV4a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB89415 | Human polypeptide SEQ ID NO | 1 . . . 397 | 395/397 (99%) | 0.0 |
| | 1791 - Homo sapiens, 397 aa. | 1 . . . 397 | 395/397 (99%) | |
| | [WO200190304-A2, 29-NOV-2001] | | | |
| AAU23202 | Novel human enzyme polypeptide | 1 . . . 397 | 395/397 (99%) | 0.0 |
| | #288 - Homo sapiens, 438 aa. | 42 . . . 438 | 395/397 (99%) | |
| | [WO200155301-A2, 02-AUG-2001] | | | |
| AAB53323 | Human colon cancer antigen protein | 1 . . . 397 | 395/397 (99%) | 0.0 |
| | sequence SEQ ID NO: 863 - Homo | 42 . . . 438 | 395/397 (99%) | |
| | sapiens, 438 aa. [WO200055351-A1, | | | |
| | 21-SEP-2000] | | | |
| ABR48490 | Human Ketothiolase - Homo sapiens, | 4 . . . 397 | 391/394 (99%) | 0.0 |
| | 394 aa. [WO200294864-A2, 28-NOV-2002] | 1 . . . 394 | 392/394 (99%) | |
| ABB60752 | Drosophila melanogaster polypeptide | 5 . . . 395 | 233/392 (59%) | e−131 |
| | SEQ ID NO 9048 - Drosophila | 6 . . . 397 | 292/392 (74%) | |
| | melanogaster, 398 aa. | | | |
| | [WO200171042-A2, 27-SEP-2001] | | | |

In a BLAST search of public sequence databases, the NOV4a protein was found to have homology to the proteins shown in the BLASTP data in Table D10.

TABLE D10
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P42765 | 3-ketoacyl-CoA thiolase, | 1 . . . 397 | 397/397 (100%) | 0.0 |
| | mitochondrial (EC 2.3.1.16) (Beta- | 1 . . . 397 | 397/397 (100%) | |
| | ketothiolase) (Acetyl-CoA | | | |
| | oxoacyl-CoA thiolase) (T1) - Homo | | | |
| | sapiens (Human), 397 aa. | | | |
| Q9BUT6 | Acetyl-coenzyme A acyltransferase 2 | 1 . . . 397 | 395/397 (99%) | 0.0 |
| | (Mitochondrial 3-oxoacyl-coenzyme | 1 . . . 397 | 395/397 (99%) | |
| | A thiolase) - Homo sapiens (Human), | | | |
| | 397 aa. | | | |
| CAD67660 | Sequence 75 from Patent | 4 . . . 397 | 391/394 (99%) | 0.0 |
| | WO02094864 - Homo sapiens | 1 . . . 394 | 392/394 (99%) | |
| | (Human), 394 aa. | | | |
| Q8BWT1 | 3-ketoacyl-CoA thiolase - Mus | 1 . . . 397 | 347/397 (87%) | 0.0 |
| | musculus (Mouse), 397 aa. | 1 . . . 397 | 381/397 (95%) | |
| Q8JZR8 | Similar to acetyl-coenzyme A | 1 . . . 396 | 346/396 (87%) | 0.0 |
| | acyltransferase 2 (Mitochondrial 3- | 1 . . . 396 | 380/396 (95%) | |
| | oxoacyl-coenzyme A thiolase) - Mus | | | |
| | musculus (Mouse), 397 aa. | | | |

PFam analysis predicts that the NOV4a protein contains the domains shown in the Table D11.

TABLE D11
Domain Analysis of NOV4a

| | | Identities/ | |
|---|---|---|---|
| | NOV4a | Similarities for | |
| Pfam Domain | Match Region | the Matched Region | Expect Value |
| thiolase | 4 . . . 266 | 127/274 (46%) | 1.4e−147 |
| | | 242/274 (88%) | |
| thiolase_C | 271 . . . 395 | 81/142 (57%) | 1.3e−77 |
| | | 119/142 (84%) | |
| ketoacyl-synt_C | 276 . . . 397 | 29/188 (15%) | 0.31 |
| | | 76/188 (40%) | |

### EXAMPLE D5

### Human Acetyl-Coenzyme A acyltransferase 2 Gene Variants and SNPs

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmafion: SNPs are confirmed employing a validated method know as Pyrosequencing. Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265.

In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

Results

The DNA and protein sequences for the novel single nucleotide polymorphic variants of the Acetyl-Coenzyme A acyltransferase 2-like gene of CuraGen Acc. No. CG181387-01 are reported in Table D10. Variants are reported individually but any combination of all or a select subset of variants are also included. In Table D10, the positions of the variant bases and the variant amino acid residues are underlined. In summary, there are 6 variants reported in Table D10. Variant 13380063 is an A to G SNP at 697 bp of the nucleotide sequence that results in a Met to Val change at amino acid 217 of protein sequence, variant 13380064 is an A to G SNP at 1250 bp of the nucleotide sequence that results in no change in the protein sequence since the SNP is not in the amino acid coding region, variant 13380065 is an A to G SNP at 1451 bp of the nucleotide sequence that results in no change in the protein sequence since the SNP is not in the amino acid coding region, variant 13380066 is a C to T SNP at 1466 bp of the nucleotide sequence that results in no change in the protein sequence since the SNP is not in the amino acid coding region, variant 13380067 is an A to C SNP at 1525 bp of the nucleotide sequence that results in no change in the protein sequence since the SNP is not in the amino acid coding region, and variant 13380068 is a T to C SNP at 1550 bp of the nucleotide sequence that results in no change in the protein sequence since the SNP is not in the amino acid coding region.

TABLE D10
Variants of nucleotide sequence Acc.
No. CG181387-01 (SEQ ID NO: 29)

| | Nucleotides | | | Amino Acids | | |
|---|---|---|---|---|---|---|
| Variant | Position | Initial | Modified | Position | Initial | Modified |
| 13380063 | 697 | A | G | 217 | Met | Val |
| 13380064 | 1250 | A | G | 0 | | |
| 13380065 | 1451 | A | G | 0 | | |
| 13380066 | 1466 | C | T | 0 | | |
| 13380067 | 1525 | A | C | 0 | | |
| 13380068 | 1550 | T | C | 0 | | |

TABLE D11
Sequences of Variants

Table D11A1.
Nucleotide sequence of variant 13380063 NOV4a1n (underlined). A/G
(SEQ ID NO:155)

| | |
|---|---|
| 1 | GCGTCCCCCACACCACAGACCCGCGCCGCCGACGACCCAGCAGCCGCCATGCGTCTGCTCCGAGGTGTGTTTGTAGTTGC |
| 81 | TGCTAAGCGAACGCCCTTTGGAGCTTACGGAGGCCTTCTGAAAGACTTCACTGCTACTGACTTGTCTGAATTTGCTGCCA |
| 161 | AGGCTGCCTTGTCTGCTGGCAAAGTCTCACCTGAAACAGTTGACAGTGTGATTATGGGCAATGTCCTGCAGAGTTCTTCA |
| 241 | GATGCTATATATTTGGCAAGGCATGTTGGTTTGCGTGTGGGAATCCCAAAGGAGACCCCAGCTCTCACGATTAATAGGCT |
| 321 | CTGTGGTTCTGGTTTTCAGTCCATTGTGAATGGATGTCAGGAAATTTGTGTTAAAGAAGCTGAAGTTGTTTTATGTGGAG |
| 401 | GAACCGAAAGCATGAGCCAAGCTCCCTACTGTGTCAGAAATGTGCGTTTTGGAACCAAGCTTGGATCAGATATCAAGCTG |
| 481 | GAAGATTCTTTATGGGTATCATTAACAGATCAGCATGTCCAGCTCCCCATGGCAATGACTGCAGAGAATCTTACTGTAAA |
| 561 | ACACAAAATAAGCAGAGAAGAATGTGACAAATATGCCCTGCAGTCACAGCAGAGATGGAAAGCTGCTAATGATGCTGGCT |
| 641 | ACTTTAATGATGAAATGGCACCAATTGAAGTGAAGACAAAGAAAGGAAAACAGACAGTGCAGGTAGACGAGCATGCTCGG |
| 721 | CCCCAAACCACCCTGGAACAGTTACAGAAACTTCCTCCAGTATTCAAGAAAGATGGAACTGTTACTGCAGGGAATGCATC |
| 801 | GGGTGTAGCTGATGGTGCTGGAGCTGTTATCATAGCTAGTGAAGATGCTGTTAAGAAACATAACTTCACACCACTGGCAA |
| 881 | GAATTGTGGGCTACTTTGTATCTGGATGTGATCCCTCTATCATGGGTATTGGTCCTGTCCCTGCTATCAGTGGGGCACTG |
| 961 | AAGAAAGCAGGACTGAGTCTTAAGGACATGGATTTGGTAGAGGTGAATGAAGCTTTTGCTCCCCAGTACTTGGCTGTTGA |
| 1041 | GAGGAGTTTGGATCTTGACATAAGTAAAACCAATGTGAATGGAGGAGCCATTGCTTTGGGTCACCCACTGGGAGGATCTG |
| 1121 | CATCAAGAATTACTGCACACCTGGTTCACGAATTAAGGCGTCGAGGTGGAAAATATGCCGTTGGATCAGCTTGTATTGGA |
| 1201 | GGTGGCCAAGGTATTGCTGTCATCATTCAGAGCACAGCCTGAAGAGACCAGTGAGCTCACTGTGACCCATCCTTACTCTA |
| 1281 | CTTGGCCAGGCCACAGTAAAACAAGTGACCTTCAGAGCAGCTGCCACAACTGGCCATGCCCTGCCATTGAAACAGTGATT |
| 1361 | AAGTTTGATCAAGCCATGGTGACACAAAAATGCATTGATCATGAATAGGAGCCCATGCTAGAAGTACATTCTCTCAGATT |
| 1441 | TGAACCAGTGAAATATGATGTATTTCTGAGCTAAAACTCAACTATAGAAGACATTAAAAGAAATCGTATTCTTGCCAAGT |
| 1521 | AACCACCACTTCTGCCTTAGATAATATGATTATAAGGAAATCAAATAAATGTTGCCTTAACTTC |

Table D11A2.
Protein sequence of variant NOV4a1p (underlined). (SEQ ID NO:156)

| | |
|---|---|
| 1 | MRLLRGVFVVAAKRTPFGAYGGLLKDFTATDLSEFAAKAALSAGKVSPETVDSVIMGNVLQSSSDAIYLARHVGLRVGIP |
| 81 | KETPALTINRLCGSGFQSIVNGCQEICVKEAEVVLCGGTESMSQAPYCVRNVRFGTKLGSDIKLEDSLWVSLTDQHVQLP |
| 161 | MANTAENLTVKHKISREECDKYALQSQQRWKAANDAGYFNDEMAPIEVKTKKGKQTVQVDEHARPQTTLEQLQKLPPVFK |
| 241 | KDGTVTAGNASGVADGAGAVIIASEDAVKKHNFTPLARIVGYFVSGCDPSIMGIGPVPAISGALKKAGLSLKDMDLVEVN |
| 321 | EAFAPQYLAVERSLDLDISKTNVNGGAIALGHPLGGSGSRITAHLVHELRRRGGKYAVGSACIGGGQGIAVIIQSTA |

Table D11A3. Alteration effect
Met to Val
Table D11B1.
Nucleotide sequence of variant 13380064 NOV4a2n (underlined). A/G
(SEQ ID NO:157)

| | |
|---|---|
| 1 | GCGTCCCCCACACCACAGACCCGCGCCGCCGACGACCCAGCAGCCGCCATGCGTCTGCTCCGAGGTGTGTTTGTAGTTGC |
| 81 | TGCTAAGCGAACGCCCTTTGGAGCTTACGGAGGCCTTCTGAAAGACTTCACTGCTACTGACTTGTCTGAATTTGCTGCCA |
| 161 | AGGCTGCCTTGTCTGCTGGCAAAGTCTCACCTGAAACAGTTGACAGTGTGATTATGGGCAATGTCCTGCAGAGTTCTTCA |
| 241 | GATGCTATATATTTGGCAAGGCATGTTGGTTTGCGTGTGGGAATCCCAAAGGAGACCCCAGCTCTCACGATTAATAGGCT |
| 321 | CTGTGGTTCTGGTTTTCAGTCCATTGTGAATGGATGTCAGGAAATTTGTGTTAAAGAAGCTGAAGTTGTTTTATGTGGAG |
| 401 | GAACCGAAAGCATGAGCCAAGCTCCCTACTGTGTCAGAAATGTGCGTTTTGGAACCAAGCTTGGATCAGATATCAAGCTG |
| 481 | GAAGATTCTTTATGGGTATCATTAACAGATCAGCATGTCCAGCTCCCCATGGCAATGACTGCAGAGAATCTTACTGTAAA |
| 561 | ACACAAAATAAGCAGAGAAGAATGTGACAAATATGCCCTGCAGTCACAGCAGAGATGGAAAGCTGCTAATGATGCTGGCT |
| 641 | ACTTTAATGATGAAATGGCACCAATTGAAGTGAAGACAAAGAAAGGAAAACAGACAATGCAGGTAGACGAGCATGCTCGG |
| 721 | CCCCAAACCACCCTGGAACAGTTACAGAAACTTCCTCCAGTATTCAAGAAAGATGGAACTGTTACTGCAGGGAATGCATC |
| 801 | GGGTGTAGCTGATGGTGCTGGAGCTGTTATCATAGCTAGTGAAGATGCTGTTAAGAAACATAACTTCACACCACTGGCAA |
| 881 | GAATTGTGGGCTACTTTGTATCTGGATGTGATCCCTCTATCATGGGTATTGGTCCTGTCCCTGCTATGAGTGGGGCACTG |
| 961 | AAGAAAGCAGGACTGAGTCTTAAGGACATGGATTTGGTAGAGGTGAATGAAGCTTTTGCTCCCCAGTACTTGGCTGTTGA |
| 1041 | GAGGAGTTTGGATCTTGACATAAGTAAAACCAATGTGAATGGAGGAGCCATTGCTTTGGGTCACCCACTGGGAGGATCTG |
| 1121 | GATCAAGAATTACTGCACACCTGGTTCACGAATTAAGGCGTCGAGGTGGAAAATATGCCGTTGGATCAGCTTGCATTGGA |
| 1201 | GGTGGCCAAGGTATTGCTGTCATCATTCAGAGCACAGCCTGAAGAGACCGGTGAGCTCACTGTGACCCATCCTTACTCTA |
| 1281 | CTTGGCCAGGCCACAGTAAAACAAGTGACCTTCAGAGCAGCTGCCACAACTGGCCATGCCCTGCCATTGAAACAGTGATT |
| 1361 | AAGTTTGATCAAGCCATGGTGACACAAAAATGCATTGATCATGAATAGGAGCCCATGCTAGAAGTACATTCTCTCAGATT |
| 1441 | TGAACCAGTGAAATATGATGTATTTCTGAGCTAAAACTCAACTATAGAAGACATTAAAAGAAATCGTATTCTTGCCAAGT |
| 1521 | AACCACCACTTCTGCCTTAGATAATATGATTATAAGGAAATCAAATAAATGTTGCCTTAACTTC |

Table D11B2.
Protein sequence of variant NOV4a2p (underlined). (SEQ ID NO:158)

| | |
|---|---|
| 1 | MRLLRGVFVVAAKRTPFGAYGGLLKDFTATDLSEFAAKAALSAGKVSPETVDSVIMGNVLQSSSDAIYLARHVGLRVGIP |
| 81 | KETPALTINRLCGSGFQSIVNGCQEICVKEAEVVLCGGTESMSQAPYCVRNVRFGTKLGSDIKLEDSLWVSLTDQHVQLP |
| 161 | MANTAENLTVKHKISREECDKYALQSQQRWKAANDAGYFNDEMAPIEVKTKKGKQTMQVDEHARPQTTLEQLQKLPPVFK |
| 241 | KDGTVTAGNASGVADGAGAVIIASEDAVKKHNFTPLARIVGYFVSGCDPSIMGIGPVPAISGALKKAGLSLKDMDLVEVN |
| 321 | EAFAPQYLAVERSLDLDISKTNVNGGAIALGHPLGGSGSRITAHLVHELRRRGGKYAVGSACIGGGQGIAVIIQSTA |

Table C11 B3. Alteration effect
None
Table D11C1.
Nucleotide sequence of variant 13380065 NOV4a3n (underlined). A/G
(SEQ ID NO:159)

| | |
|---|---|
| 1 | GCGTCCCCCACACCACAGACCCGCGCCGCCGACGACCCAGCAGCCGCCATGCGTCTGCTCCGAGGTGTGTTTGTAGTTGC |
| 81 | TGCTAAGCGAACGCCCTTTGGAGCTTACGGAGGCCTTCTGAAAGACTTCACTGCTACTGACTTGTCTGAATTTGCTGCCA |
| 161 | AGGCTGCCTTGTCTGCTGGCAAAGTCTCACCTGAAACAGTTGACAGTGTGATTATGGGCAATGTCCTGCAGAGTTCTTCA |
| 241 | GATGCTATATATTTGGCAAGGCATGTTGGTTTGCGTGTGGGAATCCCAAAGGAGACCCCAGCTCTCACGATTAATAGGCT |
| 321 | CTGTGCTTCTGGTTTTCAGTCCATTGTGAATGGATGTCAGGAAATTTCTGTTAAAGAAGCTGAAGTTGTTTTATGTGGAG |
| 401 | GAACCGAAAGCATGAGCCAAGCTCCCTACTGTGTCAGAAATGTGCGTTTTGGAACCAAGCTTGCATCAGATATCAAGCTG |
| 481 | GAAGATTCTTTATGGGTATCATTAACAGATCAGCATGTCCAGCTCCCCATGGCAATGACTGCAGAGAATCTTACTGTAAA |
| 561 | ACACAAAATAAGCAGAGAAGAATGTGACAAATATGCCCTGCAGTCACAGCAGAGATGGAAAGCTGCTAATGATGCTGGCT |
| 641 | ACTTTAATGATGAAATGGCACCAATTGAAGTGAAGACAAAGAAAGGAAAACAGACAATGCAGGTAGACGAGCATGCTCGG |
| 721 | CCCCAAACCACCCTGGAACAGTTACAGAAACTTCCTCCAGTATTCAAGAAAGATGGAACTGTTACTGCAGGGAATGCATC |
| 801 | GGGTGTAGCTGATGGTGCTGGAGCTGTTATCATAGCTAGTGAAGATGCTGTTAAGAAACATAACTTCACACCACTGGCAA |
| 881 | GAATTGTGGGCTACTTTGTATCTGGATGTGATCCCTCTATCATGGGTATTGGTCCTGTCCCTGCTATCAGTGGGGCACTG |
| 961 | AAGAAAGCAGGACTGAGTCTTAAGGACATGGATTTGGTAGAGGTGAATGAAGCTTTTGCTCCCCAGTACTTGGCTGTTGA |
| 1041 | GAGGAGTTTGGATCTTGACATAAGTAAAACCAATGTGAATGGAGGAGCCATTGCTTTGGGTCACCCACTGGGAGGATCTG |
| 1121 | GATCAAGAATTACTGCACACCTGGTTCACGAATTAAGGCGTCGAGGTGGAAAATATGCCGTTGGATCAGCTTGCATTGGA |
| 1201 | GGTGGCCAAGGTATTGCTGTCATCATTCAGAGCACAGCCTGAAGAGACCAGTGAGCTCACTGTGACCCATCCTTACTCTA |
| 1281 | CTTGGCCAGGCCACAGTAAAACAAGTGACCTTCAGAGCAGCTGCCACAACTGGCCATGCCCTGCCATTGAAACAGTGATT |
| 1361 | AAGTTTGATCAAGCCATGGTGACACAAAAATGCATTGATCATGAATAGGAGCCCATGCTAGAAGTACATTCTCTCAGATT |
| 1441 | TGAACCAGTGGAATATGATGTATTTCTGAGCTAAAACTCAACTATAGAAGACATTAAAAGAAATCGTATTCTTGCCAAGT |
| 1521 | AACCACCACTTCTGCCTTAGATAATATGATTATAAGGAAATCAAATAAATGTTGCCTTAACTTC |

Table D11C2.
Protein sequence of variant NOV4a3p (underlined). (SEQ ID NO:160)

| | |
|---|---|
| 1 | MRLLRGVFVVAAKRTPFGAYGGLLKDFTATDLSEFAAKAALSAGKVSPETVDSVIMGNVLQSSSDAIYLARHVGLRVGIP |
| 81 | KETPALTINRLCGSGFQSIVNGCQEICVKEAEVVLCGGTESMSQAPYCVRNVRFGTKLGSDIKLEDSLWVSLTDQHVQLP |
| 161 | MANTAENLTVKHKISREECDKYALQSQQRWKAANDAGYFNDEMAPIEVKTKKGKQTMQVDEHARPQTTLEQLQKLPPVFK |
| 241 | KDGTVTAGNASGVADGAGAVIIASEDAVKKHNFTPLARIVGYFVSGCDPSIMGIGPVPAISGALKKAGLSLKDMDLVEVN |
| 321 | EAFAPQYLAVERSLDLDISKTNVWGGAIALGHPLGGSGSRITAHLVHELRRRGGKYAVGSACIGGGQGIAVIIQSTA |

Table D11C3. Alteration effect
None
Table D11D1.
Nucleotide sequence of variant 13380066 NOV4a4n (underlined). C/T
(SEQ ID NO:161)

| | |
|---|---|
| 1 | GCGTCCCCCACACCACAGACCCGCGCCGCCGACGACCCAGCAGCCGCCATGCGTCTGCTCCGAGGTGTGTTTGTAGTTGC |
| 81 | TGCTAAGCGAACGCCCTTTGGAGCTTACGGAGGCCTTCTGAAAGACTTCACTGCTACTGACTTGTCTGAATTTGCTGCCA |
| 161 | AGGCTGCCTTGTCTGCTGGCAAAGTCTCACCTGAAACAGTTGACAGTGTGATTATGGGCAATGTCCTGCAGAGTTCTTCA |
| 241 | GATGCTATATATTTGGCAAGGCATGTTGGTTTGCGTGTGGGAATCCCAAAGGAGACCCCAGCTCTCACGATTAATAGGCT |
| 321 | CTGTGGTTCTGGTTTTCAGTCCATTGTGAATGGATGTCAGGAAATTTGTGTTAAAGAAGCTGAAGTTGTTTTATGTGGAG |
| 401 | GAACCGAAAGCATGAGCCAAGCTCCCTACTGTGTCAGAAATGTGCGTTTTGGAACCAAGCTTGGATCAGATATCAAGCTG |
| 481 | GAAGATTCTTTATGGGTATCATTAACAGATCAGCATGTCCAGCTCCCCATGGCAATGACTGCAGAGAATCTTACTGTAAA |
| 561 | ACACAAAATAAGCAGAGAAGAATGTGACAAATATGCCCTGCAGTCACAGCAGAGATGGAAAGCTGCTAATGATGCTGGCT |
| 641 | ACTTTAATGATGAAATGGCACCAATTGAAGTGAAGACAAAGAAAGGAAAACAGACAATGCAGGTAGACGAGCATGCTCGG |
| 721 | CCCCAAACCACCCTGGAACAGTTACAGAAACTTCCTCCAGTATTCAAGAAAGATGGAACTGTTACTGCAGGGAATGCATC |
| 801 | GGGTGTAGCTGATGGTGCTGGAGCTGTTATCATAGCTAGTGAAGATGCTGTTAAGAAACATAACTTCACACCACTGGCAA |
| 881 | GAATTGTGGGCTACTTTGTATCTGGATGTGATCCCTCTATCATGGGTATTGGTCCTGTCCCTGCTATCAGTGGGGCACTG |
| 961 | AAGAAAGCAGGACTGAGTCTTAAGGACATGGATTTGGTAGAGGTGAATGAAGCTTTTGCTCCCCAGTACTTGGCTGTTGA |
| 1041 | GAGGAGTTTGGATCTTGACATAAGTAAAACCAATGTGAATGGAGGAGCCATTGCTTTGGGTCACCCACTGGGAGGATCTG |
| 1121 | GATCAAGAATTACTGCACACCTGGTTCACGAATTAAGGCGTCGAGGTGGAAAATATGCCGTTGGATCAGCTTGCATTGGA |
| 1201 | GGTGGCCAAGGTATTGCTGTCATCATTCAGAGCACAGCCTGAAGAGACCAGTGAGCTCACTGTGACCCATCCTTACTCTA |
| 1281 | CTTGGCCAGGCCACAGTAAAACAAGTGACCTTCAGAGCAGCTGCCACAACTGGCCATGCCCTGCCATTGAAACAGTGATT |
| 1361 | AAGTTTGATCAAGCCATGGTGACACAAAAATGCATTGATCATGAATAGGAGCCCATGCTAGAAGTACATTCTCTCAGATT |
| 1441 | TGAACCAGTGAAATATGATGTATTTTTGAGCTAAAACTCAACTATAGAAGACATTAAAAGAAATCGTATTCTTGCCAAGT |
| 1521 | AACCACCACTTCTGCCTTAGATAATATGATTATAAGGAAATCAAATAAATGTTGCCTTAACTTC |

Table D11D2.
Protein sequence of variant NOV4a4p (underlined). (SEQ ID NO:162)

| | |
|---|---|
| 1 | MRLLRGVFVVAAKRTPFGAYGGLLKDFTATDLSEFAAKAALSAGKVSPETVDSVIMGNVLQSSSDAIYLARHVGLRVGIP |
| 81 | KETPALTINRLCGSGFQSIVNGCQEICVKEAEVVLCGGTESMSQAPYCVRNVRFGTKLGSDIKLEDSLWVSLTDQHVQLP |
| 161 | MANTAENLTVKHKISREECDKYALQSQQRWKAANDAGYFNDEMAPIEVKTKKGKQTMQVDEHARPOTTLEQLQKLPPVFK |
| 241 | KDGTVTAGNASGVADGAGAVIIASEDAVKKHNFTPLARIVGYFVSGCDPSIMGIGPVPAISGALKKAGLSLKDMDLVEVN |
| 321 | EAFAPQYLAVERSLDLDISKTNVNGGAIALGHPLGGSGSRITAHLVHELRRRGGKYAVGSACIGGGOGIAVIIQSTA |

Table D11D3. Alteration effect
None
Table D11E1.
Nucleotide sequence of variant 13380067 NOV4a5n (underlined). A/C
(SEQ ID NO:163)

| | |
|---|---|
| 1 | GCGTCCCCCACACCACAGACCCGCGCCGCCGACGACCCAGCAGCCGCCATGCGTCTGCTCCGAGGTGTGTTTGTAGTTGC |
| 81 | TGCTAAGCGAACGCCCTTTGGAGCTTACGGAGGCCTTCTGAAAGACTTCACTGCTACTGACTTGTCTGAATTTGCTGCCA |
| 161 | AGGCTGCCTTGTCTGCTGGCAAAGTCTCACCTGAAACAGTTGACAGTGTGATTATGGGCAATGTCCTGCAGAGTTCTTCA |
| 241 | GATGCTATATATTTGGCAAGGCATGTTGGTTTGCGTGTGGGAATCCCAAAGGAGACCCCAGCTCTCACGATTAATAGGCT |
| 321 | CTGTGGTTCTGGTTTTCAGTCCATTGTGAATGGATGTCAGGAAATTTGTGTTAAAGAAGCTGAAGTTGTTTTATGTGGAG |
| 401 | GAACCGAAAGCATGAGCCAAGCTCCCTACTGTGTCAGAAATGTGCGTTTTGGAACCAAGCTTGGATCAGATATCAAGCTG |
| 481 | GAAGATTCTTTATGGGTATCATTAACAGATCAGCATGTCCAGCTCCCCATGGCAATGACTGCAGAGAATCTTACTGTAAA |
| 561 | ACACAAAATAAGCAGAGAAGAATGTGACAAATATGCCCTGCAGTCACAGCAGAGATGGAAAGCTGCTAATGATGCTGGCT |
| 641 | ACTTTAATGATGAAATGGCACCAATTGAAGTGAAGACAAAGAAAGGAAAACAGACAATGCAGGTAGACGAGCATGCTCGG |
| 721 | CCCCAAACCACCCTGGAACAGTTACAGAAACTTCCTCCAGTATTCAAGAAAGATGGAACTGTTACTGCAGGGAATGCATC |
| 801 | GGGTGTAGCTGATGGTGCTGGAGCTGTTATCATAGCTAGTGAAGATGCTGTTAAGAAACATAACTTCACACCACTGGCAA |
| 881 | GAATTGTGGGCTACTTTGTATCTGGATGTGATCCCTCTATCATGGGTATTGGTCCTGTCCCTGCTATCAGTGGGGCACTG |
| 961 | AAGAAAGCAGGACTGAGTCTTAAGGACATGGATTTGGTAGAGGTGAATGAAGCTTTTGCTCCCCAGTACTTGGCTGTTGA |
| 1041 | GAGGAGTTTGGATCTTGACATAAGTAAAACCAATGTGAATGGAGGAGCCATTGCTTTGGGTCACCCACTGGGAGGATCTG |
| 1121 | GATCAAGAATTACTGCACACCTGGTTCACGAATTAAGGCGTCGAGGTGGAAAATATGCCGTTGGATCAGCTTGCATTGGA |
| 1201 | GGTGGCCAAGGTATTGCTGTCATCATTCAGAGCACAGCCTGAAGAGACCAGTGAGCTCACTGTGACCCATCCTTACTCTA |
| 1281 | CTTGGCCAGGCCACAGTAAAACAAGTGACCTTCAGAGCAGCTGCCACAACTGGCCATGCCCTGCCATTGAAACAGTGATT |
| 1361 | AAGTTTGATCAAGCCATGGTGACACAAAAATGCATTGATCATGAATAGGAGCCCATGCTAGAAGTACATTCTCTCAGATT |
| 1441 | TGAACCAGTGAAATATGATGTATTTCTGAGCTAAAACTCAACTATAGAAGACATTAAAAGAAATCGTATTCTTGCCAAGT |
| 1521 | AACCCCCACTTCTGCCTTAGATAATATGATTATAAGGAAATCAAATAAATGTTGCCTTAACTTC |

Table D11E2.
Protein sequence of variant NOV4a5p (underlined). (SEQ ID NO:164)

| | |
|---|---|
| 1 | MRLLRGVFVVAAKRTPFGAYGGLLKDFTATDLSEFAAKAALSAGKVSPETVDSVIMGNVLQSSSDAIYLARHVGLRVGIP |
| 81 | KETPALTINRLCGSGFQSIVNGCQEICVKEAEVVLCGGTESMSQAPYCVRNVRFGTKLGSDIKLEDSLWVSLTDQHVQLP |
| 161 | MAMTAENLTVKHKISREECDKYALQSQQRWKAANDAGYFNDEMAPIEVKTKKGKQTMQVDEHARPQTTLEQLQKLPPVFK |
| 241 | KDGTVTAGNASGVADGAGAVIIASEDAVKKHNFTPLARIVGYFVSGCDPSIMGIGPVPAISGALKKAGLSLKDMDLVEVN |
| 321 | EAFAPQYLAVERSLDLDISKTNVNGGAIALGHPLGGSGSRITAHLVHELRRRGGKYAVGSACIGGGQGIAVIIQSTA |

Table D11E3. Alteration effect
None
Table D11F1.
Nucleotide sequence of variant 13380068 NOV4a6n (underlined). T/C
(SEQ ID NO:165)

| | |
|---|---|
| 1 | GCGTCCCCCACACCACAGACCCGCGCCGCCGACGACCCAGCAGCCGCCATGCGTCTGCTCCGAGGTGTGTTTGTAGTTGC |
| 81 | TGCTAAGCGAACGCCCTTTGGAGCTTACGGAGGCCTTCTGAAAGACTTCACTGCTACTGACTTGTCTGAATTTGCTGCCA |
| 161 | AGGCTGCCTTGTCTGCTGGCAAAGTCTCACCTGAAACAGTTGACAGTGTGATTATGGGCAATGTCCTGCAGAGTTCTTCA |
| 241 | GATGCTATATATTTGGCAAGGCATGTTGGTTTGCGTGTGGGAATCCCAAAGGAGACCCCAGCTCTCACGATTAATAGGCT |
| 321 | CTGTGGTTCTGGTTTTCAGTCCATTGTGAATGGATGTCAGGAAATTTGTGTTAAAGAAGCTGAAGTTGTTTTATGTGGAG |
| 401 | GAACCGAAAGCATGAGCCAAGCTCCCTACTGTGTCAGAAATGTGCGTTTTGGAACCAAGCTTGGATCAGATATCAAGCTG |
| 481 | GAAGATTCTTTATGGGTATCATTAACAGATCAGCATGTCCAGCTCCCCATGGCAATGACTGCAGAGAATCTTACTGTAAA |
| 561 | ACACAAAATAACCAGAGAAGAATGTGACAAATATGCCCTGCAGTCACAGCAGAGATGGAAAGCTGCTAATGATGCTGGCT |
| 641 | ACTTTAATGATGAAATGGCACCAATTGAAGTGAAGACAAAGAAAGGAAAACAGACAATGCAGGTAGACGAGCATGCTCGG |
| 721 | CCCCAAACCACCCTGGAACAGTTACAGAAACTTCCTCCAGTATTCAAGAAAGATGGAACTGTTACTGCAGGGAATGCATC |
| 801 | GGGTGTAGCTGATGGTGCTGGAGCTGTTATCATAGCTAGTGAAGATGCTGTTAAGAAACATAACTTCACACCACTGGCAA |
| 881 | GAATTGTGGGCTACTTTGTATCTGGATGTGATCCCTCTATCATGGGTATTGGTCCTGTCCCTGCTATCAGTGGGGCACTG |
| 961 | AAGAAAGCAGGACTGAGTCTTAAGGACATGGATTTGGTAGAGGTGAATGAAGCTTTTGCTCCCCAGTACTTGGCTGTTGA |
| 1041 | GAGGAGTTTGGATCTTGACATAAGTAAAACCAATGTGAATGGAGGAGCCATTGCTTTGGGTCACCCACTGGGAGGATCTG |
| 1121 | GATCAAGAATTACTGCACACCTGGTTCACGAATTAAGGCGTCGAGGTGGAAAATATGCCGTTGGATCAGCTTGCATTGGA |
| 1201 | GGTGGCCAAGGTATTGCTGTCATCATTCAGAGCACAGCCTGAAGAGACCAGTGAGCTCACTGTGACCCATCCTTACTCTA |
| 1281 | CTTGGCCAGGCCACAGTAAAACAAGTGACCTTCAGAGCAGCTGCCACAACTGGCCATGCCCTGCCATTGAAACAGTGATT |
| 1361 | AAGTTTGATCAAGCCATGGTGACACAAAAATGCATTGATCATGAATAGGAGCCCATGCTAGAAGTACATTCTCTCAGATT |
| 1441 | TGAACCAGTGAAATATGATGTATTTCTGAGCTAAAACTCAACTATAGAAGACATTAAAAGAAATCGTATTCTTGCCAAGT |
| 1521 | AACCACCACTTCTGCCTTAGATAATATGACTATAAGGAAATCAAATAAATGTTGCCTTAACTTC |

Table D11F2.
Protein sequence of variant NOV4a6p (underlined). (SEQ ID NO:166)

| | |
|---|---|
| 1 | MRLLRGVFVVAAKRTPFGAYGGLLKDFTATDLSEFAAKAALSAGKVSPETVDSVIMGNVLOSSSDAIYLARHVGLRVGIP |
| 81 | KETPALTINRLCGSGFQSIVNGCQEICVKEAEVVLCGGTESMSQAPYCVRNVRFGTKLGSDIKLEDSLWVSLTDQHVQLP |
| 161 | MANTAENLTVKHKISREECDKYALQSQQRWKAANDAGYFNDEMAPIEVKTKKGKQTMQVDEHARPQTTLEQLQKLPPVFK |
| 241 | KDGTVTAGNASGVADGAGAVIIASEDAVKKHNFTPLARIVGYFVSGCDPSIMGIGPVPAISGALKKAGLSLKDMDLVEVN |
| 321 | SAFAPQYLAVERSLDLDISKTNVNGGAIALGHPLGGSGSRITAHLVHELRRRGGKYAVGSACIGGGQGIAVIIQSTA |

Table D11F3. Alteration effect
None

### EXAMPLE D6

### Expression Profile of the Human Acetyl-Coenzyme A acyltransferase 2 Gene

The protocol for quantitative expression analysis is disclosed in Example Q9.

Expression of gene CG181387-01 and CG181387-02 was assessed using the primer-probe set Ag6643, described in Table D13. Results of the RTQ-PCR runs are shown in Tables D14 and D15.

TABLE D13
Probe Name Ag6643

| | | | Start | SEQ ID |
|---|---|---|---|---|
| Primers | Sequences | Length | Position | No |
| Forward | 5′-tactgtaaaacacaaaataagcagagaag-3′ | 29 | 552 | 194 |
| Probe | TET-5′-aatatgccctgcagtcacagcagaga-3′- | 26 | 590 | 195 |
| | TAMRA | | | |
| Reverse | 5′-atcattaaagtagccagcatcattag-3′ | 26 | 626 | 196 |

TABLE D14
General screening panel v1.6

| | Rel. Exp. (%) |
|---|---|
| | Ag6643, Run |
| Tissue Name | 277256952 |
| Adipose | 12.8 |
| Melanoma* Hs688(A).T | 19.5 |
| Melanoma* Hs688(B).T | 18.4 |
| Melanoma* M14 | 11.5 |
| Melanoma* LOXIMVI | 11.3 |
| Melanoma* SK-MEL-5 | 17.1 |
| Squamous cell carcinoma SCC-4 | 0.5 |
| Testis Pool | 6.0 |
| Prostate ca.* (bone met) PC-3 | 4.3 |
| Prostate Pool | 4.0 |
| Placenta | 3.0 |
| Uterus Pool | 1.6 |
| Ovarian ca. OVCAR-3 | 6.6 |
| Ovarian ca. SK-OV-3 | 0.4 |
| Ovarian ca. OVCAR-4 | 4.0 |
| Ovarian ca. OVCAR-5 | 13.6 |
| Ovarian ca. IGROV-1 | 6.3 |
| Ovarian ca. OVCAR-8 | 1.4 |
| Ovary | 5.3 |
| Breast ca. MCF-7 | 7.7 |
| Breast ca. MDA-MB-231 | 13.1 |
| Breast ca. BT 549 | 28.3 |
| Breast ca. T47D | 4.9 |
| Breast ca. MDA-N | 8.8 |
| Breast Pool | 5.4 |
| Trachea | 1.7 |
| Lung | 1.7 |
| Fetal Lung | 9.3 |
| Lung ca. NCI-N417 | 6.2 |
| Lung ca. LX-1 | 19.8 |
| Lung ca. NCI-H146 | 31.9 |
| Lung ca. SHP-77 | 35.6 |
| Lung ca. A549 | 21.8 |
| Lung ca. NCI-H526 | 9.0 |
| Lung ca. NCI-H23 | 12.9 |
| Lung ca. NCI-H460 | 10.0 |
| Lung ca. HOP-62 | 3.8 |
| Lung ca. NCI-H522 | 24.5 |
| Liver | 18.6 |
| Fetal Liver | 27.9 |
| Liver ca. HepG2 | 9.3 |
| Kidney Pool | 6.9 |
| Fetal Kidney | 23.3 |
| Renal ca. 786-0 | 7.7 |
| Renal ca. A498 | 1.1 |
| Renal ca. ACHN | 4.2 |
| Renal ca. UO-31 | 16.6 |
| Renal ca. TK-10 | 7.2 |
| Bladder | 25.3 |
| Gastric ca. (liver met.) NCI-N87 | 13.6 |
| Gastric ca. KATO III | 100.0 |
| Colon ca. SW-948 | 9.5 |
| Colon ca. SW480 | 22.4 |
| Colon ca.* (SW480 met) SW620 | 14.9 |
| Colon ca. HT29 | 13.9 |
| Colon ca. HCT-116 | 5.4 |
| Colon ca. CaCo-2 | 45.1 |
| Colon cancer tissue | 13.3 |
| Colon ca. SW1116 | 2.7 |
| Colon ca. Colo-205 | 5.6 |
| Colon ca. SW-48 | 15.0 |
| Colon Pool | 5.1 |
| Small Intestine Pool | 3.5 |
| Stomach Pool | 2.6 |
| Bone Marrow Pool | 2.6 |
| Fetal Heart | 12.7 |
| Heart Pool | 11.0 |
| Lymph Node Pool | 5.6 |
| Fetal Skeletal Muscle | 7.2 |
| Skeletal Muscle Pool | 5.0 |
| Spleen Pool | 2.5 |
| Thymus Pool | 3.9 |
| CNS cancer (glio/astro) U87-MG | 7.8 |
| CNS cancer (glio/astro) U-118-MG | 21.3 |
| CNS cancer (neuro; met) SK-N-AS | 22.1 |
| CNS cancer (astro) SF-539 | 9.3 |
| CNS cancer (astro) SNB-75 | 32.5 |
| CNS cancer (glio) SNB-19 | 5.8 |
| CNS cancer (glio) SF-295 | 9.2 |
| Brain (Amygdala) Pool | 4.1 |
| Brain (cerebellum) | 4.8 |
| Brain (fetal) | 3.9 |
| Brain (Hippocampus) Pool | 5.7 |
| Cerebral Cortex Pool | 4.5 |
| Brain (Substantia nigra) Pool | 3.4 |
| Brain (Thalamus) Pool | 6.2 |
| Brain (whole) | 6.1 |
| Spinal Cord Pool | 5.6 |
| Adrenal Gland | 11.9 |
| Pituitary gland Pool | 2.1 |
| Salivary Gland | 1.0 |
| Thyroid (female) | 6.6 |
| Pancreatic ca. CAPAN2 | 10.9 |
| Pancreas Pool | 6.3 |

TABLE D15
Panel 5 Islet

| | Rel. Exp. (%) |
|---|---|
| | Ag6643, Run |
| Tissue Name | 279519414 |
| 97457_Patient-02go_adipose | 22.5 |
| 97476_Patient-07sk_skeletal muscle | 0.0 |
| 97477_Patient-07ut_uterus | 6.0 |
| 97478_Patient-07pl_placenta | 9.8 |
| 99167_Bayer Patient 1 | 20.9 |
| 97482_Patient-08ut_uterus | 3.5 |
| 97483_Patient-08pl_placenta | 6.6 |
| 97486_Patient-09sk_skeletal muscle | 9.4 |
| 97487_Patient-09ut_uterus | 6.7 |
| 97488_Patient-09pl_placenta | 4.5 |
| 97492_Patient-10ut_uterus | 12.5 |
| 97493_Patient-10pl_placenta | 19.9 |
| 97495_Patient-11go_adipose | 9.6 |
| 97496_Patient-11sk_skeletal muscle | 15.4 |
| 97497_Patient-11ut_uterus | 12.3 |
| 97498_Patient-11pl_placenta | 3.1 |
| 97500_Patient-12go_adipose | 29.9 |
| 97501_Patient-12sk_skeletal muscle | 51.4 |
| 97502_Patient-12ut_uterus | 13.6 |
| 97503_Patient-12pl_placenta | 10.4 |
| 94721_Donor 2 U - A_Mesenchymal Stem Cells | 37.1 |
| 94722_Donor 2 U - B_Mesenchymal Stem Cells | 24.5 |
| 94723_Donor 2 U - C_Mesenchymal Stem Cells | 36.1 |
| 94709_Donor 2 AM - A_adipose | 45.1 |
| 94710_Donor 2 AM - B_adipose | 25.7 |
| 94711_Donor 2 AM - C_adipose | 21.3 |
| 94712_Donor 2 AD - A_adipose | 49.7 |
| 94713_Donor 2 AD - B_adipose | 70.7 |
| 94714_Donor 2 AD - C_adipose | 60.7 |
| 94742_Donor 3 U - A_Mesenchymal Stem Cells | 12.2 |
| 94743_Donor 3 U - B_Mesenchymal Stem Cells | 12.7 |
| 94730_Donor 3 AM - A_adipose | 65.5 |
| 94731_Donor 3 AM - B_adipose | 68.3 |
| 94732_Donor 3 AM - C_adipose | 74.2 |
| 94733_Donor 3 AD - A_adipose | 100.0 |
| 94734_Donor 3 AD - B_adipose | 90.8 |
| 94735_Donor 3 AD - C_adipose | 29.3 |
| 77138_Liver_HepG2untreated | 59.5 |
| 73556_Heart_Cardiac stromal cells (primary) | 5.6 |
| 81735_Small Intestine | 32.8 |
| 72409_Kidney_Proximal Convoluted Tubule | 40.3 |
| 82685_Small intestine_Duodenum | 50.3 |
| 90650_Adrenal_Adrenocortical adenoma | 6.0 |
| 72410_Kidney_HRCE | 15.4 |
| 72411_Kidney_HRE | 16.0 |
| 73139_Uterus_Uterine smooth muscle cells | 5.1 |

General screening panel v1.6 Summary: Ag6643 Highest expression of this gene was detected in a gastric cancer KATO III cell line (CT=24.2). Moderate to high levels of expression of this gene were also seen in cluster of cancer cell lines derived from pancreatic, gastric, colon, lung, liver, renal, breast, ovarian, prostate, melanoma and brain cancers. Thus, expression of this gene could be used as a marker to detect the presence of these cancers. Therapeutic modulation of this gene, expressed protein and/or use of antibodies or small molecule drugs targeting the gene or gene product may be useful in the treatment of pancreatic, gastric, colon, lung, liver, renal, breast, ovarian, prostate, squamous cell carcinoma, melanoma and brain cancers.

Among tissues with metabolic or endocrine function, this gene was expressed at moderate to high levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product may be useful in the treatment of endocrine/metabolically related diseases, such as obesity and diabetes.

In addition, this gene was expressed at moderate levels in all regions of the central nervous system examined, including amygdala, hippocampus, substantia nigra, thalamus, cerebellum, cerebral cortex, and spinal cord. Therapeutic modulation of this gene, expressed protein and/or use of antibodies or small molecule drugs targeting the gene or gene product may be useful in the treatment of central nervous system disorders such as Alzheimer's disease, Parkinson's disease, epilepsy, multiple sclerosis, schizophrenia and depression.

Panel 5 Islet Summary: Ag6643 Highest expression of this gene was detected in differentiated adipose (CT=28.2). High expression of this gene was seen in midway and fully differentiated adipose tissue, in addition moderate levels of expression of this gene was also detected in islet cells, skeletal muscle, small intestine and heart.

### EXAMPLE D7

### Assays for Modulators of Acetyl-Coenzyme A Acyltransferase 2

One potential assay that may be used to screen for modulators of Acetyl-Coenzyme A acyltransferase 2 is to measure acetoacetyl-CoA cleavage by an optical assay following the decrease of the enol and chelate form of acetoacetyl-CoA by absorption measurement at 305 nm as described Berndt H, Schlegel H G. Kinetics and properties of beta-ketothiolase from Clostridium pasteurianum. Arch Microbiol. 1975 Mar. 12; 103 (1):21-30, PMID: 240336).

Another potential assay that may be used to screen for modulators of Acetyl-Coenzyme A acyltransferase 2 is the measurement of NAD+/NADH production in a reaction coupled with the conversion reaction of 3-hydroxyacyl CoA to 3-oxoacyl CoA by the next mitochondrial enzyme (Barycki J J, O'Brien L K, Bratt J M, Zhang R, Sanishvili R, Strauss A W, Banaszak L J. Biochemical characterization and crystal structure determination of human heart short chain L-3-hydroxyacyl-CoA dehydrogenase provide insights into catalytic mechanism. Biochemistry. 1999 May 4; 38 (18):5786-98. PMID: 10231530).

Our results indicate that a modulator of ACAA2 activity, such as an inhibitor, activator, antagonist, or agonist of ACAA2 may be useful for treatment of such disorders as obesity, diabetes, and insulin resistance, as well as for enhancement of insulin secretion.

E. NOV5—PHOSPHOGLYCERATE MUTASE 2

Phosphoglycerate mutase is a dimeric enzyme containing, in different tissues, various proportions of a slow-migrating (PGM2) isoenzyme and a fast-migrating brain (PGM1) isoenzyme, which are encoded by different genes. PGM1 is a ubiquitously expressed isoenzyme with highest expression in the brain. In contrast, PGM2 is expressed specifically in skeletal muscle. Phosphoglycerate mutase is an enzyme involved in the second step of glycolysis (conversion of glyceraldehydes 3-phopshate to pyruvate). Complete deficiency in PGM2 leads to a muscle phenotype associated with mild myopathy and exercise intolerance (Tsujino S, Shanske S, Sakoda S, Fenichel G, DiMauro S. The molecular genetic basis of muscle phosphoglycerate mutase (PGAM) deficiency. (1993) Am. J. Hum. Genet. 52 (3):472-7 PMID: 8447317). This phenotype may promote energy expenditure in a diabetic/obese condition.

We have found that most of the enzymes involved in the second step of glycolysis are up-regulated in diabetic skeletal muscle. This is consistent with the literature data which demonstrated an increase in glycolytic enzyme activity and a decrease in oxidative enzyme activity in the skeletal muscle from diabetic/obese patients (Simoneau J A, Kelley D E. Altered glycolytic and oxidative capacities of skeletal muscle contribute to insulin resistance in NIDDM. (1997) J. Appl. Physiol. 83 (1):166-71 PMID: 9216960). Disbalance of glycolytic/oxidative capacity may increase the lipid content and consequentially lead to the development of insulin resistance in skeletal muscle.

Our differential expression (GeneCalling®) data shows the up-regulation of PGM1 in soleus muscle in diabetic compared to non-diabetic animals. PGM 1 was found expressed more in glycolytic then in oxidative muscle fiber. RTQ-PCR results demonstrate that PGM2 is specifically expressed in muscle tissue and up-regulated in insulin resistant skeletal muscle from patients with Gestational Diabetes. Up-regulation of both PGM1 and PGM2 detected in our studies would contribute to increase in glycolytic muscle capacity described in diabetic conditions.

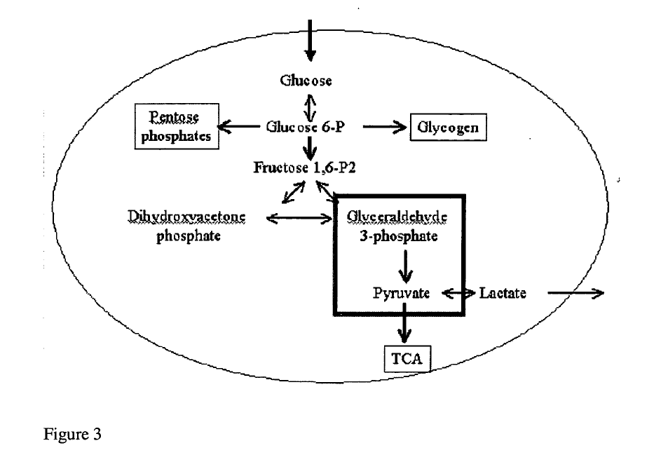

FIG. 3 suggests how alterations in expression of the human Phosphoglycerate mutase 2 and associated gene products function in the etiology and pathogenesis of obesity and/or diabetes. The scheme incorporates the unique findings of our studies in conjunction with what has been reported in the literature.

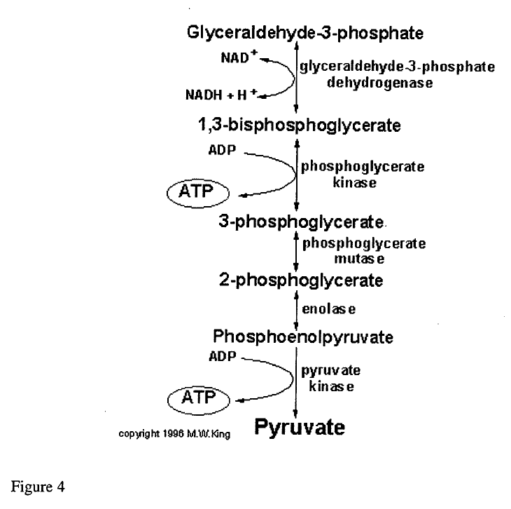

FIG. 4 summarizes the biochemistry surrounding the human Phosphoglycerate mutase 2 and potential assays that may be used to screen for antibody therapeutics or small molecule drugs to treat obesity and/or diabetes. In the presence of 2,3-biphosphoglycerate, phosphoglycerate mutase catalyses the interconversion between 3-phosphoglycerate and 2-phosphoglycerate, the third step in conversion of glyceraldehydes-3-phosphate to pyruvate:
3-Phosphoglycerate⇄2-phospoglycerate

Activation of glycolysis can contribute to disproportional increase in glycolytic capacity in diabetic skeletal muscle and development of insulin resistance. Because of specific expression of PGM2 in skeletal muscle, its inhibition may restore the energy balance and favor the oxidative muscle phenotype with minimal effect on glycolysis in other tissues. The outcome of inhibiting the action of the human Phosphoglycerate mutase 2 would be a reduction of Insulin Resistance, a major problem in obesity and/or diabetes. Thus, an antagonist or an inhibitor of PGM2 may be used for the treatment of obesity and/or diabetes.

Cell lines expressing the Phosphoglycerate mutase 2 are described in the RTQ-PCR results described herein. These and other Phosphoglycerate mutase 2 expressing cell lines could be used for screening purposes. The assay for measurement of phosphoglycerate mutase activity and the selective inhibitor for PGM2 have been described in literature (see for example White M F, Fothergill-Gilmore L A. Development of a mutagenesis, expression and purification system for yeast phosphoglycerate mutase. Investigation of the role of active-site His181. Eur J. Biochem. 1992 Jul. 15; 207 (2):709-14. PMID: 1386023, Rigden D J, Walter R A, Phillips S E, Fothergill-Gilmore L A. Polyanionic inhibitors of phosphoglycerate mutase: combined structural and biochemical analysis. J Mol. Biol. 1999 Jun. 18; 289 (4):691-9. PMID: 10369755).

Furthermore, our results indicate that a modulator of Phosphoglycerate mutase 2 activity, such as an inhibitor, activator, antagonist, or agonist of Phosphoglycerate mutase 2 may be useful for treatment of such disorders as obesity, diabetes, and insulin resistance, as well as for enhancement of insulin secretion.

Discovery Process

The disregulation of PGM1 isoform in diet induced obesity study suggests that the increase in phosphoglycerate mutase activity in skeletal muscle may contribute to the development of insulin resistance and diabetes. Based on expression data, we propose PGM2, major skeletal muscle isoform of phosphoglycerate mutase, CG186640-O₂— encoded protein and any variants, thereof, as being suitable as diagnostic markers, targets for an antibody therapeutic and targets for a small molecule drugs for Obesity and Diabetes. Dysregulation of PGM1 gene in GeneCalling® study described below is supportive for disease rationale for phosphoglycerate mutase isoform 2.

The following sections describe the study design(s) and the techniques used to identify the Phosphoglycerate mutase 2—encoded protein and any variants, thereof, as being suitable as diagnostic markers, targets for an antibody therapeutic and targets for a small molecule drugs for Obesity and Diabetes.

### EXAMPLE E1

### Mouse Dietary-Induced Obesity

A protocol for Mouse Dietary-Induced Obesity study is disclosed in Example Q1.

The predominant cause for obesity in clinical populations is excess caloric intake. This so-called diet-induced obesity (DIO) is mimicked in animal models (mouse strain C57BL/6J) by feeding high fat diets of greater than 40% fat content. The DIO study was established to identify the gene expression changes contributing to the development and progression of diet-induced obesity. In addition, the study design sought to identify the factors that lead to the ability of certain individuals to resist the effects of a high fat diet and thereby prevent obesity. The sample groups for the study had body weights +1 S.D., +4 S.D. and +7 S.D. of the chow-fed controls. In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues revealed genes and pathways that can be used as therapeutic targets for obesity. Protocol for differential gene expression analysis, GeneCalling®, is disclosed in Example Q7.

Results

A fragment of the mouse Phosphoglycerate mutase 1 gene was initially found to be up-regulated by 2.7 fold in the soleus muscle of hyperglycemic (hgsd7, diabetic) mice relative to euglycemic (sd1, normal control) mice using CuraGen's GeneCalling® method of differential gene expression (Table E1 shows the alignment of the PGM1 and PGM2 protein sequences with PGM1 protein sequence shown separately in Table E2). A differentially expressed mouse gene fragment migrating, at approximately 153 nucleotides in length was definitively identified as a component of the mouse Phosphoglycerate mutase 1 cDNA. The method of comparative PCR was used for conformation of the gene assessment. The electropherographic peaks corresponding to the gene fragment of the mouse Phosphoglycerate mutase 1 were ablated when a gene-specific primer (shown in Table E3) competes with primers in the linker-adaptors during the PCR amplification. The peaks at 153 nt in length were ablated in the sample from both the hyperglycemic and euglycemic mice. A gene fragment of the mouse Phosphoglycerate mutase 1 was also found to be upregulated by approximately 2 fold in the gastrocnemius (glycolytic) muscle relative to slow twitch (oxidative) muscle fiber of obese hyperglycemic mice. These data show that phosphoglycerate mutase may be involved in the development of diabetes/obesity and its modulation, such as inhibition, may be beneficial for the treatment of these diseases.

[image]

TABLE E2

Protein sequence of Phosphoglycerate mutase 1
(PGM1) (CG115294-02; SEQ ID NO:197)

(SEQ ID NO:197)

CG115294-02
AAYKLVLIRHGESAWNLENRFSGWYDADLSPAGHEEAKRGGQALRDAGYE
FDICFTSVQKRAIRTLWTVLDAIDQMWLPVVRTWRLNERHYGGLTGLNKA
ETAAKHGEAQVKIWRRSYDVPPPPMEPDHPFYSNISKDRRYADLTEDQLP
SCESLKDTIARALPFWNEEIVPQIKEGKRVLIAAHGNSLRGIVKHLEGLS
EEAIMELNLPTGIPIVYELDKNLKPIKPMQFLGDEETVRKAMEAV
AAQGKAKK

TABLE E3

The direct sequence of the 153 nucleotide-long gene fragment and the
gene-specific primers used for competitive PCR are indicated on the
cDNA sequence of the Phosphoglycerate mutase 1 fragment (SEQ ID
NO:198) are shown below in bold. The gene-specific primers at the 5′
and 3′ ends of the fragment are underlined.
Gene Sequence (fragment from 80 to 233 in bold, band size: 154)

```
    1 AGGCGACGCG GCGGCCAGGC GTTGCGAGAT GCTGGCTATG AATTTGACAT CTGCTTCACC
  61 TCTGTGCAGA AGAGAGCAAT CCGGACCCTC TGGACAGTCC TGGATGCCAT TGACCAGATG
121 TGGTTGCCAG TGGTCAGGAC TTGGCGCCTC AATGAGCGAC ACTATGGCGG TCTGACAGGT
181 CTCAACAAAG CAGAAACTQC TGCTAAGCAA TGGTGAGGCC CAGGTAAAGA TCTGGAAACG
241 ATCTTATGAT GTCCCACCGG CTCCCATTGG ACCCTGATTA ACCCTTTCTA CAGCAACATT
301 CAGCAAGGAA TCGCAGGTAC GCAGAACCTT ACTGAAAGAC CCGCTTCCCC TCCTGT
```

(gene length is 356, only region from 1 to 356 shown)

### EXAMPLE E2

### Identification of Human Sequence of Phosphoglycerate Mutase 2

The sequence of Human Phosphoglycerate mutase 2 (Acc. No. CG 186640-O₂) was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof.

Table E4 shows an alignment (ClustalW) of the protein sequences of the human (CG186640-O₂; SEQ ID NO:46), rat (M31835; SEQ ID NO:199) and mouse (AF029843; BC010750; SEQ ID NO:200) versions of the Phosphoglycerate mutase 2. Table E5 shows sequences of rat (M31835; SEQ ID NO:199) and mouse (AF029843; BC010750; SEQ ID NO:200) versions of the Phosphoglycerate mutase 2.

[image]

[image]

TABLE E5

sequences of rat (M31835; SEQ ID NO:199) and mouse (AF029843;
BC010750; SEQ ID NO:200) versions of the Phosphoglycerate mutase 2.

>PGM2_rat (M3 1835; SEQ ID NO:199)
MATHRLVMVRHGESSWNQENRFCGWFDAELSEKGAEEAKRGATAIKDAKIEFDICYTSVLKRAIR
TLWTILDVTDQMWVPVVRTWRLNERHYGGLTGLNKAETAAKHGEEQVKIWRRSFDTPPPPMDE
KHNYYASISKDRRYAGLKPEELPTCESLKDTIARALPFWNEEIAPKIKAGKRVLIAAHGNSLRGIVK
HLEGMSDQAIMELNLPTGIPIVYELNQELKPTKPMRFLGDEETVRKAMEAVAAQGKAK
>PGM2_mouse (AF029843; BC010750; SEQ ID NO:200)
MTTHRLVMVRHGESLWNQENRFCGWFDAELSEKGAEEAKRGATAIKDAKIEFDICYTSVLKRAIR
TLWTILDVTDQMWVPVVRTWRLNERHYGGLTGLNKAETAAKHGEEQVKIWRRSFDTPPPPMDE
KHNYYTSISKDRRYAGLKPEELPTCESLKDTIARALPFWNEEIAPKIKAGQRVLIAAHGNSLRGIVK
HLEGMSDQAIMELNLPTGIPIVYELDQNLKPTKPMRFLGDEETVRKAMEAVAAQGKAK

The laboratory cloning was performed using one or more of the methods summarized in Example Q8. The NOV5 clone was analyzed, and the nucleotide and encoded polypeptide sequences are shown in Table E6.

TABLE E6
NOV5 Sequence Analysis

| | | |
|---|---|---|
| NOV5a, CG186640-02 | SEQ ID NO:45 | 834 bp |
| DNA Sequence | ORF Start: ATG at 36 | ORF Stop: TGA at 795 |

CCCGTCCAGAGTCCTCTGTGGTCCCTGCTGCCACCATGGCCACTCACCGCCTCGTGATGGTCCGGCAC
GGCGAGACGACATGGAACCAGGAGAACCGTTTCTGTGGCTGGTTCGATGCAGAGCTGAGTGAAAAGGG
GACCGAGGAGGCCAAGCGGGGAGCCAAGGCCATCAAGGATGCCAAGATGGAGTTTGACATCTGCTACA
CGTCAGTGCTGAAGCGGGCCATCCGAACCCTCTGGGCCATCCTGGACGGCACGGACCAGATGTGGCTG
CCTGTGGTGCGCACTTGGCGCTTCAATGAGCGGCATTACGGGGGCCTCACAGGCTTCAACAAGGCAGA
AACGGCCGCCAAGCACGGGGAGGAGCAGGTAAGATCTTGGAGGCGCTCCTTCGACATCCCGCCGCCCC
CGATGGACGAGAAGCACCCCTACTACAACTCCATTAGCAAGGAGCGTCGGTACGCAGGCCTGAAGCCC
GGGGAACTCCCCACCTGCGAGAGCCTCAAGGACACCATTGCCCGGGCCCTGCCCTTCTGGAACGAGGA
GATTGTTCCCCAGATCAAGGCCGGCAAGCGAGTGCTCATTGCAGCCCACGGGAACAGCCTGCGGGGCA
TTGTCAAGCACCTGGAAGGGATGTCAGACCAGGCGATCATGGAGCTGAACCTGCCCACGGGGATCCCC
ATTGTGTATGAGCTGAACAAGGAGCTGAAGCCCACCAAGCCCATGCAGTTCCTGGGTGATGAGGAAAC
GGTGCGGAAGGCCATGGAGGCTGTGGCTGCCCAGGGCAAGGCCAAGTGAGGGGTGGGCTTTGGGCAAT
AAAGGCACCTCCCCCAAC

| | | | |
|---|---|---|---|
| NOV5a, CG186640-02 | | | |
| Protein Sequence | SEQ ID NO:46 | 253 aa | MW at 28849.9 kD |

MATHRLVMVRHGETTWNQENRFCGWFDAELSEKGTEEAKRGAKAIKDAKMEFDICYTSVLKRAIRTLW
AILDGTDQMWLPVVRTWRFNERHYGGLTGFNKAETAAKHGEEQVRSWRRSFDIPPPPMDEKHPYYNSI
SKERRYAGLKPGELPTCESLKDTIARALPFWNEEIVPQIKAGKRVLIAAHGNSLRGIVKHLEGMSDQA
IMELNLPTGIPIVYELNKELKPTKPMQFLGDEETVRKAMEAVAAQGKAK

| | | |
|---|---|---|
| NOV5b, 311980359 | SEQ ID NO:47 | 786 bp |
| DNA Sequence | ORF Start: at 1 | ORF Stop: TGA at 784 |

ACCATGGGACATCATCACCACCATCACGCCACTCACCGCCTCGTGATGGTCCGGCACGGCGAGAGCAC
ATGGAACCAGGAGAACCGTTTCTGTGGCTGGTTCGATGCAGAGCTGAGTGAAAAGGGGACCGAGGAGG
CCAAGCGGGGAGCCAAGGCCATCAAGGATGCCAAGATGGAGTTTGACATCTGCTACACGTCAGTGCTG
AAGCGGGCCATCCGCACCCTCTGGGCCATCCTGGACGGCACGGACCAGATGTGGCTGCCTGTGGTGCG
CACTTGGCGCCTCAATGAGCGGCATTACGGGGGCCTCACAGGCCTCAACAAGGCAGAAACGGCCGCCA
AGCACGGGGAGGAGCAGGTGAAGATCTGGAGGCGCTCCTTCGACATCCCGCCGCCCCCGATGGACGAG
AAGCACCCCTACTACAACTCCATTAGCAAGGAGCGTCGGTACGCAGGCCTGAAGCCCGGGGAACTCCC
CACCTGCGAGAGCCTCAAGGACACCATTGCCCGGGCCCTGCCCTTCTGGAACGAGGAGATTGTTCCCC
AGATCAAGGCCGGCAAGCGAGTGCTCATTGCAGCCCACGGGAACAGCCTGCGGGGCATTGTCAAGCAC
CTGGAAGGGATGTCAGACCAGGCGATCATGGAGCTGAACCTGCCCACGGGGATCCCCATTGTGTATGA
GCTGAACAAGGAGCTGAAGCCCACCAAGCCCATGCAGTTCCTGGGTGATGAGGAAACGGTGCGGAAGG
CCATGGAGGCTGTGGCTGCCCAGGGCAAGGCCAAGTGA

| | | | |
|---|---|---|---|
| NOV5b, 311980359 | | | |
| Protein Sequence | SEQ ID NO:48 | 261 aa | MW at 29746.9 kD |

TMGHHHHHHATHRLVMVRHGESTWNQENRFCGWFDAELSEKGTEEAKRGAKAIKDAKMEFDICYTSVL
KRAIRTLWAILDGTDQMWLPVVRTWRLNERHYGGLTGLNKAETAAKHGEEQVKIWRRSFDIPPPPMDE
KHPYYNSISKERRYAGLKPGELPTCESLKDTIARALPFWNEEIVPQIKAGKRVLIAAHGNSLRGIVKH
LEGMSDQAIMELNLPTGIPIVYELNKELKPTKPMQFLGDEETVRKAMEAVAAQGKAK

| | | |
|---|---|---|
| NOV5c, CG186640-01 | SEQ ID NO:49 | 898 bp |
| DNA Sequence | ORF Start: ATG at 67 | ORF Stop: TGA at 826 |

AATTCGGTACGAGGGTTGGGAAGCAGCCGTCCCCGTCCAGAGTCCTCTGTGGTCCCTGCTGCCACCAT
GGCCACTCACCGCCTCGTGATGGTCCGGCACGGCGAGAGCACATGGAACCAGGAGAACCGTTTCTGTG
GCTGGTTCGATGCAGAGCTGAGTGAAAAGGGGACCGAGGAGGCCAAGCGGGGAGCCAAGGCCATCAAG
GATGCCAAGATGGAGTTTGACATCTGCTACACGTCAGTGCTGAAGCGGGCCATCCGCACCCTCTGGGC
CATCCTGGACGGCACGGACCAGATGTGGCTGCCTGTGGTGCGCACTTGGCGCCTCAATGAGCGGCATT
ACGGGGGCCTCACAGGCCTCAACAAGGCAGAAACGGCCGCCAAGCACGGGGAGGAGCAGGTGAAGATC
TGGAGGCGCTCCTTCGACATCCCGCCGCCCCCGATGGACGAGAAGCACCCCTACTACAACTCCATTAG
CAAGGAGCGTCGGTACGCAGGCCTGAAGCCCGGGGAACTCCCCACCTGCGAGAGCCTCAAGGACACCA
TTGCCCGGGCCCTGCCCTTCTGGAACGAGGAGATTGTTCCCCAGATCAAGGCCGGCAAGCGAGTGCTC
ATTGCAGCCCACGGGAACAGCCTGCGGGGCATTGTCAAGCACCTGGAAGGGATGTCAGACCAGGCGAT
CATGGAGCTGAACCTGCCCACGGGGATCCCCATTGTGTATGAGCTGAACAAGGAGCTGAAGCCCACCA
AGCCCATGCAGTTCCTGGGTGATGAGGAAACGGTGCGGAAGGCCATGGAGGCTGTGGCTGCCCAGGGC
AAGGCCAAGTGAGGGGTGGGCTTGGGCAATAAAGGCACCTCCCCCAACAAAAAAAAAAAAAAAAAAAA
AAAAAAAAAAAAGC

| | | | |
|---|---|---|---|
| NOV5c, CG186640-01 | | | |
| Protein Sequence | SEQ ID NO:50 | 253 aa | MW at 28765.9 kD |

MATHRLVMVRHGESTWNQENRFCGWFDAELSEKGTEEAKRGAKAIKDAKMEFDICYTSVLKRAIRTLW
AILDGTDQMWLPVVRTWRLNERHYGGLTGLNKAETAAKHGEEQVKIWRRSFDIPPPPMDEKHPYYNSI
SKERRYAGLKPGELPTCESLKDTIARALPFWNEEIVPQIKAGKRVLIAAHGNSLRGIVKHLEGMSDQA
IMELNLPTGIPIVYELNKELKPTKPMQFLGDEETVRKAMEAVAAQGKAK

| | | |
|---|---|---|
| NOV5d, CG186640-03 | SEQ ID NO:51 | 763 bp |
| DNA Sequence | ORF Start: at 1 | ORF Stop: at 760 |

ACCATGGCCACTCACCGCCTCGTGATGGTCCGGCACGGCGAGAGCACATGGAACCAGGAGAACCGTTT
CTGTGGCTGGTTCGATGCAGAGCTGAGTGAAAAGGGGACCGAGGAGGCCAAGCGGGGAGCCAAGGCCA
TCAAGGATGCCAAGATGGAGTTTGACATCTGCTACACGTCAGTGCTGAAGCGGGCCATCCGCACCCTC
TGGGCCATCCTGGACGGCACGGACCAGATGTGGCTGCCTGTGGTGCGCACTTGGCGCCTCAATGAGCG
GCATTACGGGGGCCTCACAGGCCTCAACAAGGCAGAAACGGCCGCCAAGCACGGGGAGGAGCAGGTGA
AGATCTGGAGGCGCTCCTTCGACATCCCGCCGCCCCCGATGGACGAGAAGCACCCCTACTACAACTCC
ATTAGCAAGGAGCGTCGGTACGCAGGCCTGAAGCCCGGGGAACTCCCCACCTGCGAGAGCCTCAAGGA
CACCATTGCCCGGGCCCTGCCCTTCTGGAACGAGGAGATTGTTCCCCAGATCAAGGCCGGCAAGCGAG
TGCTCATTGCAGCCCACGGGAACAGCCTGCGGGGCATTGTCAAGCACCTGGAAGGGATGTCAGACCAG
GCGATCATGGAGCTGAACCTGCCCACGGGGATCCCCATTGTGTATGAGCTGAACAAGGAGCTGAAGCC
CACCAAGCCCATGCAGTTCCTGGGTGATGAGGAAACGGTGCGGAAGGCCATGGAGGCTGTGGCTGCCC
AGGGCAAGGCCAAGT

| | | | |
|---|---|---|---|
| NOV5d, CG186640-03 | | | |
| Protein Sequence | SEQ ID NO:52 | 253 aa | MW at 28738.8 kD |

TMATHRLVMVRHGESTWNQENRFCGWFDAELSEKGTEEAKRGAKAIKDAKMEFDICYTSVLKRAIRTL
WAILDGTDQMWLPVVRTWRLNERHYGGLTGLNKAETAAKHGEEQVKIWRRSFDIPPPPMDEKHPYYNS
ISKERRYAGLKPGELPTCESLKDTIARALPFWNEEIVPQIKAGKRVLIAAHGNSLRGIVKHLEGMSDQ
AIMELNLPTGIPIVYELNKELKPTKPMQFLGDEETVRKANEAVAAQGKA

| | | |
|---|---|---|
| NOV5e, CG186640-04 | SEQ ID NO:53 | 786 bp |
| DNA Sequence | ORF Start: at 1 | ORF Stop: TGA at 784 |

ACCATGGGACATCATCACCACCATCACGCCACTCACCGCCTCGTGATGGTCCGGCACGGCGAGAGCAC
ATGGAACCAGGAGAACCGTTTCTGTGGCTGGTTCGATGCAGAGCTGAGTGAAAAGGGGACCGAGGAGG
CCAAGCGGGGAGCCAAGGCCATCAAGGATGCCAAGATGGAGTTTGACATCTGCTACACGTCAGTGCTG
AAGCGGGCCATCCGCACCCTCTGGGCCATCCTGGACGGCACGGACCAGATGTGGCTGCCTGTGGTGCG
CACTTGGCGCCTCAATGAGCGGCATTACGGGGGCCTCACAGGCCTCAACAAGGCAGAAACGGCCGCCA
AGCACGGGGAGGAGCAGGTGAAGATCTGGAGGCGCTCCTTCGACATCCCGCCGCCCCCGATGGACGAG
AAGCACCCCTACTACAACTCCATTAGCAAGGAGCGTCGGTACGCAGGCCTGAAGCCCGGGGAACTCCC
CACCTGCGAGAGCCTCAAGGACACCATTGCCCGGGCCCTGCCCTTCTGGAACGAGGAGATTGTTCCCC
AGATCAAGGCCGGCAAGCGAGTGCTCATTGCAGCCCACGGGAACAGCCTGCGGGGCATTGTCAAGCAC
CTGGAAGGGATGTCAGACCAGGCGATCATGGAGCTGAACCTGCCCACGGGGATCCCCATTGTGTATGA
GCTGAACAAGGAGCTGAAGCCCACCAAGCCCATGCAGTTCCTGGGTGATGAGGAAACGGTGCGGAAGG
CCATGGAGGCTGTGGCTGCCCAGGGCAAGGCCAAGTGA

| | | | |
|---|---|---|---|
| NOV5e, CG186640-04 | | | |
| Protein Sequence | SEQ ID NO:54 | 261 aa | MW at 29746.9 kD |

TMGHHHHHHATHRLVMVRHGESTWNQENRFCGWFDAELSEKGTEEAKRGAKAIKDAKNEFDICYTSVL
KRAIRTLWAILDGTDQMWLPVVRTWRLNERHYGGLTGLNKAETAAKHGEEQVKIWRRSFDIPPPPMDE
KHPYYNSISKERRYAGLKPGELPTCESLKDTIARALPFWNEEIVPQIKAGKRVLIAAHGNSLRGIVKH
LEGMSDQAIMELNLPTGIPIVYELNKELKPTKPMQFLGDEETVRKAMEAVAAQGKAK

A ClustalW comparison of the above protein sequences yields the following sequence alignment shown in Table E7.

TABLE E7
Comparison of the NOV5 protein sequences.

| | |
|---|---|
| NOV5a | ————————MATHRLVMVRHGETTWNQENRFCGWFDAELSEKGTEEAKRGAKAIKDAKMEF |
| NOV5b | TMGHHHHHHATHRLVMVRHGESTWNQENRFCGWFDAELSEKGTEEAKRGAKAIKDAKMEF |
| NOV5c | ————————MATHRLVMVRHGESTWNQENRFCGWFDAELSEKGTEEAKRGAKAIKDAKMEF |
| NOV5d | ———————TMATHRLVMVRHGESTWNQENRFCGWFDAELSEKGTEEAKRGAKAIKDAKMEF |
| NOV5e | TMGHHHHHHATHRLVMVRHGESTWNQENRFCGWFDAELSEKGTEEAKRGAKAIKDAKMEF |
| NOV5a | DICYTSVLKRAIRTLWAILDGTDQMWLPVVRTWRFNERHYGGLTGFNKAETAAKHGEEQV |
| NOV5b | DICYTSVLKRAIRTLWAILDGTDQMWLPVVRTWRLNERHYGGLTGLNKAETAAKHGEEQV |
| NOV5c | DICYTSVLKRAIRTLWAILDGTDQMWLPVVRTWRLNERHYGGLTGLNKAETAAKHGEEQV |
| NOV5d | DICYTSVLKRAIRTLWAILDGTDQMWLPVVRTWRLNERHYGGLTGLNKAETAAKHGEEQV |
| NOV5e | DICYTSVLKRAIRTLWAILDGTDQMWLPVVRTWRLNERHYGGLTGLNKAETAAKHGEEQV |
| NOV5a | RSWRRSFDIPPPPMDEKHPYYNSISKERRYAGLKPGELPTCESLKDTIARALPFWNEEIV |
| NOV5b | KIWRRSFDIPPPPMDEKHPYYNSISKERRYAGLKPGELPTCESLKDTIARALPFWNEEIV |
| NOV5c | KIWRRSFDIPPPPMDEKHPYYNSISKERRYAGLKPGELPTCESLKDTIARALPFWNEEIV |
| NOV5d | KIWRRSFDIPPPPMDEKHPYYNSISKERRYAGLKPGELPTCESLKDTIARALPFWNEEIV |
| NOV5e | KIWRRSFDIPPPPMDEKHPYYNSISKERRYAGLKPGELPTCESLKDTIARALPFWNEEIV |
| NOV5a | PQIKAGKRVLIAAHGNSLRGIVKHLEGMSDQAIMELNLPTGIPIVYELNKELKPTKPMQF |
| NOV5b | PQIKAGKRVLIAAHGNSLRGIVKHLEGMSDQAIMELNLPTGIPIVYELNKELKPTKPMQF |
| NOV5c | PQIKAGKRVLIAAHGNSLRGIVKHLEGMSDQAIMELNLPTGIPIVYELNKELKPTKPMQF |
| NOV5d | PQIKAGKRVLIAAHGNSLRGIVKHLEGMSDQAIMELNLPTGIPIVYELNKELKPTKPMQF |
| NOV5e | PQIKAGKRVLIAAHGNSLRGIVKHLEGMSDQAIMELNLPTGIPIVYELNKELKPTKPMQF |
| NOV5a | LGDEETVRKAMEAVAAQGKAK |
| NOV5b | LGDEETVRKAMEAVAAQGKAK |
| NOV5c | LGDEETVRKAMEAVAAQGKAK |
| NOV5d | LGDEETVRKAMEAVAAQGKA— |
| NOV5e | LGDEETVRKAMEAVAAQGKAK |
| NOV5a | (SEQ ID NO:46) |
| NOV5b | (SEQ ID NO:48) |
| NOV5c | (SEQ ID NO:50) |
| NOV5d | (SEQ ID NO:52) |
| NOV5e | (SEQ ID NO:54) |

Further analysis of the NOV5a protein yielded the following properties shown in Table E8.

TABLE E8
Protein Sequence Properties NOV5a

| SignalP | No Known Signal Sequence Predicted | | | |
|---|---|---|---|---|
| analysis: | | | | |
| PSORT II | PSG: a new signal peptide prediction method | | | |
| analysis: | N-region: length 10; pos. chg 2; neg. chg 0 | | | |
| | H-region: length 2; peak value −7.76 | | | |
| | PSG score: −12.16 | | | |
| | GvH: von Heijne's method for signal seq. recognition | | | |
| | GvH score (threshold: −2.1): −12.65 | | | |
| | possible cleavage site: between 14 and 15 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 5.41 (at 204) | | | |
| | ALOM score: 5.41 (number of TMSs: 0) | | | |
| | MITDISC: discrimination of mitochondrial targeting seq | | | |
| | R content: | 2 | Hyd Moment(75): | 10.06 |
| | Hyd Moment(95): | 6.20 | G content: | 1 |
| | D/E content: | 2 | S/T content: | 3 |

Score: −4.24
Gavel: prediction of cleavage sites for mitochondrial preseq
R-2 motif at 20 VRH|GE
NUCDISC: discrimination of nuclear localization signals
pat4: none
pat7: none
bipartite: none
content of basic residues: 15.4%
NLS Score: −0.47
KDEL: ER retention motif in the C-terminus: none
ER Membrane Retention Signals:
XXRR-like motif in the N-terminus: ATHR
KKXX-like motif in the C-terminus: QGKA
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear
discrimination
Prediction: cytoplasmic
Reliability: 89
COIL: Lupas's algorithm to detect coiled-coil regions
total: 0 residues
Final Results (k = 9/23):
43.5%: cytoplasmic
30.4%: nuclear
26.1%: mitochondrial
>> prediction for CG186640-02 is cyt (k = 23)

A search of the NOVSa protein against the Geneseq database, a proprietary database that contains sequences published in patents and patent publication, yielded several homologous proteins shown in Table E9.

TABLE E9
Geneseq Results for NOV5a

| | | NOV5a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG09674 | Novel human diagnostic protein | 1 . . . 253 | 191/254 (75%) | e−113 |
| | #9665 - Homo sapiens, 265 aa. | 11 . . . 264 | 221/254 (86%) | |
| | [WO200175067-A2, 11-OCT-2001] | | | |
| ABG05571 | Novel human diagnostic protein | 1 . . . 253 | 181/258 (70%) | 7e−99 |
| | #5562 - Homo sapiens, 270 aa. | 12 . . . 269 | 209/258 (80%) | |
| | [WO200175067-A2, 11-OCT-2001] | | | |
| ABB64868 | Drosophila melanogaster polypeptide | 4 . . . 253 | 167/251 (66%) | 2e−93 |
| | SEQ ID NO 21396 - Drosophila | 42 . . . 292 | 196/251 (77%) | |
| | melanogaster, 292 aa. | | | |
| | [WO200171042-A2, 27-SEP-2001] | | | |
| ABB66376 | Drosophila melanogaster polypeptide | 16 . . . 253 | 159/239 (66%) | 6e−88 |
| | SEQ ID NO 25920 - Drosophila | 33 . . . 271 | 185/239 (76%) | |
| | melanogaster, 271 aa. | | | |
| | [WO200171042-A2, 27-SEP-2001] | | | |
| ABG19326 | Novel human diagnostic protein | 4 . . . 251 | 161/256 (62%) | 2e−84 |
| | #19317 - Homo sapiens, 333 aa. | 35 . . . 290 | 198/256 (76%) | |
| | [WO200175067-A2, 11-OCT-2001] | | | |

In a BLAST search of public sequence databases, the NOV5a protein was found to have homology to the proteins shown in the BLASTP data in Table E10.

TABLE E10
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| PMHUYM | M - human, 253 aa. | 1 . . . 253 | 250/253 (98%) | e−146 |
| P15259 | Phosphoglycerate mutase 2 (EC | 2 . . . 253 | 247/252 (98%) | e−146 |
| | 5.4.2.1) (EC 5.4.2.4) (EC 3.1.3.13) | 1 . . . 252 | 249/252 (98%) | |
| | (Phosphoglycerate mutase isozyme M) | | | |
| | (PGAM-M) (BPG-dependent PGAM 2) | | | |
| | (Muscle-specific phosphoglycerate | | | |
| | mutase) - Homo sapiens (Human), 252 aa. | | | |
| PMRTYM | phosphoglycerate mutase (EC 5.4.2.1) | 1 . . . 253 | 232/253 (91%) | e−136 |
| | M - rat, 253 aa. | 1 . . . 253 | 241/253 (94%) | |
| P16290 | Phosphoglycerate mutase 2 (EC | 2 . . . 253 | 231/252 (91%) | e−136 |
| | 5.4.2.1) (EC 5.4.2.4) (EC 3.1.3.13) | 1 . . . 252 | 240/252 (94%) | |
| | (Phosphoglycerate mutase isozyme M) | | | |
| | (PGAM-M) (BPG-dependent PGAM 2) | | | |
| | (Muscle-specific phosphoglycerate | | | |
| | mutase) - Rattus norvegicus (Rat), 252 aa. | | | |
| O70250 | Phosphoglycerate mutase 2 (EC | 3 . . . 253 | 227/251 (90%) | e−134 |
| | 5.4.2.1) (EC 5.4.2.4) (EC 3.1.3.13) | 2 . . . 252 | 237/251 (93%) | |
| | (Phosphoglycerate mutase isozyme M) | | | |
| | (PGAM-M) (BPG-dependent PGAM 2) | | | |
| | (Muscle-specific phosphoglycerate | | | |
| | mutase) - Mus musculus (Mouse), 252 aa. | | | |

PFam analysis predicts that the NOV5a protein contains the domains shown in the Table E11.

TABLE E11
Domain Analysis of NOV5a

| | | Identities/ | |
|---|---|---|---|
| | | Similarities for | |
| Pfam | NOV5a | the Matched | |
| Domain | Match Region | Region | Expect Value |
| PGAM | 4 . . . 230 | 132/230 (57%) | 1.3e−132 |
| | | 209/230 (91%) | |

### EXAMPLE E3

### Expression Profiles of the Human Phosphoglycerate Mutase 2 (PGM2) and Human Phosphoglycerate Mutase 1 (PGM1) Genes

The protocol for quantitative expression analysis is disclosed in Example Q9.

Expression of genes CG186640-02 (PGM2) and CG115294-01 (PGM1) was assessed using the primer-probe sets Ag2379 and Ag6474 described in Tables E12 and E13. Results of the RTQ-PCR runs are shown in Tables E14 and E15. Ag2379 and Ag6474 are specific for CG115294-01 and CG186640-02 respectively.

TABLE E12
Probe Name Ag2379

| | | | Start | SEQ ID |
|---|---|---|---|---|
| Primers | Sequences | Length | Position | No |
| Forward | 5′-ctacgagatgctggctatgagt-3′ | 22 | 164 | 201 |
| Probe | TET-5′-ttgacatctgcttcacctcagtgcag-3′- | 26 | 186 | 202 |
| | TAMRA | | | |
| Reverse | 5′-gatcaatggcatctagcactgt-3′ | 22 | 236 | 203 |

TABLE E13.
Probe Name Ag6474

| | | | Start | SEQ ID |
|---|---|---|---|---|
| Primers | Sequences | Length | Position | No |
| Forward | 5′-ggaggagcaggtgaagatct-3′ | 20 | 327 | 204 |
| Probe | TET-5′-cgatggacgagaagcacccctactac-3′- | 26 | 377 | 205 |
| | TAMRA | | | |
| Reverse | 5′-ccgacgctccttgctaa-3′ | 17 | 410 | 206 |

TABLE E14
General_screening_panel_v1.6

| | Rel. Exp. (%) | Rel. Exp. (%) |
|---|---|---|
| | Ag6474, Run | Ag2379, Run |
| Tissue Name | 277225783 | 277227794 |
| Adipose | 3.1 | 3.0 |
| Melanoma* Hs688(A).T | 0.0 | 52.1 |
| Melanoma* Hs688(B).T | 0.1 | 42.3 |
| Melanoma* M14 | 0.0 | 42.6 |
| Melanoma* LOXIMVI | 0.0 | 51.8 |
| Melanoma* SK-MEL-5 | 0.1 | 45.1 |
| Squamous cell | 0.0 | 29.5 |
| carcinoma SCC-4 | | |
| Testis Pool | 6.1 | 4.6 |
| Prostate ca.* | 0.1 | 40.6 |
| (bone met) PC-3 | | |
| Prostate Pool | 1.0 | 3.2 |
| Placenta | 0.0 | 6.6 |
| Uterus Pool | 0.1 | 2.1 |
| Ovarian ca. OVCAR-3 | 0.8 | 39.8 |
| Ovarian ca. SK-OV-3 | 0.0 | 25.2 |
| Ovarian ca. OVCAR-4 | 0.1 | 32.1 |
| Ovarian ca. OVCAR-5 | 0.6 | 42.3 |
| Ovarian ca. IGROV-1 | 1.4 | 32.8 |
| Ovarian ca. OVCAR-8 | 0.1 | 12.9 |
| Ovary | 0.1 | 3.8 |
| Breast ca. MCF-7 | 0.0 | 31.6 |
| Breast ca. MDA-MB-231 | 0.1 | 100.0 |
| Breast ca. BT 549 | 0.0 | 54.0 |
| Breast ca. T47D | 0.1 | 13.3 |
| Breast ca. MDA-N | 0.0 | 17.6 |
| Breast Pool | 0.0 | 4.5 |
| Trachea | 0.5 | 5.6 |
| Lung | 0.2 | 1.4 |
| Fetal Lung | 0.3 | 7.5 |
| Lung ca. NCI-N417 | 0.4 | 18.8 |
| Lung ca. LX-1 | 0.4 | 27.4 |
| Lung ca. NCI-H146 | 6.3 | 8.0 |
| Lung ca. SHP-77 | 12.5 | 43.8 |
| Lung ca. A549 | 0.1 | 43.2 |
| Lung ca. NCI-H526 | 0.2 | 6.6 |
| Lung ca. NCI-H23 | 0.2 | 24.1 |
| Lung ca. NCI-H460 | 0.0 | 18.6 |
| Lung ca. HOP-62 | 0.0 | 27.7 |
| Lung ca. NCI-H522 | 0.1 | 19.8 |
| Liver | 1.0 | 2.1 |
| Fetal Liver | 0.2 | 3.8 |
| Liver ca. HepG2 | 0.5 | 18.3 |
| Kidney Pool | 0.6 | 7.1 |
| Fetal Kidney | 0.4 | 4.7 |
| Renal ca. 786-0 | 0.0 | 56.6 |
| Renal ca. A498 | 0.0 | 21.8 |
| Renal ca. ACHN | 0.0 | 25.7 |
| Renal ca. UO-31 | 0.0 | 33.2 |
| Renal ca. TK-10 | 0.1 | 22.4 |
| Bladder | 0.1 | 8.7 |
| Gastric ca. (liver | 0.1 | 22.8 |
| met.) NCI-N87 | | |
| Gastric ca. KATO III | 0.0 | 39.2 |
| Colon ca. SW-948 | 0.0 | 18.3 |
| Colon ca. SW480 | 0.1 | 62.4 |
| Colon ca.* | 0.1 | 28.7 |
| (SW480 met) SW620 | | |
| Colon ca. HT29 | 0.1 | 26.2 |
| Colon ca. HCT-116 | 0.1 | 79.6 |
| Colon ca. CaCo-2 | 0.1 | 31.6 |
| Colon cancer tissue | 0.2 | 11.5 |
| Colon ca. SW1116 | 0.0 | 11.9 |
| Colon ca. Colo-205 | 0.0 | 14.8 |
| Colon ca. SW-48 | 0.0 | 14.8 |
| Colon Pool | 0.2 | 4.9 |
| Small Intestine Pool | 0.4 | 3.6 |
| Stomach Pool | 0.0 | 3.6 |
| Bone Marrow Pool | 0.1 | 1.9 |
| Fetal Heart | 27.0 | 6.0 |
| Heart Pool | 31.0 | 1.9 |
| Lymph Node Pool | 0.1 | 6.5 |
| Fetal Skeletal Muscle | 22.1 | 1.3 |
| Skeletal Muscle Pool | 100.0 | 0.8 |
| Spleen Pool | 0.2 | 4.5 |
| Thymus Pool | 0.7 | 5.6 |
| CNS cancer | 0.0 | 64.2 |
| (glio/astro) U87-MG | | |
| CNS cancer | 0.0 | 57.8 |
| (glio/astro) U-118-MG | | |
| CNS cancer | 0.2 | 38.7 |
| (neuro; met) SK-N-AS | | |
| CNS cancer (astro) SF-539 | 0.4 | 45.7 |
| CNS cancer (astro) SNB-75 | 0.0 | 50.7 |
| CNS cancer (glio) SNB-19 | 1.4 | 32.1 |
| CNS cancer (glio) SF-295 | 0.2 | 55.9 |
| Brain (Amygdala) Pool | 1.0 | 13.6 |
| Brain (cerebellum) | 13.0 | 33.0 |
| Brain (fetal) | 0.9 | 11.0 |
| Brain (Hippocampus) Pool | 1.6 | 12.9 |
| Cerebral Cortex Pool | 0.9 | 13.0 |
| Brain (Substantia nigra) Pool | 2.1 | 11.3 |
| Brain (Thalamus) Pool | 1.4 | 17.7 |
| Brain (whole) | 1.0 | 15.2 |
| Spinal Cord Pool | 3.3 | 9.5 |
| Adrenal Gland | 0.3 | 11.0 |
| Pituitary gland Pool | 1.3 | 1.7 |
| Salivary Gland | 0.4 | 4.1 |
| Thyroid (female) | 0.9 | 3.6 |
| Pancreatic ca. CAPAN2 | 0.0 | 19.5 |
| Pancreas Pool | 0.4 | 3.6 |

TABLE E15
Panel 5 Islet

| | Rel. Exp. (%) | Rel. Exp. (%) |
|---|---|---|
| | Ag6474, Run | Ag2379, Run |
| Tissue Name | 268366291 | 263467358 |
| 97457_Patient-02go_adipose | 0.0 | 4.4 |
| 97476_Patient-07sk_skeletal | 8.3 | 4.7 |
| muscle | | |
| 97477_Patient-07ut_uterus | 0.0 | 6.2 |
| 97478_Patient-07pl_placenta | 0.0 | 10.5 |
| 99167_Bayer Patient 1 | 0.5 | 100.0 |
| 97482_Patient-08ut uterus | 0.0 | 8.0 |
| 97483_Patient-08pl_placenta | 0.0 | 8.4 |
| 97486_Patient-09sk_skeletal | 17.6 | 1.2 |
| muscle | | |
| 97487_Patient-09ut_uterus | 0.1 | 9.7 |
| 97488_Patient-09pl_placenta | 0.0 | 5.5 |
| 97492_Patient-10ut_uterus | 0.0 | 9.6 |
| 97493_Patient-10pl_placenta | 0.0 | 16.4 |
| 97495_Patient-11go_adipose | 0.0 | 4.4 |
| 97496_Patient-11sk_skeletal | 69.7 | 2.2 |
| muscle | | |
| 97497_Patient-11ut_uterus | 0.0 | 13.1 |
| 97498_Patient-11pl_placenta | 0.0 | 8.9 |
| 97500_Patient-12go_adipose | 0.0 | 9.7 |
| 97501_Patient-12sk_skeletal | 100.0 | 6.5 |
| muscle | | |
| 97502_Patient-12ut_uterus | 0.1 | 10.8 |
| 97503_Patient-12pl_placenta | 0.0 | 9.1 |
| 94721_Donor 2 U - | 0.0 | 35.6 |
| A_Mesenchymal Stem Cells | | |
| 94722_Donor 2 U - | 0.0 | 32.5 |
| B_Mesenchymal Stem Cells | | |
| 94723_Donor 2 U - | 0.2 | 22.2 |
| C_Mesenchymal Stem Cells | | |
| 94709_Donor 2 AM - A_adipose | 0.0 | 60.3 |
| 94710_Donor 2 AM - B_adipose | 0.0 | 42.3 |
| 94711_Donor 2 AM - C_adipose | 0.0 | 27.4 |
| 94712_Donor 2 AD - A_adipose | 0.2 | 35.8 |
| 94713_Donor 2 AD - B_adipose | 0.1 | 59.0 |
| 94714_Donor 2 AD - C_adipose | 0.3 | 56.6 |
| 94742_Donor 3 U - | 0.0 | 26.2 |
| A_Mesenchymal Stem Cells | | |
| 94743_Donor 3 U - | 0.0 | 36.1 |
| B_Mesenchymal Stem Cells | | |
| 94730_Donor 3 AM - A_adipose | 0.0 | 54.7 |
| 94731_Donor 3 AM - B_adipose | 0.0 | 42.9 |
| 94732_Donor 3 AM - C_adipose | 0.0 | 51.1 |
| 94733_Donor 3 AD - A_adipose | 0.0 | 96.6 |
| 94734_Donor 3 AD - B_adipose | 0.0 | 49.0 |
| 94735_Donor 3 AD - C_adipose | 0.0 | 80.1 |
| 77138_Liver_Hep G2untreated | 0.2 | 46.3 |
| 73556_Heart_Cardiac stromal | 0.0 | 12.8 |
| cells (primary) | | |
| 81735_Small Intestine | 0.2 | 11.0 |
| 72409_Kidney_Proximal | 0.0 | 18.4 |
| Convoluted Tubule | | |
| 82685_Small intestine_Duodenum | 0.0 | 10.9 |
| 90650_Adrenal_A | 0.0 | 14.6 |
| drenocortical adenoma | | |
| 72410_Kidney_HRCE | 0.2 | 72.7 |
| 72411_Kidney_HRE | 0.1 | 34.2 |
| 73139_Uterus_Uterine | 0.0 | 19.1 |
| smooth muscle cells | | |

General screening panel v1.6 Summary: PGM2 is specifically expressed in skeletal muscle, which is consistent with the literature data. PGM1 is ubiquitously expressed with higher in expression in the cerebellum compared to PGM2 (CTs=26.8 vs. 29.5) and conversely is lower in skeletal muscle than PGM2 (CTs=32.2 vs 26.6). Specific expression of PGM2 in skeletal muscle suggests that a drug would target preliminary skeletal muscle and would not interfere significantly with glycolysis in other tissues, specifically in brain and pancreatic islets.

Panel 5 Islet Summary: In Panel 5I, PGM2 is expressed only in skeletal muscle which is in agreement with the expression pattern of Panel 1.4. Moreover, PGM2 is significantly up-regulated in diabetic muscle (patient 12) relative to non-diabetic skeletal muscle (patients 7, patient 9, patient 11). These results further support the hypothesis that increases in phosphoglycerate mutase level/activity may contribute to the development of diabetes. Therefore, inhibition of PGM2 may be beneficial for the treatment of diabetes. PGM1 is more widely expressed with high expression in islet cells (Bayer patient 1).

### EXAMPLE E4

### Assays for Modulators of Phosphoglycerate Mutase 2

One potential assay that may be used to screen for modulators of Phosphoglycerate mutase 2 is to measure the production of glycerate-3-phosphate formed in following reaction catalysed by phosphoglycerate mutase:
2-phospho-D-glycerate+2,3-diphosphoglycerate <=>3-phospho-D-glycerate+2,3-diphosphoglycerate

Production of glycerate-3-phosphate could be measured by reaction coupled to phophoglycerate kinase and glyceraldehydephosphate dehydrogenase as described in Rosa R, Blouquit Y, Calvin M C, Prome D, Prome J C, Rosa J. Isolation, characterization, and structure of a mutant 89 Arg—Cys bisphosphoglycerate mutase. Implication of the active site in the mutation. J Biol. Chem. 1989 May 15; 264 (14):7837-43; PMID: 2542247.

Our results indicate that a modulator of Phosphoglycerate mutase 2 activity, such as an inhibitor, activator, antagonist, or agonist of Phosphoglycerate mutase 2 may be useful for treatment of such disorders as obesity, diabetes, and insulin resistance, as well as for enhancement of insulin secretion.

F. NOV6—Adenosine A1 Receptor

Adenosine A1 receptor (Adora1) is a G-protein coupled receptor found at the plasma membrane of multiple cell types. Adora1 is believed to be involved in heart contractility, adipose tissue lipolysis, glomerular filtration and tubulo-glomerular feedback in the kidney, and sympathetic and parasympathetic activity in the nervous system. (Fredholm B B, Ijzerman A P, Jacobson K A, Klotz K N, Linden J, International Union of Pharmacology. XXV. Nomenclature and classification of adenosine receptors. Pharmacol Rev. 2001 December; 53 (4):527-52. Review. PMID: 11734617) It is a member of the adenosine receptor family comprised of adenosine A1, A2A, A2B and A3 receptors. Adenosine is the preferred endogenous ligand for these receptors. However, the adenosine receptors are not highly homologous, with the greatest percent identity found between the A1 and the A2B receptors (56% sequence identity at the protein level). The low level of sequence identity is illustrated by the fact that selective agonists and antagonists have been identified for the all of the adenosine receptors with the exception of A2B, which lacks a selective antagonist (Fredholm B B, IJzerman A P, Jacobson K A, Klotz K N, Linden J, International Union of Pharmacology. XXV. Nomenclature and classification of adenosine receptors. Pharmacol Rev. 2001 December; 53 (4):527-52. Review. PMID: 11734617).

Adenosine is the main agonist for this receptor family. Under normal conditions, adenosine is continuously formed intracellularly in a variety of cell types, as well as extracellularly in a variety of tissue compartments. The continuous synthesis and breakdown of adenosine in any cell type or tissue compartment varies with physiological circumstances, and this variance implies that the biology of adenosine receptors will be complicated, as well as tissue and cell-type specific. Adenosine A1 receptor knockout mice that do not respond to Adora1 agonists have been reported (Johansson B, Halldner L, Dunwiddie T V, Masino S A, Poelchen W, Gimenez-Llort L, Escorihuela R M, Fernandez-Teruel A, Wiesenfeld-Hallin Z, Xu X J, Hardemark A, Betsholtz C, Herlenius E, Fredholm B B. Hyperalgesia, anxiety, and decreased hypoxic neuroprotection in mice lacking the adenosine A1 receptor. Proc Natl Acad Sci USA. 2001 Jul. 31; 98 (16):9407-12. PMID: 11470917). The mice were viable, fertile and without any gross abnormalities. Although Adora1 is hypothesized to be important for cardiovascular function, arterial blood pressure and heart rates were indistinguishable between A1 receptor knockouts and wild type mice (Johansson et. al.). To date, our knowledge of the role of adenosine A1 receptors in normal physiology is incomplete.

The distribution of A1 receptors in tissues has been studied using selective radioligands (Fredholm et. al.). High expression has been reported in the brain (cortex, cerebellum, hippocampus), dorsal horn of the spinal cord, eye and adrenal gland (Fredholm et. al.). Intermediate levels of Adora1 were reported in other brain regions, skeletal muscle, liver, kidney, adipose tissue, salivary glands, esophagus, colon and testis. The specific role of Adora1 in all of these diverse cell types is not currently known.

Insulin-secreting beta cells are located in the pancreatic islets of Langerhans. Insulin secretion can be measured while perfusing the isolated pancreas with glucose (the physiologic signal). Structural analogues of adenosine have been used to perfuse the isolated rat pancreas, with a resultant decrease in insulin secretion. (Hillaire-Buys D, Chapal J, Bertrand G, Petit P, Loubatieres-Mariani M M. Purinergic receptors on insulin-secreting cells. Fundam Clin Pharmacol 1994 8 (2):117-27. PMID: 8020870). However, this result did not confirm Adora1 specificity (because adenosine acts on all adenosine receptor subtypes) and was not specific for human islet cells.

Aminophylline is a non-selective but potent adenosine receptor family antagonist. Intravenous administration of aminophylline stimulated insulin secretion in patients with Type 2 diabetes, suggesting that an adenosine receptor family antagonist may be involved in turning on insulin secretion (Arias A M, Bisschop P H, Ackerman M T, Nijpels G, Endert E, Romijn J A, Sauerwein H P. Aminophylline stimulates insulin secretion in patients with Type 2 diabetes mellitus. Metabolism 2001 September; 50 (9):1030-5. PMID:11555834). The present invention is for the use of a specific Adora1 antagonist to enhance insulin secretion and lower blood glucose. U.S. Pat. No. 6,407,076 describes compounds which are agonists of adenosine A1 receptor and function as inhibitors of lipolysis and may also have the ability to lower elevated blood glucose.

Adenosine A1 receptor is coupled to the Gαᵢ family of G protein effector systems that inhibit cAMP production (Fredholm et. al.). Cyclic AMP has long been known as a potentiator of neurotransmitter-induced insulin secretion (Harndahl L, Jing X J, Ivarsson R, Degerman E, Ahren B, Manganiello V C, Renstrom E, Holst L S. Important role of phosphodiesterase 3B for the stimulatory action of cAMP on pancreatic beta-cell exocytosis and release of insulin. J Biol. Chem. 2002 Oct. 4; 277 (40):37446-55. PMID: 12169692). The present invention is for the use of a specific Adora1 antagonist to enhance glucose-stimulated insulin secretion (as opposed to neurotransmitter-stimulated insulin secretion).

We have shown that Adora1 is expressed in a wide variety of human tissues, with highest levels seen in brain and testis. We also documented that Adora1 is expressed in human pancreatic islet cells.

Type 2 diabetes in man is characterized by increased fasting and post-prandial circulating free fatty acids, and by an insulin secretory defect. The increased levels of free fatty acids are hypothesized to be a major contributor to the insulin secretory defect. The pancreatic islets of Langerhans contain beta cells that secrete insulin in response to increases in blood glucose. Culturing islet cells in vitro with oleate, one of the three most abundant circulating fatty acids in man, provides a means of studying the beta cell secretory defect in Type 2 diabetes. We discovered that islet cells cultured for 5 days in vitro with oleate have a significant deficit in glucose-stimulated insulin secretion and a 1.9-fold upregulation of Adora1 mRNA. Thus, the increased expression of Adora1 in oleate-treated islets reflects increased receptor activation, decreased cAMP levels in the islet cell, and a resultant diminution of insulin secretion. A preferred method of the invention is the use of the adenosine A1 receptor for identifying an antagonist that would be beneficial in the treatment of Type 2 diabetes. As such the current invention embodies the use of recombinantly expressed and/or endogenously expressed protein in various screens to identify such therapeutic antibodies and/or therapeutic small molecules.

In one embodiment, the present invention describes the specific upregulation of Adora1 mRNA in rat islet cells cultured in oleate. Islets cultured in oleate have a significant deficit in insulin secretion. The upregulation and activation of Adora1 in oleate-cultured islets may be the cause of the suppressed insulin secretion.

In particular the invention relates to the use of Adora1 protein as a diagnostic and/or target for small molecule drugs and antibody therapeutics. We documented that Adora1 is expressed in a wide variety of human tissues, with highest expression in brain and testis. We also discovered novel expression of Adora1 in human pancreatic islet cells. Furthermore, we have discovered that adenosine A1 receptor mRNA is upregulated 1.9-fold in islets treated with oleate, which has been identified in the art to suppress glucose-stimulated insulin secretion, versus control islets. The increased expression of Adora1 in oleate-treated islets reflects increased receptor activation, decreased cAMP levels in the islet cell, and a resultant diminution of insulin secretion. Preferably, in one aspect, agonist activation of Adora1 decreases cellular cAMP accumulation. Thus, the present invention describes a role for activation of Adora1 in the inhibition of glucose-stimulated (but not neurotransmitter-stimulated) insulin secretion.

Not to be limited by a particular mechanism of action, we have discovered that inhibition of Adora1 may have beneficial effects for treating Type 2 diabetes. Specifically, Adora1 is expressed in several metabolic tissues, including pancreatic islets of Langerhans. Thus, we have shown that culture of islets in 2 mM oleate for 5 days results in increased expression of islet cell Adora1. Increased expression and/or activation of Adora1 may contribute to the insulin secretory defect in oleate-treated islets and patients with Type 2 diabetes. The finding that suppression of glucose-stimulated insulin secretion in islet cells is correlated with up-regulation of adenosine A1 receptor mRNA in pancreatic islet cells, indicates a role for Adora1 in insulin secretion and glucose homeostasis. Therefore, an antagonist of Adora1 is useful for the treatment of diabetes. In a particular embodiment of the invention, Adora1 is a target for screening antagonists of Adora1 expression or activity. As such the current invention embodies the use of recombinantly expressed and/or endogenously expressed protein in various screens to identify adenosine A1 receptor antagonist therapeutic antibodies and/or therapeutic small molecules beneficial in the treatment of Type 2 diabetes.

Furthermore, our results indicate that a modulator of Adora1 activity, such as an inhibitor, activator, antagonist, or agonist of Adora1 may be useful for treatment of such disorders as obesity, diabetes, and insulin resistance, as well as for enhancement of insulin secretion.

Discovery Process

The following sections describe the study design(s) and the techniques used to identify the Adenosine A1 receptor—encoded protein and any variants, thereof, as being suitable as diagnostic markers, targets for an antibody therapeutic and targets for a small molecule drugs for Obesity and Diabetes.

### EXAMPLE F1

### Rat Pancreatic Islets Study

A protocol Rat Pancreatic Islet study is disclosed in Example Q2.

Greater than 80% of Type 2 diabetes in man is associated with obesity. An important clinical goal in the early phases of Type II diabetes is to increase insulin secretion from the beta cells of the pancreas. Numerous agents have been identified that can modulate insulin secretion experimentally and in therapeutic situations. When applied to isolated rat pancreatic islets, the changes in gene expression can be correlated with insulin secretion. In this study, acute and chronic changes in gene expression were examined from islets treated with an agent after short (4 hour) and long-term (5 days) exposure, respectively, compared with the basal state (11 mM glucose). The agents included elevated (25 mM) glucose, glucose (11 mM) and exendin-4 (1 nM), glucose (11 mM) and glybenclamide (50 uM) and glucose (11 mM) and oleate (2 mM). A characteristic of obesity-related Type 2 diabetes is an increase in both fasting and post-prandial circulating free fatty acids. The increased levels of free fatty acids are hypothesized to be a major contributor to the insulin secretory defect seen in this disease. Culturing islet cells in vitro with oleate, one of the three most abundant circulating fatty acids in humans provides a means of studying the beta cell secretory defect in Type 2 diabetes. Protocol for differential gene expression analysis, GeneCalling®, is disclosed in Example Q7.

Results

A fragment of the rat Adenosine A1 Receptor gene was initially found to be up-regulated by 1.9 fold in a sample derived from oleate treated islet cells, 5 days after exposure to the oleate, a known suppressor of insulin secretion, relative to islet cells treated with glucose alone using CuraGen's GeneCalling® method of differential gene expression. A differentially expressed rat gene fragment migrating, at approximately 370 nucleotides in length was definitively identified as a component of the rat Adenosine A1 Receptor cDNA. The method of competitive PCR was used for confirmation of the gene assessment. The electropherographic peaks corresponding to the gene fragment of the rat Adenosine A1 Receptor were ablated when a gene-specific primer (shown Table F1) competes with primers in the linker-adaptors during the PCR amplification. The peaks at 370 nt in length were ablated in the sample from both the oleate treated and basal state islet cells.

TABLE F1

The sequence of the 370 nucleotide-long gene fragment and the gene-
specific primers used for competitive PCR are indicated on the cDNA
sequence of the rat adenosine A1 receptor fragment (from 1003 to
2337; SEQ ID NO:207) and are shown below in bold. The gene-specific
primers at the 5′ and 3′ ends of the fragment are
underlined.

| | |
|---|---|
| 1003 | GCCAAGTCGC TGGCCCTCAT CCTCTTCCTC TTTGCCCTCA GCTGGCTGCC GCTGCATATC |
| 1063 | TTGAACTGTA TCACCCTCTT CTGCCCCACC TGCCAGAAAC CCAGCATTCT GATCTACATC |
| 1123 | GCCATCTTCC TCACACACGG CAACTCCGCC ATGAACCCCA TCGTCTATGC CTTCCGGATC |
| 1183 | CACAAGTTCC GGGTCACCTT TCTGAAGATT TGGAATGACC ACTTCCGATG CCAGCCTAAG |
| 1243 | CCTCCCATCG ATGAAGACCT CCCAGAGGAG AAAGCTGAGG ACTAGACTCT GCCTTGCTCC |
| 1303 | GTCTAGCCCA TGCCCAGCGG CTCTCTGTTC AACTCCCACG TCCTCCCTGT CCCACCCTGT |
| 1363 | CCCACTGTCC CTCCTCAGTT TTCCCAGCTG GGGTGTAGGC TGTGGCATAG CGCGCATCTT |
| 1423 | TTCTTAAAGC TTTTACTTTG AGACGTCATG GAAAACTTAA GAGGTACACA TGGAGAAGAC |
| 1483 | ATGATCACAG AAGGGAAACA GCATAGAAGC ATCGATGCCT GCAGCTAGTG CTGGAGCTGG |
| 1543 | AGCTGGAGTT GAGTTTGACA TGATACAGGG ACTGCAGGAA TGAACTAGTA TTCCTCTCCT |
| 1603 | TCTTCCTCAC CCTCACCCTC CACAGAATCC ACACCAACCT CCTCATAATC CTTCTCTAGG |
| 1663 | GCAGCCATGT CCTCACGGGC CTCAGAGAAC TCTCCCTCCT CCATGCCCTC ACCCACGTAC |
| 1723 | CAGTGCACAA AGGCACGCTT GGCATACATC AGATCAAACT TGTGATCCAG GCGAGCCCAA |
| 1783 | GCCTCAGCAA TGGCTGTGGT GTTGCTCAGC ATACACACAG CTCTCTGGAC CTTGGCCAGG |
| 1843 | TCGCCACCAG GTACCACAGT GGGAGGCTGG TAATTAATGC CAACCTTGAA GCCAGTGGGG |
| 1903 | CACCAGTCCA CAAACTGGAT GGTACGCTTG GTCTTGATGG TGGCAATGGC AGCATTGACA |
| 1963 | TCTTTGGGGA CCACATCACC ACGGTACAGC AGGCAGCAAG CCATGTATTT ACCATGGCGA |
| 2023 | GGGTCACATT TCACCATCTG GTTGGCTGGC TCAAAGCAGG CATTGGTGAT CTCTGCTACA |
| 2083 | GAAAGCTGTT CATGGTAGGC TTTCTCAGCA GAGATGACAG GGGCATAAGT GGCCAGAGGG |
| 2143 | AAGTGGATGC GAGGGTAGGG CACCAGGTTG GTCTGGAATT CTGTCAGATC AACATTCAGG |
| 2203 | GCCCCATCAA ATCTGAGGGA AGCAGTGATG GAAGACACAA TCTGGCTAAT AAGGCGGTTA |
| 2263 | AGGTTAGTGT AGGTTGGGCG CTCAATGTCG AGGTTTCTAC GACAGATGTC ATAGATGGCC |
| 2323 | TCATTGTCTA CCATG |

In addition, a second fragment of the rat Adenosine A1 Receptor gene was found to be up-regulated by 1.8 fold in the same sample of oleate treated islet cells above, (5 days after exposure to the oleate). This differentially expressed rat gene fragment migrating at approximately 383.4 nucleotides in length was also definitively identified as a component of the rat Adenosine A1 Receptor cDNa by the method of competitive PCR using a gene-specific primer (shown Table F2) to compete with primers in the linker-adaptors during the PCR amplification. The electropherographic peaks corresponding to the gene fragment at 383 nt in length were ablated in the sample from both the oleate treated and basal state islet cells.

TABLE F2

The sequence of the 383 nucleotide-long gene fragment (from 2178 to
2559) and the gene-specific primers used for competitive PCR are
indicated on the cDNA sequence of the rat adenosine A1 receptor
fragment (SEG ID NO:208) are shown in bold. The gene-specific primers
at the 5′ and 3′ ends of the fragment are underlined.

1697 CCTCCTCCAT GCCCTCACCC ACGTACCAGT GCACAAAGGC ACGCTTGGCA TACATCAGAT
1757 CAAACTTGTG ATCCAGGCGA GCCCAAGCCT CAGCAATGGC TGTGGTGTUG CTCAGCATAC
1817 ACACAGCTCT CTGGACCTTG GCCAGGTCGC CACCAGGTAC CACAGTGGGA GGCTGGTAAT
1877 TAATGCCAAC CTTGAAGCCA GTGGGGCACC AGTCCACAAA CTGGATGGTA CGCTTGGTCT
1937 TGATGGTGGC AATGGCAGCA TTGACATCTT TGGGGACCAC ATCACCACGG TACAGCAGGC
1997 AGCAAGCCAT GTATTTACCA TGGCGAGGGT CACATTTCAC CATCTGGTTG GCTGGCTCAA
2057 AGCAGGCATT GGTGATCTCT GCTACAGAAA GCTGTTCATG GTAGGCTTTC TCAGCAGAGA
2117 TGACAGGGGC ATAAGTGGCC AGAGGGAAGT GGATGCGAGG GTAGGGCACC AGGTTGGTCT
2177 GGAATTCTGT CAGATCAACA TTCAGGGCCC CATCAAATCT GAGGGAAGCA GTGATGGAAG
2237 ACACAATCTG GCTAATAAGG CGGTTAAGGT TAGTGTAGGT TGGGCGCTCA ATGTCGAGGT
2297 TTCTACGACA GATGTCATAG ATGGCCTCAT TGTCTACCAT GAAGGCACAA TCAGAGTGCT
2357 CCAGGGTGGT GTGGGTGGTG AGGATGGAAT TGTAGGGCTC AACCACAGCA GTGGAAACCT
2417 GGGGGGCTGG GTAAATGGAG AACTCCAGCT TGGACTTCTT TCCGTAGTCG ACAGAGAGCC
2477 TCTCCATCAG CAGGGAGGTG AACCCAGAGC CAGTTCCCCC ACCAAAGCTG TGGAAAACCA
2537 AGAAGCCCTG GAGACCCGTG CACTGGTCAG CCAGCTTGCG AATT

### EXAMPLE F2

### Identification of Human Adenosine A1 Receptor Gene Sequences

The sequence of Human Adenosine A1 Receptor Gene (Acc. No. CG58655-01) was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full-length DNA sequence, or some portion thereof. The protocol for identification of human sequence(s) is disclosed in Example Q8.

Table F3 shows protein alignment (ClustalW) of the CG58655-01 (SEQ ID NO:56), and rat (scb_gb-m64299_{—}1; SEQ ID NO:209) homologs of the Adenosine A1 receptor. Table F4 shows protein sequence of a rat (scb_gb-m64299_{—}1; SEQ ID NO:209) homolog of the Adenosine A1 receptor.

[image]

TABLE F4

Protein sequence of a rat (scb_gb-m64299_1; SEQ ID NO:209) homolog of
the Adenosine A1 receptor.

>m64299_Rat
MPPYISAFGAAYIGIEVLIALVSVPGNVLVIWAVKVNQALRDATFCFIVSLAVADVAVGALVIPLAILIN
IGPQTYFHTCLMVACPVLILTQSSILALLAIAVDRYLRVKIPLRYKTVVTQRRAAVAIAGCWILSLVVGL
TPMFGWNNLSVVEQDWRANGSVGEPVIKCEFEKVISMEYMVYFNFFVWVLPPLLLMVLIYLEVFYLIRK
QLNKKVSASSGDPQKYYGKELKIAKSLALILFLFALSWLPLHILNCITLFCPTCQKPSILIYIAIFLTHGN
SAMNPIVYAFRIHKFRVTFLKIWNDHFRCQPKPPIDEDLPEEKAED

The laboratory cloning was performed using one or more of the methods summarized in Example Q8. The NOV6 clone was analyzed, and the nucleotide and encoded polypeptide sequences are shown in Table P5.

TABLE F5
NOV6 Sequence Analysis

| | | |
|---|---|---|
| NOV6a, CG58655-01 | SEQ ID NO:55 | 1003 bp |
| DNA Sequence | ORF Start: ATG at 5 | ORF Stop: TAG at 983 |

CGCCATGCCGCCCTCCATCTCAGCTTTCCAGGCCGCCTACATCGGCATCGAGGTGCTCATCGCCCTGG
TCTCTGTGCCCGGGAACGTGCTGGTGATCTGGGCGGTGAAGGTGAACCAGGCGCTGCGGGATGCCACC
TTCTGCTTCATCGTGTCGCTGGCGGTGGCTGATGTGGCCGTGGGTGCCCTGGTCATCCCCCTCGCCAT
CCTCATCAACATTGGGCCACAGACCTACTTCCACACCTGCCTCATGGTTGCCTGTCCGGTCCTCATCC
TCACCCAGAGCTCCATCCTGGCCCTGCTGGCAATTGCGGTGGACCGCTACCTCCGGGTCAAGATCCCT
CTCCGGTACAAGATGGTGGTGACCCCCCGGAGGGCGGCGGTGGCCATAGCCGGCTGCTGGATCCTCTC
CTTCGTGGTGGGACTGACCCCTATGTTTGGCTGGAACAATCTGAGTGCGGTGGAGCGGGCCTGGGCAG
CCAACGGCAGCATGGGGGAGCCCGTGATCAAGTGCGAGTTCGAGAAGGTCATCAGCATGGAGTACATG
GTCTACTTCAACTTCTTTGTGTGGGTGCTGCCCCCGCTTCTCCTCATGGTCCTCATCTACCTGGAGGT
CTTCTACCTAATCCGCAAGCAGCTCAACAAGAAGGTGTCGGCCTCCTCCGGCGACCCGCAGAAGTACT
ATGGGAAGGAGCTGAAGATCGCCAAGTCGCTGGCCCTCATCCTCTTCCTCTTTGCCCTCAGCTGGCTG
CCTTTGCACATCCTCAACTGCATCACCCTCTTCTGCCCGTCCTGCCACAAGCCCAGCATCCTTACCTA
CATTGCCATCTTCCTCACGCACGGCAACTCGGCCATGAACCCCATTGTCTATGCCTTCCGCATCCAGA
AGTTCCGCGTCACCTTCCTTAAGATTTGGAATGACCATTTCCGCTGCCAGCCTGCACCTCCCATTGAC
GAGGATCTCCCAGAAGAGAGGCCTGATGACTAGACCCCGCCTTCCGCTCCC

| | | | |
|---|---|---|---|
| NOV6a, CG58655-01 | | | |
| Protein Sequence | SEQ ID NO:56 | 326 aa | MW at 36511.2 kD |

MPPSISAFQAAYIGIEVLIALVSVPGNVLVIWAVKVNQALRDATFCFIVSLAVADVAVGALVIPLAIL
INIGPQTYFHTCLMVACPVLILTQSSILALLAIAVDRYLRVKIPLRYKMVVTPRRAAVAIAGCWILSF
VVGLTPMFGWNNLSAVERAWAANGSMGEPVIKCEFEKVISMEYMVYFNFFVWVLPPLLLMVLIYLEVF
YLIRKQLNKKVSASSGDPQKYYGKELKIAKSLALILFLFALSWLPLHILNCITLFCPSCHKPSILTYI
AIFLTHGNSANNPIVYAFRIQKFRVTFLKIWNDHFRCQPAPPIDEDLPEERPDD

| | | |
|---|---|---|
| NOV6b, 268368558 | SEQ ID NO:57 | 1010 bp |
| DNA Sequence | ORF Start: at 3 | ORF Stop: TAG at 993 |

CACCGAATTCCACCATGCCGCCCTCCATCTCAGCTTTCCAGGCCGCCTACATCGGCATCGAGGTGCTC
ATCGCCCTGGTCTCTGTGCCCGGGAACGTGCTGGTGATCTGGGCGGTGAAGGTGAACCAGGCGCTGCG
GGATGCCACCTTCTGCTTCATCGTGTCGCTGGCGGTGGCTGATGTGGCCGTGGGTGCCCTGGTCATCC
CCCTCGCCATCCTCATCAACATTGGGCCACAGACCTACTTCCACACCTGCCTCATGGTTGCCTGTCCG
GTCCTCATCCTCACCCAGAGCTCCATCCTGGCCCTGCTGGCAATTGCTGTGGACCGCTACCTCCGGGT
CAAGATCCCTCTCCGGTACAAGATGGTGGTGACCCCCCGGAGGGCGGCGGTGGCCATAGCCGGCTGCT
GGATCCTCTCCTTCGTGGTGGGACTGACCCCTATGTTTGGCTGGAACAATCTGAGTGCGGTGGAGCGG
GCCTGGGCAGCCAACGGCAGCATGGGGGAGCCCGTGATCAAGTGCGAGTTCGAGAAGGTCATCAGCAT
GGAGTACATGGTCTACTTCAACTTCTTTGTGTGGGTGCTGCCCCCGCTTCTCCTCATGGTCCTCATCT
ACCTGGAGGTCTTCTACCTAATCCGCAAGCAGCTCAACAAGAAGGTGTCGGCCTCCTCCGGCGACCCG
CAGAAGTACTATGGGAAGGAGCTGAAGATCGCCAAGTCGCTGGCCCTCATCCTCTTCCTCTTTGCCCT
CAGCTGGCTGCCTTTGCACATCCTCAACTGCATCACCCTCTTCTGCCCGTCCTGCCACAAGCCCAGCA
TCCTTACCTACATTGCCATCTTCCTCACGCACGGCAACTCGGCCATGAACCCCATTGTCTATGCCTTC
CGCATCCAGAAGTTCCGCGTCACCTTCCTTAAGATTTGGAATGACCATTTCCGCTGCCAGCCTGCACC
TCCCATTGACGAGGATCTCCCAGAAGAGAGGCCTGATGACTAGGGTGGCGGCCGCTAT

| | | | |
|---|---|---|---|
| NOV6b, 268368558 | | | |
| Protein Sequence | SEQ ID NO:58 | 330 aa | MW at 36910.6 kD |

PNSTMPPSISAFQAAYIGIEVLIALVSVPGNVLVIWAVKVNQALRDATFCFIVSLAVADVAVGALVIP
LAILINIGPQTYFHTCLMVACPVLILTQSSILALLAIAVDRYLRVKIPLRYKMVVTPRRAAVAIAGCW
ILSFVVGLTPMFGWNNLSAVERAWAANGSMGEPVIKCEFEKVISMEYMVYFNFFVWVLPPLLLMVLIY
LEVFYLIRKQLNKKVSASSGDPQKYYGKELKIAKSLALILFLFALSWLPLHILNCITLFCPSCHKPSI
LTYIAIFLTHGNSANNPIVYAFRIQKFRVTFLKIWNDHFRCQPAPPIDEDLPEERPDD

A ClustalW comparison of the above protein sequences yields the following sequence alignment shown in Table F6.

TABLE F6
Comparison of the NOV6 protein sequences.

| | |
|---|---|
| NOV6a | ————MPPSISAFQAAYIGIEVLIALVSVPGNVLVIWAVKVNQALRDATFCFIVSLAVADV |
| NOV6b | PNSTMPPSISAFQAAYIGIEVLIALVSVPGNVLVIWAVKVNQALRDATFCFIVSLAVADV |
| NOV6a | AVGALVIPLAILINIGPQTYFHTCLMVACPVLILTQSSILALLAIAVDRYLRVKIPLRYK |
| NOV6b | AVGALVIPLAILINIGPQTYFHTCLMVACPVLILTQSSILALLAIAVDRYLRVKIPLRYK |
| NOV6a | MVVTPRRAAVAIAGCWILSFVVGLTPMFGWNNLSAVERAWAANGSMGEPVIKCEFEKVIS |
| NOV6b | MVVTPRRAAVAIAGCWILSFVVGLTPMFGWNNLSAVERAWAANGSMGEPVIKCEFEKVIS |
| NOV6a | MEYMVYFMFFVWVLPPLLLMVLIYLEVFYLIRKQLNKKVSASSGDPQKYYGKELKIAKSL |
| NOV6b | MEYMVYFMFFVWVLPPLLLMVLIYLEVFYLIRKQLNKKVSASSGDPQKYYGKELKIAKSL |
| NOV6a | ALILFLFALSWLPLHILNCITLFCPSCHKPSILTYIAIFLTNGNSAMNPIVYAFRIQKFR |
| NOV6b | ALILFLFALSWLPLHILNCITLFCPSCHKPSILTYIAIFLTHGNSAMNPIVYAFRIQKFR |
| NOV6a | VTFLKIWNDHFRCQPAPPIDEDLPEERPDD |
| NOV6b | VTFLKIWNDHFRCQPAPPIDEDLPEERPDD |
| NOV6a | (SEQ ID NO:56) |
| NOV6b | (SEQ ID NO:58) |

Further analysis of the NOV6a protein yielded the following properties shown in Table F7.

TABLE F7
Protein Sequence Properties NOV6a

| SignalP | Cleavage site between residues 3 1 and 32 | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 15; peak value 6.60 | | | |
| | PSG score: 2.20 | | | |
| | GvH: von Heijne's method for signal seq. recognition | | | |
| | GvH score (threshold: −2.1): −4.41 | | | |
| | possible cleavage site: between 59 and 60 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 6 | | | |
| | INTEGRAL | Likelihood = | −5.84 | Transmembrane 15-31 |
| | INTEGRAL | Likelihood = | −8.76 | Transmembrane 53-69 |
| | INTEGRAL | Likelihood = | −7.22 | Transmembrane 87-103 |
| | INTEGRAL | Likelihood = | −6.53 | Transmembrane 124-140 |
| | INTEGRAL | Likelihood = | −8.01 | Transmembrane 185-201 |
| | INTEGRAL | Likelihood = | −6.05 | Transmembrane 236-252 |
| | PERIPHERAL | Likelihood = | 4.72 (at 267) | |
| | ALOM score: −8.76 (number of TMSs: 6) | | | |
| | MTOP: Prediction of membrane topology (Hartmann et al.) | | | |
| | Center position for calculation: 22 | | | |
| | Charge difference: 2.0 C(1.0)-N(−1.0) | | | |
| | C > N: C-terminal side will be inside | | | |
| | >>>Caution: Inconsistent mtop result with signal peptide | | | |
| | >>> membrane topology: type 3b | | | |
| | MITDISC: discrimination of mitochondrial targeting seq | | | |
| | R content: | 0 | Hyd Moment(75): | 0.93 |
| | Hyd Moment(95): | 4.05 | G content: | 1 |
| | D/E content: | 1 | S/T content: | 2 |

Score: −5.83
Gavel: prediction of cleavage sites for mitochondrial preseq
R-2 motif at 133 RRA|AV
NUCDISC: discrimination of nuclear localization signals
pat4: none
pat7: none
bipartite: none
content of basic residues: 8.3%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
55.6%: endoplasmic reticulum
11.1%: Golgi
11.1%: vacuolar
11.1%: vesicles of secretory system
11.1%: mitochondrial
>> prediction for CG58655-01 is end (k = 9)

A search of the NOV6a protein against the Geneseq database, a proprietary database that contains sequences published in patents and patent publication, yielded several homologous proteins shown in Table F8.

TABLE F8
Geneseq Results for NOV6a

| | | NOV6a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP96789 | Human COPD related protein SEQ | 1 . . . 326 | 326/326 (100%) | 0.0 |
| | ID NO: 39 - Homo sapiens, 326 aa. | 1 . . . 326 | 326/326 (100%) | |
| | [WO200297127-A2, 05-DEC-2002] | | | |
| ABP81771 | Human adenosine A1 receptor | 1 . . . 326 | 326/326 (100%) | 0.0 |
| | protein SEQ ID NO: 24 - Homo | 1 . . . 326 | 326/326 (100%) | |
| | sapiens, 326 aa. [WO200261087- | | | |
| | A2, 08-AUG-2002] | | | |
| AAR84192 | Human A1 adenosine receptor - | 1 . . . 326 | 326/326 (100%) | 0.0 |
| | Homo sapiens, 326 aa. | 1 . . . 326 | 326/326 (100%) | |
| | [GB2289218-A, 15-NOV-1995] | | | |
| AAR87655 | Human adenosine receptor A1 | 1 . . . 326 | 326/326 (100%) | 0.0 |
| | subtype - Homo sapiens, 326 aa. | 1 . . . 326 | 326/326 (100%) | |
| | [GB2288733-A, 01-NOV-1995] | | | |
| AAR93989 | Human ventricle A1 adenosine | 1 . . . 326 | 326/326 (100%) | 0.0 |
| | receptor - Homo sapiens, 326 aa. | 1 . . . 326 | 326/326 (100%) | |
| | [WO9511681-A1, 04-MAY-1995] | | | |

In a BLAST search of public sequence databases, the NOV6a protein was found to have homology to the proteins shown in the BLASTP data in Table F9.

TABLE F9
Public BLASTP Results for NOV6a

| | | NOV6a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P30542 | Adenosine A1 receptor - Homo | 1 . . . 326 | 326/326 (100%) | 0.0 |
| | sapiens (Human), 326 aa. | 1 . . . 326 | 326/326 (100%) | |
| P47745 | Adenosine A1 receptor - Cavia | 1 . . . 326 | 311/326 (95%) | 0.0 |
| | porcellus (Guinea pig), 326 aa. | 1 . . . 326 | 318/326 (97%) | |
| A38144 | 326 aa. | 1 . . . 326 | 315/326 (96%) | e−179 |
| Q8BGU7 | Adenosine A1 receptor - Mus | 1 . . . 326 | 309/326 (94%) | e−179 |
| | musculus (Mouse), 326 aa. | 1 . . . 326 | 315/326 (95%) | |
| P28190 | Adenosine A1 receptor - Bos | 1 . . . 326 | 307/326 (94%) | e−179 |
| | taurus (Bovine), 326 aa. | 1 . . . 326 | 315/326 (96%) | |

PFam analysis predicts that the NOV6a protein contains the domains shown in the Table F10.

TABLE F10
Domain Analysis of NOV6a

| | | Identities/ | |
|---|---|---|---|
| Pfam | NOV6a | Similarities for | |
| Domain | Match Region | the Matched Region | Expect Value |
| 7tm_5 | 4 . . . 278 | 46/347 (13%) | 0.74 |
| | | 165/347 (48%) | |
| 7tm_1 | 26 . . . 288 | 81/287 (28%) | 3.6e−65 |
| | | 204/287 (71%) | |

### EXAMPLE F3

### Human Adenosine A1 Receptor Gene Variants and SNPs

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing. Detailed protocols for Pyrosequencing can be found in: Alderbom et al. Determination of Single Nucleotide Polymorphisms by Real-time Pyrophosphate DNA Sequencing. (2000). Genome Research. 10, Issue 8, August. 1249-1265.

In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

Results

The DNA and protein sequences for the novel single nucleotide polymorphic variants of the Adenosine A1 Recepto-like gene of CuraGen Acc. No. CG58655-01 are reported in Table F11. Variants are reported individually but any combination of all or a select subset of variants are also included. In Table F11, the positions of the variant bases and the variant amino acid residues are underlined. In summary, there are 1 variants reported in Table F11. Variant 13381538 is a T to C SNP at 717 bp of the nucleotide sequence that results in a Leu to Pro change at amino acid 238 of protein sequence.

TABLE F11
Variant of nucleotide sequence Acc.
No. CG58655-01 (SEQ ID NO: 55)

| | Nucleotides | | | Amino Acids | | |
|---|---|---|---|---|---|---|
| Variant | Position | Initial | Modified | Position | Initial | Modified |
| 13381538 | 717 | T | C | 238 | Leu | Pro |

TABLE F12
Sequence of Variants

Nucleotide sequence of variant 13381538 NOV6a1n
(underlined). T/C (SEQ ID NO:167)

| | |
|---|---|
| 1 | CGCCATGCCGCCCTCCATCTCAGCTTTCCAGGCCGCCTACATCGGCATCGAGGTGCTCATCGCCCTGGTCTCTGTGCCCG |
| 81 | GGAACGTGCTGGTGATCTGGGCGGTGAAGGTGAACCAGGCGCTGCGGGATGCCACCTTCTGCTTCATCGTGTCGCTGGCG |
| 161 | GTGGCTGATGTGGCCGTGGGTGCCCTGGTCATCCCCCTCGCCATCCTCATCAACATTGGGCCACAGACCTACTTCCACAC |
| 241 | CTGCCTCATGGTTGCCTGTCCGGTCCTCATCCTCACCCAGAGCTCCATCCTGGCCCTGCTGGCAATTGCGGTGGACCGCT |
| 321 | ACCTCCGGGTCAAGATCCCTCTCCGGTACAAGATGGTGGTGACCCCCCGGAGGGCGGCGGTGGCCATAGCCGGCTGCTGG |
| 401 | ATCCTCTCCTTCGTGGTGGGACTGACCCCTATGTTTGGCTGGAACAATCTGAGTGCGGTGGAGCGGGCCTGGGCAGCCAA |
| 481 | CGGCAGCATGGGGGAGCCCGTGATCAAGTGCGAGTTCGAGAAGGTCATCAGCATGGAGTACATGGTCTACTTCAACTTCT |
| 561 | TTGTGTGGGTGCTGCCCCCGCTTCTCCTCATGGTCCTCATCTACCTGGAGGTCTTCTACCTAATCCGCAAGCAGCTCAAC |
| 641 | AAGAAGGTGTCGGCCTCCTCCGGCGACCCGCAGAAGTACTATGGGAAGGAGCTGAAGATCGCCAAGTCGCTGGCCCCCAT |
| 721 | CCTCTTCCTCTTTGCCCTCAGCTGGCTGCCTTTGCACATCCTCAACTGCATCACCCTCTTCTGCCCGTCCTGCCACAAGC |
| 801 | CCAGCATCCTTACCTACATTGCCATCTTCCTCACGCACGGCAACTCGGCCATGAACCCCATTGTCTATGCCTTCCGCATC |
| 881 | CAGAAGTTCCGCGTCACCTTCCTTAAGATTTGGAATGACCATTTCCGCTGCCAGCCTGCACCTCCCATTGACGAGGATCT |
| 961 | CCCAGAAGAGAGGCCTGATGACTAGACCCCGCCTTCCGCTCCC |

Protein sequence of variant NOV6a1p (underlined). (SEQ ID NO:168)

| | |
|---|---|
| 1 | MPPSISAFQAAYIGIEVLIALVSVPGNVLVIWAVKVNQALRDATFCFIVSLAVADVAVGALVIPLAILINIGPQTYFHTC |
| 81 | LMVACPVLILTQSSILALLAIAVDRYLRVKIPLRYKMVVTPRRAAVAIAGCWILSFVVGLTPMFGWNNLSAVERAWAANG |
| 161 | SMGEPVIKCEFEKVISMEYMVYFNFFVWVLPPLLLMVLIYLEVFYLIRKQLNKKVSASSGDPQKYYGKELKIAKSLAPIL |
| 241 | FLFALSWLPLHILNCITLFCPSCHKPSILTYIAIFLTHGNSAMNPIVYAFRIQKFRVTFLKIWNDHFRCQPAPPIDEDLP |
| 321 | EERPDD |

Alteration effect

Leu to Pro

### EXAMPLE F4

### Expression Profile of the Human Adenosine A1 Receptor Gene (CG58655-01)

The protocol for quantitative expression analysis is disclosed in Example Q9.

Expression of gene CG58655-01 was assessed using the primer-probe set Ag6342, described in Table F 13. Results of the RTQ-PCR runs are shown in Tables F 14, F 15, F 16, and F17.

TABLE F13
Probe Name Ag6342

| | | | Start | SEQ ID |
|---|---|---|---|---|
| Primers | Sequences | Length | Position | No |
| Forward | 5′-ccaccgcactcagattgtt-3′ | 19 | 543 | 210 |
| Probe | TET-5′agccaaacataggggtcagtcccac-3′- | 25 | 564 | 211 |
| | TAMRA | | | |
| Reverse | 5′-atagccggctgctggat-3′ | 17 | 602 | 212 |

TABLE F14
General screening panel v1.5

| | Rel. Exp. (%) |
|---|---|
| | Ag6342, Run |
| Tissue Name | 259475625 |
| Adipose | 0.8 |
| Melanoma* Hs688(A).T | 7.0 |
| Melanoma* Hs688(B).T | 6.5 |
| Melanoma* M14 | 3.5 |
| Melanoma* LOXIMVI | 0.3 |
| Melanoma* SK-MEL-5 | 0.9 |
| Squamous cell carcinoma SCC-4 | 0.1 |
| Testis Pool | 29.3 |
| Prostate ca.* (bone met) PC-3 | 0.0 |
| Prostate Pool | 0.4 |
| Placenta | 18.6 |
| Uterus Pool | 0.6 |
| Ovarian ca. OVCAR-3 | 61.1 |
| Ovarian ca. SK-OV-3 | 2.7 |
| Ovarian ca. OVCAR-4 | 22.1 |
| Ovarian ca. OVCAR-5 | 24.5 |
| Ovarian ca. IGROV-1 | 36.3 |
| Ovarian ca. OVCAR-8 | 19.5 |
| Ovary | 0.2 |
| Breast ca. MCF-7 | 7.9 |
| Breast ca. MDA-MB-231 | 0.8 |
| Breast ca. BT 549 | 0.3 |
| Breast ca. T47D | 1.0 |
| Breast ca. MDA-N | 1.3 |
| Breast Pool | 0.6 |
| Trachea | 2.5 |
| Lung | 0.2 |
| Fetal Lung | 2.5 |
| Lung ca. NCI-N417 | 4.5 |
| Lung ca. LX-1 | 1.3 |
| Lung ca. NCI-H146 | 4.3 |
| Lung ca. SHP-77 | 0.0 |
| Lung ca. A549 | 1.7 |
| Lung ca. NCI-H526 | 1.5 |
| Lung ca. NCI-H23 | 0.6 |
| Lung ca. NCI-H460 | 0.0 |
| Lung ca. HOP-62 | 3.1 |
| Lung ca. NCI-H522 | 2.5 |
| Liver | 0.5 |
| Fetal Liver | 1.0 |
| Liver ca. HepG2 | 0.2 |
| Kidney Pool | 0.6 |
| Fetal Kidney | 2.9 |
| Renal ca. 786-0 | 67.4 |
| Renal ca. A498 | 46.7 |
| Renal ca. ACHN | 100.0 |
| Renal ca. UO-31 | 13.7 |
| Renal ca. TK-10 | 73.7 |
| Bladder | 11.4 |
| Gastric ca. (liver met.) NCI-N87 | 2.9 |
| Gastric ca. KATO III | 0.0 |
| Colon ca. SW-948 | 1.0 |
| Colon ca. SW480 | 5.8 |
| Colon ca.* (SW480 met) SW620 | 0.7 |
| Colon ca. HT29 | 0.1 |
| Colon ca. HCT-116 | 6.3 |
| Colon ca. CaCo-2 | 17.8 |
| Colon cancer tissue | 0.1 |
| Colon ca. SW1116 | 5.6 |
| Colon ca. Colo-205 | 0.0 |
| Colon ca. SW-48 | 0.0 |
| Colon Pool | 1.0 |
| Small Intestine Pool | 0.5 |
| Stomach Pool | 0.3 |
| Bone Marrow Pool | 0.3 |
| Fetal Heart | 9.4 |
| Heart Pool | 2.5 |
| Lymph Node Pool | 2.2 |
| Fetal Skeletal Muscle | 1.6 |
| Skeletal Muscle Pool | 1.5 |
| Spleen Pool | 4.9 |
| Thymus Pool | 2.0 |
| CNS cancer (glio/astro) U87-MG | 1.2 |
| CNS cancer (glio/astro) U-118-MG | 3.0 |
| CNS cancer (neuro; met) SK-N-AS | 0.8 |
| CNS cancer (astro) SF-539 | 10.5 |
| CNS cancer (astro) SNB-75 | 45.4 |
| CNS cancer (glio) SNB-19 | 36.6 |
| CNS cancer (glio) SF-295 | 15.2 |
| Brain (Amygdala) Pool | 60.3 |
| Brain (cerebellum) | 75.3 |
| Brain (fetal) | 22.4 |
| Brain (Hippocampus) Pool | 40.3 |
| Cerebral Cortex Pool | 45.1 |
| Brain (Substantia nigra) Pool | 53.2 |
| Brain (Thalamus) Pool | 56.6 |
| Brain (whole) | 32.3 |
| Spinal Cord Pool | 47.3 |
| Adrenal Gland | 0.4 |
| Pituitary gland Pool | 1.0 |
| Salivary Gland | 3.6 |
| Thyroid (female) | 4.3 |
| Pancreatic ca. CAPAN2 | 0.9 |
| Pancreas Pool | 8.5 |

TABLE F15
General screening panel v1.7

| | Rel. Exp. (%) |
|---|---|
| | Ag6342, Run |
| Tissue Name | 318350017 |
| Adipose | 21.2 |
| HUVEC | 0.0 |
| Melanoma* Hs688(A).T | 0.0 |
| Melanoma* Hs688(B).T | 0.2 |
| Melanoma (met) SK-MEL-5 | 0.2 |
| Testis | 17.0 |
| Prostate ca. (bone met) PC-3 | 0.1 |
| Prostate ca. DU145 | 5.3 |
| Prostate pool | 0.2 |
| Uterus pool | 0.0 |
| Ovarian ca. OVCAR-3 | 18.2 |
| Ovarian ca. (ascites) SK-OV-3 | 2.2 |
| Ovarian ca. OVCAR-4 | 26.1 |
| Ovarian ca. OVCAR-5 | 0.9 |
| Ovarian ca. IGROV-1 | 100.0 |
| Ovarian ca. OVCAR-8 | 6.8 |
| Ovary | 0.2 |
| Breast ca. MCF-7 | 1.1 |
| Breast ca. MDA-MB-231 | 3.1 |
| Breast ca. BT-549 | 0.1 |
| Breast ca. T47D | 1.2 |
| Breast pool | 0.3 |
| Trachea | 2.2 |
| Lung | 1.4 |
| Fetal Lung | 1.5 |
| Lung ca. NCI-N417 | 2.0 |
| Lung ca. LX-1 | 0.3 |
| Lung ca. NCI-H146 | 4.1 |
| Lung ca. SHP-77 | 0.0 |
| Lung ca. NCI-H23 | 11.2 |
| Lung ca. NCI-H460 | 1.8 |
| Lung ca. HOP-62 | 1.1 |
| Lung ca. NCI-H522 | 3.6 |
| Lung ca. DMS-114 | 1.5 |
| Liver | 0.2 |
| Fetal Liver | 0.2 |
| Kidney pool | 7.2 |
| Fetal Kidney | 3.0 |
| Renal ca. 786-0 | 0.5 |
| Renal ca. A498 | 32.5 |
| Renal ca. ACHN | 80.1 |
| Renal ca. UO-31 | 8.1 |
| Renal ca. TK-10 | 59.0 |
| Bladder | 0.6 |
| Gastric ca. (liver met.) NCI-N87 | 0.2 |
| Stomach | 0.0 |
| Colon ca. SW-948 | 0.0 |
| Colon ca. SW480 | 0.1 |
| Colon ca. (SW480 met) SW620 | 1.9 |
| Colon ca. HT29 | 0.1 |
| Colon ca. HCT-116 | 6.3 |
| Colon cancer tissue | 0.2 |
| Colon ca. SW1116 | 1.9 |
| Colon ca. Colo-205 | 0.0 |
| Colon ca. SW-48 | 0.0 |
| Colon | 0.4 |
| Small Intestine | 0.0 |
| Fetal Heart | 0.8 |
| Heart | 0.6 |
| Lymph Node pool 1 | 0.3 |
| Lymph Node pool 2 | 1.5 |
| Fetal Skeletal Muscle | 1.6 |
| Skeletal Muscle pool | 0.2 |
| Skeletal Muscle | 0.6 |
| Spleen | 1.5 |
| Thymus | 0.2 |
| CNS cancer (glio/astro) SF-268 | 1.5 |
| CNS cancer (glio/astro) T98G | 1.4 |
| CNS cancer (neuro; met) SK-N-AS | 0.0 |
| CNS cancer (astro) SF-539 | 7.6 |
| CNS cancer (astro) SNB-75 | 8.7 |
| CNS cancer (glio) SNB-19 | 9.3 |
| CNS cancer (glio) SF-295 | 0.5 |
| Brain (Amygdala) | 27.9 |
| Brain (Cerebellum) | 52.1 |
| Brain (Fetal) | 28.1 |
| Brain (Hippocampus) | 20.6 |
| Cerebral Cortex pool | 18.8 |
| Brain (Substantia nigra) | 10.3 |
| Brain (Thalamus) | 25.0 |
| Brain (Whole) | 80.7 |
| Spinal Cord | 12.8 |
| Adrenal Gland | 0.3 |
| Pituitary Gland | 0.6 |
| Salivary Gland | 3.3 |
| Thyroid | 1.8 |
| Pancreatic ca. PANC-1 | 2.8 |
| Pancreas pool | 1.7 |

TABLE F16
Panel 5 Islet

| | Rel. Exp. (%) | Rel. Exp. (%) | Rel. Exp. (%) |
|---|---|---|---|
| | Ag6342, Run | Ag6342, Run | Ag6342, Run |
| Tissue Name | 259472132 | 263594805 | 271406445 |
| 97457_Patient-02go_adipose | 34.4 | 9.6 | 8.4 |
| 97476_Patient-07sk_skeletal muscle | 0.0 | 1.4 | 1.9 |
| 97477_Patient-07ut_uterus | 0.0 | 3.1 | 0.9 |
| 97478_Patient-07pl_placenta | 88.9 | 79.0 | 68.8 |
| 99167_Bayer Patient 1 | 28.5 | 15.3 | 19.2 |
| 97482_Patient-08ut_uterus | 0.0 | 2.0 | 0.0 |
| 97483_Patient-08pl_placenta | 36.9 | 27.7 | 27.5 |
| 97486_Patient-09sk_skeletal muscle | 11.1 | 1.7 | 4.0 |
| 97487_Patient-09ut_uterus | 0.0 | 0.0 | 2.0 |
| 97488_Patient-09pl_placenta | 100.0 | 64.2 | 63.3 |
| 97492_Patient-10ut_uterus | 8.2 | 1.9 | 3.8 |
| 97493_Patient-10pl_placenta | 96.6 | 100.0 | 100.0 |
| 97495_Patient-11go_adipose | 34.4 | 3.3 | 4.1 |
| 97496_Patient-11sk_skeletal muscle | 9.7 | 6.4 | 2.0 |
| 97497_Patient-11ut_uterus | 12.7 | 3.1 | 6.1 |
| 97498_Patient-11pl_placenta | 52.9 | 67.4 | 62.0 |
| 97500_Patient-12go_adipose | 28.1 | 24.0 | 18.6 |
| 97501_Patient-12sk_skeletal muscle | 20.0 | 9.9 | 10.1 |
| 97502_Patient-12ut_uterus | 8.1 | 2.6 | 5.5 |
| 97503_Patient-12pl_placenta | 67.8 | 47.0 | 40.3 |
| 94721_Donor 2 U - A_Mesenchymal Stem Cells | 75.8 | 68.3 | 53.2 |
| 94722_Donor 2 U - B_Mesenchymal Stem Cells | 22.4 | 46.0 | 57.4 |
| 94723_Donor 2 U - C_Mesenchymal Stem Cells | 62.0 | 51.8 | 57.0 |
| 94709_Donor 2 AM - A_adipose | 3.1 | 21.8 | 14.3 |
| 94710_Donor 2 AM - B_adipose | 0.0 | 5.3 | 9.4 |
| 94711_Donor 2 AM - C_adipose | 8.7 | 14.8 | 8.2 |
| 94712_Donor 2 AD - A_adipose | 19.1 | 17.7 | 14.0 |
| 94713_Donor 2 AD - B_adipose | 13.0 | 29.7 | 22.5 |
| 94714_Donor 2 AD - C_adipose | 10.7 | 35.6 | 12.2 |
| 94742_Donor 3 U - A_Mesenchymal Stem Cells | 23.3 | 29.1 | 5.9 |
| 94743_Donor 3 U - B_Mesenchymal Stem Cells | 33.0 | 8.9 | 28.3 |
| 94730_Donor 3 AM - A_adipose | 0.0 | 5.1 | 7.0 |
| 94731_Donor 3 AM - B_adipose | 0.0 | 7.5 | 0.0 |
| 94732_Donor 3 AM - C_adipose | 4.3 | 3.3 | 3.7 |
| 94733_Donor 3 AD - A_adipose | 0.0 | 1.1 | 12.4 |
| 94734_Donor 3 AD - B_adipose | 3.7 | 1.8 | 2.1 |
| 94735_Donor 3 AD - C_adipose | 23.2 | 2.6 | 7.0 |
| 77138_Liver_HepG2untreated | 0.0 | 1.7 | 3.8 |
| 73556_Heart_Cardiac stromal cells (primary) | 0.0 | 0.0 | 0.0 |
| 81735_Small Intestine | 4.2 | 8.3 | 7.6 |
| 72409_Kidney_Proximal Convoluted Tubule | 36.9 | 22.7 | 15.8 |
| 82685_Small intestine Duodenum | 0.0 | 3.8 | 0.0 |
| 90650_Adrenal_Adrenocortical adenoma | 0.0 | 0.0 | 2.0 |
| 72410_Kidney_HRCE | 48.3 | 32.3 | 44.1 |
| 72411_Kidney_HRE | 54.0 | 46.3 | 33.2 |
| 73139_Uterus_Uterine smooth muscle cells | 12.1 | 14.5 | 9.8 |

TABLE F17
Human Metabolic

| Tissue Name | A |
|---|---|
| 137857 psoas-AA.M.Diab.-hi BMI-6 | 0.2 |
| 135760 psoas-HI.M.Diab.-hi BMI-21 | 4.1 |
| 134827 psoas-CC.M.Diab.-hi BMI-4 | 0.2 |
| 137860 psoas-AA.M.Diab.-med BMI-8 | 0.6 |
| 137834 psoas-CC.M.Diab.-med BMI-2 | 0.5 |
| 137828 psoas-CC.M.Diab.-med BMI-1 | 0.7 |
| 135763 psoas-HI.M.Diab.-med BMI-23 | 0.9 |
| 142740 psoas-AS.M.Diab.-low BMI-20 | 0.4 |
| 134834 psoas-AA.M.Diab.-low BMI-17 | 0.5 |
| 137850 psoas-AS.M.Norm-hi BMI-34 | 1.7 |
| 135769 psoas-HI.M.Norm-hi BMI-31 | 0.2 |
| 135766 psoas-AA.M.Norm-hi BMI-25 | 0.2 |
| 142746 psoas-AA.M.Norm-med BMI-37 | 0.3 |
| 142745 psoas-HI.M.Norm-med BMI-35 | 0.1 |
| 137855 psoas-AA.M.Norm-med BMI-47 | 0.2 |
| 137844 psoas-CC.M.Norm-med BMI-26 | 0.1 |
| 142742 psoas-CC.M.Norm-low BMI-40 | 0.7 |
| 137873 psoas-AS.M.Norm-low BMI-28 | 0.6 |
| 137853 psoas-HI.M.Norm-low BMI-41 | 0.7 |
| 135775 psoas-CC.M.Norm-low BMI-39 | 0.6 |
| 137858 diaphragm-AA.M.Diab.-hi BMI-6 | 0.4 |
| 135772 diaphragm-AS.M.Diab-hi BMI-9 | 0.3 |
| 135761 diaphragm-HI.M.Diab.-hi BMI-21 | 1.3 |
| 134828 diaphragm-CC.M.Diab.-hi BMI-4 | 0.3 |
| 137835 diaphragm-CC.M.Diab.-med BMI-2 | 0.2 |
| 135764 diaphragm-HI.M.Diab.-med BMI-23 | 0.2 |
| 134835 diaphragm-AA.M.Diab.-low BMI-17 | 0.4 |
| 142738 diaphragm-CC.M.Norm-hi BMI-29 | 0.7 |
| 139517 diaphragm-AS.M.Norm-hi BMI-34 | 0.9 |
| 137848 diaphragm-HI.M.Norm-hi BMI-31 | 0.4 |
| 137843 diaphragm-AA.M.Norm-hi BMI-25 | 0.2 |
| 137879 diaphragm-AA.M.Norm-med BMI-47 | 0.2 |
| 137872 diaphragm-CC.M.Norm-med BMI-26 | 0.2 |
| 135773 diaphragm-HI.M.Norm-med BMI-35 | 0.7 |
| 139542 diaphragm-HI.M.Norm-low BMI-4 1 | 1.5 |
| 137877 diaphragm-CC.M.Norm-low BMI-39 | 0.7 |
| 137874 diaphragm-AS.M.Norm-low BMI-28 | 0.4 |
| 141340 subQadipose-AA.M.Diab.-hi BMI-6 | 2.8 |
| 137836 subQadipose-HI.M.Diab.-hi BMI-21 | 0.1 |
| 135771 subQadipose-AS.M.Diab-hi BMI-9 | 0.2 |
| 141329 pancreas-CC.M.Diab.-hi BMI-4 | 1.1 |
| 137862 subQadipose-CC.M.Diab.-med BMI-1 | 0.4 |
| 135762 subQadipose-HI.M.Diab.-med BMI-23 | 0.0 |
| 141338 subQadipose-AS.M.Diab.-low BMI-20 | 0.1 |
| 139547 subQadipose-HI.M.Diab.-low BMI-22 | 0.2 |
| 135757 subQadipose-CC.M.Diab.-low BMI-13 | 0.1 |
| 134832 subQadipose-AA.M.Diab.-low BMI-17 | 0.6 |
| 141332 subQadipose-HI.M.Norm-hi BMI-31 | 0.5 |
| 135767 subQadipose-CC.M.Norm-hi BMI-29 | 0.1 |
| 135765 subQadipose-AS.M.Norm-hi BMI-34 | 1.1 |
| 141339 subQadipose-HI.M.Norm-med BMI-35 | 0.8 |
| 141334 subQadipose-CC.M.Norm-med BMI-26 | 0.4 |
| 139544 subQadipose-AA.M.Norm-med BMI-47 | 1.5 |
| 137875 subQadipose-AA.M.Norm-med BMI-37 | 0.5 |
| 141331 subQadipose-AS.M.Norm-low BMI-28 | 0.5 |
| 137878 subQadipose-HI.M.Norm-low BMI-41 | 0.1 |
| 137876 subQadipose-CC.M.Norm-low BMI-39 | 0.1 |
| 137859 vis.adipose-AA.M.Diab.-hi BMI-6 | 0.0 |
| 135770 vis.adipose-AS.M.Diab-hi BMI-9 | 0.4 |
| 135759 vis.adipose-HI.M.Diab.-hi BMI-21 | 0.1 |
| 143502 vis.adipose-CC.M.Diab.-med BMI-2 | 2.3 |
| 139510 vis.adipose-AA.M.Diab.-med BMI-8 | 0.1 |
| 137861 vis.adipose-CC.M.Diab.-med-l | 0.2 |
| 137839 vis.adipose-HI.M.Diab.-med BMI-23 | 0.0 |
| 139546 vis.adipose-HI.M.Diab.-low BMI-22 | 0.2 |
| 137831 vis.adipose-CC.M.Diab.-low BMI-13 | 0.0 |
| 139522 vis.adipose-HI.M.Norm-hi BMI-31 | 0.5 |
| 139516 vis.adipose-AS.M.Norm-hi BMI-34 | 0.1 |
| 137846 vis.adipose-CC.M.Norm-hi BMI-29 | 0.1 |
| 137841 vis.adipose-AA.M.Norm-hi BMI-25 | 0.3 |
| 139543 vis.adipose-AA.M.Norm-med BMI-47 | 0.7 |
| 139532 vis.adipose-AA.M.Norm-med BMI-37 | 0.1 |
| 139530 vis.adipose-HI.M.Norm-med BMI-35 | 0.2 |
| 139539 vis.adipose-HI.M.Norm-low BMI-41 | 0.0 |
| 139535 vis.adipose-CC.M.Norm-low BMI-40 | 0.1 |
| 137852 vis.adipose-CC.M.Norm-low BMI-39 | 0.7 |
| 135768 vis.adipose-AS.M.Norm-low BMI-28 | 0.1 |
| 141327 liver-CC.M.Diab.-hi BMI-4 | 0.1 |
| 139514 liver-HI.M.Diab.-hi BMI-21 | 0.2 |
| 139526 liver-CC.M.Diab.-med BMI-2 | 0.3 |
| 139511 liver-AA.M.Diab.-med BMI-8 | 0.4 |
| 137840 liver-HI.M.Diab.-med BMI-23 | 0.1 |
| 137827 liver-CC.M.Diab.-med BMI-1 | 0.0 |
| 137838 liver-HI.M.Diab.-low BMI-22 | 0.0 |
| 135758 liver-CC.M.Diab.-low BMI-13 | 0.0 |
| 139519 liver-CC.M.Norm-hi BMI-29 | 0.4 |
| 139518 liver-AA.M.Norm-hi BMI-25 | 0.2 |
| 137849 liver-AS.M.Norm-hi BMI-34 | 0.0 |
| 137847 liver-HI.M.Norm-hi BMI-31 | 0.0 |
| 142741 liver-AA.M.Norm-med BMI-37 | 0.0 |
| 141341 liver-HI.M.Norm-med BMI-35 | 0.1 |
| 141335 liver-CC.M.Norm-med BMI-26 | 0.1 |
| 139540 liver-HI.M.Norm-low BMI-41 | 2.1 |
| 139534 liver-CC.M.Norm-low BMI-39 | 2.6 |
| 139521 liver-AS.M.Norm-low BMI-28 | 0.3 |
| 141328 pancreas-CC.M.Diab.-hi BMI-4 | 1.6 |
| 139525 pancreas-AS.M.Diab.-hi BMI-9 | 0.2 |
| 137856 pancreas-AA.M.Diab.-hi BMI-6 | 4.8 |
| 137837 pancreas-HI.M.Diab.-hi BMI-21 | 1.8 |
| 141337 pancreas-CC.M.Diab.-med BMI-2 | 0.0 |
| 139527 pancreas-CC.M.Diab.-med BMI-1 | 0.1 |
| 139515 pancreas-HI.M.Diab.-med BMI-23 | 8.4 |
| 139512 pancreas-AA.M.Diab.-med BMI-8 | 3.9 |
| 142739 pancreas-AS.M.Diab.-low BMI-20 | 0.9 |
| 139513 pancreas-CC.M.Diab.-low BMI-13 | 0.9 |
| 142743 pancreas-AA.M.Norm-hi BMI-25 | 2.9 |
| 139523 pancreas-HI.M.Norm-hi BMI-31 | 0.3 |
| 139520 pancreas-CC.M.Norm-hi BMI-29 | 0.5 |
| 142744 pancreas-HI.M.Norm-med BMI-35 | 0.4 |
| 139545 pancreas-AA.M.Norm-med BMI-47 | 0.3 |
| 139531 pancreas-AA.M.Norm-med BMI-37 | 0.0 |
| 137871 pancreas-CC.M.Norm-med BMI-26 | 1.5 |
| 139541 pancreas-Hi.M.Norm-low BMI-41 | 0.2 |
| 139537 pancreas-CC.M.Norm-low BMI-40 | 6.7 |
| 139533 pancreas-CC.M.Norm-low BMI-39 | 0.0 |
| 137845 pancreas-AS.M.Norm-low BMI-28 | 1.0 |
| 143530 small intestine-AA.M.Diab.-hi BMI-6 | 0.3 |
| 143529 small intestine-CC.M.Diab.-hi BMI-4 | 0.2 |
| 143538 small intestine-HI.M.Diab.-med BMI-23 | 3.6 |
| 143531 small intestine-AA.M.Diab.-med BMI-8 | 0.2 |
| 143528 small intestine-CC.M.Diab.-med BMI-2 | 0.3 |
| 143537 small intestine-HI.M.Diab.-low BMI-22 | 0.3 |
| 143535 small intestine-AS.M.Diab.-low BMI-20 | 0.4 |
| 143534 small intestine-AA.M.Diab.-low BMI-17 | 0.2 |
| 143544 small intestine-AS.M.Norm-hi BMI-34 | 0.2 |
| 143543 small intestine-HI.M.Norm-hi BMI-31 | 0.8 |
| 143542 small intestine-CC.M.Norm-hi BMI-29 | 0.1 |
| 143539 small intestine-AA.M.Norm-hi BMI-25 | 3.1 |
| 143548 small intestine-AA.M.Norm-med BMI-47 | 0.1 |
| 143547 small intestine-AA.M.Norm-med BMI-37 | 0.1 |
| 143540 small intestine-CC.M.Norm-med BMI-26 | 0.1 |
| 143550 small intestine-CC.M.Norm-low BMI-40 | 0.0 |
| 143549 small intestine-CC.M.Norm-low BMI-39 | 0.2 |
| 143546 small intestine-HI.M.Norm-low BMI-41 | 0.0 |
| 143525 hypothalamus-HI.M.Diab.-hi BMI-21 | 0.2 |
| 143515 hypothalamus-CC.M.Diab.-hi BMW | 0.0 |
| 143513 hypothalamus-AA.M.Diab.-hi BMI-6 | 35.1 |
| 143507 hypothalamus-AS.M.Diab.-hi BMI-9 | 42.0 |
| 143506 hypothalamus-CC.M.Diab.-med BMI-1 | 83.5 |
| 143505 hypothalamus-HI.M.Diab.-med BMI-23 | 0.4 |
| 143509 hypothalamus-AA.M.Diab.-low BMI-17 | 100.0 |
| 143508 hypothalamus-CC.M.Diab.-low BMI-13 | 37.9 |
| 143503 hypothalamus-AS.M.Diab.-low BMI-20 | 60.3 |
| 143522 hypothalamus-HI.M.Norm-hi BMI-31 | 0.0 |
| 143516 hypothalamus-AS.M.Norm-hi BMI-34 | 0.0 |
| 143511 hypothalamus-CC.M.Norm-hi BMI-29 | 33.9 |
| 143504 hypothalamus-AA.M.Norm-hi BMI-25 | 19.9 |
| 143517 hypothalamus-AA.M.Norm-med BMI-47 | 0.3 |
| 143514 hypothalamus-HI.M.Norm-med BMI-35 | 16.8 |
| 143521 hypothalamus-AS.M.Norm-low BMI-28 | 0.1 |
| 143512 hypothalamus-CC.M.Norrn-low BMI-40 | 0.1 |
| 145454 Patient-25pl (CC.Diab.low BMI.no insulin) | 17.6 |
| 110916 Patient-18pl (HI.Diab.obese.no insulin) | 6.0 |
| 110913 Patient-18go (HI.Diab.obese.no insulin) | 0.5 |
| 110911 Patient-17pl (CC.Diab.low BMI.no insulin) | 18.8 |
| 110908 Patient-17go (CC.Diab.low BMI.no insulin) | 1.7 |
| 100752 Patient-15sk (CC.Diab.obese.no insulin) | 1.9 |
| 97828 Patient-13pl (CC.Diab.overwt.no insulin) | 9.7 |
| 160114 Patient 27-ut (CC.Diab.obese.insulin) | 0.5 |
| 160113 Patient 27-pl (CC.Diab.obese.insulin) | 21.5 |
| 160112 Patient 27-sk (CC.Diab.obese.insulin) | 0.5 |
| 160111 Patient 27-go (CC.Diab.obese.insulin) | 1.3 |
| 145461 Patient-26sk (CC.Diab.obese.insulin) | 2.1 |
| 145441 Patient-22sk (CC.Diab.low BMI.insulin) | 6.8 |
| 145438 Patient-22pl (CC.Diab.low BMI.insulin) | 14.8 |
| 145427 Patient-20pl (CC.Diab.overwt.insulin) | 18.8 |
| 97503 Patient- 12pl (CC.Diab.unknown BMI.insulin) | 2.5 |
| 145443 Patient-23pl (CC.Non-diab.overwt) | 11.9 |
| 145435 Patient-21pl (CC.Non-diab.overwt) | 20.3 |
| 110921 Patient- 19pl (CC.Non-diab.low BMI) | 7.9 |
| 110918 Patient-19go (CC.Non-diab.low BMI) | 1.1 |
| 97481 Patient-08sk (CC.Non-diab.obese) | 0.9 |
| 97478 Patient-07pl (CC.Non-diab.obese) | 7.9 |
| 160117 Human Islets-male, obese | 0.2 |
| 145474 PANC1 (pancreas carcinoma) 1 | 2.0 |
| 154911 Capan2 (pancreas adenocarcinoma) | 2.8 |
| 141190 SW579 (thyroid carcinoma) | 0.1 |
| 145489 SK-N-MC (neuroblastoma) 1 | 0.0 |
| 145495 SK-N-SH (neuroblastoma) 1 | 2.1 |
| 145498 U87 MG (glioblastoma) 2 | 10.1 |
| 145484 HEp-2 (larynx carcinoma) 1 | 0.2 |
| 145479 A549 (lung carcinoma) | 2.4 |
| 145488 A427 (lung carcinoma) 2 | 4.0 |
| 145472 FHs 738Lu (normal lung) 1 | 8.1 |
| 141187 SKW6.4 (B lymphocytes) | 0.0 |
| 154644 IM-9 (immunoglobulin secreting lymphoblast) | 0.0 |
| 154645 MOLT-4 (acute lymphoblastic leukemia | 1.0 |
| derived from peripheral blood) | |
| 154648 U-937 (histiocystic lymphoma) | 0.0 |
| 154647 Daudi (Burkitt's lymphoma) | 0.1 |
| 145494 SK-MEL-2 (melanoma) 2 | 1.5 |
| 141176 A375 (melanoma) | 3.4 |
| 154642 SW 1353 (humerus chondrosarcoma) | 12.2 |
| 141179 HT-1080 (fibrosarcoma) | 2.0 |
| 145491 MG-63 (osteosarcoma) 1 | 48.6 |
| 141186 MCF7 (breast carcinoma) | 4.2 |
| 141193 T47D (breast carcinoma) | 2.3 |
| 154641 BT-20 (breast carcinoma) | 35.8 |
| 141175 293 (kidney transformed with adenovirus 5 | 0.7 |
| 141182 HUH hepatomal | 0.0 |
| 141184 HUH7 hepatomal | 0.0 |
| 145478 HT1376 (bladder carcinoma) | 3.6 |
| 145481 SCaBER (bladder carcinoma) | 0.0 |
| 141192 SW620 (lymph node metastatsis, colon | 1.6 |
| carcinoma) 2 | |
| 141180 HT29 (colon carcinoma) 1 | 0.0 |
| 141188 SW480 (colon carcinoma) 1 | 2.5 |
| 154646 CAOV-3 (ovary adenocarcinoma) | 16.4 |
| 141194 HeLa (cervix carcinoma)- 2 | 6.2 |
| 145482 HeLa S3 (cervix carcinoma) 1 | 0.3 |
| 145486 DU145 (prostate carcinoma) | 53.6 |
| 154643 PC-3 (prostate adenocarcinoma) | 0.0 |
| 154649 HCT-8 (ileocecal adenocarcinoma) | 8.3 |

Column A - Rel. Exp. (%) Ag6342, Run 324668027

General screening panel v1.7 Summary: (Ag6342) The highest expression of this gene was detected in the ovarian cancer cell line IGROV-1 (CT=24). This gene is overexpressed in several ovarian cancer cell lines as compared to normal tissue. This gene was overexpressed in several renal cancer cell lines as compared to normal tissue. Therapeutic modulation of this gene, expressed protein and/or use of antibodies or small molecule drugs targeting the gene or gene product may be useful in the treatment of renal and ovarian cancers.

Among tissues with metabolic or endocrine function, this gene was significantly expressed at high to moderate levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product will be useful in the treatment of endocrine/metabolically related diseases, such as obesity and diabetes.

Human Metabolic Summary: (Ag6342) The highest expression of this gene was detected in the hypothalamus of a diabetic patient. This gene is expressed at increased levels in hypothalamus of diabetic patients. This gene was expressed at low levels in visceral adipose, skeletal muscle and pancreas. Therapeutic modulation of this gene, expressed protein and/or use of antibodies or small molecule drugs targeting the gene or gene product will be useful in the treatment of endocrine/metabolically related diseases, such as obesity and diabetes.

Panel 5 Islet Summary: (Ag6342) The expression of this gene was downregulated in mid-differentiated and differentiated adipose cells. Therapeutic modulation of this gene, expressed protein and/or use of antibodies or small molecule drugs targeting the gene or gene product will be useful in the treatment of obesity. Pursuant to our invention, this data shows for the first time, that this receptor subtype is expressed in human islet cells (Bayer patient 1).

### EXAMPLE F5

### Assays Screening for Modulators of Adenosine A1 Receptor

Adenosine A1 receptor activation mediates the inhibition of cAMP formation through Gᵢ protein signalling. This is relevant for insulin secretion because an increase in intracellular cAMP potentiates insulin secretion. Type 2 diabetes is caused by too little insulin secretion. Therefore, an antagonist of Adora1 will act to increase insulin secretion and the antagonist would be suitable in treatment of Type 2 diabetes.

Although the disclosed sequences of Adenosine A1 receptor are the preferred isoforms, any of the other isoforms may be used for similar purposes. Furthermore, under varying assay conditions, conditions may dictate that another isoform may supplant the listed isoforms. The CG58655-01 gene described herein, encoding the human Adenosine A1 receptor, represents a full-length physical clone and may be used directly for expression and screening purposes.

Assays for screening for modulators of human Adora1 can be formulated utilizing the non-exhaustive list of cell lines that express Adora1 from the RTQ-PCR results shown herein.

Adenosine A1 receptor activation mediates the inhibition of adenylyl cyclase and cAMP formation through Gᵢ protein signalling. To assay the inhibition of adenosine A1 receptor, the measurement of intracellular cAMP can be utilized. Cyclic AMP measurements can be made using the Biotrak cAMP assay system (Amersham Biosciences, Piscataway, N.J., USA). (Harndahl L, Jing X J, Ivarsson R, Degerman E, Ahren B, Manganiello V C, Renstrom E, Holst L S. Important role of phosphodiesterase 3B for the stimulatory action of cAMP on pancreatic beta-cell exocytosis and release of insulin. J Biol. Chem. 2002 Oct. 4; 277 (40):37446-55. PMID: 12169692). An increase in cAMP would be indicative of a positive screen. To evaluate the efficacy of the compound(s) thus identified, the effect on glucose-stimulated insulin secretion can be assayed in vitro with dispersed rat islets as used herein, or can be assayed in vivo by the effects on blood glucose in known rodent models of Type 2 diabetes.

Our results indicate that a modulator of Adora1 activity, such as an inhibitor, activator, antagonist, or agonist of Adora1 may be useful for treatment of such disorders as obesity, diabetes, and insulin resistance, as well as for enhancement of insulin secretion.

G. NOV7—3-Hydroxy-3-Methylglutaryl Co enzyme A Lyase (HMG-CoA Lyase or HMG-CoA or HL)

3-Hydroxy-3-Methylglutaryl Coenzyme A Lyase catalyzes the final step of ketogenesis, an important pathway of mammalian energy metabolism. HMG-CoA Lyase deficiency known as hydroxymethylglutaricaciduria is an autosomal recessive inborn error in man leading to episodes of hypoglycemia and coma.

HMG-CoA Lyase has the following catalytic activity:
(s)-3-hydroxy-3-methylglutaryl-CoA=acetyl-CoA+acetoacetate

HMG-CoA affects biochemical pathways relevant to the etiology and pathogenesis of obesity and/or diabetes. The scheme incorporates the unique findings of these discovery studies in conjunction with what has been reported in the literature. The outcome of inhibiting the action of the human HMG CoA lyase would be a reduction of Insulin Resistance, a major problem in obesity and/or diabetes. HMG-CoA lyase uses HMG-CoA as a substrate to produce acetoacetate and acetyl-CoA. This is the final step in ketogenesis and leucine metabolism. Importantly, acetyl-CoA from this reaction can be fed back into the TCA cycle but also into lipogenic pathways.

We discovered that Mitochondrial 3-Hydroxy-3methylglutaryl coenzyme A lyase (mHMG-CoA lyase) is upregulated in the liver of SHR and WKY rats after triglitazone treatment. mHMG-CoA lyase is the final step in ketogenesis and leucine catabolism which has 3-hydroxy-methylglutaryl-CoA as its substrate, and produces acetoacetate (ketone body) and acetyl-CoA. This process takes place in the liver especially during weight loss and the amount of acetyl-CoA produced during both fatty acid oxidation and ketogenesis often exceeds the capacity of the TCA cycle. Moreover, excess citrate shunts acetyl-CoA back into the cytoplasm where it is used for cholesterol and fatty acid biosynthesis. Therefore, inhibiting this enzyme during weight loss may slow down ketone body formation and the generation of acetyl-CoA, and thus prevent the saturation of the TCA cycle.

The above formula summarizes the biochemistry surrounding the human HMG-CoA Lyase and potential assays that may be used to screen for antibody therapeutics or small molecule drugs to treat obesity and/or diabetes. Cell lines expressing the HMG-CoA Lyase can be obtained from the RTQ-PCR results shown herein. These and other HMG-CoA Lyase expressing cell lines could be used for screening purposes.

Furthermore, our results indicate that a modulator of HMG CoA Lyase activity, such as an inhibitor, activator, antagonist, or agonist of HMG CoA Lyase may be useful for treatment of such disorders as obesity, diabetes, and insulin resistance, as well as for enhancement of insulin secretion.

Discovery Process

The following sections describe the study design(s) and the techniques used to identify the HMG-CoA Lyase—encoded protein and any variants, thereof, as being suitable as diagnostic markers, targets for an antibody therapeutic and targets for a small molecule drugs for Obesity and Diabetes.

### EXAMPLE G1

### Insulin Resistance Study

A protocol Insulin Resistance study is disclosed in Example Q5.

The spontaneously hypertensive rat (SHR) is a strain exhibiting features of the human Metabolic Syndrome X. The phenotypic features include obesity, hyperglycemia, hypertension, dyslipidemia and dysfibrinolysis. Tissues were removed from adult male rats and a control strain (Wistar-Kyoto) to identify the gene expression differences that underlie the pathologic state in the SHR and in animals treated with various anti-hyperglycemic agents such as troglitizone. Tissues included sub-cutaneous adipose, visceral adipose and liver.

A gene fragment of the rat HMG-COA LYASE was initially found to be upregulated by 1.6 fold in the liver of WKY rats treated with Troglitazone LD10 relative to WKY rats treated with 0.02% DMSO as control using CuraGen's GeneCalling® method of differential gene expression. A differentially expressed rat gene fragment migrating at approximately 426.4 nucleotides in length was definitively identified as a component of the rat HMG CoA Lyase cDNA in the Troglitazone treated and the untreated WKY control rats. The method of competitive PCR was used for confirmation of the gene assessment. The chromatographic peaks corresponding to the gene fragment of the rat HMG CoA Lyase were ablated when a gene-specific primer (shown in Table G1) competes with primers in the linker-adaptors during the PCR amplification. The peaks at 426.4 nt in length were ablated in the sample from both the Troglitazone treated and the untreated WKY control rats. The altered expression of these genes in the animal model support the role of HMG CoA Lyase in the pathogenesis of obesity and/or diabetes.

TABLE G1
The sequence of the 427 nucleotide-long gene fragment and the gene-specific
primers used for competitive PCR are indicated on the cDNA sequence of the
rat HMG CoA Lyase fragment (SEQ ID NO:213) and are shown below in bold.
The gene-specific primers at the 5′ and 3′ ends of the fragment are
underlined.
Gene Sequence identified in WKY Troglitazone LD10 vs. 0.02% DMSO (Identified
fragment from 612 to 1038 in bold. band size: 427)

GGCCCCCGAG ATGGTCTGCA GAATGAAAAG AGTATCGTGC CGACGCCAGT GAAAATCAAA CTGATAGACA
TGCTATCCGA AGCAGGGCTC CCGGTCATCG AGOCCACCAG CTTTGTCTCT CCCAAGTGGG TGCCGCAGAT
GGCTGACCAC TCTGACGTCT TGAAGGGCAT TCAGAAGTTT CCCGGCATCA ACTACCCGGT CCTGACACCA
AACATGAAAG GCTTTGAGGA AGCGGTAGCT GCAGGTGCCA AGGAAGTGAG CATCTTTGGG GCTGCGTCCG
AGCTCTTCAC CCGGAAGAAT GTGAACTGCT CTATAGAGGA GAGTTTCCAG CGCTTTGATG GGGTCATGCA
GGCCGCGAGG GCTGCCAGCA TCTCTGTGAG AGGGTATGTC TCCTGTGCCC TCGGATGTCC CTACGACGGG
AAGGTCTCCC CGGCTAAAGT TGCTGAGGTC GCCAAGAAGT TGTACTCAAT GGGCTGCTAT GAGATGTCCC
TTGGGGACAC CATTGGCGTA GGCACGCCAG GACTCATGAA AGACATGCTG ACTGCTGTCC TGCATGAAGT
GCCTGTGGCC GCATTGGCTG TCCACTGCCA TGACACCTAT GGCCAAGCTC TGGCCAACAC GTTGGTGGCC
CTGCAGATGG GAGTGAGCGT TGTGGACTCC TCGGTGGCAG GACTCGGAGG CTGTCCCTAT GCAAAGGGGG
CGTCAGGAAA CTTGGCTACC GAGGACCTGG TCTACATGCT GACTGGCTTA GGGATTCACA CGGGTGTGAA
CCTCCAGAAG CTCCTAGAAG CCGGGGACTT CATCTGTCAA GCCCTGAACA GAAAAACCAG TTCCAAAGTG
GCACAGGCCA CCTGCAAACT CTGAGCCCCT TGTTCACGTA AACCGGAACT GTGGGAGTTG GGTGTACACA
ATGATTCCTG GATGGGGAAA TGGAATGAAG GCAAATGAGC CGGCCTCACA GAGGTCCCTC TCCTACATAG
AAGGGCTAGA GCTGCCAGCA CGCCCGGACC AGCTCCCCAG AGCTGCGTGC CTAAGCACTG CTTGGCTGGC
CCTGGGTGAG TCCACTAGCC AGCAGAGCTG ACATCCATGT GCCACGACCG CGGGTCCCAT GTTCTACCTC
TGAGGACAGC AGCGCCTTTG CTGAAATGGT GGGCTCAATC TACTGCGGTG GCCGACTGCC AACTCCAGCG
TCTCTGGGAA ATCTCTGTAC GTGATTCTTG AAAACAGCTT ATGTAATTAA AGGTTTAATT TTCTAATATC

Additionally, a gene fragment of the rat HMG CoA Lyase was initially found to be upregulated by 2.6 fold in the liver of SHR rats treated with Troglitazone LD10 relative to SHR rats treated with 0.02% DMSO as control using CuraGen's GeneCalling® method of differential gene expression. A differentially expressed rat gene fragment migrating at approximately 48.2 nucleotides in length was definitively identified as a component of the rat HMG CoA Lyase cDNA in the Troglitazone treated and the untreated SHR control rats. The method of competitive PCR was used for confirmation of the gene assessment. The chromatographic peaks corresponding to the gene fragment of the rat HMG CoA Lyase were ablated when a gene-specific primer (shown in Table G2) competes with primers in the linker-adaptors during the PCR amplification. The peaks at 48.2 nt in length were ablated in the sample from both the Troglitazone treated and the untreated WKY control rats. The altered expression of these genes in the animal model support the role of HMG CoA Lyase in the pathogenesis of obesity and/or diabetes.

TABLE G2
The sequence of the 48 nucleotide-long gene fragment and the gene-specific
primers used for competitive PCR are indicated on the cDNA sequence of the rat
HMG CoA Lyase fragment (SEQ ID NO:214) and are shown below in bold.
The gene-specific primers at the 5′ and 3′ ends of the fragment are underlined.
Gene Sequence identified in SHR Troglitazone LD10 vs. 0.02% DMSO (Identified
fragment from 612 to 659 in bold. band size: 48)

GGCCCCCGAGATGGTCTGCAGAATGAAAAGAGTATCGTGCCGACGCCAGTGAAAATCAAACTGATAGACATGCTATCCGA
AGCAGGGCTCCCGGTCATCGAGGCCACCAGCTTTGTCTCTCCCAAGTGGGTGCCGCAGATGGCTGACCACTCTGACGTCT
TGAAGGGCATTCAGAAGTTTCCCGGCATCAACTACCCGGTCCTGACACCAAACATGAAAGGCTTTGAGGAAGCGGTAGCT
GCAGGTGCCAAGGAAGTGAGCATCTTTGGGGCTGCGTCCGAGCTCTTCACCCGGAAGAATGTGAACTGCTCTATAGAGGA
GAGTTTCCAGCGCTTTGATGGGGTCATGCAGGCCGCGAGGGCTGCCAGCATCTCTGTGAGAGGGTATGTCTCCTGTGCCC
TCGGATCTCCCTACGAGGGGAAGGTCTCCCCGGCTAAAGTTGCTGAGGTCGCCAAGAAGTTGTACTCAATGGGCTGCTAT
GAGATCTCCCTTGGGGACACCATTGGCGTAGGCACGCCAGGACTCATGAAAGACATGCTGACTGCTGTCCTGCATGAAGT
GCCTGTGGCCGCATTGGCTGTCCACTGCCATGACACCTATGGCCAAGCTCTGGCCAACACGTTGGTGGCCCTGCAGATGG
GAGTGAGCGTTGTGGACTCCTCGGTGGCAGGACTCGGAGGCTGTCCCTATGCAAAGGGGGCGTCAGGAAACTTGGCTACC
GAGGACCTGGTCTACATCCTCACTGGCTTAGGGATTCACACGGGTGTGAACCTCCAGAAGCTCCTAGAAGCCGGGGACTT
CATCTGTCAAGCCCTGAACAGAAAAACCAGTTCCAAAGTGGCACAGGCCACCTGCAAACTCTGAGCCCCTTGTTCACCTA
AACCGGAACTGTGGGAGTTGGGTGTACACAATGATTCCTGGATGGGGAAATGGAATGAAGGCAAATGAGCCGGCCTCACA
GAGGTCCCTCTCCTACATAGAAGGGCTAGAGCTGCCAGCACGCCCGGAC

### EXAMPLE G2

### Identification of Human HMG CoA Lyase Sequence

The sequence of Human HMG CoA Lyase (Acc. No CG96859-03) was derived by laboratory screening of cDNA library by the two-hybrid approach. cDNA fragments covering either the full length of the DNA sequence, or part of the sequence, or both, were sequenced. In silico prediction was based on sequences available in CuraGen Corporation's proprietary sequence databases or in the public human sequence databases, and provided either the full-length DNA sequence, or some portion thereof. The protocol for identification of human sequence(s) is disclosed in Example Q8.

Table G3 shows an alignment (ClustalW) of the protein sequences of CG96859-03 (SEQ ID NO:60), another public form of HMG CoA lyase with one aa difference (P35914; SEQ ID NO:215), a novel splice form of HMG CoA lyase (CG96859-O₂; SEQ ID NO:66), and the mouse (S65036.1; SEQ ID NO:216) and rat (Y10054; SEQ ID NO:217) orthologues of HMG CoA Lyase. Table G4 shoes protein sequences of the public form of HMG CoA lyase (P35914; SEQ ID NO:215), the mouse (S65036.1; SEQ ID NO:216), and rat (Y10054; SEQ ID NO:217) orthologues of HMG CoA Lyase.

[image]

TABLE G4
Protein sequences of the public form of HMG CoA lyase (P35914; SEQ ID NO:
215), the mouse (S65036.1; SEQ ID NO:216), and rat (Y10054; SEQ ID NO:217)
orthologues of HMG CoA Lyase.

(SEQ ID NO:215)

>p35914_HMG_CoA_lyase
MAAMRKALPRRLVGLASLRAVSTSSMGTLPKRVKIVEVGPRDGLQNEKNIVSTPVKIKLIDMLSEAGLSV
IETTSFVSPKWVPQMGDHTEVLKGIQKFPGINYPVLTPNLKGFEAAVAAGAKEVVIFGAASELFTKKNIN
CSIEESFQRFDAILKAAQSANISVRGYVSCALGCPYEGKISPAKVAEVTKKFYSMGCYEISLGDTIGVGT
PGIMKDMLSAVMQEVPLAALAVHCHDTYGQALANTLMALQMGVSVVDSSVAGLGGCPYAQGASGNLATED
LVYMLEGLGIHTGVNLQKLLEAGNFICQALNRKTSSKVAQATCKL

(SEQ ID NO:216)

>S65036.1_mouse_HMG_lyase
MASVRKAFPRRLVGLTSLRAVSTSSMGTLPKQVKIVEVGPRDGLQNEKSIVPTPVKIRLIDMLSEAGLPV
IEATSFVSPNWVPQMADHSDVLKGIQKFPGINYPVLTPNMKGFEEAVAAGAKEVSVFGAVSELFTRKNAN
CSIEESFQRFAGVMQAAQAASISVRGYVSCALGCPYEGKVSPAKVAEVAKKLYSMGCYEISLGDTIGVGT
PGLMKDMLTAVMHEVPVTALGVHCHDTIGQALANTLVALQMGVSVVDSSVAGLGGCPYAKGASGNLATED
LVYMLNGLGIHTGVNLQKLLEAGDFICQALNRKTSSKVAQATCKL

(SEQ ID NO:217)

>y10054_HMG_CoA_lyase_rat
RLRAKMATVRKAFPQRLVGLASLRAASTSSMGTLPKRVKIVEVGPRDGLQNEKSIVPTPVKIKLIDMLSEAGLPV
IEATSFVSPKWVPQMADHSDVLKGIQKFPGINYPVLTPNMKGFEEAVMGAKEVSIFGAASELFTRKNVN
CSIEESFQRFDGVMQAARAASISVRGYVSCALGCPYEGKVSPAKVAEVAKKLYSMGCYEISLGDTIGVGT
PGLMKDMLTAVLHEVPVAALAVHCHDTYGQALANTLVALQMGVSVVDSSVAGLGGCPYAKGASGNLATED
LVYMLTGLGIHTGVNLQKLLEAGDFICQALNRKTSSKVAQATCKL

In addition to the human version of the HMG CoA Lyase identified as being differentially expressed in the experimental study, other variants have been identified by direct sequencing of cDNAs derived from many different human tissues and from sequences in public databases. Two splice-form variants have been identified at CuraGen. The two alternative spliced forms are CG96859-02 and CG96859-05. The preferred variant of all those identified, to be used for screening purposes, is CG96859-03.

The laboratory cloning was performed using one or more of the methods summarized in Example Q8. The NOV7 clone was analyzed, and the nucleotide and encoded polypeptide sequences are shown in Table G5.

TABLE G5
NOV7 Sequence Analysis

| | | |
|---|---|---|
| NOV7a, CG96859-03 | SEQ ID NO:59 | 1041 bp |
| DNA Sequence | ORF Start: ATG at 15 | ORF Stop: TGA at 990 |

AAATTCCGGCCAAGATGGCAGCAATGAGGAAGGCGCTTCCGCGGCGACTGGTGGGCTTGGCGTCCCTC
CGGGCTGTCAGCACCTCATCTATGGGCACTTTACCAAAGCGGGTGAAAATTGTGGAAGTTGGTCCCCG
AGATGGACTACAAAATGAAAAGAATATCGTATCTACTCCAGTGAAAATCAAGCTGATAGACATGCTTT
CTGAAGCAGGACTCTCTGTTATAGAAACCACCAGCTTTGTGTCTCCTAAGTGGGTTCCCCAGATGGGT
GACCACACTGAAGTCTTGAAGGGCATTCAGAAGTTTCCTGGCATCAACTACCCAGTCCTGACCCCAAA
TTTGAAAGGCTTCGAGGCAGCGGTTGCTGCTGGAGCCAAGGAAGTAGTCATCTTTGGAGCTGCCTCAG
AGCTCTTCACCAAGAAGAACATCAATTGTTCCATAGAGGAGAGTTTTCAGAGGTTTGACGCAATCCTG
AAGGCAGCGCAGTCAGCCAATATTTCTGTGCGGGGGTACGTCTCCTGTGCTCTTGGCTGCCCTTATGA
AGGGAAGATCTCCCCAGCTAAAGTAGCTGAGGTCACCAAGAAGTTCTACTCAATGGGCTGCTACGAGA
TCTCCCTGGGGGACACCATTGGTGTGGGCACCCCAGGGATCATGAAAGACATGCTGTCTGCTGTCATG
CAGGAAGTGCCTCTGGCTGCCCTGGCTGTCCACTGCCATGACACCTATGGTCAAGCCCTGGCCAACAC
CTTGATGGCCCTGCAGATGGGAGTGAGTGTCGTGGACTCTTCTGTGGCAGGACTTGGAGGCTGTCCCT
ACGCACAGGGGGCATCAGGAAACTTGGCCACAGAAGACCTGGTCTACATGCTAGAGGGCTTGGGCATT
CACACGGGTGTGAATCTCCAGAAGCTTCTGGAAGCTGGAAACTTTATCTGTCAAGCCCTGAACAGAAA
AACTAGCTCCAAAGTGGCTCAGGCTACCTGTAAACTCTGAGCCCCTTGCCCACCTGAAGGCCTGGGGA
TGATGTGGAAATAAGGGGCAT

| | | | |
|---|---|---|---|
| NOV7a, CG96859-03 | | | |
| Protein Sequence | SEQ ID NO:60 | 325 aa | MW at 34359.8 kD |

MAAMRKALPRRLVGLASLRAVSTSSMGTLPKRVKIVEVGPRDGLQNEKNIVSTPVKIKLIDMLSEAGL
SVIETTSFVSPKWVPQMGDHTEVLKGIQKFPGINYPVLTPNLKGFEAAVAAGAKEVVIFGAASELFTK
KNINCSIEESFQRFDAILKAAQSANISVRGYVSCALGCPYEGKISPAKVAEVTKKFYSMGCYEISLGD
TIGVGTPGIMKDMLSAVMQEVPLAALAVHCHDTYGQALANTLMALQMGVSVVDSSVAGLGGCPYAQGA
SGNLATEDLVYMLEGLGIHTGVNLQKLLEAGNFICQALNRKTSSKVAQATCKL

| | | |
|---|---|---|
| NOV7b, 223317153 | SEQ ID NO:61 | 982 bp |
| DNA Sequence | ORF Start: at 64 | ORF Stop: TGA at 967 |

TAACTTTATTATTAAAAATTAAAGAGGTATATATTAATGTATCGATTAAATAAGGAGGAATAAACCAT
GGGCACTTTACCAAAGCGGGTGAAAATTGTGGAAGTTGGTCCCCGAGATGGACTACAAAATGAAAAGA
ATATCGTATCTACTCCAGTGAAAATCAAGCTGATAGACATGCTTTCTGAAGCAGGACTCTCTGTTATA
GAAACCACCAGCTTTGTGTCTCCTAAGTGGGTTCCCCAGATGGGTGACCACACTGAAGTCTTGAAGGG
CATTCAGAAGTTTCCTGGCATCAACTACCCAGTCCTGACCCCAAATTTGAAAGGCTTCGAGGCAGCGG
TTGCTGCTGGAGCCAAGGAAGTAGTCATCTTTGGAGCTGCCTCAGAGCTCTTCACCAAGAAGAACATC
AATTGTTCCATAGAGGAGAGTTTTCAGAGGTTTGACGCAATCCTGAAGGCAGCGCAGTCAGCCAATAT
TTCTGTGCGGGGGTACGTCTCCTGTGCTCTTGGCTGCCCTTATGAAGGGAAGATCTCCCCAGCTAAAG
TAGCTGAGGTCACCAAGAAGTTCTACTCAATGGGCTGCTACGAGATCTCCCTGGGGGACACCATTGGT
GTGGGCACCCCAGGGATCATGAAAGACATGCTGTCTGCTGTCATGCAGGAAGTGCCTCTGGCTGCCCT
GGCTGTCCACTGCCATGACACCTATGGTCAAGCCCTGGCCAACACCTTGATGGCCCTGCAGATGGGAG
TGAGTGTCGTGGACTCTTCTGTGGCAGGACTTGGAGGCTGTCCCTACGCACAGGGGGCATCAGGAAAC
TTGGCCACAGAAGACCTGGTCTACATGCTAGAGGGCTTGGGCATTCACACGGGTGTGAATCTCCAGAA
GCTTCTGGAAGCTGGAAACTTTATCTGTCAAGCCCTGAACAGAAAAACTAGCTCCAAAGTGGCTCAGG
CTACCTCTAAACTCTGATCTAGAACAAAAA

| | | | |
|---|---|---|---|
| NOV7b, 223317153 | | | |
| Protein Sequence | SEQ ID NO:62 | 301 aa | MW at 31819.7 kD |

TMGTLPKRVKIVEVGPRDGLQNEKNIVSTPVKIKLIDMLSEAGLSVIETTSFVSPKWVPQMGDHTEVL
KGIQKFPGINYPVLTPNLKGFEAAVAAGAKEVVIFGAASELFTKKNINCSIEESFQRFDAILKAAQSA
NISVRGYVSCALGCPYEGKISPAKVAEVTKKFYSMGCYEISLGDTIGVGTPGIMKDMLSAVMQEVPLA
ALAVHCHDTYGQALANTLMALQMGVSVVDSSVAGLGGCPYAQGASGNLATEDLVYMLEGLGIHTGVNL
QKLLEAGNFICQALNRKTSSKVAQATSKL

| | | |
|---|---|---|
| NOV7c, CG96859-01 | SEQ ID NO:63 | 1568 bp |
| DNA Sequence | ORF Start: ATG at 15 | ORF Stop: TGA at 990 |

GAATTCCGGCCAAGATGGCAGCAATGAGGAAGGCGCTTCCGCGGCGACTGGTGGGCTTGGCGTCCCTC
CGGGCTGTCAGCACCTCATCTATGGGCACTTTACCAAAGCGGGTGAAAATTGTGGAAGTTGGTCCCCG
AGATGGACTACAAAATGAAAAGAATATCGTATCTACTCCAGTGAAAATCAAGCTGATAGACATGCTTT
CTGAAGCAGGACTCTCTGTTATAGAAACCACCAGCTTTGTGTCTCCTAAGTGGGTTCCCCAGATGGGT
GACCACACTGAAGTCTTGAAGGGCATTCAGAAGTTTCCTGGCATCAACTACCCAGTCCTGACCCCAAA
TTTGAAAGGCTTCGAGGCAGCGGTTGCTGCTGGAGCCAAGGAAGTAGTCATCTTTGGAGCTGCCTCAG
AGCTCTTCACCAAGAAGAACATCAATTGTTCCATAGAGGAGAGTTTTCAGAGGTTTGACGCAATCCTG
AAGGCAGCGCAGTCAGCCAATATTTCTGTGCGGGGGTACGTCTCCTGTGCTCTTGGCTGCCCTTATGA
AGGGAAGATCTCCCCAGCTAAAGTAGCTGAGGTCACCAAGAAGTTCTACTCAATGGGCTGCTACGAGA
TCTCCCTGGGGGACACCATTGGTGTGGGCACCCCAGGGATCATGAAAGACATGCTATCTGCTGTCATG
CAGGAAGTGCCTCTGGCTGCCCTGGCTGTCCACTGCCATGACACCTATGGTCAAGCCCTGACCAACAC
CTTGATGGCCCTGCAGATGGGAGTGAGTGTCGTGGACTCTTCTGTGGCAGGACTTGGAGGCTGTCCCT
ACGCACAGGGGGCATCAGGAAACTTGGCCACAGAAGACCTGGTCTACATGCTAGAGGGCTTGGGCATT
CACACGGGTGTGAATCTCCAGAAGCTTCTGGAAGCTGGAAACTTTATCTGTCAAGCCCTGAACAGAAA
AACTAGCTCCAAAGTGGCTCAGGCTACCTGTAAACTCTGAGCCCCTTGCCCACCTGAAGCCCTGGGGA
TGATGTGGAAATAGGGGCACACACAGATGATTCATGGATGGGGACATGGAAATGAGAATAGGTTAAAT
GGTGCAGGTACCTCATAGCCAGCTCTACACAGAGGTCTCTCCTGGCAGAAAGCAGGCGAAGGGCAGGA
GGAGCTGCTTGGCAGAAGGACCTCCTGCCCAGACCTGAGGAGTGAGAGGCTTTGAGGGCTGAAGTCTC
CCTTTGTTACGGACCCTGGCCCAGGAGTTGAATGCCTGAGGACGTGTGGGAACCCCGTTCCCTACTTA
GCATGATCCTTGAGTCTCCTCTCTGGATGGAATCCGCGAGCTGGCCACCTGGCCACCCTCTACACGGC
TCCACCCTGCCATGGCCGTGGGGCCCTTGCTCTCTGACTTCTCAGGACACAGGTCATGGAGGTTCTTC
CCAAGCTGGCAGAGGCCATTTGTGGAAAGTGGAGAGCTACGTGGTGGCCGTCTGCCAACTCCAGCATC
TCTGGAAAATCTCCACGCTGAATGTGATTTTTGAAAACAGCTTATGTAATTAAAGGTTGAATGGCACA
TCAT

| | | | |
|---|---|---|---|
| NOV7c, CG96859-01 | | | |
| Protein Sequence | SEQ ID NO:64 | 325 aa | MW at 34389.8 kD |

MAAMRKALPRRLVGLASLRAVSTSSMGTLPKRVKIVEVGPRDGLQNEKNIVSTPVKIKLIDMLSEAGL
SVIETTSFVSPKWVPQMGDHTEVLKGIQKFPGINYPVLTPNLKGFEAAVAAGAKEVVIFGAASELFTK
KNINCSIEESFQRFDAILKAAQSANISVRGYVSCALGCPYEGKISPAKVAEVTKKFYSMGCYEISLGD
TIGVGTPGIMKDMLSAVMQEVPLAALAVHCHDTYGQALTNTLMALQMGVSVVDSSVAGLGGCPYAQGA
SGNLATEDLVYMLEGLGIHTGVNLQKLLEAGNFICQALNRKTSSKVAQATCKL

| | | |
|---|---|---|
| NOV7d, CG96859-02 | SEQ ID NO:65 | 1355 bp |
| DNA Sequence | ORF Start: ATG at 15 | ORE Stop: TGA at 777 |

GAATTCCGGCCAAGATGGCAGCAATGAGGAAGGCGCTTCCGCGGCGACTGGTGGGCTTGGCGTCCCTC
CGGGCTGTCAGCACCTCATCTATGGGCACTTTACCAAAGCGGGTGAAAATTGTGGAAGTTGGTCCCCG
AGATGGACTACAAAATGAAAAGAATATCGTATCTACTCCAGTGAAAATCAAGCTGATAGACATGCTTT
CTGAAGCAGGACTCTCTGTTATAGAAACCACCAGCTTTGTGTCTCCTAAGTGGGTTCCCCAGATGGGT
GACCACACTGAAGTCTTGAAGGGCATTCAGAAGTTTCCTGGCATCAACTACCCAGTCCTGACCCCAAA
TTTGAAAGGCTTCGAGGCAGCGGTCACCAAGAAGTTCTACTCAATGGGCTGCTACGAGATCTCCCTGG
GGGACACCATTGGTGTGGGCACCCCAGGGATCATGAAAGACATGCTGTCTGCTGTCATGCAGGAAGTG
CCTCTGGCTGCCCTGGCTGTCCACTGCCATGACACCTATGGTCAAGCCCTGGCCAACACCTTGATGGC
CCTGCAGATGGGAGTGAGTGTCGTGGACTCTTCTGTGGCAGGACTTGGAGGCTGTCCCTACGCACAGG
GGGCATCAGGAAACTTGGCCACAGAAGACCTGGTCTACATGCTAGAGGGCTTGGGCATTCACACGGGT
GTGAATCTCCAGAAGCTTCTGGAAGCTGGAAACTTTATCTGTCAAGCCCTGAACAGAAAAACTAGCTC
CAAAGTGGCTCAGGCTACCTGTAAACTCTGAGCCCCTTGCCCACCTGAAGCCCTGGGGATGATGTGGA
AATAGGGGCACACACAGATGATTCATGGATGGGGACATGGAAATGAGAATAGGTTAAATGGTGCAGGT
ACCTCATAGCCAGCTCTACACAGAGGTCTCTCCTGGCAGAAAGCAGGCGAAGGGCAGGAGGAGCTGCT
TGGCAGAAGGACCTCCTGCCCAGACCTGAGGAGTGAGAGGCTTTGAGGGCTGAAGTCTCCCTTTGTTA
CGGACCCTGGCCCAGGAGTTGAATGCCTGAGGACGTGTGGGAACCCCGTTCCCTACTTAGCATGATCC
TTGAGTCTCCTCTCTGGATGGAATCCGCGAGCTGGCCACCTGGCCACCCTCTACACGGCTCCACCCTG
CCATGGCCGTGGGGCCCTTGCTCTCTGACTTCTCAGGACACAGGTCATGGAGGTTCTTCCCAAGCTGG
CAGAGGCCATTTGTGGAAAGTGGAGAGCTACGTGGTGGCCGTCTGCCAACTCCAGCATCTCTGGAAAA
TCTCCACGCTGAATGTGATTTTTGAAAACAGCTTATGTAATTAAAGGTTGAATGGCACATCAT

| | | | |
|---|---|---|---|
| NOV7d, CG96859-02 | | | |
| Protein Sequence | SEQ ID NO:66 | 254 aa | MW at 26909.3 kD |

MAAMRKALPRRLVGLASLRAVSTSSMGTLPKRVKIVEVGPRDGLQNEKNIVSTPVKIKLIDMLSEAGL
SVIETTSFVSPKWVPQMGDHTEVLKGIQKFPGINYPVLTPNLKGFEAAVTKKFYSMGCYEISLGDTIG
VGTPGIMKDMLSAVMQEVPLAALAVHCHDTYGQALANTLMALQMGVSVVDSSVAGLGGCPYAQGASGN
LATEDLVYMLEGLGIHTGVNLQKLLEAGNFICQALNRKTSSKVAQATCKL

| | | |
|---|---|---|
| NOV7e, CG96859-04 | SEQ ID NO:67 | 788 bp |
| DNA Sequence | ORF Start: ATG at 2 | ORF Stop: TGA at 764 |

GATGGCAGCAATGAGGAAGGCGCTTCCGCGGCGACTGGTGGGCTTGGCGTCCCTCCGGGCTGTCAGCA
CCTCATCTATGGGCACTTTACCAAAGCGGGTGAAAATTGTGGAAGTTGGTCCCCGAGATGGACTACAA
AATGAAAGGAATATCGTATCTACTCCAGTGAAAATCAAGCTGATAGACATGCTTTCTGAAGCAGGACT
CTCTGTTATAGAAACCACCAGCTTTGTGTCTCCTAAGTGGGTTCCCCAGATGGGTGACCACACTGAAG
TCTTGAAGGGCATTCAGAAGTTTCCTGGCATCAACTACCCAGTCCTGACCCCAAATTTGAAAGGCTTC
GAGGCAGCGGTCACCAAGAAGTTCTACTCAATGGGCTGCTACGAGATCTCCCTGGGGGACACCATTGG
TGTGGGCACCCCAGGGATCATGAAAGACATGCTGTCTGCTGTCATGCAGGAAGTGCCTCTGGCTGCCC
TGGCTGTCCACTGCCATGACACCTATGGTCAAGCCCTGGCCAACACCTTGATGGCCCTGCAGATGGGA
GTGAGTGTCGTGGACTCTTCTGTGGCAGGACTTGGAGGCTGTCCCTACGCACAGGGGGCATCAGGAAA
CTTGGCCACAGAAGACCTGGTCTACATGCTAGAGGGCTTGGGCATTCACACGGGTGTGAATCTCCAGA
AGCTTCTGGAAGCTGGAAACTTTATCTGTCAAGCCCTGAACAGAAAAACTAGCTCCAAAGTGGCTCAG
GCTACCTGTAAACTCTGAGCCCCTTGCCCACCTGAAGCCC

| | | | |
|---|---|---|---|
| NOV7e, CG96859-04 | | | |
| Protein Sequence | SEQ ID NO:68 | 254 aa | MW at 26937.3 kD |

MAAMRKALPRRLVGLASLRAVSTSSMGTLPKRVKIVEVGPRDGLQNERNIVSTPVKIKLIDMLSEAGL
SVIETTSFVSPKWVPQMGDHTEVLKGIQKFPGINYPVLTPNLKGFEAAVTKKFYSMGCYEISLGDTIG
VGTPGIMKDMLSAVMQEVPLAALAVHCHDTYGQALANTLMALQMGVSVVDSSVAGLGGCPYAQGASGN
LATEDLVYMLEGLGIHTGVNLQKLLEAGNFICQALNRKTSSKVAQATCKL

| | | |
|---|---|---|
| NOV7f, CG96859-05 | SEQ ID NO:69 | 893 bp |
| DNA Sequence | ORF Start: ATG at 2 | ORF Stop: TGA at 869 |

GATGGCAGCAATGAGGAAGGCGCTTCCGCGGCGACTGGTGGGCTTGGCGTCCCTCCGGGCTGTCAGCA
CCTTATCTATGGGCACTTTACCAAAGCGGGTGAAAATTGTGGAAGTTGGTCCCCGAGATGGACTACAA
AATGAAAAGAATATCGTATCTACTCCAGTGAAAATCAAGCTGATAGACATGCTTTCTGAAGCAGGACT
CTCTGTTATAGAAACCACCAGCTTTGTGTCTCCTAAGTGGGTTCCCCAGATGGGTGACCACACTGAAG
TCTTGAAGGGCATTCAGAAGTTTCCTGGCATCAACTACCCAGTCCTGACCCCAAATTTGAAAGGCTTC
GAGGCAGCGGTTGCTGCTGGAGCCAAGGAAGTAGTCATCTTTGGAGCTGCCTCAGAGCTCTTCACCAA
GAAGAACATCAATTGTTCCATAGAGGAGAGTTTTCAGAGGTTTGACGCAATCCTGAAGGCAGCGCAGT
CAGCCAATATTTCTGTGCGGGGGTACGTCTCCTGTGCTCTTGGCTGCCCTTATGAAGGGAAGATCTCC
CCAGCTAAAGTAGCTGAGGAAGTGCCTCTGGCTGCCCTGGCTGTCCACTGCCATGACACCTATGGTCA
AGCCCTGGCCAACACCTTGATGGCCCTGCAGATGGGAGTGAGTGTCGTGGACTCTTCTGTGGCAGGAC
TTGGAGGCTGTCCCTACGCACAGGGGGCATCAGGAAACTTGGCCACAGAAGACCTGGTCTACATGCTA
GAGGGCTTGGGCATTCACACGGGTGTGAATCTCCAGAAGCTTCTGGAAGCTGGAAACTTTATCTGTCA
AGCCCTGAACAGAAAAACTAGCTCCAAAGTGGCTCAGGCTACCTGTAAACTCTGAGCCCCTTGCCCAC
CTGAAGCCC

| | | | |
|---|---|---|---|
| NOV7f, CG96859-05 | | | |
| Protein Sequence | SEQ ID NO:70 | 289 aa | MW at 30531.3 kD |

MAAMRKALPRRLVGLASLRAVSTLSMGTLPKRVKIVEVGPRDGLQNEKNIVSTPVKIKLIDMLSEAGL
SVIETTSFVSPKWVPQMGDHTEVLKGIQKFPGINYPVLTPNLKGFEAAVAAGAKEVVIFGAASELFTK
KNINCSIEESFQRFDAILKAAQSANISVRGYVSCALGCPYEGKISPAKVAEEVPLAALAVHCHDTYGQ
ALANTLMALQMGVSVVDSSVAGLGGCPYAQGASGNLATEDLVYMLEGLGIHTGVNLQKLLEAGNFICQ
ALNRKTSSKVAQATCKL

| | | |
|---|---|---|
| NOV7g, CG96859-06 | SEQ ID NO:71 | 1353 bp |
| DNA Sequence | ORF Start: ATG at 202 | ORF Stop: at 1171 |

CCCCAAAATTCGTAACAACTCCGCCCCATTGACGCAAATGGGCGGTAGGCGTGTACGGTGGGAGGTCT
ATATAAGCAGAGCTCTCTGGCTAACTAGAGAACCCACTGCTTACTGGCTTATCGAAATTAATACGACT
CACTATAGGGAGACCCAAGCTGGCTAGCGTTTAAACTTAAGCTTGGTACCGAGCTCGGATCCACCATG
GCAGCAATGAGGAAGGCGCTTCCGCGGCGACTGGTGGGCTTGGCGTCCCTCCGGGCTGTCAGCACCTC
ATCTATGGGCACTTTACCAAAGCGGGTGAAAATTGTGGAAGTTGGTCCCCGAGATGGACTACAAAATG
AAAAGAATATCGTATCTACTCCAGTGAAAATCAAGCTGATAGACATGCTTTCTGAAGCAGGACTCTCT
GTTATAGAAACCACCAGCTTTGTGTCTCCTAAGTGGGTTCCCCAGATGGGTGACCACACTGAAGTCTT
GAAGGGCATTCAGAAGTTTCCTGGCATCAACTACCCAGTCCTGACCCCAAATTTGAAAGGCTTCGAGG
CAGCGGTTGCTGCTGGAGCCAAGGAAGTAGTCATCTTTGGAGCTGCCTCAGAGCTCTTCACCAAGAAG
AACATCAATTGTTCCATAGAGGAGAGTTTTCAGAGGTTTGACGCAATCCTGAAGGCAGCGCAGTCAGC
CAATATTTCTGTGCGGGGGTACGTCTCCTGTGCTCTTGGCTGCCCTTATGAAGGGAAGATCTCCCCAG
CTAAAGTAGCTGAGGTCACCAAGAAGTTCTACTCAATGGGCTGCTACGAGATCTCCCTGGGGGACACC
ATTGGTGTGGGCACCCCAGGGATCATGAAAGACATGCTGTCTGCTGTCATGCAGGAAGTGCCTCTGGC
TGCCCTGGCTGTCCACTGCCATGACACCTATGGTCAAGCCCTGGCCAACACCTTGATGGCCCTGCAGA
TGGGAGTGAGTGTCGTGGACTCTTCTGTGGCAGGACTTGGAGGCTGTCCCTACGCACAGGGGGCATCA
GGAAACTTGGCCACAGAAGACCTGGTCTACATGCTAGAGGGCTTGGGCATTCACACGGGTGTGAATCT
CCAGAAGCTTCTGGAAGCTGGAAACTTTATCTGTCAAGCCCTGAACAGAAAAACTAGCTCCAAAGTGG
CTCAGGCTACCTGTAAACTCTGAGCGGCCGCTCGAGTCTAGAGGGCCCGTTTAAACCCGCTGATCAGC
CTCGACTGTGCCTTCTAGTTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCGCATTGTCTGAG

| | | | |
|---|---|---|---|
| NOV7g, CG96859-06 | | | |
| Protein Sequence | SEQ ID NO:72 | 323 aa | MW at 34118.4 kD |

MAAMRKALPRRLVGLASLRAVSTSSMGTLPKRVKIVEVGPRDGLQNEKNIVSTPVKIKLIDMLSEAGL
SVIETTSFVSPKWVPQMGDHTEVLKGIQKFPGINYPVLTPNLKGFEAAVAAGAKEVVIFGAASELFTK
KNINCSIEESFQRFDAILKAAQSANISVRGYVSCALGCPYEGKISPAKVAEVTKKFYSMGCYEISLGD
TIGVGTPGIMKDMLSAVMQEVPLAALAVHCHDTYGQALANTLMALQMGVSVVDSSVAGLGGCPYAQGA
SGNLATEDLVYMLEGLGIHTGVNLQKLLEAGNFICQALNRKTSSKVAQATC

| | | |
|---|---|---|
| NOV7h, CG96859-07 | SEQ ID NO:73 | 1969 bp |
| DNA Sequence | ORF Start: at 64 | ORF Stop: TGA at 967 |

TAACTTTATTATTAAAAATTAAAGAGGTATATATTAATGTATCGATTAAATAAGGAGGAATAAACCAT
GGGCACTTTACCAAAGCGGGTGAAAATTGTGGAAGTTGGTCCCCGAGATGGACTACAAAATGAAAAGA
ATATCGTATCTACTCCAGTGAAAATCAAGCTGATAGACATGCTTTCTGAAGCAGGACTCTCTGTTATA
GAAACCACCAGCTTTGTGTCTCCTAAGTGGGTTCCCCAGATGGGTGACCACACTGAAGTCTTGAAGGG
CATTCAGAAGTTTCCTGGCATCAACTACCCAGTCCTGACCCCAAATTTGAAAGGCTTCGAGGCAGCGG
TTGCTGCTGGAGCCAAGGAAGTAGTCATCTTTGGAGCTGCCTCAGAGCTCTTCACCAAGAAGAACATC
AATTGTTCCATAGAGGAGAGTTTTCAGAGGTTTGACGCAATCCTGAAGGCAGCGCAGTCAGCCAATAT
TTCTGTGCGGGGGTACGTCTCCTGTGCTCTTGGCTGCCCTTATGAAGGGAAGATCTCCCCAGCTAAAG
TAGCTGAGGTCACCAAGAAGTTCTACTCAATGGGCTGCTACGAGATCTCCCTGGGGGACACCATTGGT
GTGGGCACCCCAGGGATCATGAAAGACATGCTGTCTGCTGTCATGCAGGAAGTGCCTCTGGCTGCCCT
GGCTGTCCACTGCCATGACACCTATGGTCAAGCCCTGGCCAACACCTTGATGGCCCTGCAGATGGGAG
TGAGTGTCGTGGACTCTTCTGTGGCAGGACTTGGAGGCTGTCCCTACGCACAGGGGGCATCAGGAAAC
TTGGCCACAGAAGACCTGGTCTACATGCTAGAGGGCTTGGGCATTCACACGGGTGTGAATCTCCAGAA
GCTTCTGGAAGCTGGAAACTTTATCTGTCAAGCCCTGAACAGAAAAACTAGCTCCAAAGTGGCTCAGG
CTACCTGTAAACTCTGA

| | | | |
|---|---|---|---|
| NOV7h, CG96859-07 | | | |
| Protein Sequence | SEQ ID NO:74 | 301 aa | MW at 31835.7 kD |

TMGTLPKRVKIVEVGPRDGLQNEKNIVSTPVKIKLIDMLSEAGLSVIETTSFVSPKWVPQMGDHTEVL
KGIQKFPGINYPVLTPNLKGFEAAVAAGAKEVVIFGAASELFTKKNINCSIEESFQRFDAILKAAQSA
NISVRGYVSCALGCPYEGKISPAKVAEVTKKFYSMGCYEISLGDTIGVGTPGIMKDMLSAVMQEVPLA
ALAVHCHDTYGQALANTLMALQMGVSVVDSSVAGLGGCPYAQGASGNLATEDLVYMLEGLGIHTGVNL
QKLLEAGNFICQALNRKTSSKVAQATCKL

| | | |
|---|---|---|
| NOV7i, GG96859-08 | SEQ ID NO:75 | 969 bp |
| DNA Sequence | ORF Start: at 64 | ORF Stop: TGA at 967 |

TAACTTTATTATTAAAAATTAAAGAGGTATATATTAATGTATCGATTAAATAAGGAGGAATAAACCAT
GGGCACTTTACCAAAGCGGGTGAAAATTGTGGAAGTTGGTCCCCGAGATGGACTACAAAATGAAAAGA
ATATCGTATCTACTCCAGTGAAAATCAAGCTGATAGACATGCTTTCTGAAGCAGGACTCTCTGTTATA
GAAACCACCAGCTTTGTGTCTCCTAAGTGGGTTCCCCAGATGGGTGACCACACTGAAGTCTTGAAGGG
CATTCAGAAGTTTCCTGGCATCAACTACCCAGTCCTGACCCCAAATTTGAAAGGCTTCGAGGCAGCGG
TTGCTGCTGGAGCCAAGGAAGTAGTCATCTTTGGAGCTGCCTCAGAGCTCTTCACCAAGAAGAACATC
AATTGTTCCATAGAGGAGAGTTTTCAGAGGTTTGACGCAATCCTGAAGGCAGCGCAGTCAGCCAATAT
TTCTGTGCGGGGGTACGTCTCCTGTGCTCTTGGCTGCCCTTATGAAGGGAAGATCTCCCCAGCTAAAG
TAGCTGAGGTCACCAAGAAGTTCTACTCAATGGGCTGCTACGAGATCTCCCTGGGGGACACCATTGGT
GTGGGCACCCCAGGGATCATGAAAGACATGCTGTCTGCTGTCATGCAGGAAGTGCCTCTGGCTGCCCT
GGCTGTCCACTGCCATGACACCTATGGTCAAGCCCTGGCCAACACCTTGATGGCCCTGCAGATGGGAG
TGAGTGTCGTGGACTCTTCTGTGGCAGGACTTGGAGGCTGTCCCTACGCACAGGGGGCATCAGGAAAC
TTGGCCACAGAAGACCTGGTCTACATGCTAGAGGGCTTGGGCATTCACACGGGTGTGAATCTCCAGAA
GCTTCTGGAAGCTGGAAACTTTATCTGTCAAGCCCTGAACAGAAAAACTAGCTCCAAAGTGGCTCAGG
CTACCTGTAAACTCTGA

| | | | | |
|---|---|---|---|---|
| NOV7i, CG96859-08 | | | | +TL,43 |
| Protein Sequence | SEQ ID NO:76 | 301 aa | MW at 31835.7 kD | |

TMGTLPKRVKIVEVGPRDGLQNEKNIVSTPVKIKLIDMLSEAGLSVIETTSFVSPKWVPQMGDHTEVL
KGIQKFPGINYPVLTPNLKGFEAAVAAGAKEVVIFGAASELFTKKNINCSIEESFQRFDAILKAAQSA
NISVRGYVSCALGCPYEGKISPAKVAEVTKKFYSMGCYEISLGDTIGVGTPGIMKDMLSAVNQEVPLA
ALAVHCHDTYGQALANTLMALQMGVSVVDSSVAGLGGCPYAQGASGNLATEDLVYMLEGLGIHTGVNL
QKLLEAGNFICQALNRKTSSKVAQATCKL

| | | |
|---|---|---|
| NOV7j, CG96859-09 | SEQ ID NO:77 | 987 bp |
| DNA Sequence | ORF Start: at 64 | ORF Stop: TGA at 985 |

TAACTTTATTATTAAAAATTAAAGAGGTATATATTAATGTATCGATTAAATAAGGAGGAATAAACCAT
GGGCCACCATCACCACCATCACACTTTACCAAAGCGGGTGAAAATTGTGGAAGTTGGTCCCCGAGATG
GACTACAAAATGAAAAGAATATCGTATCTACTCCAGTGAAAATCAAGCTGATAGACATGCTTTCTGAA
GCAGGACTCTCTGTTATAGAAACCACCAGCTTTGTGTCTCCTAAGTGGGTTCCCCAGATGGGTGACCA
CACTGAAGTCTTGAAGGGCATTCAGAAGTTTCCTGGCATCAACTACCCAGTCCTGACCCCAAATTTGA
AAGGCTTCGAGGCAGCGGTTGCTGCTGGAGCCAAGGAAGTAGTCATCTTTGGAGCTGCCTCAGAGCTC
TTCACCAAGAAGAACATCAATTGTTCCATAGAGGAGAGTTTTCAGAGGTTTGACGCAATCCTGAAGGC
AGCGCAGTCAGCCAATATTTCTGTGCGGGGGTACGTCTCCTGTGCTCTTGGCTGCCCTTATGAAGGGA
AGATCTCCCCAGCTAAAGTAGCTGAGGTCACCAAGAAGTTCTACTCAATGGGCTGCTACGAGATCTCC
CTGGGGGACACCATTGGTGTGGGCACCCCAGGGATCATGAAAGACATGCTGTCTGCTGTCATGCAGGA
AGTGCCTCTGGCTGCCCTGGCTGTCCACTGCCATGACACCTATGGTCAAGCCCTGGCCAACACCTTGA
TGGCCCTGCAGATGGGAGTGAGTGTCGTGGACTCTTCTGTGGCAGGACTTGGAGGCTGTCCCTACGCA
CAGGGGGCATCAGGAAACTTGGCCACAGAAGACCTGGTCTACATGCTAGAGGGCTTGGGCATTCACAC
GGGTGTGAATCTCCAGAAGCTTCTGGAAGCTGGAAACTTTATCTGTCAAGCCCTGAACAGAAAAACTA
GCTCCAAAGTGGCTCAGGCTACCTGTAAACTCTGA

| | | | |
|---|---|---|---|
| NOV7j, CG96859-09 | | | |
| Protein Sequence | SEQ ID NO:78 | 307 aa | MW at 32658.6 kD |

TMGHHHHHHTLPKRVKIVEVGPRDGLQNEKNIVSTPVKIKLIDMLSEAGLSVIETTSFVSPKWVPQMG
DHTEVLKGIQKFPGINYPVLTPNLKGFEAAVAAGAKEVVIFGAASELFTKKNINCSIEESFQRFDAIL
KAAQSANISVRGYVSCALGCPYEGKISPAKVAEVTKKFYSMGCYEISLGDTIGVGTPGIMKDMLSAVM
QEVPLAALAVHCHDTYGQALANTLMALQMGVSVVDSSVAGLGGCPYAQGASGNLATEDLVYMLEGLGI
HTGVNLQKLLEAGNFICQALNRKTSSKVAQATCKL

A ClustalW comparison of the above protein sequences yields the following sequence alignment shown in Table G6.

TABLE G6
Comparison of the NOV7 protein sequences.

| | |
|---|---|
| NOV7a | MAANRKALPRRLVGLASLRAVSTSSMGTLPKRVKIVEVGPRDGLQNEKNIVSTPVKIKLI |
| NOV7b | ————————————————————————TMGTLPKRVKIVEVGPRDGLQNEKNIVSTPVKIKLI |
| NOV7c | MAAMRKALPRRLVGLASLRAVSTSSMGTLPKRVKIVEVGPRDGLQNEKNIVSTPVKIKLI |
| NOV7d | MAAMRKALPRRLVGLASLRAVSTSSMGTLPKRVKIVEVGPRDGLQNEKNIVSTPVKIKLI |
| NOV7e | MAAMRKALPRRLVGLASLRAVSTSSMGTLPKRVKIVEVGPRDGLQNERNIVSTPVKIKLI |
| NOV7f | MAAMRKALPRRLVGLASLRAVSTLSMGTLPKRVKIVEVGPRDGLQNEKNIVSTPVKIKLI |
| NOV7g | MAAMRKALPRRLVGLASLRAVSTSSMGTLPKRVKIVEVGPRDGLQNEKNIVSTPVKIKLI |
| NOV7h | ————————————————————————TMGTLPKRVKIVEVGPRDGLQNEKNIVSTPVKIKLI |
| NOV7i | ————————————————————————TMGTLPKRVKIVEVGPRDGLQNEKNIVSTPVKIKLI |
| NOV7j | ——————————————————TMGHHHHHHTLPKRVKIVEVGPRDGLQNEKNIVSTPVKIKLI |
| NOV7a | DMLSEAGLSVIETTSFVSPKWVPQMGDHTEVLKGIQKFPGINYPVLTPNLKGFEAAVAAG |
| NOV7b | DMLSEAGLSVIETTSFVSPKWVPQMGDHTEVLKGIQKFPGINYPVLTPNLKGFEAAVAAG |
| NOV7c | DMLSEAGLSVIETTSFVSPKWVPQMGDHTEVLKGIQKFPGINYPVLTPNLKGFEAAVAAG |
| NOV7d | DMLSEAGLSVIETTSFVSPKWVPQMGDHTEVLKGIQKFPGINYPVLTPNLKGFEAAVTK— |
| NOV7e | DMLSEAGLSVIETTSFVSPKWVPQMGDHTEVLKGIQKFPGINYPVLTPNLKGFEAAVTK— |
| NOV7f | DMLSEAGLSVIETTSFVSPKWVPQMGDHTEVLKGIQKFPGINYPVLTPNLKGFEAAVAAG |
| NOV7g | DMLSEAGLSVIETTSFVSPKWVPQMGDHTEVLKGIQKFPGINYPVLTPNLKGFEAAVAAG |
| NOV7h | DMLSEAGLSVIETTSFVSPKWVPQMGDHTEVLKGIQKFPGINYPVLTPNLKGFEAAVAAG |
| NOV7i | DMLSEAGLSVIETTSFVSPKWVPQMGDHTEVLKGIQKFPGINYPVLTPNLKGFEAAVAAG |
| NOV7j | DMLSEAGLSVIETTSFVSPKWVPQMGDHTEVLKGIQKFPGINYPVLTPNLKGFEAAVAAG |
| NOV7a | AKEVVIFGAASELFTKKNINCSIEESFQRFDAILKAAQSANISVRGYVSCALGCPYEGKI |
| NOV7b | AKEVVIFGAASELFTKKNINCSIEESFQRFDAILKAAQSANISVRGYVSCALGCPYEGKI |
| NOV7c | AKEVVIFGAASELFTKKNINCSIEESFQRFDAILKAAQSANISVRGYVSCALGCPYEGKI |
| NOV7d | ———————————————————————————————————————————————————————————— |
| NOV7e | ———————————————————————————————————————————————————————————— |
| NOV7f | AKEVVIFGAASELFTKKNINCSIEESFQRFDAILKAAQSANISVRGYVSCALGCPYEGKI |
| NOV7g | AKEVVIFGAASELFTKKNINCSIEESFQRFDAILKAAQSANISVRGYVSCALGCPYEGKI |
| NOV7h | AKEVVIFGAASELFTKKNINCSIEESFQRFDAILKAAQSANISVRGYVSCALGCPYEGKI |
| NOV7i | AKEVVIFGAASELFTKKNINCSIEESFQRFDAILKAAQSANISVRGYVSCALGCPYEGKI |
| NOV7j | AKEVVIFGAASELFTKKNINCSIEESFQRFDAILKAAQSANISVRGYVSCALGCPYEGKI |
| NOV7a | SPAKVAEVTKKFYSMGCYEISLGDTIGVGTPGIMKDMLSAVMQEVPLAALAVHCHDTYGQ |
| NOV7b | SPAKVAEVTKKFYSMGCYEISLGDTIGVGTPGIMKDMLSAVMQEVPLAALAVHCHDTYGQ |
| NOV7c | SPAKVAEVTKKFYSMGCYEISLGDTIGVGTPGIMKDMLSAVMQEVPLAALAVHCHDTYGQ |
| NOV7d | ——————————KFYSMGCYEISLGDTIGVGTPGIMKDMLSAVMQEVPLAALAVHCHDTYGQ |
| NOV7e | ——————————KFYSMGCYEISLGDTIGVGTPGIMKDMLSAVMQEVPLAALAVHCHDTYGQ |
| NOV7f | SPAKVAE————————————————————————————————————EVPLAALAVHCHDTYGQ |
| NOV7g | SPAKVAEVTKKFYSMGCYEISLGDTIGVGTPGIMKDMLSAVMQEVPLAALAVHCHDTYGQ |
| NOV7h | SPAKVAEVTKKFYSMGCYEISLGDTIGVGTPGIMKDMLSAVMQEVPLAALAVHCHDTYGQ |
| NOV7i | SPAKVAEVTKKFYSMGCYEISLGDTIGVGTPGIMKDMLSAVMQEVPLAALAVHCHDTYGQ |
| NOV7j | SPAKVAEVTKKFYSMGCYEISLGDTIGVGTPGIMKDMLSAVMQEVPLAALAVHCHDTYGQ |
| NOV7a | ALANTLMALQMGVSVVDSSVAGLGGCPYAQGASGNLATEDLVYMLEGLGIHTGVNLQKLL |
| NOV7b | ALANTLMALQMGVSVVDSSVAGLGGCPYAQGASGNLATEDLVYMLEGLGIHTGVNLQKLL |
| NOV7c | ALTNTLMALQMGVSVVDSSVAGLGGCPYAQGASGNLATEDLVYMLEGLGIHTGVNLQKLL |
| NOV7d | ALANTLMALQMGVSVVDSSVAGLGGCPYAQGASGNLATEDLVYMLEGLGIHTGVNLQKLL |
| NOV7e | ALANTLMALQMGVSVVDSSVAGLGGCPYAQGASGNLATEDLVYMLEGLGIHTGVNLQKLL |
| NOV7f | ALANTLMALQMGVSVVDSSVAGLGGCPYAQGASGNLATEDLVYMLEGLGIHTGVNLQKLL |
| NOV7g | ALANTLMALQMGVSVVDSSVAGLGGCPYAQGASGNLATEDLVYMLEGLGIHTGVNLQKLL |
| NOV7h | ALANTLMALQMGVSVVDSSVAGLGGCPYAQGASGNLATEDLVYMLEGLGIHTGVNLQKLL |
| NOV7i | ALANTLMALQMGVSVVDSSVAGLGGCPYAQGASGNLATEDLVYMLEGLGIHTGVNLQKLL |
| NOV7j | ALANTLMALQMGVSVVDSSVAGLGGCPYAQGASGNLATEDLVYMLEGLGIHTGVNLQKLL |
| NOV7a | EAGNFICQALNRKTSSKVAQATCKL |
| NOV7b | EAGNFICQALNRKTSSKVAQATSKL |
| NOV7c | EAGNFICQALNRKTSSKVAQATCKL |
| NOV7d | EAGNFICQALNRKTSSKVAQATCKL |
| NOV7e | EAGNFICQALNRKTSSKVAQATCKL |
| NOV7f | EAGNFICQALNRKTSSKVAQATCKL |
| NOV7g | EAGNFICQALNRKTSSKVAQATC—— |
| NOV7h | EAGNFICQALNRKTSSKVAQATCKL |
| NOV7i | EAGNFICQALNRKTSSKVAQATCKL |
| NOV7j | EAGNFICQALNRKTSSKVAQATCKL |
| NOV7a | (SEQ ID NO:60) |
| NOV7b | (SEQ ID NO:62) |
| NOV7c | (SEQ ID NO:64) |
| NOV7d | (SEQ ID NO:66) |
| NOV7e | (SEQ ID NO:68) |
| NOV7f | (SEQ ID NO:70) |
| NOV7g | (SEQ ID NO:72) |
| NOV7h | (SEQ ID NO:74) |
| NOV7i | (SEQ ID NO:76) |
| NOV7j | (SEQ ID NO:78) |

Further analysis of the NOV7a protein yielded the following properties shown in Table G7.

TABLE G7
Protein Sequence Properties NOV7a

| SignalP | Cleavage site between residues 25 and 26 | | | |
|---|---|---|---|---|
| analysis: | | | | |
| PSORT II | PSG: a new signal peptide prediction method | | | |
| analysis: | N-region: length 11; pos. chg 4; neg. chg 0 | | | |
| | H-region: length 7; peak value 0.99 | | | |
| | PSG score: −3.41 | | | |
| | GvH: von Heijne's method for signal seq. recognition | | | |
| | GvH score (threshold: −2.1): −2.69 | | | |
| | possible cleavage site: between 23 and 24 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 1.80 (at 115) | | | |
| | ALOM score: 1.80 (number of TMSs: 0) | | | |
| | MITDISC: discrimination of mitochondrial targeting seq | | | |
| | R content: | 5 | Hyd Moment(75): | 10.64 |
| | Hyd Moment(95): | 9.40 | G content: | 2 |
| | D/E content: | 1 | S/T content: | 6 |

```
Score: 1.44
Gavel: prediction of cleavage sites for mitochondrial preseq
R-2 motif at 42 KRV|KI
NUCDISC: discrimination of nuclear localization signals
pat4: none
pat7: PKRVKIV (5) at 30
bipartite: none
content of basic residues: 9.8%
NLS Score: −0.04
KDEL: ER retention motif in the C-terminus: none
ER Membrane Retention Signals:
XXRR-like motif in the N-terminus: AAMR
KKXX-like motif in the C-terminus: ATCK
SKL: peroxisomal targeting signal in the C-terminus: CKL
PTS2: 2nd peroxisomal targeting signal: none
VAC: possible vacuolar targeting motif: found
TLPK at 28
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear
discrimination
Prediction: cytoplasmic
Reliability: 94.1
COIL: Lupas's algorithm to detect coiled-coil regions
total: 0 residues
Final Results (k = 9/23):
87.0%: mitochondrial
  4.3%: Golgi
  4.3%: cytoplasmic
  4.3%: nuclear
>> prediction for CG96859-03 is mit (k = 23)
```

A search of the NOV7a protein against the Geneseq database, a proprietary database that contains sequences published in patents and patent publication, yielded several homologous proteins shown in Table G8.

TABLE G8
Geneseq Results for NOV7a

| | | NOV7a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB99771 | Protein related to | 1 . . . 325 | 324/325 (99%) | 0.0 |
| | hydroxymethylglutaryl-CoA lyase- | 1 . . . 325 | 324/325 (99%) | |
| | like enzyme - Homo sapiens, 325 aa. | | | |
| | [WO200299093-A2, 12-DEC-2002] | | | |
| AAU75774 | Human 3-hydroxy-3-methylglutaryl | 1 . . . 325 | 324/325 (99%) | 0.0 |
| | coenzyme A lyase (HMGCL) protein - | 1 . . . 325 | 324/325 (99%) | |
| | Homo sapiens, 325 aa. | | | |
| | [WO200198315-A2, 27-DEC-2001] | | | |
| AAU01613 | Gene #24 human secreted protein | 30 . . . 321 | 234/292 (80%) | e−137 |
| | homologous amino acid sequence - | 1 . . . 292 | 266/292 (90%) | |
| | Homo sapiens, 293 aa. | | | |
| | [WO200123547-A1, 05-APR-2001] | | | |
| ABB99772 | Sequence of human | 20 . . . 322 | 215/303 (70%) | e−126 |
| | hydroxymethylglutaryl-CoA lyase- | 50 . . . 352 | 259/303 (84%) | |
| | like enzyme - Homo sapiens, 355 aa. | | | |
| | [WO200299093-A2, 12-DEC-2002] | | | |
| ABB99770 | Sequence of human | 20 . . . 322 | 215/303 (70%) | e−126 |
| | hydroxymethylglutaryl-CoA lyase- | 35 . . . 337 | 259/303 (84%) | |
| | like enzyme - Homo sapiens, 340 aa. | | | |
| | [WO200299093-A2, 12-DEC-2002] | | | |

In a BLAST search of public sequence databases, the NOV7a protein was found to have homology to the proteins shown in the BLASTP data in Table G9.

TABLE G9
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P35914 | Hydroxymethylglutaryl-CoA lyase, | 1 . . . 325 | 325/325 (100%) | 0.0 |
| | mitochondrial precursor (EC 4.1.3.4) | 1 . . . 325 | 325/325 (100%) | |
| | (HMG-CoA lyase) (HL) (3-hydroxy- | | | |
| | 3-methylglutarate-CoA lyase) - | | | |
| | Homo sapiens (Human), 325 aa. | | | |
| A45470 | hydroxymethylglutaryl-CoA lyase | 1 . . . 325 | 324/325 (99%) | 0.0 |
| | (EC 4.1.3.4) - human, 325 aa. | 1 . . . 325 | 324/325 (99%) | |
| Q8HXZ6 | 3 -hydroxymethyl-3-methylglutaryl- | 1 . . . 325 | 313/325 (96%) | e−176 |
| | coenzyme A lyase - Macaca | 1 . . . 325 | 315/325 (96%) | |
| | fascicularis (Crab eating macaque) | | | |
| | (Cynomolgus monkey), 325 aa. | | | |
| CAB40160 | DJ886K2.2 (Hydroxymethylglutaryl- | 21 . . . 325 | 305/305 (100%) | e−172 |
| | CoA lyase) - Homo sapiens (Human), | 1 . . . 305 | 305/305 (100%) | |
| | 305 aa (fragment). | | | |
| P97519 | Hydroxymethylglutaryl-CoA lyase, | 1 . . . 325 | 289/325 (88%) | e−167 |
| | mitochondrial precursor (EC 4.1.3.4) | 1 . . . 325 | 311/325 (94%) | |
| | (HMG-CoA lyase) (HL) (3-hydroxy- | | | |
| | 3-methylglutarate-CoA lyase) - | | | |
| | Rattus norvegicus (Rat), 325 aa. | | | |

PFam analysis predicts that the NOV7a protein contains the domains shown in the Table

TABLE G10
Domain Analysis of NOV7a

| | | Identities/ | |
|---|---|---|---|
| | NOV7a | Similarities for | |
| Pfam Domain | Match Region | the Matched Region | Expect Value |
| HMGL-like | 41 . . . 314 | 103/307 (34%) | 4.1e−118 |
| | | 249/307 (81%) | |

### EXAMPLE G3

### Single Nucleotide Polymorphisms (SNPs) of CG96859-03

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing. Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265.

In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

Results

The DNA and protein sequences for the novel single nucleotide polymorphic variants of the HYDROXYMETHYLGLUTARYL-COA LYASE-like gene of CuraGen Acc. No. CG96859-03 are reported in Table G11. Variants are reported individually but any combination of all or a select subset of variants are also included. In Table G11, the positions of the variant bases and the variant amino acid residues are underlined. In summary, there are 1 variants reported in Table G11. Variant 13379476 is a C to T SNP at 725 bp of the nucleotide sequence that results in no change in the protein sequence (silent).

TABLE G11
Variant of nucleotide sequence Acc.
No. CG96859-03 (SEQ ID NO: 59)

| | Nucleotides | | | Amino Acids | | |
|---|---|---|---|---|---|---|
| Variant | Position | Initial | Modified | Position | Initial | Modified |
| 13379476 | 725 | C | T | 237 | Thr | Thr |

TABLE G12
Variant Sequences

(SEQ ID NO:169)

TableG12A1.
Nucleotide sequence of variant 13379476 NOV7a1n (underlined). C/T

| | |
|---|---|
| 1 | AAATTCCGGCCAAGATGGCAGCAATGAGGAAGGCGCTTCCGCGGCGACTGGTGGGCTTGGCGTCCCTCCGGGCTGTCAGC |
| 81 | ACCTCATCTATGGGCACTTTACCAAAGCGGGTGAAAATTGTGGAAGTTGGTCCCCGAGATGGACTACAAAATGAAAAGAA |
| 161 | TATCGTATCTACTCCAGTGAAAATCAAGCTGATAGACATGCTTTCTGAAGCAGGACTCTCTGTTATAGAAACCACCAGCT |
| 241 | TTGTGTCTCCTAAGTGGGTTCCCCAGATGGGTGACCACACTGAAGTCTTGAAGGGCATTCAGAAGTTTCCTGGCATCAAC |
| 321 | TACCCAGTCCTGACCCCAAATTTGAAAGGCTTCGAGGCAGCGGTTGCTGCTGGAGCCAAGGAAGTAGTCATCTTTGGAGC |
| 401 | TGCCTCAGAGCTCTTCACCAAGAAGAACATCAATTGTTCCATAGAGGAGAGTTTTCAGAGGTTTGACGCAATCCTGAAGG |
| 481 | CAGCGCAGTCAGCCAATATTTCTGTGCGGGGGTACGTCTCCTGTGCTCTTGGCTGCCCTTATGAAGGGAAGATCTCCCCA |
| 561 | GCTAAAGTAGCTGAGGTCACCAAGAAGTTCTACTCAATGGGCTGCTACGAGATCTCCCTGGGGGACACCATTGGTGTGGG |
| 641 | CACCCCAGGGATCATGAAAGACATGCTGTCTGCTGTCATGCAGGAAGTGCCTCTGGCTGCCCTGGCTGTCCACTGCCATG |
| 721 | ACACTTATGGTCAAGCCCTGGCCAACACCTTGATGGCCCTGCAGATGGGAGTGAGTGTCGTGGACTCTTCTGTGGCAGGA |
| 801 | CTTGGAGGCTGTCCCTACGCACAGGGGGCATCAGGAAACTTGGCCACAGAAGACCTGGTCTACATGCTAGAGGGCTTGGG |
| 881 | CATTCACACGGGTGTGAATCTCCAGAAGCTTCTGGAAGCTGGAAACTTTATCTGTCAAGCCCTGAACAGAAAAACTAGCT |
| 961 | CCAAAGTGGCTCAGGCTACCTGTAAACTCTGAGCCCCTTGCCCACCTGAAGGCCTGGGGATGATGTGGAAATAAGGGGCA |
| 1041 | T |

(SEQ ID NO:170)

TableG12A2.
Protein sequence of variant NOV7a1n (underlined).

| | |
|---|---|
| 1 | MAAMRKALPRRLVGLASLRAVSTSSMGTLPKRVKIVEVGPRDGLQNEKNIVSTPVKIKLIDMLSEAGLSVIETTSFVSPK |
| 81 | WVPQMGDHTEVLKGIQKFPGINYPVLTPNLKGFEAAVAAGAKEVVIFGAASELFTKKNINCSIEESFQRFDAILKAAQSA |
| 161 | NISVRGYVSCALGCPYEGKISPAKVAEVTKKEYSMGCYEISLGDTIGVGTPGIMKDMLSAVMQEVPLAALAVHCHDTYGQ |
| 241 | ALANTLMALQMGVSVVDSSVAGLGGCPYAQGASGNLATEDLVYMLEGLGIHTGVNLQKLLEAGNFICQALNRKTSSKVAQ |
| 321 | ATCKL |

TableG12A3.
Alteration effect
No change.

### EXAMPLE G4

### Expression Profiles of HMG CoA Lyase (CG96859-03)—like Protein

The protocol for quantitative expression analysis is disclosed in Example Q9.

Expression of gene CG96859-03 was assessed using the primer-probe set Ag8471, described in Table G13. Results of the RTQ-PCR runs are shown in Table G14.

TABLE G13
Probe Name Ag8471

| | | | Start | SEQ ID |
|---|---|---|---|---|
| Primers | Sequences | Length | Position | No |
| Forward | 5′-aagctggtggtttctataacagaga-3′ | 25 | 801 | 218 |
| Probe | TET-5′-tcctgcttcagaaagcatgtctatcagc-3′- | 28 | 827 | 219 |
| | TAMRA | | | |
| Reverse | 5′-aagaatatcgtatctactccagtgaaaat-3′ | 29 | 858 | 220 |

TABLE G14
General_screening_panel_v1.7

| Tissue Name | A |
|---|---|
| Adipose | 42.3 |
| HUVEC | 25.7 |
| Melanoma* Hs688(A).T | 0.0 |
| Melanoma* Hs688(B).T | 54.7 |
| Melanoma (met) SK-MEL-5 | 28.7 |
| Testis | 12.1 |
| Prostate ca. (bone met) PC-3 | 0.9 |
| Prostate ca. DU145 | 32.8 |
| Prostate pool | 9.2 |
| Uterus pool | 3.1 |
| Ovarian ca. OVCAR-3 | 8.4 |
| Ovarian ca. (ascites) SK-OV-3 | 1.6 |
| Ovarian ca. OVCAR-4 | 0.0 |
| Ovarian ca. OVCAR-5 | 40.6 |
| Ovarian ca. IGROV-1 | 0.0 |
| Ovarian ca. OVCAR-8 | 44.4 |
| Ovary | 14.6 |
| Breast ca. MCF-7 | 31.0 |
| Breast ca. MDA-MB-231 | 28.5 |
| Breast ca. BT 549 | 30.4 |
| Breast ca. T47D | 35.4 |
| Breast pool | 0.0 |
| Trachea | 30.8 |
| Lung | 38.4 |
| Fetal Lung | 22.4 |
| Lung ca. NCI-N417 | 6.3 |
| Lung ca. LX-1 | 10.7 |
| Lung ca. NCI-H146 | 4.7 |
| Lung ca. SHP-77 | 29.9 |
| Lung ca. NCI-H23 | 31.2 |
| Lung ca. NCI-H460 | 13.4 |
| Lung ca. HOP-62 | 100.0 |
| Lung ca. NCI-H522 | 23.2 |
| Lung ca. DMS-114 | 7.6 |
| Liver | 71.7 |
| Fetal Liver | 72.2 |
| Kidney pool | 83.5 |
| Fetal Kidney | 18.9 |
| Renal ca. 786-0 | 64.2 |
| Renal ca. A498 | 10.5 |
| Renal ca. ACHN | 24.0 |
| Renal ca. UO-31 | 48.0 |
| Renal ca. TK-10 | 24.0 |
| Bladder | 14.9 |
| Gastric ca. (liver met.) NCI-N87 | 0.4 |
| Stomach | 1.4 |
| Colon ca. SW-948 | 12.9 |
| Colon ca. SW480 | 0.2 |
| Colon ca. (SW480 met) SW620 | 33.0 |
| Colon ca. HT29 | 57.8 |
| Colon ca. HCT-116 | 40.3 |
| Colon cancer tissue | 1.4 |
| Colon ca. SW1116 | 11.2 |
| Colon ca. Colo-205 | 26.4 |
| Colon ca. SW-48 | 14.4 |
| Colon | 22.5 |
| Small Intestine | 4.1 |
| Fetal Heart | 6.0 |
| Heart | 7.4 |
| Lymph Node pool 2 | 35.6 |
| Fetal Skeletal Muscle | 9.8 |
| Skeletal Muscle pool | 5.4 |
| Skeletal Muscle | 42.6 |
| Spleen | 16.6 |
| Thymus | 9.9 |
| CNS cancer (glio/astro) SF-268 | 9.4 |
| CNS cancer (glio/astro) T98G | 13.9 |
| CNS cancer (neuro; met) SK-N-AS | 0.3 |
| CNS cancer (astro) SF-539 | 88.9 |
| CNS cancer (astro) SNB-75 | 44.1 |
| CNS cancer (glio) SNB-19 | 33.9 |
| CNS cancer (glio) SF-295 | 9.1 |
| Brain (Amygdala) | 10.2 |
| Brain (Cerebellum) | 27.7 |
| Brain (Fetal) | 14.9 |
| Brain (Hippocampus) | 13.3 |
| Cerebral Cortex pool | 9.2 |
| Brain (Substantia nigra) | 4.0 |
| Brain (Thalamus) | 17.1 |
| Brain (Whole) | 29.5 |
| Spinal Cord | 11.8 |
| Adrenal Gland | 56.6 |
| Pituitary Gland | 17.6 |
| Salivary Gland | 15.1 |
| Thyroid | 59.0 |
| Pancreatic ca. PANC-1 | 19.9 |
| Pancreas pool | 6.1 |

Column A - Rel. Exp. (%) Ag8471, Run 406009117

Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

General_screening_panel_v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

General_screening_panel_v1.7 Summary: Ag8471 The highest expression of this gene was detected in a lung cancer sample (CT=26). This gene is widely expressed. Among tissues with metabolic or endocrine function, this gene is expressed at high to moderate levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product will useful in the treatment of endocrine/metabolically related diseases, such as obesity and diabetes.

### EXAMPLE G5

### Screening Assays for Modulators of HMG CoA Lyase

A non-exhaustive list of cell lines that express the HMG CoA Lyase can be obtained from the differential gene expression (RTQ-PCR) results presented herein.

Potential methods for measurement of HMG-CoA lyase enzymatic activity include citrate synthase-coupled assay of Stegink and Coon, (Stereospecificity and other properties of highly purified beta-hydroxy-beta-methylglutaryl coenzyme A cleavage enzyme from bovine liver, J Biol. Chem. 1968 Oct. 25; 243 (20):5272-9), further modified by Kramer and Miziorko (Purification and characterization of avian liver 3-hydroxy-3-methylglutaryl coenzyme A lyase, J Biol. Chem. 1980 Nov. 25; 255 (22):11023-8).

Our results indicate that a modulator of HMG CoA Lyase activity, such as an inhibitor, activator, antagonist, or agonist of HMG CoA Lyase may be useful for treatment of such disorders as obesity, diabetes, and insulin resistance, as well as for enhancement of insulin secretion.

Protocols

Following Examples describe procedures, protocols and technologies described in this application.

### EXAMPLE Q1

### Mouse Diet-Induced Obesity (DIO) Study (BP24.02)

Overview

The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design sought to identify the factors that led to the ability of certain individuals to resist the effects of a high fat diet and thereby prevent obesity.

The sample groups for the study normally had body weights +1 S.D., +4 S.D. and +7 S.D. of the chow-fed controls. In addition, the biochemical profile of the +7 S.D. mice normally revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues revealed genes and pathways that can be used as therapeutic targets for obesity and/or diabetes.

Protocol

5 groups of mice were used with 3 mice per group. Occasionally, more than 3 mice were used in a single group in order to preserve correct parameters for the study. In such case only 3 mice would be sacrificed for tissues. The groups were grouped based on the following parameters:
Group 1. Chow fed mice
Group 2. Mice fed a high fat diet who were 1 standard deviation in weight above the chow fed mice.
Group 3. Mice fed a high fat diet who were 4 standard deviations in weight above the chow fed mice.
Group 4. Mice fed a high fat diet who were 7 standard deviations in weight above the chow fed mice and who had normal glucose levels.
Group 5. Mice fed a high fat diet who were 7 standard deviation in weight above the chow fed mice and who were hyperglycemic.

In each group of mice, there were 3 mice that were sacrificed and tissues harvested for the study.

### EXAMPLE Q2

### Rat Pancreatic Islet Study (BP24.03)

Overview

An important clinical goal in the early phases of Type II diabetes is to increase insulin secretion from the beta cells of the pancreas. Numerous agents have been identified that can modulate insulin secretion experimentally and in therapeutic situations. When applied to isolated rat pancreatic islets, the changes in gene expression can be correlated with insulin secretion. In this study, acute and chronic changes in gene expression were examined from islets treated with an agent after short (4 hour) and long-term (5 days) exposure, respectively, compared with the basal state (11 mM glucose). The agents included elevated (25 mM) glucose, glucose (11 mM) and exendin-4 (1 nM), glucose (11 mM) and glybenclamide (50 uM) and glucose (11 mM) and oleate (2 mM).

Protocol

All samples were isolated rat islets. They differed only in the treatment that they received. The following samples were in the 4 hour group:

1) 11 mM glucose
2) 25 mM glucose
3) 11 mM glucose & JTT 608
4) 11 mM glucose & Carbacol
5) 11 mM glucose & Exendin4

Isolated rat islets were treated with either 11 mM glucose (basal state) or 25 mM glucose (elevated glucose.). Then there were 3 additional sets of rat islets that were treated with 11 mM glucose and one of the 3 agents: JTT 608, Carbacol, or Exendin-4.

The following samples were in the 5 day group:

1) 11 mM glucose
2) 25 mM glucose
3) 11 mM glucose & 1 nM Exendin
4) 11 mM glucose & 50 uM Glybenclamide
5) 11 mM glucose & 2 mM Oleic Acid

Isolated rat islets were treated with either 11 mM glucose (basal state) or 25 mM glucose (elevated glucose.). Then there were 3 additional sets of rat islets that were treated with 11 mM glucose and one of the 3 agents: exendin, glybenclamide, or oleic acid.

From all 10 samples, each was split into 2 replicates. The 2 replicates were run for differential gene expression analysis (GeneCalling®).

### EXAMPLE Q3

### Rat Insulin Sensitivity Study (BP24.05)

Protocol

ZDF rats or their lean littermates were treated with a variety of agents that are known to alter insulin sensitivity. Metformin, vanadate, and AICAR enhance tissue response to insulin, while the free fatty acids generated by Liposyn (intravenous lipid infusion) treatment reduces the response. A variety of tissues were harvested, including gastrocnemius and soleus muscles, liver, retroperitoneal and epididymal WAT, and IBAT.

Only gastrocnemius and soleus muscles were processed for differential gene expression analysis (GeneCalling®).

There were 5 groups of samples:

1) Metformin vehicle (vehicle M)
2) Metformin treated rats
3) AICAR and vanadate vehicle (vehicle AV)
4) AICAR treated rats
5) Vanadate treated rats

Treatment was for 4 hours and glucose values before and after treatment were obtained. Each sample was done in triplicate (5 groups X 3 rats X 2 tissues).

In the second part of the study rats were given an intravenous lipid infusion which should reduce tissue response to insulin in treated rats.

In the intravenous lipid infusion part of the study 2 groups were used:

1) Rats treated with lipid infusion vehicle.
2) Rats treated with lipid infusion.

In each group, there were 3 rats (done in triplicate) and soleus and gastrocnemius samples were processed for differential gene expression analysis (GeneCalling®) (2 groups X 3 samples X 2 tissues).

### EXAMPLE Q4

### Mouse TZD Response Study (BP24.07)

Overview and Protocol

The peroxisome proliferator-activated receptor gamma (PPARg) is the member of the nuclear hormone receptor subfamily of transcription factors that plays a major role in regulation of metabolism. The thiazolidinedione (TZD) drugs, including rosiglitazone, are synthetic agonists of PPARg receptors that can normalize elevated plasma glucose levels in obese, diabetic rodents and are often quite efficacious therapeutic agents for the treatment of noninsulin-dependent diabetes mellitus in humans. Diabetic animals demonstrate differential responses to TZD treatment. To understand the basis for this differential response we compared changes in gene expression between diabetic animals that responded favorably and that did not respond to TZD treatment. Female db/db mice were treated daily with 10 mg per kilogram body weight rosiglitazone for 7 days. On day 8, the mice were bled for blood glucose. Treated mice were grouped into either a responder group that demonstrated a significant decrease of their hyperglycemia and a non-responder group that demonstrated no change in their blood glucose level. Gene expression in skeletal muscle and adipose tissues was compared between untreated diabetic mice and the two sub-groups of TZD treated mice.

3 tissues were collected for differential gene expression analysis (GeneCalling®): liver, thigh muscle, and uterine white adipose.

3 groups of samples were used:

1) Vehicle treated
2) Rosiglitazone responders
3) Rosiglitazone non-responders

Each group had 3 mice in it. Total of 27 samples were processed for differential gene expression analysis (GeneCalling®).

### EXAMPLE Q5

### Insulin Resistance Study (MB.01)

The spontaneoulsy hypertensive rat (SHR) is a strain exhibiting features of the human Metabolic Syndrome X. The phenotypic features include obesity, hyperglycemia, hypertension, dyslipidemia and dysfibrinolysis. Tissues were removed from adult male rats and a control strain (Wistar_Kyoto) to identify the gene expression differences that underlie the pathologic state in the SHR and in animals treated with various anti-hyperglycemic agents such as troglitizone. Tissues included sub-cutaneous adipose, visceral adipose, brain, muscle, and liver. Each tissue was collected in triplicate for differential gene expression analysis (GeneCalling®).

### EXAMPLE Q6

### Genetically Obese Mice vs Genetically Lean Mice Study (MB.04)

Overview

A number of genetic models of obesity have been studied, most prominently in mouse and rat, but only a few causative genes have been identified. In this study, a set of mouse genetic models were studied in order to identify by positional expression cloning the genes for obesity in mice, which genes may be relevant to human obesity.

Protocol

7 strains of mice were used. Some exhibited a lean phenotype, some were normal weight mice, and some of the mice were genetically obese.

The following strains of mice used in the study:

1) CAST/E1—lean phenotype
2) SM/J black—lean phenotype
3) SWR/J—normal phenotype
4) C57L/J—normal phenotype
5) C57BL/6J—normal phenotype
6) AKR/J—obese phenotype
7) NZB/BINJ—obese phenotype

Various tissues were processed for differential gene expression analysis (GeneCalling®): brain, liver, muscle, and adipose.

Samples were processed in triplicate (i.e. 3 brain samples from 3 CAST/E1 mice). Total of 7 strains X 4 tissues X 3 samples=84 samples were used.

### EXAMPLE Q7

### Method of Identifying the Differentially Expressed Gene and Gene Product (GeneCalling®)

The GeneCalling® technology is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). GeneCalling® technology is also disclose in U.S. Pat. No. 5,871,697. cDNA was derived from various samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The three methods routinely used to confirm the identity of the gene fragment found to have altered expression in models of or patients with obesity and/or diabetes are described below.

A). Direct Sequencing

The differentially expressed gene fragment is isolated, cloned into a plasmid, and sequenced. Afterwards, the sequence information is used to design an oligonucleotide corresponding to either or both termini of the gene fragment. This oligonucleotide, when used in a competitive PCR reaction, will ablate the electropherographic band from which the sequence is derived.

B). Competitive PCR

In competitive PCR, the electropherographic peaks corresponding to the gene fragment of the gene of interest are ablated when a gene-specific primer (designed from the sequenced band or available databases) competes with primers in the linker-adaptors during the PCR amplification.

C). PCR with Perfect or Mismatched 3′ Nucleotides (TraPping)

This method utilizes a competitive PCR approach using a degenerate set of primers that extend one or two nucleotides into the gene-specific region of the fragment beyond the flanking restriction sites. As in the competitive PCR approach, primers that lead to the ablation of the electropherographic band add additional sequence information. In conjunction with the size of the gene fragment and the 12 nucleotides of sequence derived from the restriction sites, this additional sequence data can uniquely define the gene after database analysis. TraPping is disclosed in a published PCT application Pub. No. WO 01/49886.

### EXAMPLE Q8

### Identification of Human Sequences

The laboratory cloning was performed using one or more of the methods summarized below:

SeqCalling™Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen Corporation's SeqCalling technology that is disclosed in full in U.S. Ser. No. 09/417,386 filed Oct. 13, 1999 (as well as in PCT application Pub. No.: WO 00/40757), and 09/614,505 filed Jul. 11, 2000. Sequence traces were evaluated manually and edited for corrections if appropriate. cDNA sequences from all samples were assembled together, sometimes including public human sequences, using bioinformatics programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

Information associated with genomic clones, public genes and ESTs sharing sequence identity with the disclosed sequences and CuraGen Corporation's Electronic Northern bioinformatic tool.

RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

Exon Linking: The cDNA coding for the CG101190-01 sequence was cloned by the polymerase chain reaction (PCR) using the primers designed based on known cDNA sequences or in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Physical Clone: The PCR product derived by exon linking, covering the entire open reading frame, was cloned into the pCR2.1 vector from Invitrogen to provide clones used for expression and screening purposes.

### EXAMPLE Q9

### Quantitative Expression Analysis (RTQ-PCR) of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s: 18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tm must be 10° C. greater than primer Tm; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 950 C for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1× TaqMan® Universal Master mix (Applied Biosystems; catalog No. 4324020), following the manufacturer's instructions. PCR amplification and analysis were done as described above.

Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

General_screening_panel_v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

Panels 2D, 2.2, 2.3 and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDR1), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

HASS Panel v 1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

ARDAIS Panel v1.0 and v1.1

The ARDAIS Panel v1.0 and v1.1 included 2 controls and 22 test samples including: human lung adenocarcinomas, lung squamous cell carcinomas, and in some cases matched adjacent normal tissues (NAT) obtained from Ardais (Lexington, Mass.). Unmatched malignant and non-malignant RNA samples from lungs with gross histopathological assessment of tumor differentiation grade and stage and clinical state of the patient were obtained from Ardais.

ARDAIS Prostate v1.0

ARDAIS Prostate v1.0 panel included 2 controls and 68 test samples of human prostate malignancies and in some cases matched adjacent normal tissues (NAT) obtained from Ardais (Lexington, Mass.). RNA from unmatched malignant and non-malignant prostate samples with gross histopathological assessment of tumor differentiation grade and stage and clinical state of the patient were also obtained from Ardais.

ARDAIS Kidney v1.0

ARDAIS Kidney v1.0 panel included 2 control wells and 44 test samples of human renal cell carcinoma and in some cases matched adjacent normal tissue (NAT) obtained from Ardais (Lexington, Mass.). RNA from unmatched renal cell carcinoma and normal tissue with gross histopathological assessment of tumor differentiation grade and stage and clinical state of the patient were also obtained from Ardais.

ARDAIS Breast v1.0

ARDAIS Breast v1.0 panel included 2 control wells and 71 test samples of human breast malignancies and in some cases matched adjacent normal tissue (NAT) obtained from Ardais (Lexington, Mass.). RNA from unmatched malignant and non-malignant breast samples with gross histopathological assessment of tumor differentiation grade and stage and clinical state of the patient were also obtained from Ardais.

Panel 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Panels 4D, 4R, and 4.1D

Panels 4D, 4R, and 4.1D included 2 control wells and 94 test samples of RNA (Panel 4R) or cDNA (Panels 4D and 4.1 D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDR1, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta ˜1-5 ng/ml, TNF alpha ˜5-10 ng/ml, IFN gamma ˜20-50 ng/ml, IL-4˜5-10 ng/ml, IL-9˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (Hyclone, Logan, Utah), 100 mM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 mg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of ˜2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+ lymphocytes were isolated by depleting mononuclear cells of CD8+, CD56+, CD14+ and CD19+cells using CD8, CD56, CD14 and CD19 Miltenyi beads and positive selection. CD45RO Miltenyi beads were then used to separate the CD45+RO CD4+ lymphocytes from CD45+RA CD4+ lymphocytes. CD45+RA CD4+, CD45+RO CD4+ and CD8+ lymphocytes were cultured in culture media at 10⁶ cells/ml in culture plates precoated overnight with 0.5 mg/ml anti-CD28 (Pharmingen, San Diego, Calif.) and 3 μg/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDR1). Tonsil cells were pelleted and resupended at 10⁶ cells/ml in culture media. Cells were activated using 5 μg/ml PWM (Sigma-Aldrich Corp., St. Louis, Mo.) or ˜0 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 10⁵-10⁶ cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (i ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 4-5 days, the Th1, Th2 and Trl lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×1 05 cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 ml buffer (Promega, Madison, Wis.) 5 μl DTT, 7 μl RNAsin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

AI_comprehensive panel_v1.0

Autoimmunity (AI) comprehensive panel v1.0 included two controls and 89 cDNA test samples isolated from male (M) and female (F) surgical and postmortem human tissues that were obtained from the Backus Hospital and Clinomics (Frederick, Md.). Tissue samples included: normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

Pulmonary and General inflammation (PGI) panel v1.0

Pulmonary and General inflammation (PGI) panel v1.0 included two controls and 39 test samples isolated as surgical or postmortem samples. Tissue samples include: five normal lung samples obtained from Maryland Brain and Tissue Bank, University of Maryland (Baltimore, Md.), International Bioresource systems, IBS (Tuscon, Ariz.), and Asterand (Detroit, Mich.), five normal adjacent intestine tissues (NAT) from Ardais (Lexington, Mass.), ulcerative colitis samples (UC) from Ardais (Lexington, Mass.); Crohns disease colon from NDR1, National Disease Research Interchange (Philadelphia, Pa.); emphysematous tissue samples from Ardais (Lexington, Mass.) and Genomic Collaborative Inc. (Cambridge, Mass.), asthmatic tissue from Maryland Brain and Tissue Bank, University of Maryland (Baltimore, Md.) and Genomic Collaborative Inc (Cambridge, Mass.) and fibrotic tissue from Ardais (Lexinton, Mass.) and Genomic Collaborative (Cambridge, Mass.).

Cellular OA/RA Panel

Cellular OA.RA panel includes 2 control wells and 35 test samples comprised of cDNA generated from total RNA isolated from human cell lines or primary cells representative of the human joint and its inflammatory condition. Cell types included normal human osteoblasts (Nhost) from Clonetics (Cambrex, East Rutherford, N J), human chondrosarcoma SW1353 cells from ATCC (Manossas, Va.)), human fibroblast-like synoviocytes from Cell Applications, Inc. (San Diego, Calif.) and MH7A cell line (a rheumatoid fibroblast-like synoviocytes transformed with SV40 T antigen) from Riken Cell bank (Tsukuba Science City, Japan). These cell types were activated by incubating with various cytokines (IL-1 beta 1-10 ng/ml, TNF alpha ˜5-50 ng/ml, or prostaglandin E2 for Nhost cells) for 1, 6, 18 or 24 h. All these cells were starved for at least 5 h and cultured in their corresponding basal medium with ˜0.1 to 1% FBS.

Minitissue OA/RA Panel

The OA/RA mini panel includes two control wells and 31 test samples comprised of cDNA generated from total RNA isolated from surgical and postmortem human tissues obtained from the University of Calgary (Alberta, Canada), NDR1 (Philadelphia, Pa.), and Ardais Corporation (Lexington, Mass.). Joint tissue samples include synovium, bone and cartilage from osteoarthritic and rheumatoid arthritis patients undergoing reconstructive knee surgery, as well as, normal synovium samples (RNA and tissue). Visceral normal tissues were pooled from 2-5 different adults and included adrenal gland, heart, kidney, brain, colon, lung, stomach, small intestine, skeletal muscle, and ovary.

AI.05 Chondrosarcoma

AI.05 chondrosarcoma plates included SW1353 cells (ATCC) subjected to serum starvation and treated for 6 and 18 h with cytokines that are known to induce MMP (1, 3 and 13) synthesis (e.g. IL1beta). These treatments included: IL-1beta (10 ng/ml), IL-1beta+TNF-alpha (50 ng/ml), IL-1 beta+Oncostatin (50 ng/ml) and PMA (100 ng/ml). Supernatants were collected and analyzed for MMP 1, 3 and 13 production. RNA was prepared from these samples using standard procedures.

Panels 5D and 51

Panel 5D and 5I included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18-40 years), otherwise healthy women with and without gestational diabetes undergoing routine (elective) Caesarean section. Uterine wall smooth muscle (UT), visceral (Vis) adipose, skeletal muscle (SK), placenta (P1) greater omentum adipose (GO Adipose) and subcutaneous (SubQ) adipose samples (less than 1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr. 2 1999: 143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures.

Panel 5I also contains pancreatic islets (Diabetes Research Institute at the University of Miami School of Medicine).

Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (P1), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non-diabetic Caucasians; Patient 22, a treated diabetic Caucasian of normal weight; Patient 23, an overweight non-diabetic Caucasian; and Patients 26 and 27, obese, treated, diabetic Caucasians.

Total RNA was isolated from metabolic tissues including: hypothalamus, liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

CNS Panels

CNS Panels CNSD.01, CNS Neurodegeneration V1.0 and CNS Neurodegeneration V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −80° C. in liquid nitrogen vapor.

Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

Panel CNS Neurodegeneration V1.0

The CNS Neurodegeneration V1.0 panel included: six Alzheimer's disease (AD) brains and eight normals which included no dementia and no Alzheimer's like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value for all wells across all runs). The scaled CT value is the result of the raw CT value plus the scaling factor.

Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

### EXAMPLE Q10

### PathCalling® Technology

The sequence of NOVX was derived by laboratory screening of cDNA library by the two-hybrid approach. cDNA fragments covering either the full length of the DNA sequence, or part of the sequence, or both, were sequenced. In silico prediction was based on sequences available in CuraGen Corporation's proprietary sequence databases or in the public human sequence databases, and provided either the full-length DNA sequence, or some portion thereof.

The laboratory screening was performed using the methods that follow. cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated nucleic acid molecule comprising a nucleic acid sequence selected from the group consisting of SEQ ID NO:2n-1, wherein n is an integer between 1 and 85.

2. An isolated nucleic acid molecule comprising a nucleic acid sequence encoding an amino acid sequence selected from the group consisting of SEQ ID NO:2n, wherein n is an integer between 1 and 85.

3. An isolated polypeptide comprising an amino acid sequence selected from the group consisting of SEQ ID NO:2n, wherein n is an integer between 1 and 85.

4. An isolated polypeptide of claim 3, wherein the isolated polypeptide comprises an amino acid sequence selected from the group consisting of SEQ ID NO:2, SEQ ID NO:4, SEQ ID NO:6, SEQ ID NO:86, SEQ ID NO:88, SEQ ID NO:90, SEQ ID NO:92, SEQ ID NO:96, SEQ ID NO:98, SEQ ID NO:100, SEQ ID NO:102, SEQ ID NO:104, SEQ ID NO:106, SEQ ID NO:108, and SEQ ID NO:110.

5. An isolated polypeptide of claim 3, wherein the isolated polypeptide comprises an amino acid sequence selected from the group consisting of SEQ ID NO:8, SEQ ID NO:10, SEQ ID NO:12, SEQ ID NO:14, SEQ ID NO:16, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:136, SEQ ID NO:138, SEQ ID NO:144, SEQ ID NO:148, SEQ ID NO:152, and SEQ ID NO:154.

6. An isolated polypeptide of claim 3, wherein the isolated polypeptide comprises an amino acid sequence selected from the group consisting of SEQ ID NO:18, SEQ ID NO:20, SEQ ID NO:22, SEQ ID NO:24, SEQ ID NO:26, and SEQ ID NO:28.

7. An isolated polypeptide of claim 3, wherein the isolated polypeptide comprises an amino acid sequence selected from the group consisting of SEQ ID NO:30, SEQ ID NO:32, SEQ ID NO:34, SEQ ID NO:36, SEQ ID NO:38, SEQ ID NO:40, SEQ ID NO:42, SEQ ID NO:44, and SEQ ID NO:156

8. An isolated polypeptide of claim 3, wherein the isolated polypeptide comprises an amino acid sequence selected from the group consisting of SEQ ID NO:46, SEQ ID NO:48, SEQ ID NO:50, SEQ ID NO:52, and SEQ ID NO:54.

9. An isolated polypeptide of claim 3, wherein the isolated polypeptide comprises an amino acid sequence selected from the group consisting of SEQ ID NO:56, SEQ ID NO:58, and SEQ ID NO:168.

10. An isolated polypeptide of claim 3, wherein the isolated polypeptide comprises an amino acid sequence selected from the group consisting of SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, and SEQ ID NO:170.

11. A method for identifying compounds that modulate target polypeptide activity comprising:
(a) combining a test compound with a target polypeptide and a substrate of the target polypeptide; and
(b) determining whether the test compound modulates the activity of the target polypeptide;
wherein the target polypeptide comprises an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

12. The method of claim 11, further comprising a step of identifying the test compound that modulates the target polypeptide activity by inhibiting the target polypeptide activity as an inhibitor of the target polypeptide activity.

13. The method of claim 11, further comprising a step of identifying the test compound that modulates the target polypeptide activity by inhibiting the target polypeptide activity as an antagonist of the target polypeptide.

14. The method of claim 11, further comprising a step of identifying the test compound that modulates the target polypeptide activity by activating the target polypeptide activity as an activator of the target polypeptide activity.

15. The method of claim 11, further comprising a step of identifying the test compound that modulates the target polypeptide activity by activating the target polypeptide activity as an agonist of the target polypeptide.

16. The method of claim 11, further comprising a step of identifying the test compound that modulates the target polypeptide activity as an enhancer of insulin secretion.

17. The method of claim 11, further comprising a step of identifying the test compound that modulates the target polypeptide activity as a therapeutic for treatment of insulin resistance.

18. The method of claim 11, further comprising a step of identifying the test compound that modulates the target polypeptide activity as a therapeutic for treatment of obesity.

19. The method of claim 11, further comprising a step of identifying the test compound that modulates the target polypeptide activity as a therapeutic for treatment of diabetes.

20. The method of claim 11, wherein the target polypeptide is an isolated polypeptide.

21. The method of claim 11, wherein the target polypeptide is produced by a process comprising culturing a recombinant host cell, the recombinant host cell comprising a nucleic acid encoding the target polypeptide, under conditions promoting expression of the target polypeptide.

22. The method of claim 21, wherein the nucleic acid comprises a nucleotide sequence selected from the group consisting of:
(a) SEQ ID NO:2n-1, wherein n is an integer between 1 and 85;
(b) nucleotides encoding an amino acid sequence of the at least one domain of SEQ ID NO:2n; and
c) a nucleotide sequence encoding an amino acid sequence selected from the group consisting of SEQ ID NO:2n, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

23. The method of claim 11, wherein the target polypeptide is produced by expression of a recombinant vector comprising a nucleic acid, the nucleic acid encoding an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

24. The method of claim 23, wherein the test compound is combined with the target polypeptide in a mammalian cell grown in culture.

25. The method of claim 23, wherein the test compound is combined with the target polypeptide in vitro.

26. The method of claim 23, wherein the nucleic acid comprises a nucleotide sequence selected from the group consisting of:
(a) SEQ ID NO:2n-1, wherein n is an integer between 1 and 85;
(b) nucleotides encoding an amino acid sequence of the at least one domain of SEQ ID NO:2n; and
c) a nucleotide sequence encoding an amino acid sequence selected from the group consisting of SEQ ID NO:2n, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

27. The method of claim 11, wherein the target polypeptide is produced by expression of an endogenous nucleic acid, the endogenous nucleic acid encoding an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n; and the amino acid sequence that is at least 95% identical to the at least one domain of SEQ ID NO:2n.

28. The method of claim 27, wherein the test compound is combined with the target polypeptide in a mammalian cell grown in culture.

29. The method of claim 27, wherein the test compound is combined with the target polypeptide in vitro.

30. The method of claim 27, wherein the endogenous nucleic acid comprises a nucleotide sequence selected from the group consisting of:
(a) SEQ ID NO:2n-1, wherein n is an integer between 1 and 85;
(b) nucleotides encoding an amino acid sequence of the at least one domain of SEQ ID NO:2n; and
(c) a nucleotide sequence encoding an amino acid sequence selected from the group consisting of SEQ ID NO:2n, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

31. An antibody that immunospecifically binds to the target polypeptide,
wherein the target polypeptide comprises an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

32. The antibody of claim 31, wherein the antibody is a monoclonal antibody.

33. The antibody of claim 31, wherein the antibody is a humanized antibody.

34. The antibody of claim 31, wherein the antibody is a human antibody.

35. A method for identifying a potential therapeutic agent for use in treatment of a pathology, wherein the pathology is related to aberrant expression or aberrant physiological interactions of a target polypeptide, the method comprising:
(a) providing a cell expressing the target polypeptide and having a property or function ascribable to the target polypeptide;
(b) contacting the cell with a composition comprising a candidate test compound; and
(c) determining whether the test compound alters the property or function ascribable to the target polypeptide;
whereby, if an alteration observed in the presence of the test compound is not observed when the cell is contacted with the composition in the absence of the test compound, the test compound is identified as a potential therapeutic agent; and
wherein the target polypeptide comprises an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.

36. A method for screening for a modulator of activity of or of latency or predisposition to a pathology associated with a target polypeptide, the method comprising:
(a) administering a test compound to a test animal at an increased risk for a pathology associated with the target polypeptide, wherein the test animal recombinantly expresses the target polypeptide;
(b) measuring the activity of the target polypeptide in the test animal after administering the test compound of step (a); and
(c) comparing the activity of the target polypeptide in the test animal with the activity of the target polypeptide in a control animal not administered the test compound, wherein a change in the activity of the target polypeptide in the test animal relative to the control animal indicates that the test compound is a modulator of activity of or of latency or predisposition to, a pathology associated with the target polypeptide;
wherein the target polypeptide comprises an amino acid sequence selected from the group consisting of SEQ ID NO:2n, wherein n is an integer between 1 and 85, the amino acid sequence that is at least 95% identical to SEQ ID NO:2n, the amino acid sequence of at least one domain of SEQ ID NO:2n, and the amino acid sequence that is at least 95% identical to the at least one domain of SEQ ID NO:2n.
